(19) United States
(12) Patent Application Publication
(10) Pub. No.: US 2023/0200225 A1
(43) Pub. Date: Jun. 22, 2023

(54) NITROGEN-CONTAINING COMPOUND, ELECTRONIC COMPONENT, AND ELECTRONIC DEVICE

(71) Applicant: Shaanxi Lighte Optoelectronics Material Co., Ltd., Xi'an (CN)

(72) Inventors: Tiantian MA, Xi'an (CN); Min YANG, Xi'an (CN); Peng NAN, Xi'an (CN)

(21) Appl. No.: 18/011,779

(22) PCT Filed: Aug. 4, 2021
(86) PCT No.: PCT/CN2021/110658

§ 371 Date: Dec. 20, 2022

(30) Foreign Application Priority Data
Dec. 25, 2020 (CN) 202011564501.4

(51) Int. Cl. H10K 85/60; C07D 209/86

(52) U.S. Cl. CPC H10K 85/636; C07D 209/86; H10K 85/633; H10K 85/6572; H10K 85/6574; H10K 85/6576; H10K 85/615; H10K 30/50

(57) ABSTRACT

The present disclosure belongs to the technical field of organic materials, and provides a nitrogen-containing compound, an electronic component, and an electronic device. The nitrogen-containing compound has a structure represented by a Formula 1.

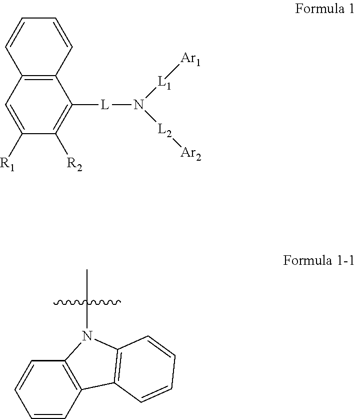

### CROSS REFERENCE TO RELATED APPLICATIONS

The present application claims the priority of Chinese Patent Application No. 202011564501.4, filed on Dec. 25, 2020, the contents of which are incorporated herein by reference in their entirety as a part of the present application.

### FIELD

The present disclosure relates to the technical field of organic materials, in particular to a nitrogen-containing compound, an electronic component using the nitrogen-containing compound and an electronic device using the electronic component.

### BACKGROUND

An organic electroluminescent device, also referred to as an organic light-emitting diode, refers to a phenomenon that an organic light-emitting material emits light when excited by a current under the action of an electric field. It is a process of converting an electrical energy into a light energy. Compared with inorganic light-emitting materials, organic light-emitting diodes OLEDs have the advantages of active light emission, large optical path range, low driving voltage, high brightness, high efficiency, low energy consumption, and simple manufacturing process. Because of these advantages, organic light-emitting materials and devices have become one of the most popular research topics of the scientific community and the industrial community.

The organic electroluminescent device generally includes an anode, a hole transport layer, an electroluminescent layer as an energy conversion layer, an electron transport layer, and a cathode which are stacked in sequence. When a voltage is applied to the anode and the cathode, the two electrodes generate an electric field., Under the action of the electric field, the electrons on the cathode side move towards the electroluminescent layer, while the holes on the anode side also move towards the light-emitting layer, so the electrons and the holes combine in the electroluminescent layer to form excitons, and the excitons are in an excited state release energy outwards, thus causing the electroluminescent layer to emit light outwards.

In the prior art, CN111146349A, CN108101897A, CN110003091A, CN111279502A and the like disclose that hole materials that can be prepared in organic electroluminescent devices are used as electron blocking layers. However, it is still necessary to continue to develop new materials to further improve the performance of electronic components.

The above information in the Background section of the present disclosure is merely used to enhance an understanding of the context of the present disclosure, and thus it may include information that does not constitute the prior art known to those of ordinary skill in the art.

### SUMMARY

The present disclosure aims to provide a nitrogen-containing compound, an electronic component and an electronic device to improve the performance of the electronic component and the electronic device.

In order to achieve the above-mentioned inventive purpose, the present disclosure adopts the following technical solutions:

According to a first aspect of the present disclosure, provided is a nitrogen-containing compound, having a structure represented by a Formula 1:

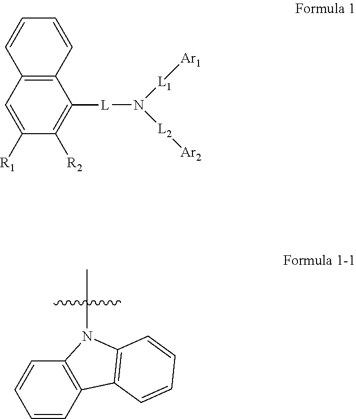

wherein R₁ and R₂ are respectively and independently selected from hydrogen and a group represented by a Formula 1-1, and only one of R₁ and R₂ is the group represented by a Formula 1-1;

L is selected from a single bond, and substituted or unsubstituted phenylene;

substituent in the L is selected from deuterium, a halogen group, and alkyl with 1 to 5 carbon atoms;

L₁ and L₂ are respectively and independently selected from a single bond, substituted or unsubstituted arylene with 6 to 30 carbon atoms, and substituted or unsubstituted heteroarylene with 3 to 30 carbon atoms;

Ar₁ and Ar₂ are selected from substituted or unsubstituted aryl with 6 to 30 carbon atoms, substituted or unsubstituted heteroaryl with 3 to 30 carbon atoms, substituted or unsubstituted alkyl with 1 to 20 carbon atoms, and substituted or unsubstituted cycloalkyl with 3 to 20 carbon atoms;

substituents in L₁, L₂, Ar₁ and Ar₂ are the same or different from each other, and are each independently selected from deuterium, a halogen group, cyano, heteroaryl with 3 to 20 carbon atoms, aryl with 6 to 20 carbon atoms, trialkylsilyl with 3 to 12 carbon atoms, triarylsilyl with 18 to 24 carbon atoms, alkyl with 1 to 10 carbon atoms, haloalkyl with 1 to 10 carbon atoms, cycloalkyl with 3 to 10 carbon atoms, heterocycloalkyl with 2 to 10 carbon atoms, alkoxy with 1 to 10 carbon atoms, alkylthio with 1 to 10 carbon atoms, aryloxy with 6 to 18 carbon atoms, arylthio with 6 to 18 carbon atoms, and phosphinyloxy with 6 to 18 carbon atoms.

The present disclosure provides a nitrogen-containing compound having a core structure formed by connecting carbazolyl to a 2-position or a 3-position of naphthyl, and bonding carbazolyl-linked naphthyl with a triarylamine structure. In this nitrogen-containing compound, the naphthyl has a stable planar structure, and it is linked to the carbazole group, which makes the compound have good thermal stability. The triarylamine structure has good hole-transport properties, and when the triarylamine structure is bonded with carbazole-linked naphthalene, the molecular rigidity is increased, the thermal stability is significantly improved, and the structure can be maintained stable at high temperature over a long period of time. The particular groups and particular linking means of the nitrogen-containing compound of the present disclosure greatly increase the steric hindrance of the compounds, and thus effectively increasing the T1 energy level of the compound molecules. When the material is used as an electron blocking layer of an organic electroluminescent device in the present disclosure, it is possible to block the outflow of excitons while ensuring the injection efficiency of holes into a light-emitting layer, reduce the driving voltage of the device, and improve the luminous efficiency and service life of the device.

In a second aspect, the present disclosure provides an electronic component, comprising an anode and a cathode which is arranged oppositely to the anode, and a functional layer disposed between the anode and the cathode; and the functional layer comprises the nitrogen-containing compound of the first aspect; and

preferably, the functional layer comprises an electron blocking layer, and the electron blocking layer comprises the nitrogen-containing compound.

In a third aspect, the present disclosure provides an electronic device, comprising the electronic component of the second aspect.

### BRIEF DESCRIPTION OF THE DRAWINGS

The above and other features and advantages of the present disclosure will become more apparent by describing the embodiments in detail with reference to the accompanying drawings.

FIG. 1 is a structural schematic diagram of an organic electroluminescent device according to one embodiment of the present disclosure.

FIG. 2 is a structural schematic diagram of a photoelectric conversion device according to one embodiment of the present disclosure.

FIG. 3 is a structural schematic diagram of an electronic device according to one embodiment of the present disclosure.

FIG. 4 is a structural schematic diagram of an electronic device according to another embodiment of the present disclosure.

FIG. 5 is a molecular structure model diagram of a nitrogen-containing compound 34 of the present disclosure.

FIG. 6 is a molecular structure model diagram of a compound B in Comparative Example.

### DESCRIPTION OF REFERENCE SIGNS

100, anode; 200, cathode; 300, functional layer; 310, hole injection layer; 321, hole transport layer; 322, electron blocking layer; 330, organic light-emitting layer; 340, hole blocking layer; 350, electron transport layer; 360, electron injection layer; 370, photoelectric conversion layer; 400, first electronic device; and 500, second electronic device.

### DETAILED DESCRIPTION

Examples will now be described more fully with reference to the accompanying drawings. However, the examples can be implemented in various forms and should not be construed as limited to the examples set forth herein; on the contrary, these examples are provided so that the present disclosure will be thorough and complete, and the concept of the examples is fully conveyed to those skilled in the art. The described features, structures, or characteristics may be combined in any suitable manner in one or more examples. In the following description, many specific details are provided to provide a thorough understanding of the examples of the present disclosure.

In the drawings, the thicknesses of regions and layers may be exaggerated for clarity. The same reference signs denote the same or similar structures in the drawings, and thus their detailed description will be omitted.

The described features, structures, or characteristics may be combined in any suitable manner in one or more examples. In the following description, many specific details are provided to provide a thorough understanding of the examples of the present disclosure.

The present disclosure provides a nitrogen-containing compound, having a structure represented by a Formula 1:

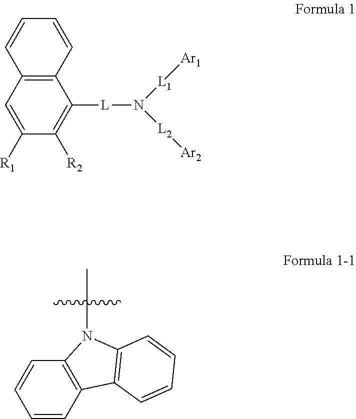

wherein R₁ and R₂ are respectively and independently selected from hydrogen or a group represented by a Formula 1-1, and only one of R₁ and R₂ is the group represented by a Formula 1-1;

L is selected from a single bond, and substituted or unsubstituted phenylene;

substituent in the L is selected from deuterium, a halogen group, and alkyl with 1 to 5 carbon atoms;

L₁ and L₂ are respectively and independently selected from a single bond, substituted or unsubstituted arylene with 6 to 30 carbon atoms, and substituted or unsubstituted heteroarylene with 3 to 30 carbon atoms;

Ar₁ and Ar₂ are selected from substituted or unsubstituted aryl with 6 to 30 carbon atoms, substituted or unsubstituted heteroaryl with 3 to 30 carbon atoms, substituted or unsubstituted alkyl with 1 to 20 carbon atoms, and substituted or unsubstituted cycloalkyl with 3 to 20 carbon atoms;

substituents in L₁, L₂, Ar₁ and Ar₂ are the same as or different from each other, and are each independently selected from deuterium, a halogen group, cyano, heteroaryl with 3 to 20 carbon atoms, aryl with 6 to 20 carbon atoms, trialkylsilyl with 3 to 12 carbon atoms, triarylsilyl with 18 to 24 carbon atoms, alkyl with 1 to 10 carbon atoms, haloalkyl with 1 to 10 carbon atoms, cycloalkyl with 3 to 10 carbon atoms, heterocycloalkyl with 2 to 10 carbon atoms, alkoxy with 1 to 10 carbon atoms, alkylthio with 1 to 10 carbon atoms, aryloxy with 6 to 18 carbon atoms, arylthio with 6 to 18 carbon atoms, and phosphinyloxy with 6 to 18 carbon atoms.

In the present disclosure, the terms “optional” and “optionally” mean that the subsequently described event or circumstance can but need not occur, and that the description includes occasions where the event or circumstance occurs or does not occur. For example, “optionally, two adjacent substituents xx form a ring;” means that the two substituents may, but need not, form a ring, including a scenario in which two adjacent substituents form a ring and a scenario in which two adjacent substituents do not form a ring.

In the present disclosure, the descriptions of “each . . . is independently”, “ . . . is respectively and independently” and “ . . . is independently selected from” can be interchanged, which should be understood in a broad sense, and may mean that specific options expressed by a same symbol in different groups do not influence each other, or that specific options expressed by a same symbol in a same group do not influence each other. For example, the meaning of

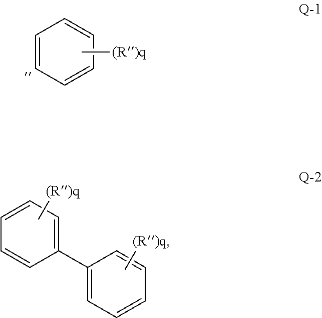

where each q is independently 0, 1, 2 or 3 and each R″ is independently selected from hydrogen, deuterium, fluorine, and chlorine” means that: a formula Q-1 represents that q substituents R″ exist on a benzene ring, each R″ can be the same or different, and options of each R″ do not influence each other; and a formula Q-2 represents that each benzene ring of biphenyl has q substituents R″, the number q of the substituents R″ on the two benzene rings can be the same or different, each R″ can be the same or different, and options of each R″ do not affect each other.

In the present disclosure, an unpositioned connecting bond refers to a single bond

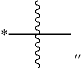 [SMILES: **]

extending from a ring system, which means that one end of the connecting bond can be connected with any position in the ring system through which the bond penetrates, and the other end of the connecting bond is connected with the remaining part of a compound molecule.

For example, as shown in the following formula (f), naphthyl represented by the formula (f) is connected to other positions of a molecule through two unpositioned connecting bonds penetrating a dicyclic ring, and its meaning includes any one possible connecting mode represented by formulae (f-1)-(f-10).

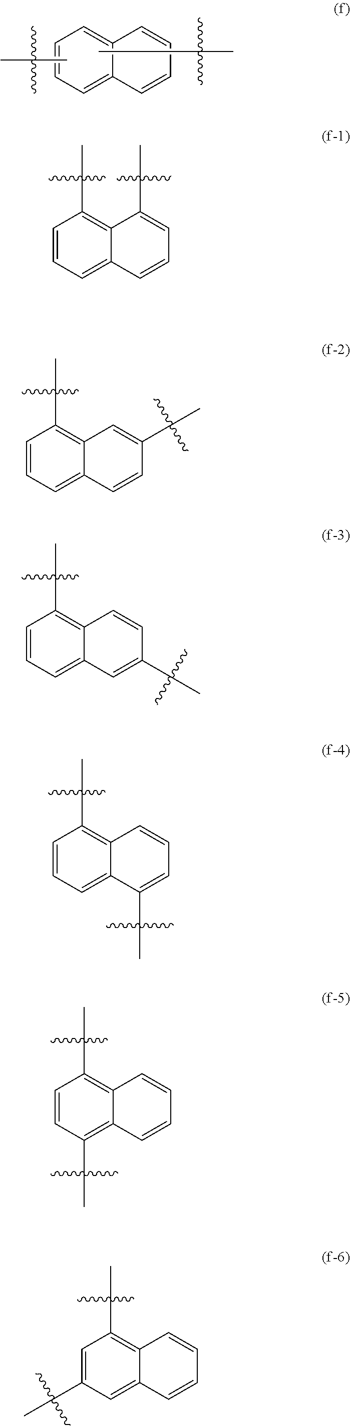

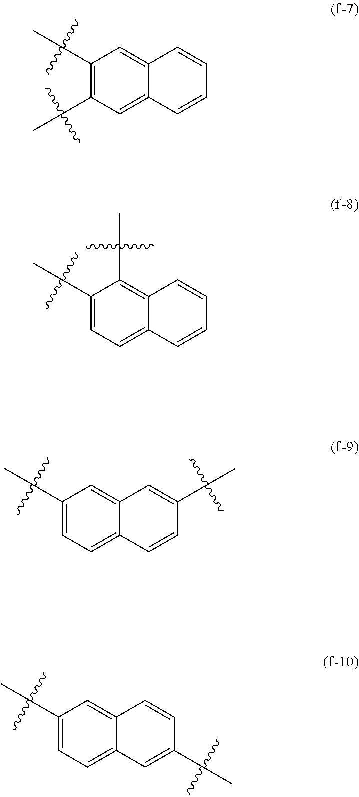 [SMILES: *c1cc2ccccc2cc1* ; *c1ccc2ccccc2c1* ; *c1ccc2ccc(*)cc2c1 ; *c1ccc2cc(*)ccc2c1]

For another example, as shown in the following formula (X′), phenanthryl represented by the formula (X′) is connected with other positions of a molecule through one unpositioned connecting bond extending from the middle of a benzene ring on one side, and its meaning includes any possible connecting mode represented by formulae (X′-1)-(X′-4).

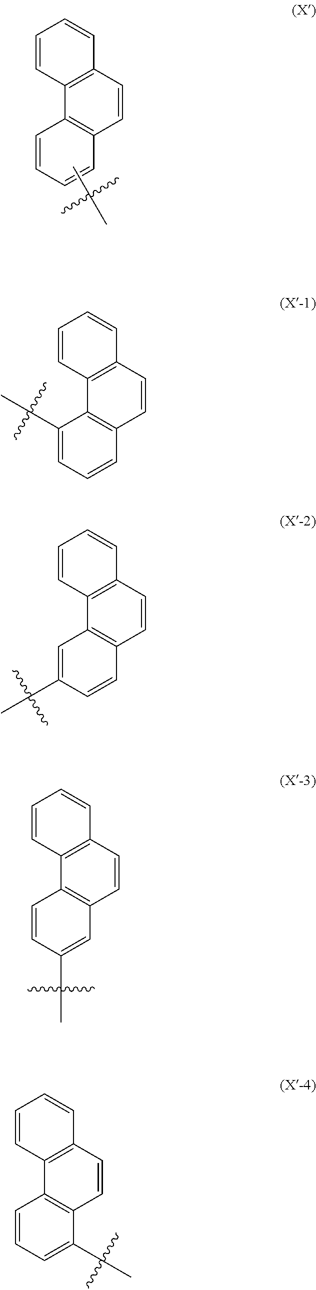

An unpositioned substituent in the present disclosure refers to a substituent connected through a single bond extending from the center of a ring system, which means that the substituent can be connected to any possible position in the ring system. For example, as shown in the following formula (Y), a substituent R′ represented by the formula (Y) is connected with a quinoline ring through one unpositioned connecting bond, and its meaning includes any possible connecting mode represented by formulae (Y-1)-(Y-7).

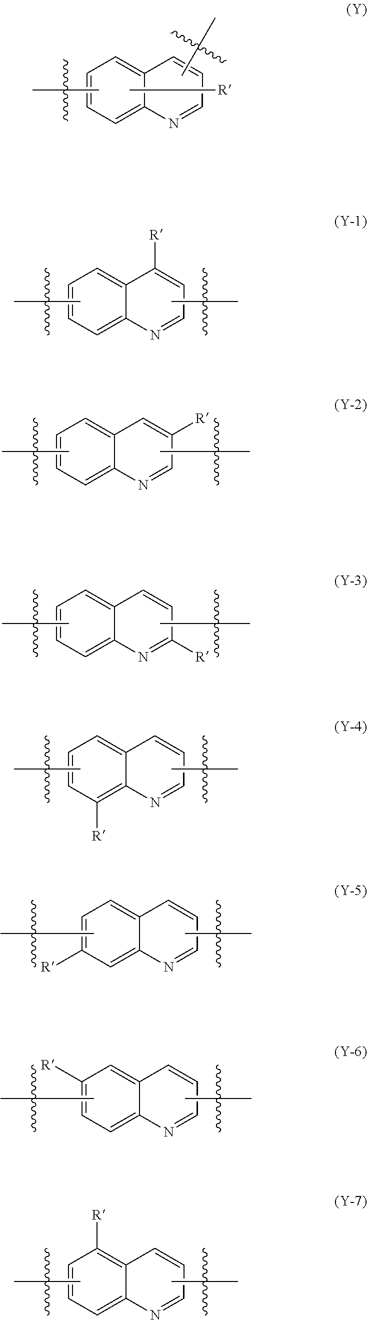

In the present disclosure, the number of carbon atoms of L, L₁, L₂, Ar₁ and Ar₂ refers to the number of all carbon atoms. For example, if Ar₁ is

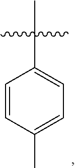 [SMILES: *c1ccc(C)cc1]

then the number of carbon atoms is 7.

In the present disclosure, if no additional specific definition is provided, “hetero” means that at least one heteroatom such as B, N, O, S, Se, Si, or P is included in one functional group and the remaining atoms are carbon and hydrogen. Unsubstituted alkyl may be “a saturated alkyl group” without any double or triple bonds.

In the present disclosure, “alkyl” may include linear alkyl or branched alkyl. The alkyl may have 1 to 20 carbon atoms, and in the present disclosure, a numerical range such as “1 to 20” refers to each integer in a given range; for example, “1 to 20 carbon atoms” refers to alkyl that may include 1 carbon atom, 2 carbon atoms, 3 carbon atoms, 4 carbon atoms, 5 carbon atoms, 6 carbon atoms, 7 carbon atoms, 8 carbon atoms, 9 carbon atoms, 10 carbon atoms, 11 carbon atoms, 12 carbon atoms, 13 carbon atoms, 14 carbon atoms, 15 carbon atoms, 16 carbon atoms, 17 carbon atoms, 18 carbon atoms, 19 carbon atoms, or 20 carbon atoms. The alkyl may also be medium alkyl with 1 to 10 carbon atoms. The alkyl may also be lower alkyl with 1 to 6 carbon atoms. In addition, the alkyl can be substituted or unsubstituted.

Optionally, the alkyl is selected from alkyl with 1 to 6 carbon atoms, and specific examples includes, but are not limited to, methyl, ethyl, n-propyl, isopropyl, n-butyl, isobutyl, sec-butyl, tert-butyl, pentyl and hexyl.

In the present disclosure, aryl refers to an optional functional group or substituent derived from an aromatic carbocyclic ring. The aryl can be monocyclic aryl (e.g., phenyl) or polycyclic aryl, in other words, the aryl can be monocyclic aryl, fused aryl, two or more monocyclic aryl conjugatedly connected by carbon-carbon bonds, monocyclic aryl and fused aryl which are conjugatedly connected by a carbon-carbon bond, and two or more fused aryl conjugatedly connected by carbon-carbon bond. That is, unless specified otherwise, two or more aromatic groups conjugatedly connected by carbon-carbon bond can also be regarded as aryl of the present disclosure. The fused aryl may, for example, include bicyclic fused aryl (e.g., naphthyl), tricyclic fused aryl (e.g., phenanthryl, fluorenyl, and anthryl), and the like. The aryl does not contain heteroatoms such as B, N, O, S, P, Se, and Si. For example, in the present disclosure, biphenyl, terphenyl, and the like are aryl. Examples of the aryl can include, but are not limited to, phenyl, naphthyl, fluorenyl, anthryl, phenanthryl, biphenyl, terphenyl, quaterphenyl, quinquephenyl, benzo[9,10]phenanthryl, pyrenyl, benzofluoranthenyl, chrysenyl, and the like. The “substituted or unsubstituted aryl” in the present disclosure can contain 6 to 30 carbon atoms, in some embodiments, the number of carbon atoms in the substituted or unsubstituted aryl can be 6 to 25, in other embodiments, the number of carbon atoms in the substituted or unsubstituted aryl can be 6 to 18, and in yet other embodiments, the number of carbon atoms in the substituted or unsubstituted aryl can be 6 to 13. For example, in the present disclosure, the number of carbon atoms in the substituted or unsubstituted aryl can be 6, 12, 13, 14, 15, 18, 20, 24, 25, 30, 31, 32, 33, 34, 35, 36, or 40, and of course, the number of carbon atoms can also be other numbers, which will not be listed herein. In the present disclosure, biphenyl can be understood as phenyl-substituted aryl and can also be understood as unsubstituted aryl.

In the present disclosure, the related arylene refers to a divalent group formed by further loss of one hydrogen atom of the aryl.

In the present disclosure, the substituted aryl can be that one or two or more hydrogen atoms in the aryl are substituted with groups such as a deuterium atom, a halogen group, cyano, aryl, heteroaryl, trialkylsilyl, alkyl, cycloalkyl, alkoxy, alkylthio, and the like. It should be understood that the number of carbon atoms of the substituted aryl refers to the total number of carbon atoms of the aryl and substituents on the aryl, e.g., substituted aryl with 18 carbon atoms means that the total number of carbon atoms of the aryl and its substituents is 18.

In the present disclosure, specific examples of aryl as a substituent include, but are not limited to, phenyl, biphenyl, naphthyl, anthryl, phenanthryl, dimethylfluorenyl, diphenylfluorenyl, spirobifluorenyl, and the like.

In the present disclosure, fluorenyl may be substituted, and the two substituents may be bonded to each other to form a spiro structure, and specific embodiments include, but are not limited to, the following structures:

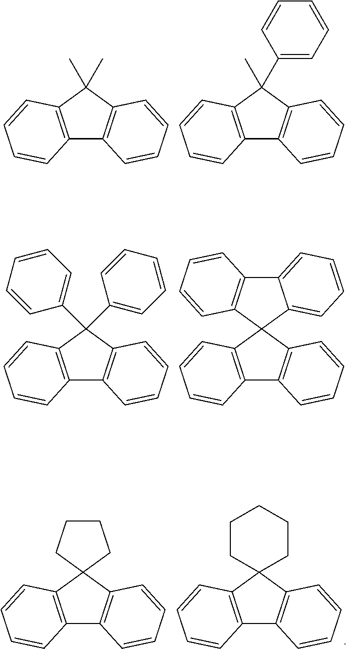

In the present disclosure, heteroaryl refers to a monovalent aromatic ring containing at least one heteroatom in the ring or its derivative, and the heteroatom can be at least one of B, O, N, P, Si, Se, and S. The heteroaryl can be monocyclic heteroaryl or polycyclic heteroaryl, in other words, the heteroaryl can be a single aromatic ring system or a multiple of aromatic ring systems conjugatedly connected by carbon-carbon bond, and any one aromatic ring system is one aromatic monocyclic ring or one aromatic fused ring. For example, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, benzothiazolyl, phenothiazinyl, silafluorenyl, dibenzofuranyl, and N-arylcarbazolyl (e.g., N-phenylcarbazolyl), N-heteroarylcarbazolyl (e.g., N-pyridylcarbazolyl), N-alkylcarbazolyl (e.g., N-methylcarbazolyl), and the like, but are not limited to this. Where, thienyl, furyl, phenanthrolinyl, etc. are heteroaryl of the single aromatic ring system, and N-arylcarbazolyl, and N-heteroarylcarbazolyl are heteroaryl of the plurality of aromatic ring systems conjugatedly connected by carbon-carbon bonds. The “substituted or unsubstituted heteroaryl” in the present disclosure can contain 3 to 30 carbon atoms, in some embodiments, the number of carbon atoms in the substituted or unsubstituted heteroaryl can be 3 to 25, in other embodiments, the number of carbon atoms in the substituted or unsubstituted heteroaryl can be 3 to 20, and in yet other embodiments, the number of carbon atoms in the substituted or unsubstituted heteroaryl can be 12 to 20. For example, the number of carbon atoms may be 3, 4, 5, 7, 12, 13, 18, 20, 24, 25 or 30, and of course, the number of carbon atoms may also be other numbers, which will not be listed herein.

In the present disclosure, the related heteroarylene refers to a divalent group formed by further loss of one hydrogen atom of the heteroaryl.

In the present disclosure, the substituted heteroaryl may be that one or two or more hydrogen atoms in the heteroaryl are substituted with groups such as a deuterium atom, a halogen group, cyano, aryl, heteroaryl, trialkylsilyl, alkyl, cycloalkyl, alkoxy, alkylthio, and the like. It should be understood that the number of carbon atoms of the substituted heteroaryl refers to the total number of carbon atoms of the heteroaryl and substituents on the heteroaryl.

In the present disclosure, specific examples of heteroaryl as a substituent include, but are not limited to, dibenzofuranyl, dibenzothienyl, carbazolyl, N-phenylcarbazolyl, phenanthrolinyl, and the like.

In the present disclosure, the halogen group may include fluorine, iodine, bromine, chlorine, and the like.

In the present disclosure, specific examples of trialkylsilyl with 3 to 12 carbon atoms include, but are not limited to, trimethylsilyl, triethylsilyl, and the like.

According to one embodiment of the present disclosure, L is selected from a single bond or phenylene.

Preferably, L is phenylene.

In one embodiment of the present disclosure, L₁ and L₂ are respectively and independently selected from a single bond, substituted or unsubstituted arylene with 6 to 20 carbon atoms, and substituted or unsubstituted heteroarylene with 5 to 20 carbon atoms.

Optionally, substituents in the L₁ and L₂ are respectively and independently selected from deuterium, a halogen group, cyano, alkyl with 1 to 5 carbon atoms, and aryl with 6 to 12 carbon atoms.

Specifically, specific examples of substituents in L₁ and L₂ include, but are not limited to, deuterium, fluorine, cyano, methyl, ethyl, n-propyl, isopropyl, tert-butyl, phenyl, naphthyl, and biphenyl.

According to another embodiment of the present disclosure, L₁ and L₂ are respectively and independently selected from a single bond, substituted or unsubstituted phenylene, substituted or unsubstituted biphenylene, substituted or unsubstituted naphthylene, substituted or unsubstituted fluorenylene, substituted or unsubstituted carbazolylene, substituted or unsubstituted dibenzofurylene, and substituted or unsubstituted dibenzothienylene.

Preferably, the L₁ and L₂ are respectively and independently selected from a single bond, substituted or unsubstituted phenylene, substituted or unsubstituted biphenylene, substituted or unsubstituted naphthylene, and substituted or unsubstituted carbazolylene.

According to another embodiment of the present disclosure, L₁ and L₂ are respectively and independently selected from a single bond, and a substituted or unsubstituted group V; the unsubstituted group V is selected from a group consisting of the following groups:

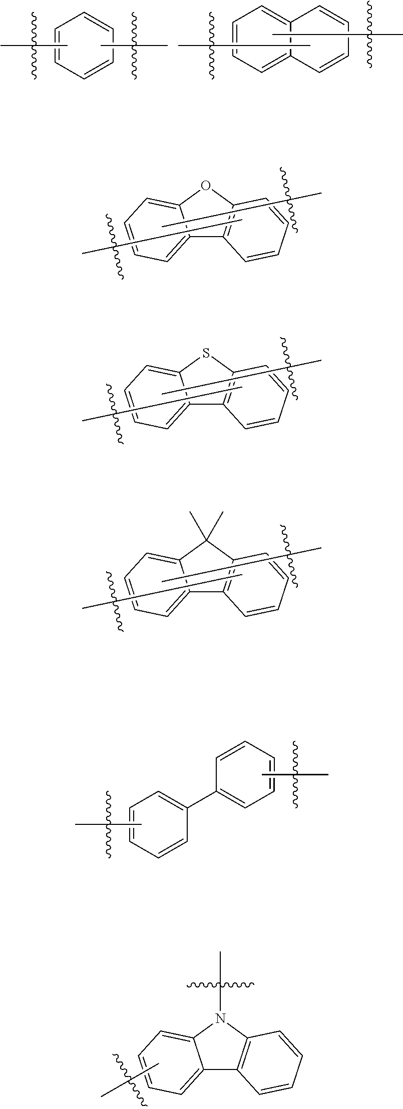

where

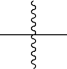

represents a chemical bond; substituted group V has one or more substituents, and the substituents are each independently selected from: deuterium, fluorine, cyano, methyl, ethyl, n-propyl, isopropyl, tert-butyl, phenyl, naphthyl, and biphenyl; when the number of the substituents in the group V is greater than 1, the substituents are the same or different.

Optionally, L₁ and L₂ are respectively and independently selected from a single bond or a group consisting of the following groups, but are not limited to:

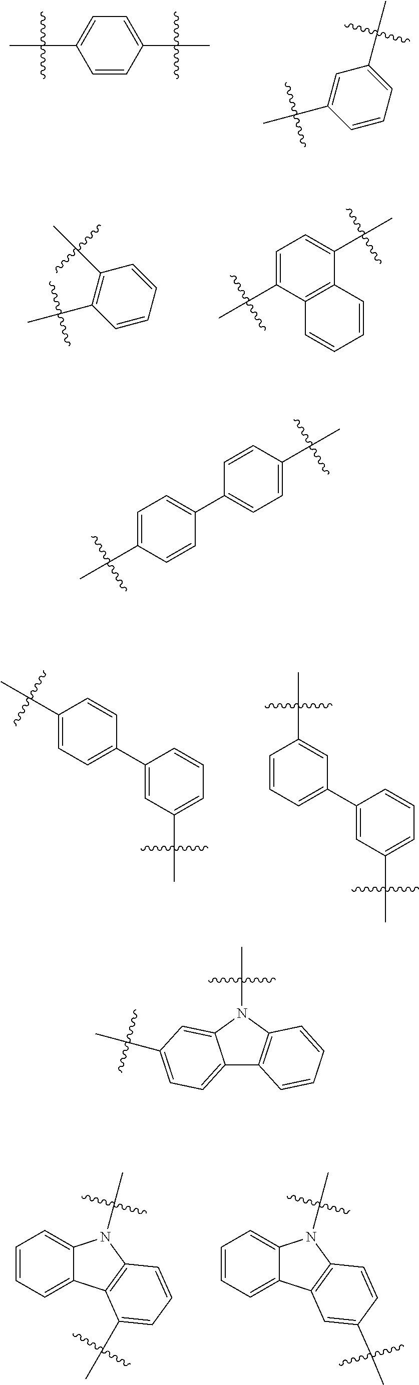

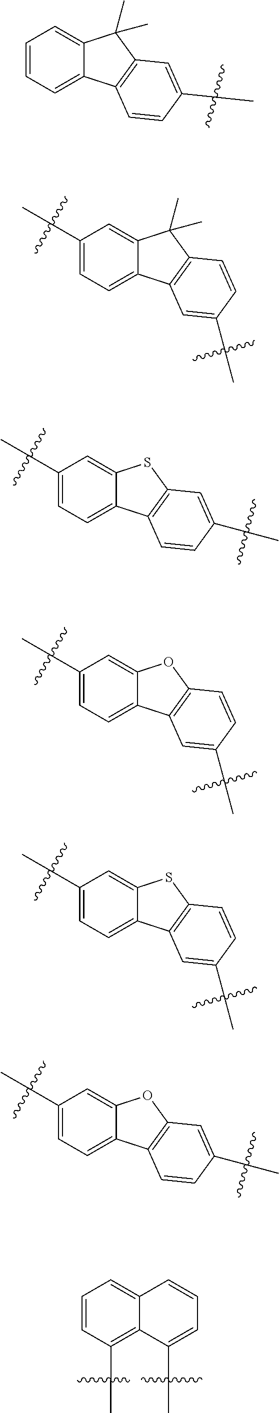

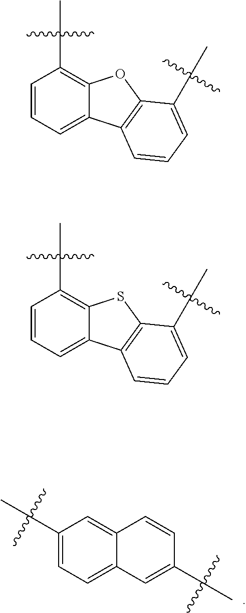 [SMILES: *c1cccc2c1oc1c(*)cccc12 ; *c1cccc2c1sc1c(*)cccc12 ; *c1ccc2cc(*)ccc2c1]

According to one embodiment of the present disclosure, Ar₁ and Ar₂ are respectively and independently selected from substituted or unsubstituted aryl with 6 to 20 carbon atoms, and substituted or unsubstituted heteroaryl with 12 to 20 carbon atoms.

Optionally, substituents the in Ar₁ and Ar₂ are respectively and independently selected from deuterium, a halogen group, cyano, alkyl with 1 to 5 carbon atoms, aryl with 6 to 12 carbon atoms, and trialkylsilyl with 3 to 6 carbon atoms.

Specifically, specific examples of substituents in the Ar₁ and Ar₂ include, but are not limited to, deuterium, fluorine, cyano, methyl, ethyl, n-propyl, isopropyl, tert-butyl, phenyl, naphthyl, biphenyl, and trimethylsilyl.

According to another embodiment of the present disclosure, Ar₁ and Ar₂ are selected from substituted or unsubstituted phenyl, substituted or unsubstituted biphenyl, substituted or unsubstituted terphenyl, substituted or unsubstituted naphthyl, substituted or unsubstituted anthryl, substituted or unsubstituted phenanthryl, substituted or unsubstituted dibenzofuranyl, substituted or unsubstituted dibenzothienyl, substituted or unsubstituted fluorenyl, and substituted or unsubstituted carbazolyl.

According to another example of the present disclosure, Ar₁ and Ar₂ are selected from a substituted or unsubstituted group W, the unsubstituted group W is selected from a group consisting of the following groups:

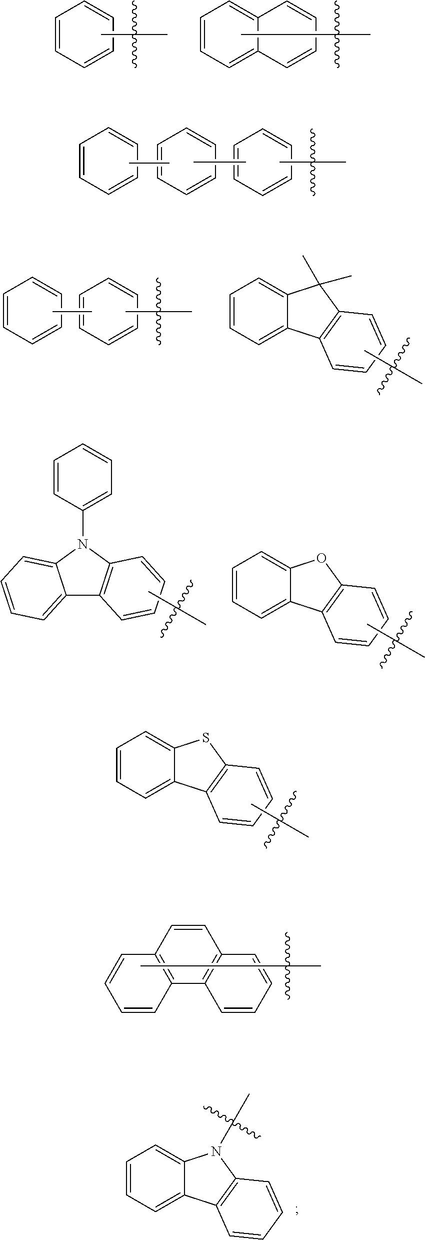

where,

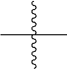

represents a chemical bond; substituted group W has one or more substituents, and the substituents are each independently selected from deuterium, fluorine, cyano, methyl, ethyl, n-propyl, isopropyl, tert-butyl, phenyl, naphthyl, biphenyl, and trimethylsilyl; when the number of the substituents in the group W is greater than 1, the substituents are the same or different.

Optionally, Ar₁ and Ar₂ are selected from a group consisting of the following groups, but are not limited to:

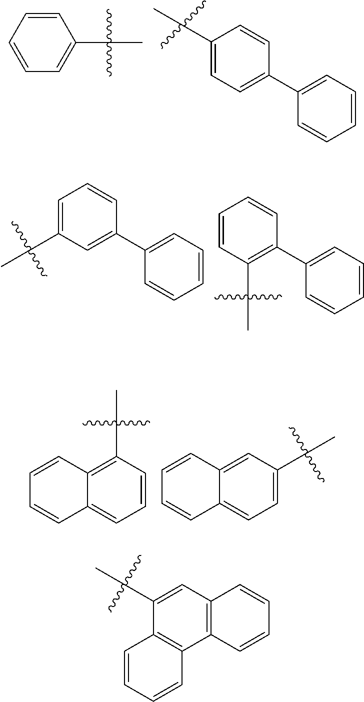

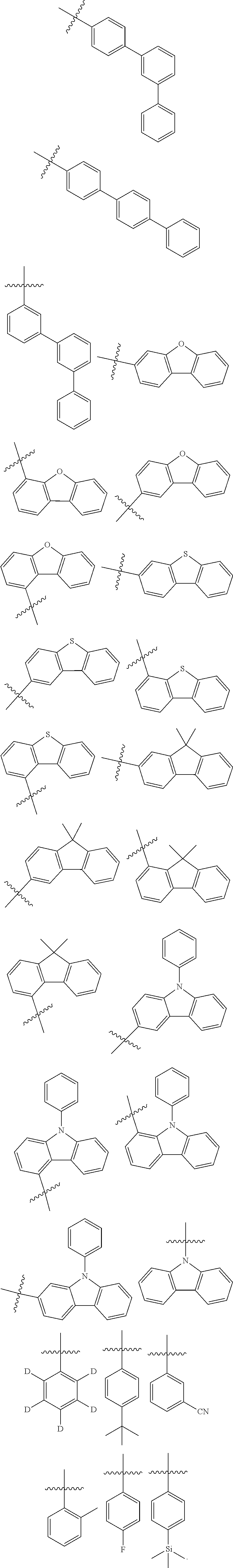

Optionally, the nitrogen-containing compound is selected from a group of consisting of the following compounds, but is not limited to:

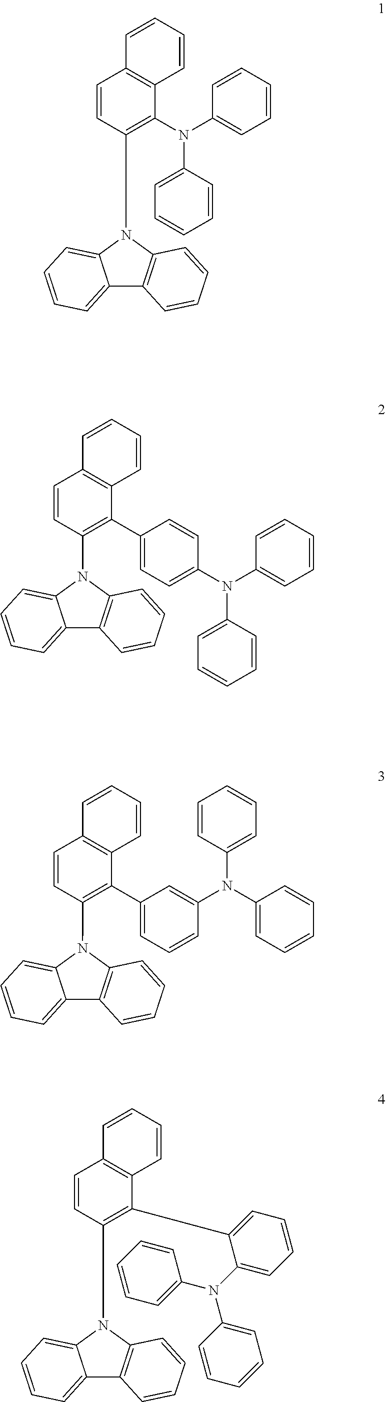 [SMILES: c1ccc(N(c2ccccc2)c2c(-n3c4ccccc4c4ccccc43)ccc3ccccc23)cc1 ; c1ccc(N(c2ccccc2)c2ccc(-c3c(Cn4c5ccccc5c5ccccc54)ccc4ccccc34)cc2)cc1 ; c1ccc(N(c2ccccc2)c2cccc(-c3c(Cn4c5ccccc5c5ccccc54)ccc4ccccc34)c2)cc1 ; c1ccc(N(c2ccccc2)c2ccccc2-c2c(-n3c4ccccc4c4ccccc43)ccc3ccccc23)cc1]

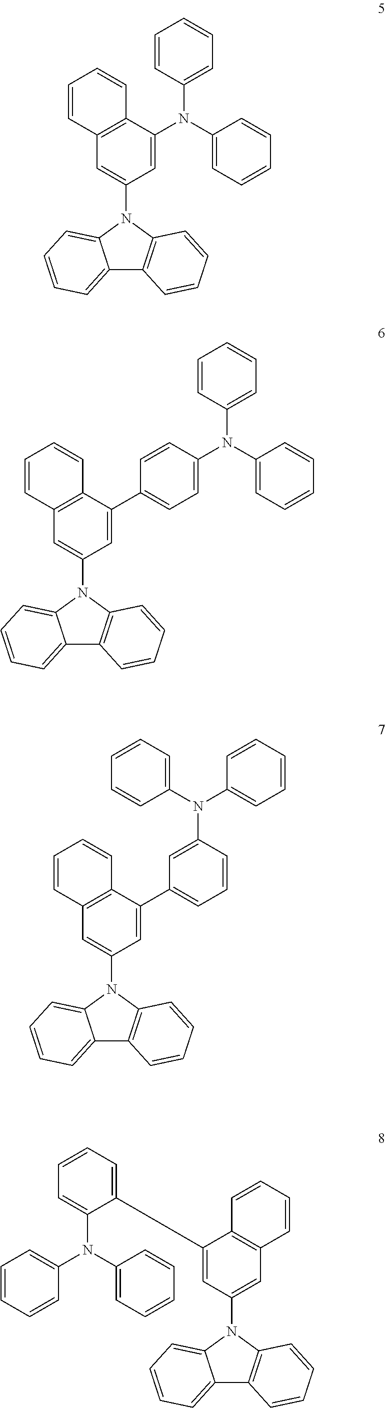 [SMILES: c1ccc(N(c2ccccc2)c2cc(-n3c4ccccc4c4ccccc43)cc3ccccc23)cc1 ; c1ccc(N(c2ccccc2)c2ccc(-c3cc(-n4c5ccccc5c5ccccc54)cc4ccccc34)cc2)cc1 ; c1ccc(N(c2ccccc2)c2cccc(-c3cc(-n4c5ccccc5c5ccccc54)cc4ccccc34)c2)cc1 ; c1ccc(N(c2ccccc2)c2ccccc2-c2cc(-n3c4ccccc4c4ccccc43)cc3ccccc23)cc1]

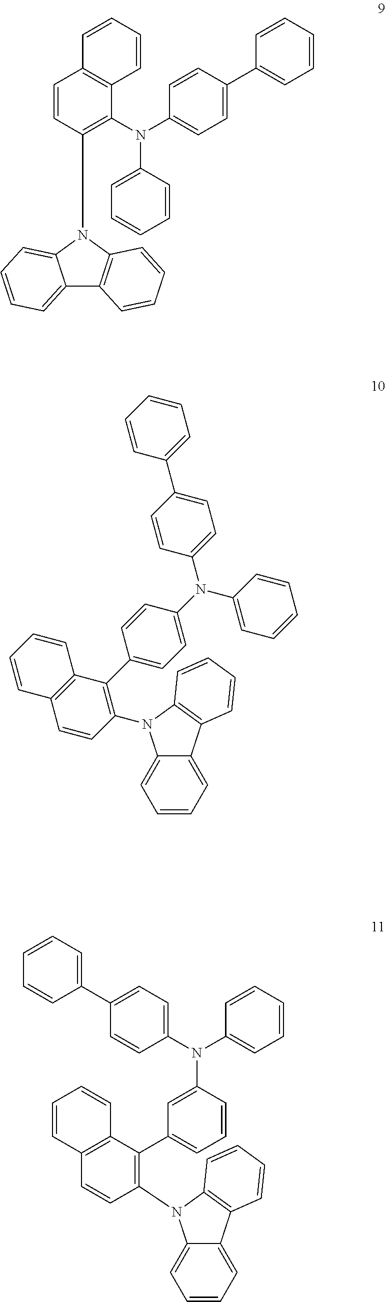 [SMILES: c1ccc(-c2ccc(N(c3ccccc3)c3c(-n4c5ccccc5c5ccccc54)ccc4ccccc34)cc2)cc1 ; c1ccc(-c2ccc(N(c3ccccc3)c3ccc(-c4c(-n5c6ccccc6c6ccccc65)ccc5ccccc45)cc3)cc2)cc1 ; c1ccc(-c2ccc(N(c3ccccc3)c3cccc(-c4c(-n5c6ccccc6c6ccccc65)ccc5ccccc45)c3)cc2)cc1]

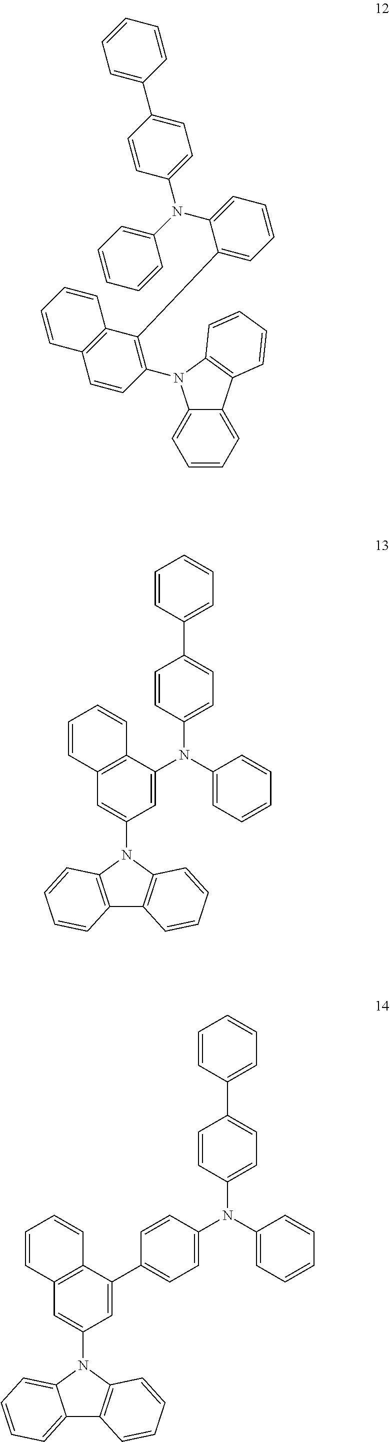 [SMILES: c1ccc(-c2ccc(N(c3ccccc3)c3ccccc3-c3c(-n4c5ccccc5c5ccccc54)ccc4ccccc34)cc2)cc1 ; c1ccc(-c2ccc(N(c3ccccc3)c3cc(-n4c5ccccc5c5ccccc54)cc4ccccc34)cc2)cc1 ; c1ccc(-c2ccc(N(c3ccccc3)c3ccc(-c4cc(-n5c6ccccc6c6ccccc65)cc5ccccc45)cc3)cc2)cc1]

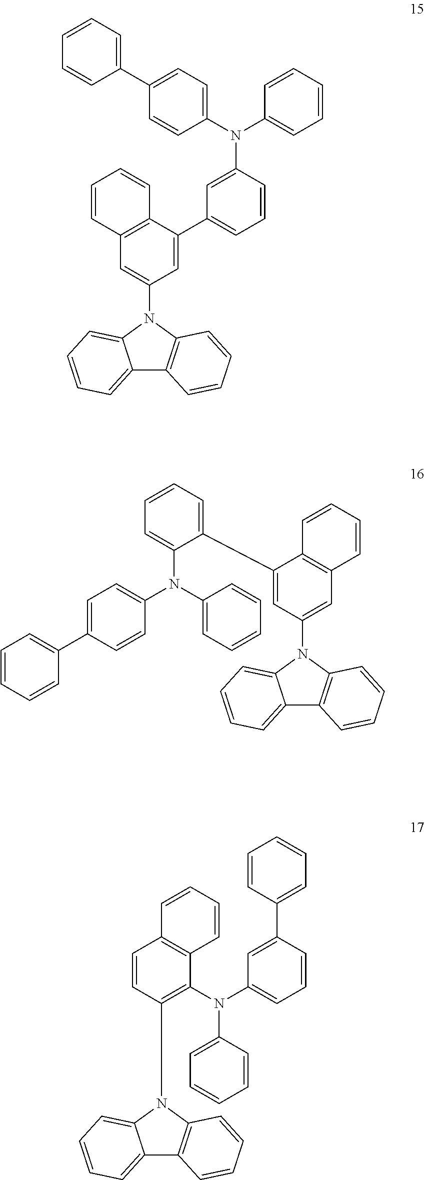 [SMILES: c1ccc(-c2ccc(N(c3ccccc3)c3cccc(-c4cc(-n5c6ccccc6c6ccccc65)cc5ccccc45)c3)cc2)cc1 ; c1ccc(-c2ccc(N(c3ccccc3)c3ccccc3-c3cc(-n4c5ccccc5c5ccccc54)cc4ccccc34)cc2)cc1 ; c1ccc(-c2cccc(N(c3ccccc3)c3c(-n4c5ccccc5c5ccccc54)ccc4ccccc34)c2)cc1]

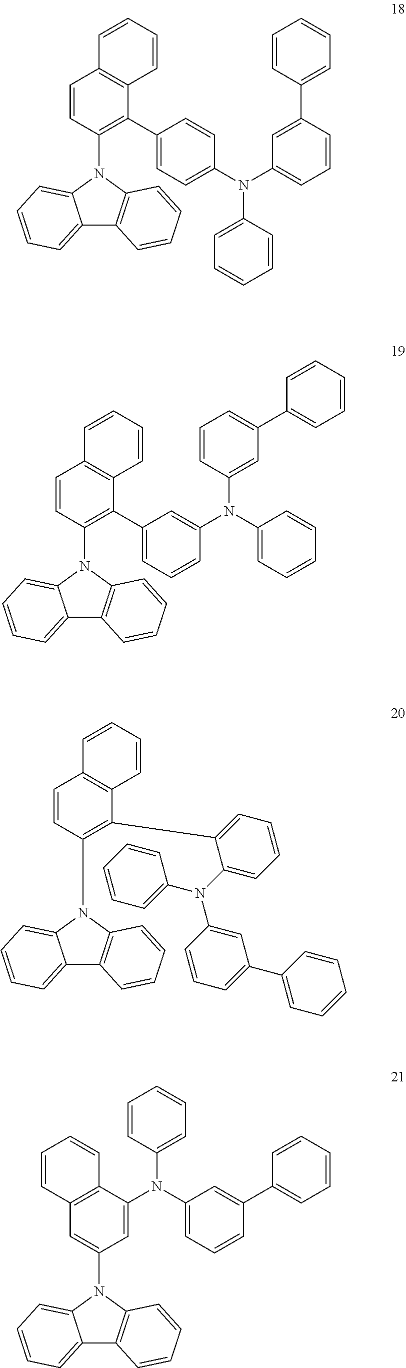 [SMILES: c1ccc(-c2cccc(N(c3ccccc3)c3ccc(-c4c(-n5c6ccccc6c6ccccc65)ccc5ccccc45)cc3)c2)cc1 ; c1ccc(-c2cccc(N(c3ccccc3)c3cccc(-c4c(-n5c6ccccc6c6ccccc65)ccc5ccccc45)c3)c2)cc1 ; c1ccc(-c2cccc(N(c3ccccc3)c3ccccc3-c3c(-n4c5ccccc5c5ccccc54)ccc4ccccc34)c2)cc1 ; c1ccc(-c2cccc(N(c3ccccc3)c3cc(-n4c5ccccc5c5ccccc54)cc4ccccc34)c2)cc1]

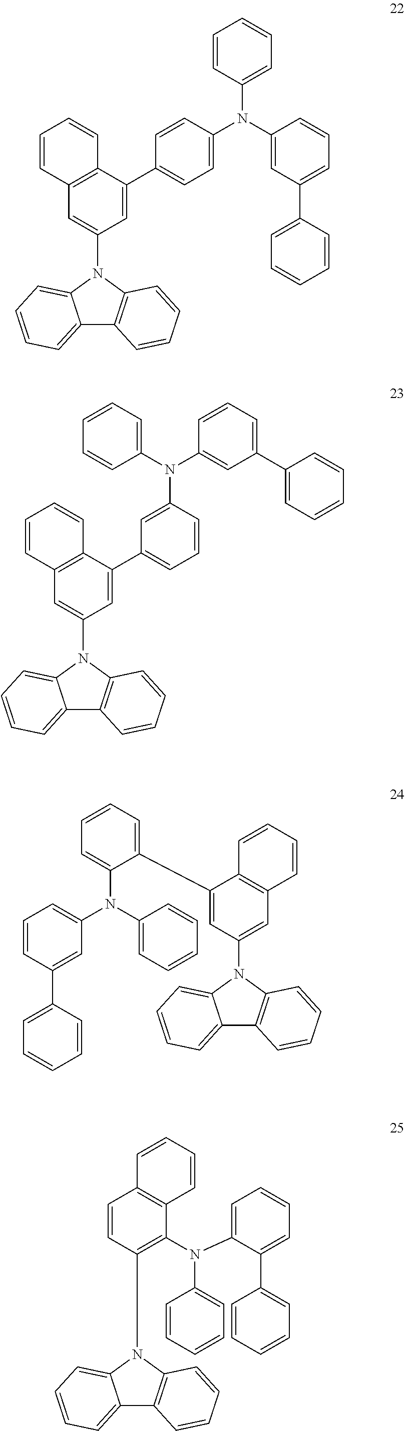 [SMILES: c1ccc(-c2cccc(N(c3ccccc3)c3ccc(-c4cc(-n5c6ccccc6c6ccccc65)cc5ccccc45)cc3)c2)cc1 ; c1ccc(-c2cccc(N(c3ccccc3)c3cccc(-c4cc(-n5c6ccccc6c6ccccc65)cc5ccccc45)c3)c2)cc1 ; c1ccc(-c2cccc(N(c3ccccc3)c3ccccc3-c3cc(-n4c5ccccc5c5ccccc54)cc4ccccc34)c2)cc1 ; c1ccc(-c2ccccc2N(c2ccccc2)c2c(-n3c4ccccc4c4ccccc43)ccc3ccccc23)cc1]

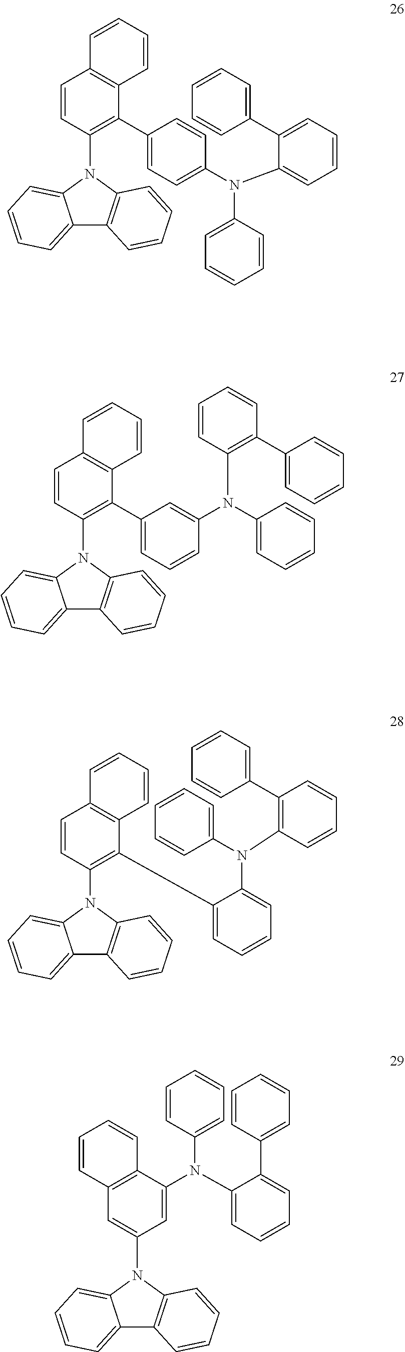 [SMILES: c1ccc(-c2ccccc2N(c2ccccc2)c2ccc(-c3c(-n4c5ccccc5c5ccccc54)ccc4ccccc34)cc2)cc1 ; c1ccc(-c2ccccc2N(c2ccccc2)c2cccc(-c3c(-n4c5ccccc5c5ccccc54)ccc4ccccc34)c2)cc1 ; c1ccc(-c2ccccc2N(c2ccccc2)c2ccccc2-c2c(-n3c4ccccc4c4ccccc43)ccc3ccccc23)cc1 ; c1ccc(-c2ccccc2N(c2ccccc2)c2cc(-n3c4ccccc4c4ccccc43)cc3ccccc23)cc1]

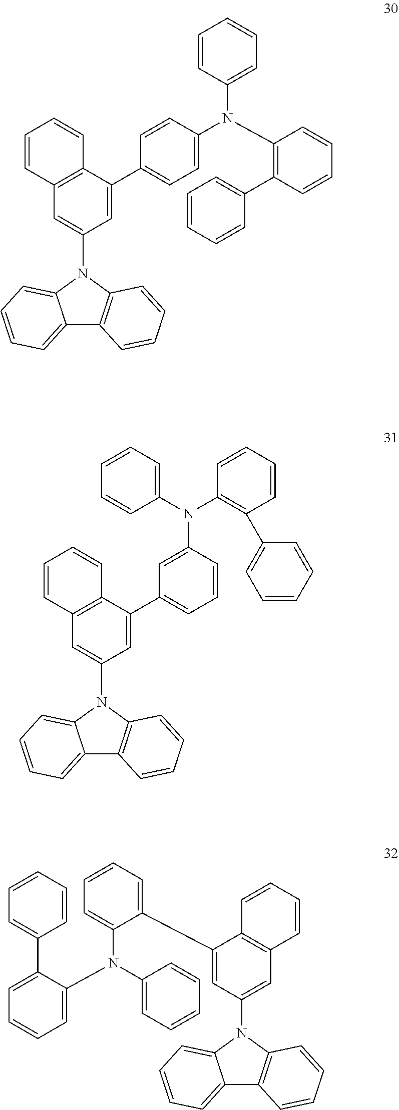 [SMILES: c1ccc(-c2ccccc2N(c2ccccc2)c2ccc(-c3cc(-n4c5ccccc5c5ccccc54)cc4ccccc34)cc2)cc1 ; c1ccc(-c2ccccc2N(c2ccccc2)c2cccc(-c3cc(-n4c5ccccc5c5ccccc54)cc4ccccc34)c2)cc1 ; c1ccc(-c2ccccc2N(c2ccccc2)c2ccccc2-c2cc(-n3c4ccccc4c4ccccc43)cc3ccccc23)cc1]

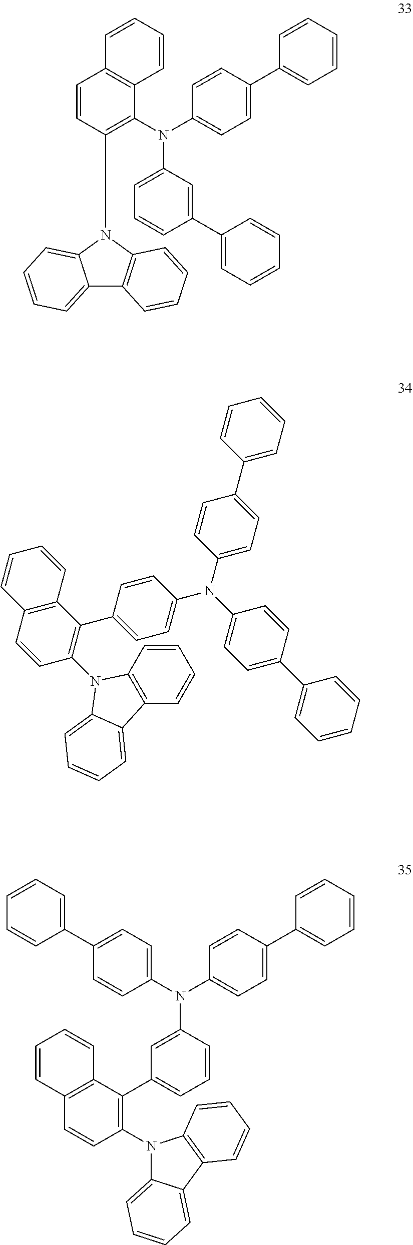 [SMILES: c1ccc(-c2ccc(N(c3cccc(-c4ccccc4)c3)c3c(-n4c5ccccc5c5ccccc54)ccc4ccccc34)cc2)cc1 ; c1ccc(-c2ccc(N(c3ccc(-c4ccccc4)cc3)c3ccc(-c4c(-n5c6ccccc6c6ccccc65)ccc5ccccc45)cc3)cc2)cc1 ; c1ccc(-c2ccc(N(c3ccc(-c4ccccc4)cc3)c3cccc(-c4c(-n5c6ccccc6c6ccccc65)ccc5ccccc45)c3)cc2)cc1]

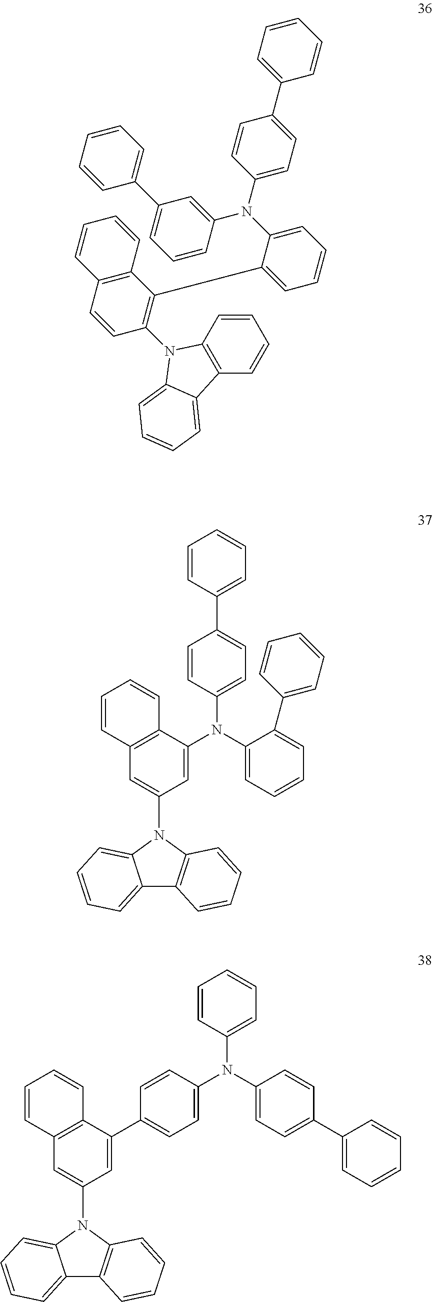 [SMILES: c1ccc(-c2ccc(N(c3cccc(-c4ccccc4)c3)c3ccccc3-c3c(-n4c5ccccc5c5ccccc54)ccc4ccccc34)cc2)cc1 ; c1ccc(-c2ccc(N(c3ccccc3-c3ccccc3)c3cc(-n4c5ccccc5c5ccccc54)cc4ccccc34)cc2)cc1 ; c1ccc(-c2ccc(N(c3ccccc3)c3ccc(-c4cc(-n5c6ccccc6c6ccccc65)cc5ccccc45)cc3)cc2)cc1]

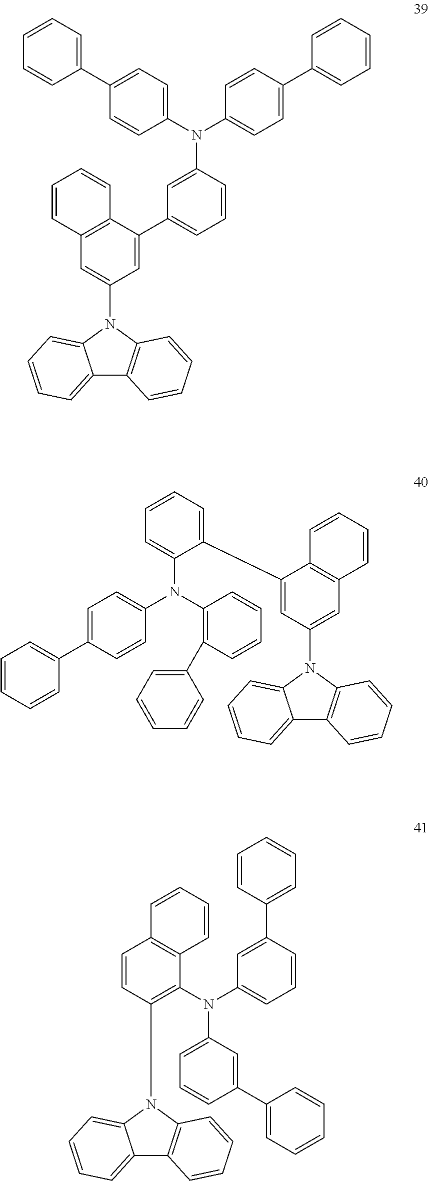 [SMILES: c1ccc(-c2ccc(N(c3ccc(-c4ccccc4)cc3)c3cccc(-c4cc(-n5c6ccccc6c6ccccc65)cc5ccccc45)c3)cc2)cc1 ; c1ccc(-c2ccc(N(c3ccccc3-c3ccccc3)c3ccccc3-c3cc(-n4c5ccccc5c5ccccc54)cc4ccccc34)cc2)cc1 ; c1ccc(-c2cccc(N(c3cccc(-c4ccccc4)c3)c3c(-n4c5ccccc5c5ccccc54)ccc4ccccc34)c2)cc1]

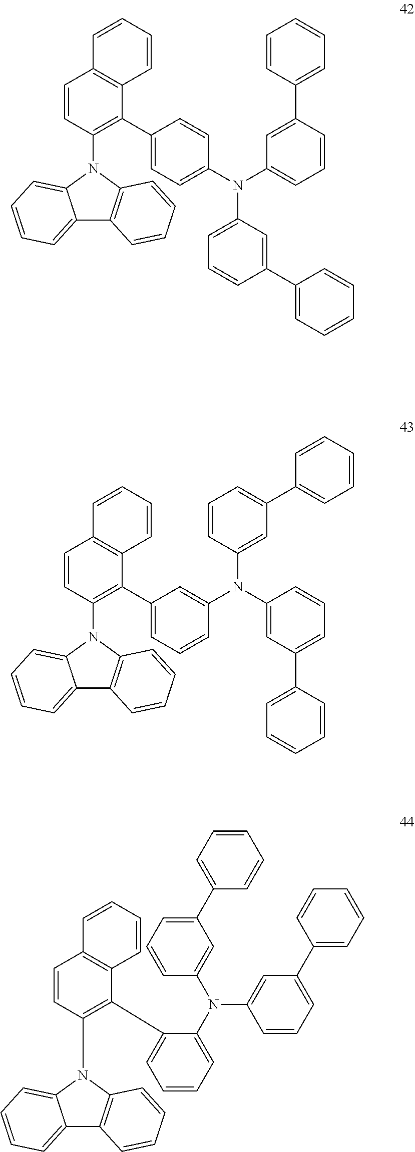 [SMILES: c1ccc(-c2cccc(N(c3ccc(-c4c(-n5c6ccccc6c6ccccc65)ccc5ccccc45)cc3)c3cccc(-c4ccccc4)c3)c2)cc1 ; c1ccc(-c2cccc(N(c3cccc(-c4ccccc4)c3)c3cccc(-c4c(-n5c6ccccc6c6ccccc65)ccc5ccccc45)c3)c2)cc1 ; c1ccc(-c2cccc(N(c3cccc(-c4ccccc4)c3)c3ccccc3-c3c(-n4c5ccccc5c5ccccc54)ccc4ccccc34)c2)cc1]

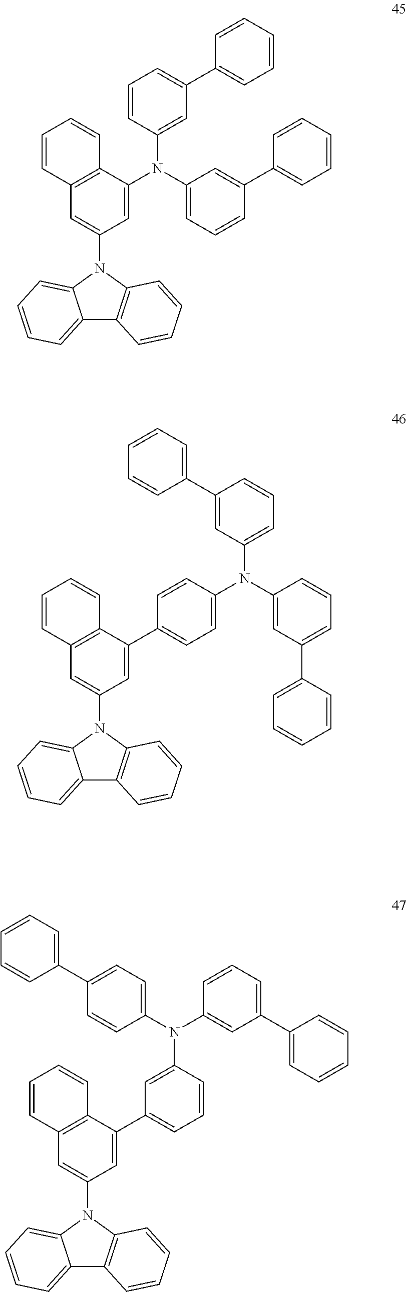 [SMILES: c1ccc(-c2cccc(N(c3cccc(-c4ccccc4)c3)c3cc(-n4c5ccccc5c5ccccc54)cc4ccccc34)c2)cc1 ; c1ccc(-c2cccc(N(c3ccc(-c4cc(-n5c6ccccc6c6ccccc65)cc5ccccc45)cc3)c3cccc(-c4ccccc4)c3)c2)cc1 ; c1ccc(-c2ccc(N(c3cccc(-c4ccccc4)c3)c3cccc(-c4cc(-n5c6ccccc6c6ccccc65)cc5ccccc45)c3)cc2)cc1]

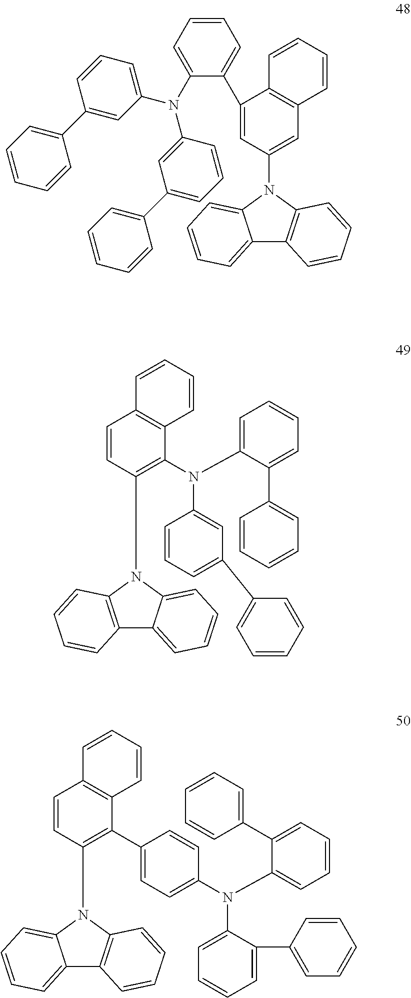 [SMILES: c1ccc(-c2cccc(N(c3cccc(-c4ccccc4)c3)c3ccccc3-c3cc(-n4c5ccccc5c5ccccc54)cc4ccccc34)c2)cc1 ; c1ccc(-c2cccc(N(c3ccccc3-c3ccccc3)c3c(-n4c5ccccc5c5ccccc54)ccc4ccccc34)c2)cc1 ; c1ccc(-c2ccccc2N(c2ccc(-c3c(-n4c5ccccc5c5ccccc54)ccc4ccccc34)cc2)c2ccccc2-c2ccccc2)cc1]

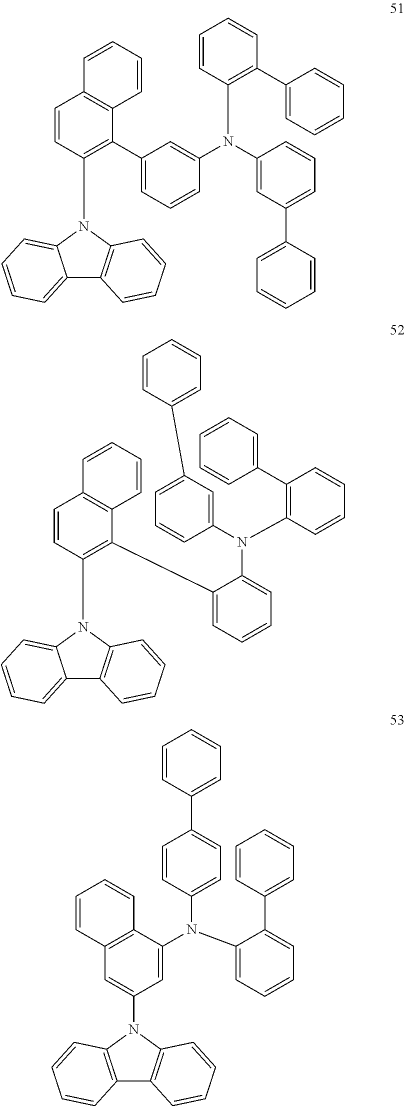 [SMILES: c1ccc(-c2cccc(N(c3cccc(-c4c(-n5c6ccccc6c6ccccc65)ccc5ccccc45)c3)c3ccccc3-c3ccccc3)c2)cc1 ; c1ccc(-c2cccc(N(c3ccccc3-c3ccccc3)c3ccccc3-c3c(-n4c5ccccc5c5ccccc54)ccc4ccccc34)c2)cc1 ; c1ccc(-c2ccc(N(c3ccccc3-c3ccccc3)c3cc(-n4c5ccccc5c5ccccc54)cc4ccccc34)cc2)cc1]

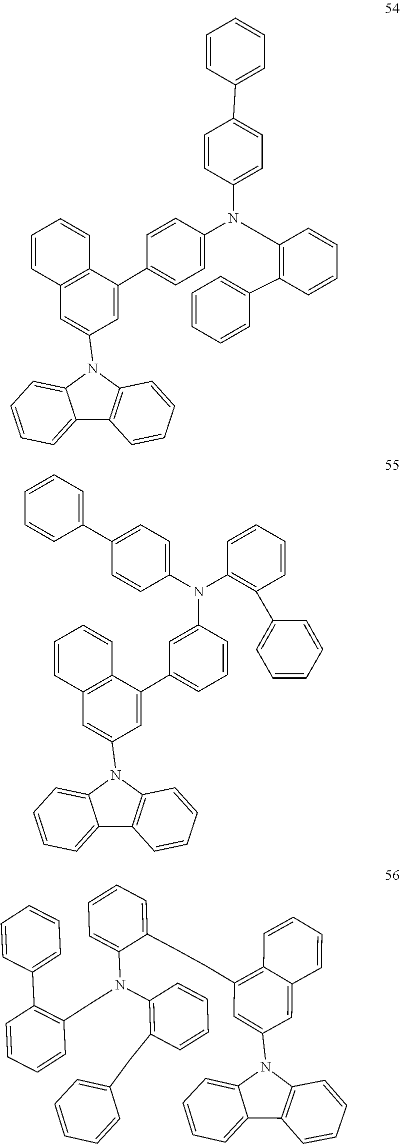

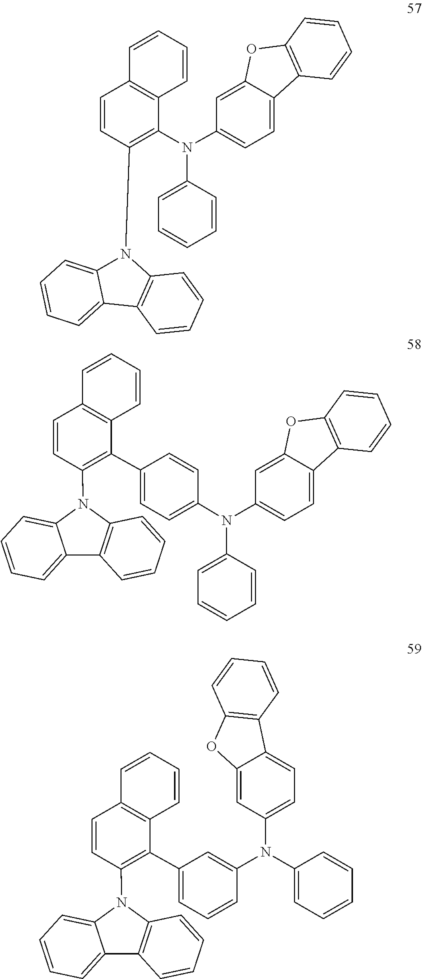

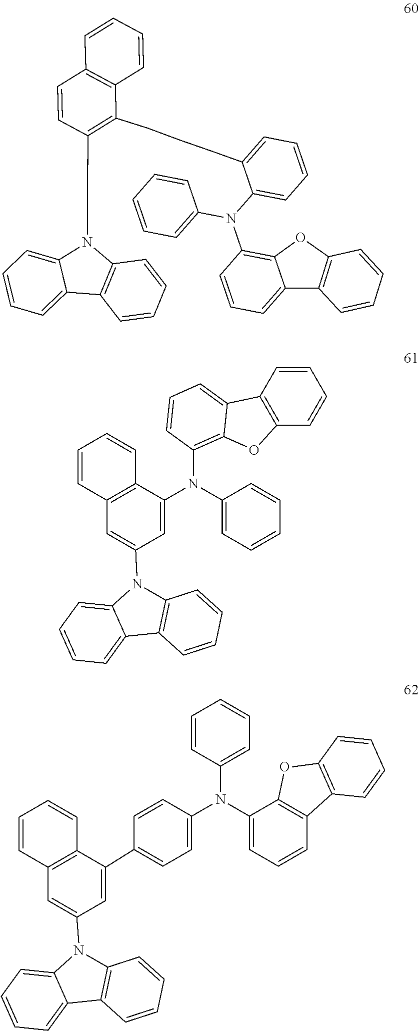 [SMILES: c1ccc(N(c2ccccc2CCCc2c(-n3c4ccccc4c4ccccc43)ccc3ccccc23)c2cccc3c2oc2ccccc23)cc1 ; c1ccc(N(c2cc(-n3c4ccccc4c4ccccc43)cc3ccccc23)c2cccc3c2oc2ccccc23)cc1 ; c1ccc(N(c2ccc(-c3cc(-n4c5ccccc5c5ccccc54)cc4ccccc34)cc2)c2cccc3c2oc2ccccc23)cc1]

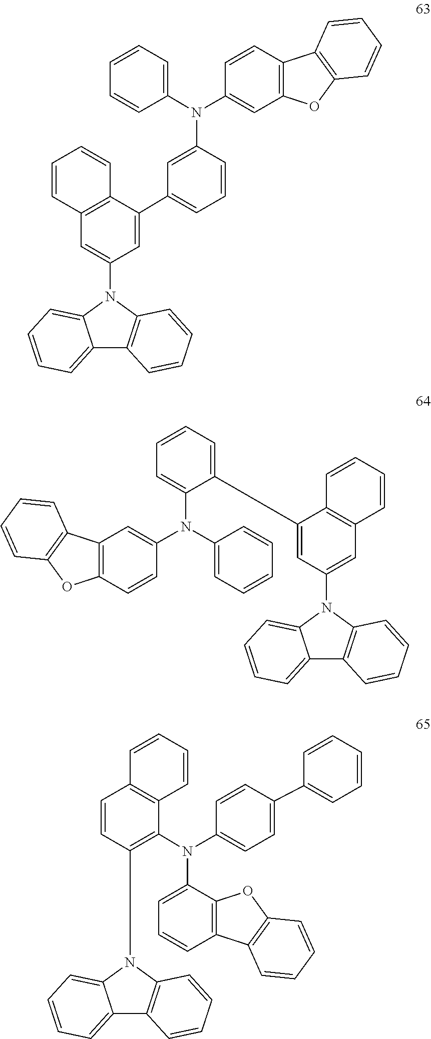 [SMILES: c1ccc(N(c2cccc(-c3cc(-n4c5ccccc5c5ccccc54)cc4ccccc34)c2)c2ccc3c(c2)oc2ccccc23)cc1 ; c1ccc(N(c2ccc3oc4ccccc4c3c2)c2ccccc2-c2cc(-n3c4ccccc4c4ccccc43)cc3ccccc23)cc1 ; c1ccc(-c2ccc(N(c3c(-n4c5ccccc5c5ccccc54)ccc4ccccc34)c3cccc4c3oc3ccccc34)cc2)cc1]

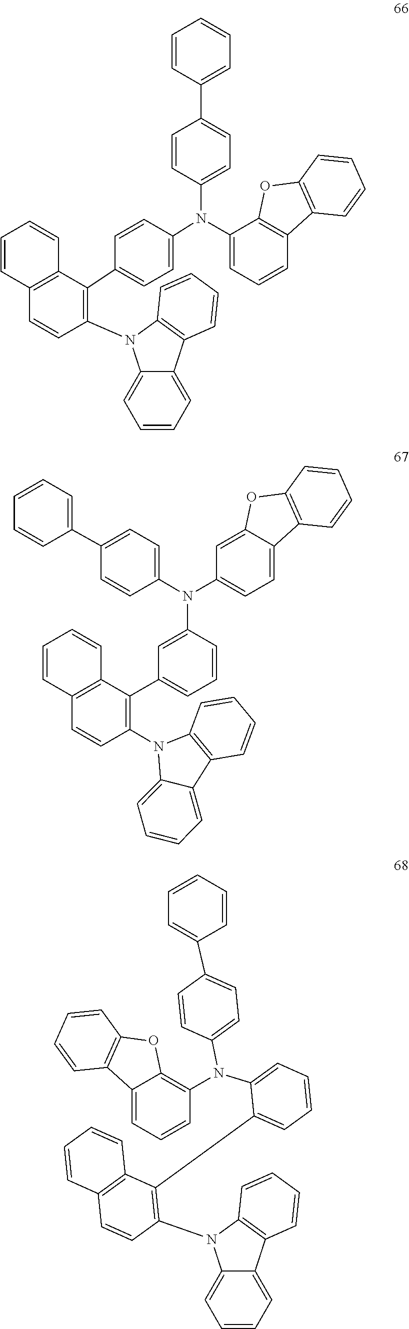

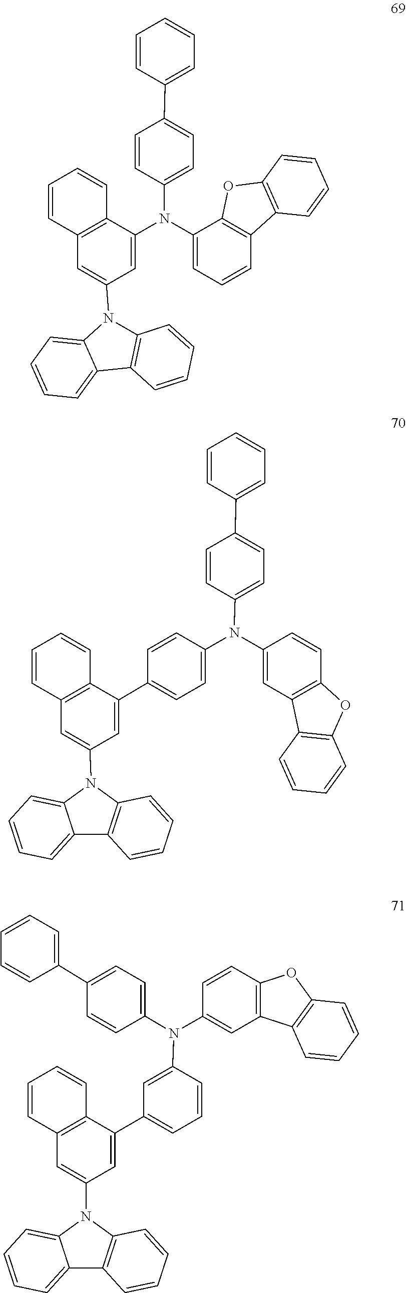 [SMILES: c1ccc(-c2ccc(N(c3cc(-n4c5ccccc5c5ccccc54)cc4ccccc34)c3cccc4c3oc3ccccc34)cc2)cc1 ; c1ccc(-c2ccc(N(c3ccc(-c4cc(-n5c6ccccc6c6ccccc65)cc5ccccc45)cc3)c3ccc4oc5ccccc5c4c3)cc2)cc1 ; c1ccc(-c2ccc(N(c3cccc(-c4cc(-n5c6ccccc6c6ccccc65)cc5ccccc45)c3)c3ccc4oc5ccccc5c4c3)cc2)cc1]

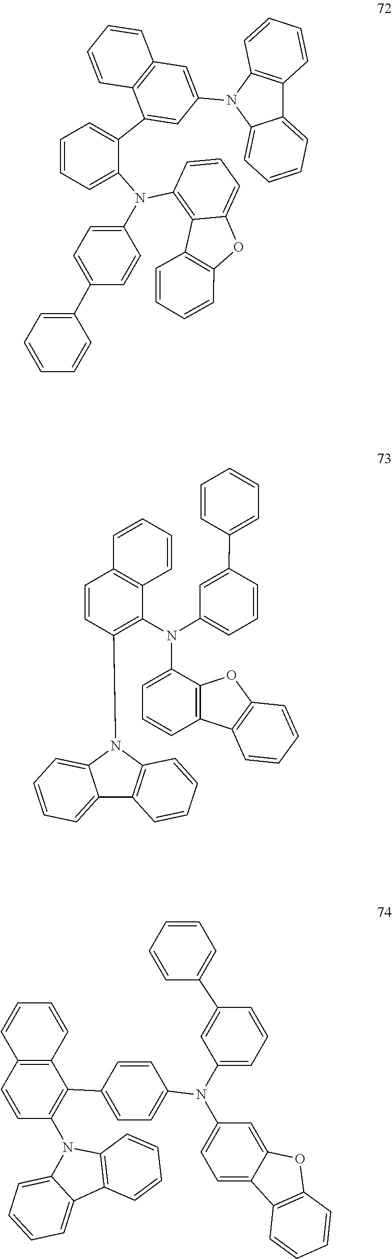 [SMILES: c1ccc(-c2ccc(N(c3ccccc3-c3cc(-n4c5ccccc5c5ccccc54)cc4ccccc34)c3cccc4oc5ccccc5c34)cc2)cc1 ; c1ccc(-c2cccc(N(c3c(-n4c5ccccc5c5ccccc54)ccc4ccccc34)c3cccc4c3oc3ccccc34)c2)cc1 ; c1ccc(-c2cccc(N(c3ccc(-c4c(-n5c6ccccc6c6ccccc65)ccc5ccccc45)cc3)c3ccc4c(c3)oc3ccccc34)c2)cc1]

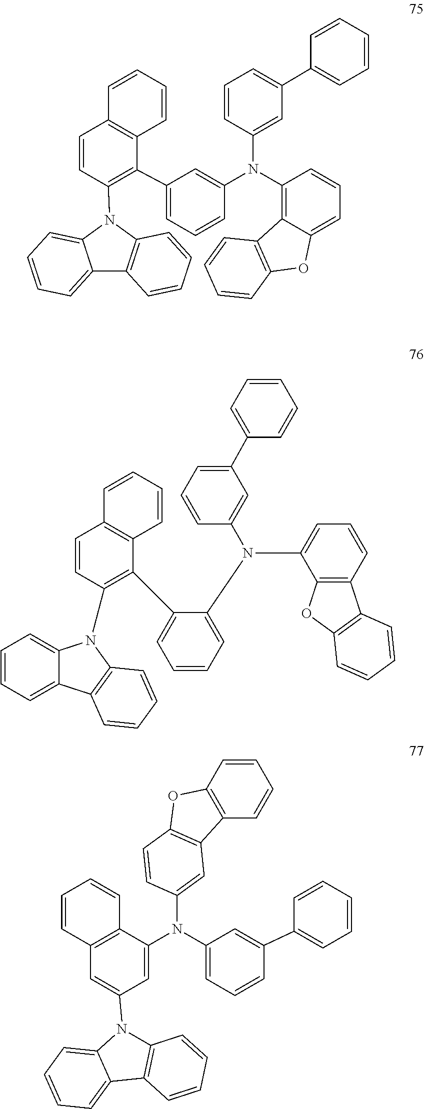 [SMILES: c1ccc(-c2cccc(N(c3cccc(-c4c(-n5c6ccccc6c6ccccc65)ccc5ccccc45)c3)c3cccc4oc5ccccc5c34)c2)cc1 ; c1ccc(-c2cccc(N(c3ccccc3-c3c(-n4c5ccccc5c5ccccc54)ccc4ccccc34)c3cccc4c3oc3ccccc34)c2)cc1 ; c1ccc(-c2cccc(N(c3ccc4oc5ccccc5c4c3)c3cc(-n4c5ccccc5c5ccccc54)cc4ccccc34)c2)cc1]

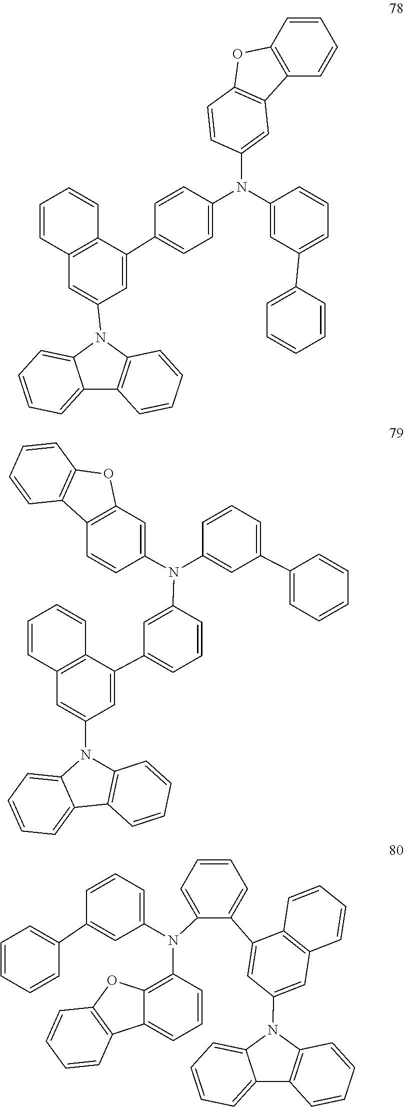

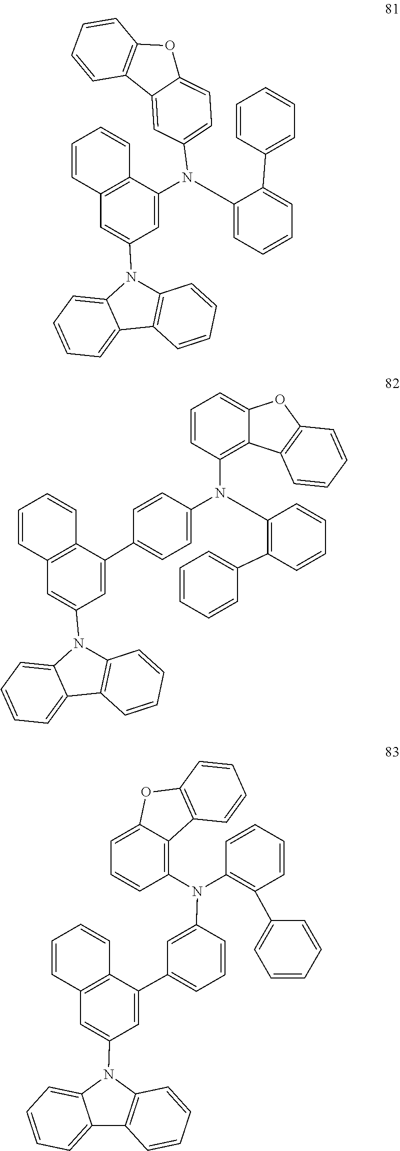 [SMILES: c1ccc(-c2ccccc2N(c2ccc3oc4ccccc4c3c2)c2cc(-n3c4ccccc4c4ccccc43)cc3ccccc23)cc1 ; c1ccc(-c2ccccc2N(c2ccc(-c3cc(-n4c5ccccc5c5ccccc54)cc4ccccc34)cc2)c2cccc3oc4ccccc4c23)cc1 ; c1ccc(-c2ccccc2N(c2cccc(-c3cc(-n4c5ccccc5c5ccccc54)cc4ccccc34)c2)c2cccc3oc4ccccc4c23)cc1]

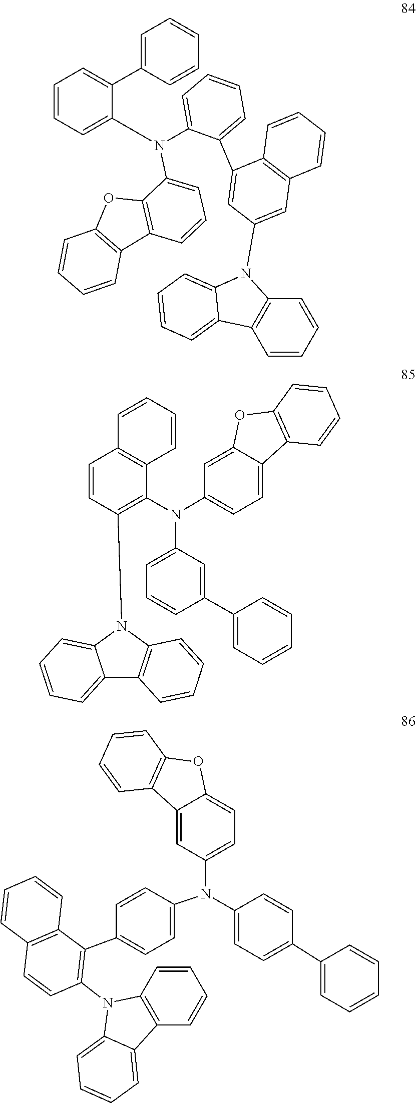

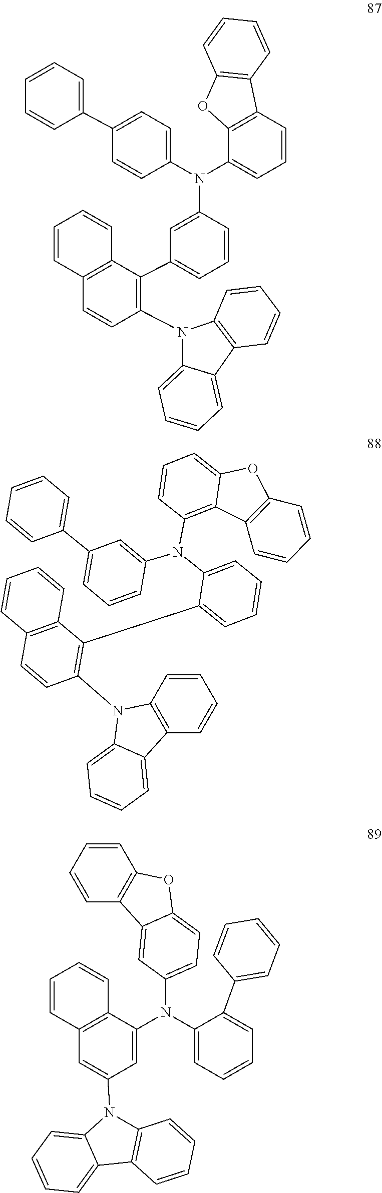

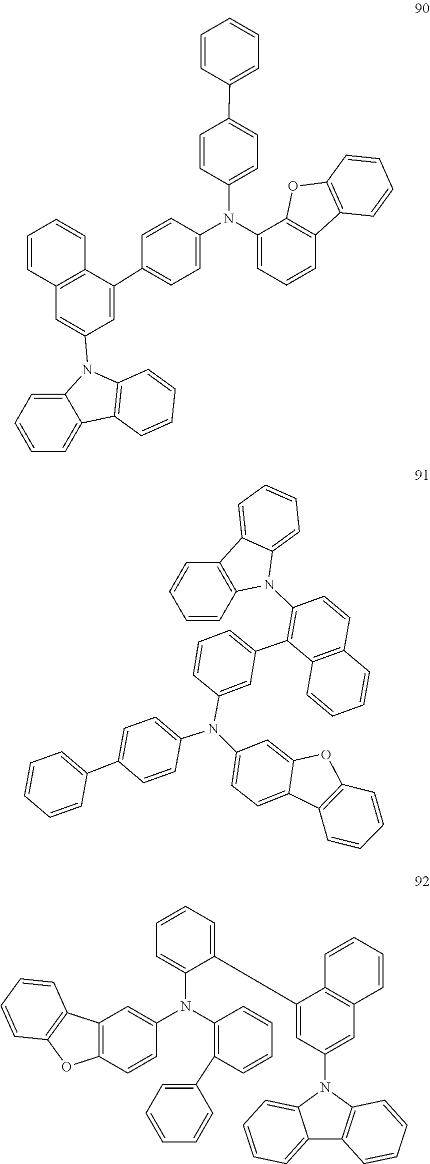 [SMILES: c1ccc(-c2ccc(N(c3ccc(-c4cc(-n5c6ccccc6c6ccccc65)cc5ccccc45)cc3)c3cccc4c3oc3ccccc34)cc2)cc1 ; c1ccc(-c2ccc(N(c3cccc(-c4c(-n5c6ccccc6c6ccccc65)ccc5ccccc45)c3)c3ccc4c(c3)oc3ccccc34)cc2)cc1 ; c1ccc(-c2ccccc2N(c2ccc3oc4ccccc4c3c2)c2ccccc2-c2cc(-n3c4ccccc4c4ccccc43)cc3ccccc23)cc1]

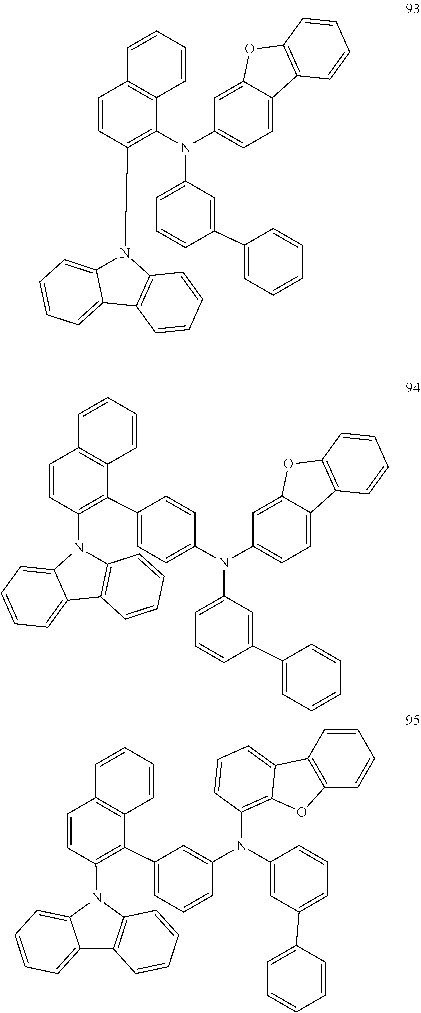

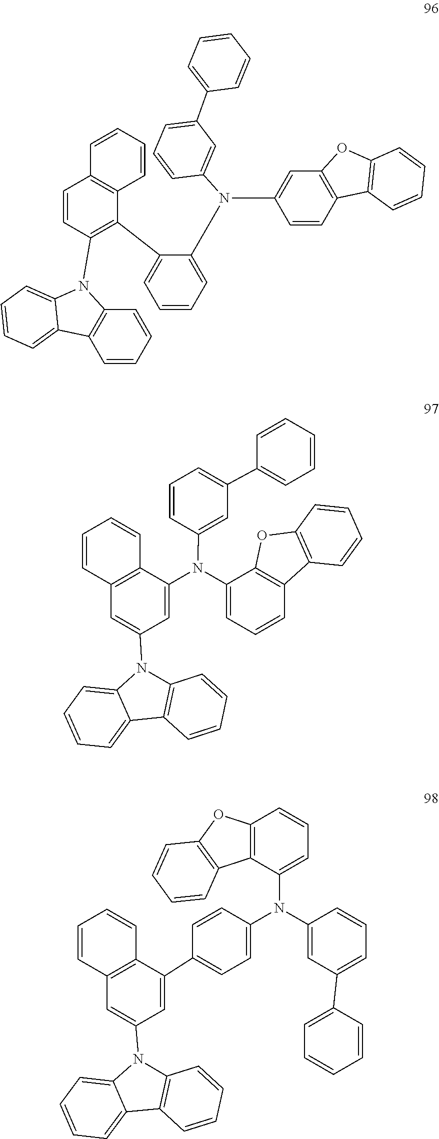 [SMILES: c1ccc(-c2cccc(N(c3ccc4c(c3)oc3ccccc34)c3ccccc3-c3c(-n4c5ccccc5c5ccccc54)ccc4ccccc34)c2)cc1 ; c1ccc(-c2cccc(N(c3cc(-n4c5ccccc5c5ccccc54)cc4ccccc34)c3cccc4c3oc3ccccc34)c2)cc1 ; c1ccc(-c2cccc(N(c3ccc(-c4cc(-n5c6ccccc6c6ccccc65)cc5ccccc45)cc3)c3cccc4oc5ccccc5c34)c2)cc1]

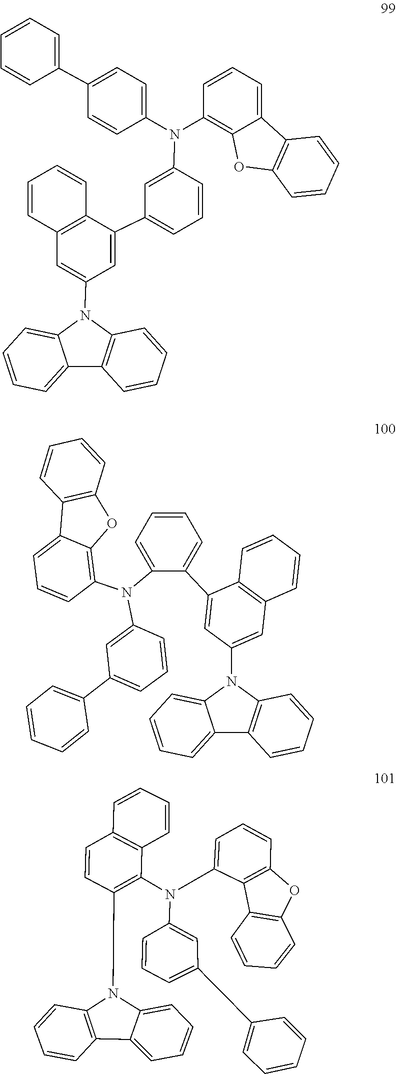

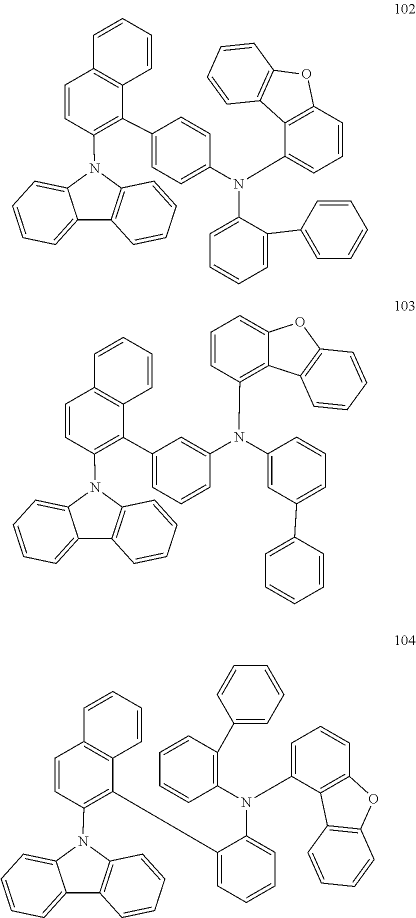

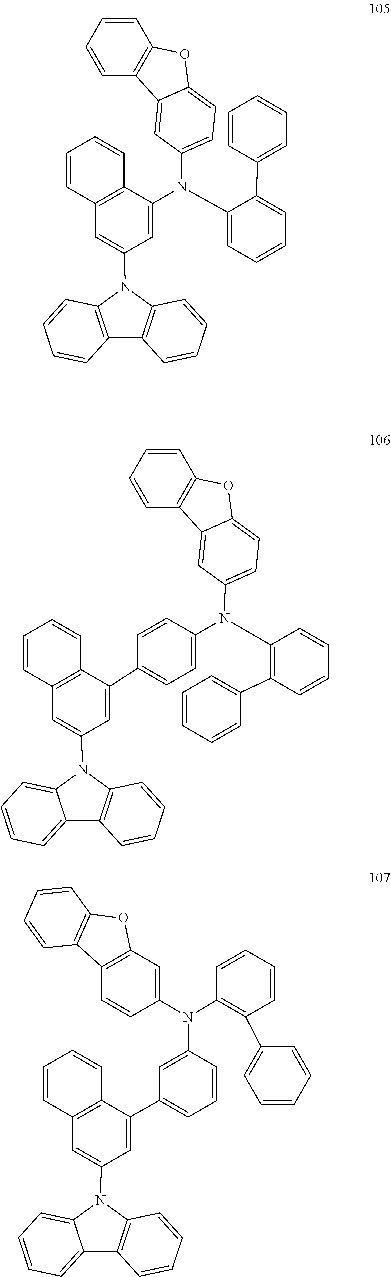

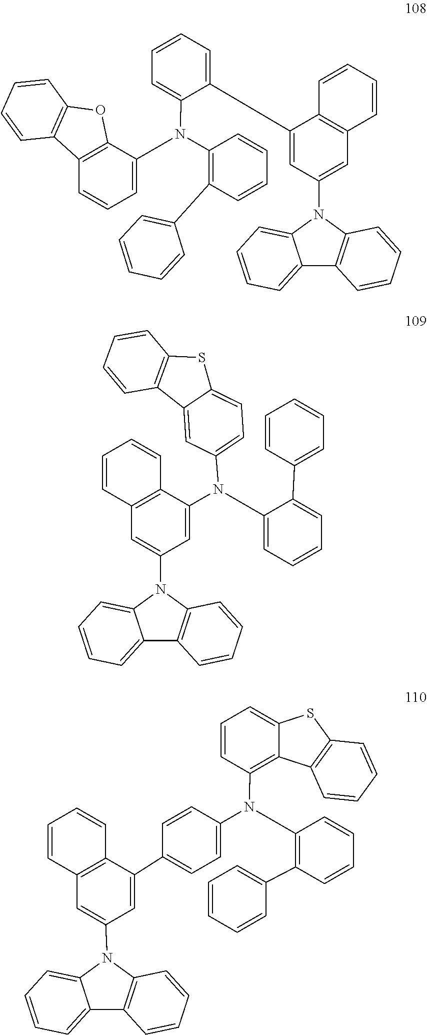 [SMILES: c1ccc(-c2ccccc2N(c2ccccc2-c2cc(-n3c4ccccc4c4ccccc43)cc3ccccc23)c2cccc3c2oc2ccccc23)cc1 ; c1ccc(-c2ccccc2N(c2ccc3sc4ccccc4c3c2)c2cc(-n3c4ccccc4c4ccccc43)cc3ccccc23)cc1 ; c1ccc(-c2ccccc2N(c2ccc(-c3cc(-n4c5ccccc5c5ccccc54)cc4ccccc34)cc2)c2cccc3sc4ccccc4c23)cc1]

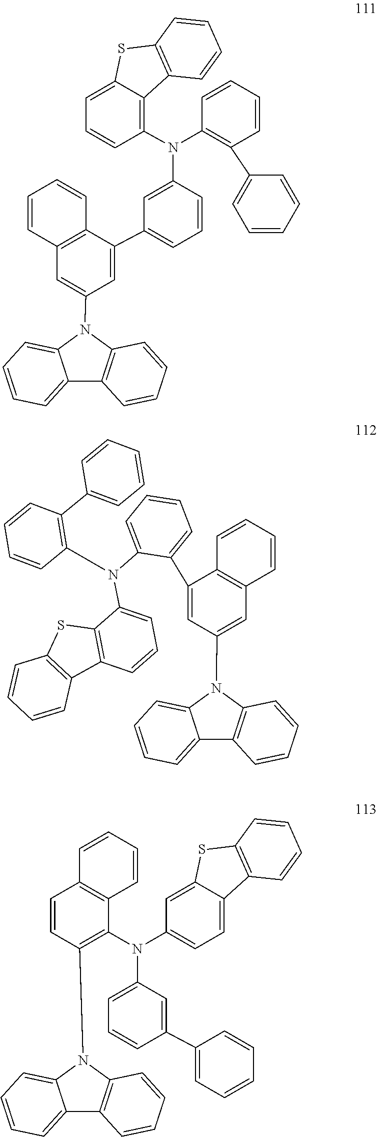

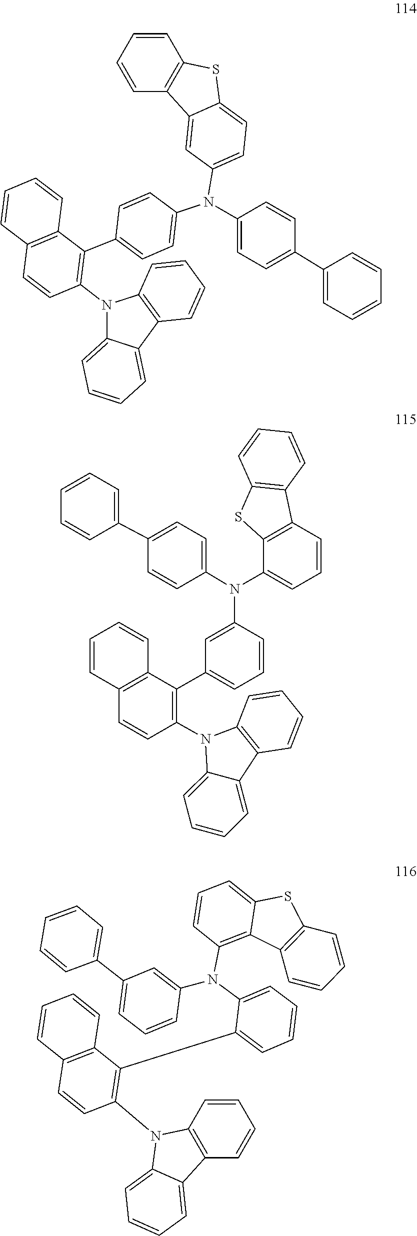

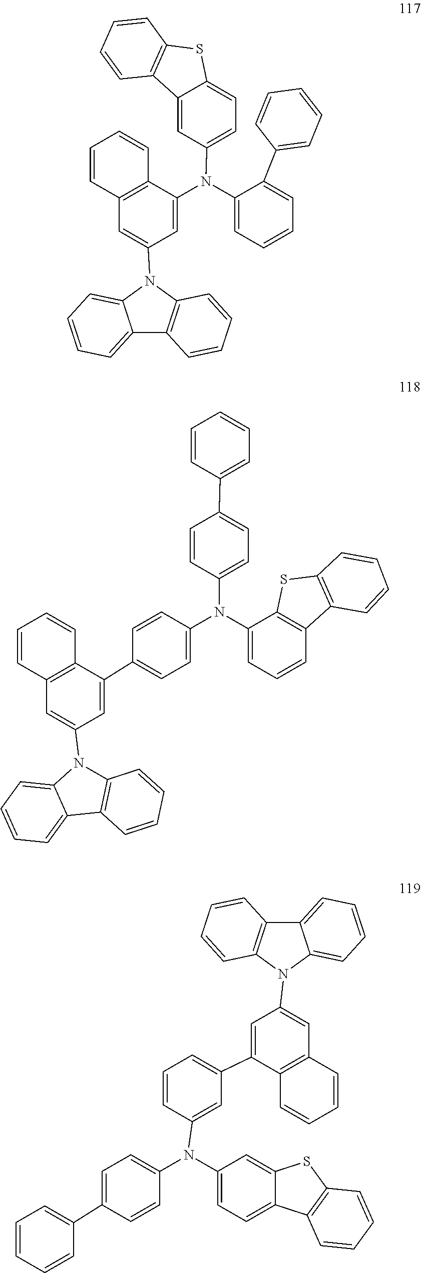 [SMILES: c1ccc(-c2ccccc2N(c2ccc3sc4ccccc4c3c2)c2cc(-n3c4ccccc4c4ccccc43)cc3ccccc23)cc1 ; c1ccc(-c2ccc(N(c3ccc(-c4cc(-n5c6ccccc6c6ccccc65)cc5ccccc45)cc3)c3cccc4c3sc3ccccc34)cc2)cc1 ; c1ccc(-c2ccc(N(c3cccc(-c4cc(-n5c6ccccc6c6ccccc65)cc5ccccc45)c3)c3ccc4c(c3)sc3ccccc34)cc2)cc1]

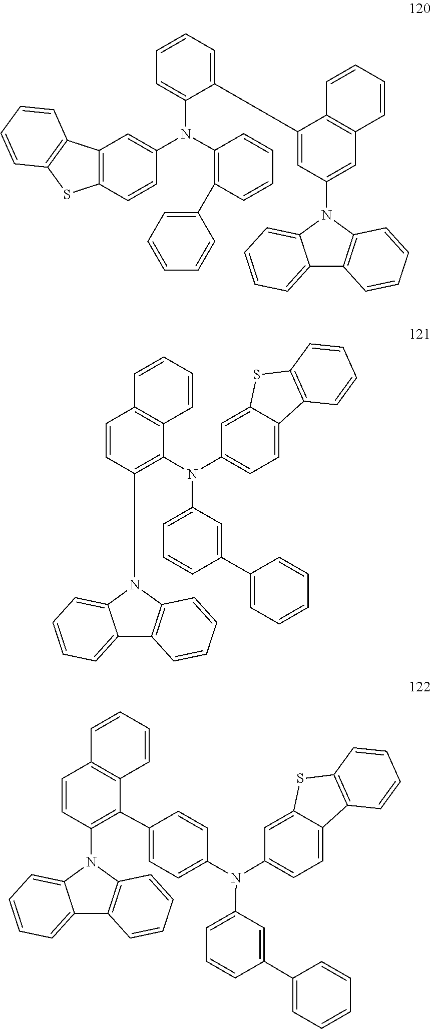 [SMILES: c1ccc(-c2ccccc2N(c2ccc3sc4ccccc4c3c2)c2ccccc2-c2cc(-n3c4ccccc4c4ccccc43)cc3ccccc23)cc1 ; c1ccc(-c2cccc(N(c3ccc4c(c3)sc3ccccc34)c3c(-n4c5ccccc5c5ccccc54)ccc4ccccc34)c2)cc1 ; c1ccc(-c2cccc(N(c3ccc(-c4c(-n5c6ccccc6c6ccccc65)ccc5ccccc45)cc3)c3ccc4c(c3)sc3ccccc34)c2)cc1]

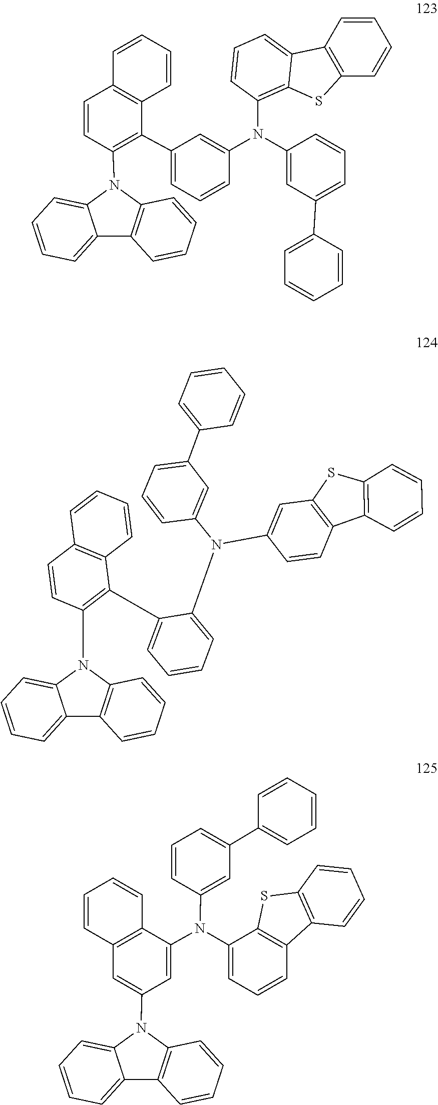 [SMILES: c1ccc(-c2cccc(N(c3cccc(-c4c(-n5c6ccccc6c6ccccc65)ccc5ccccc45)c3)c3cccc4c3sc3ccccc34)c2)cc1 ; c1ccc(-c2cccc(N(c3ccc4c(c3)sc3ccccc34)c3ccccc3-c3c(-n4c5ccccc5c5ccccc54)ccc4ccccc34)c2)cc1 ; c1ccc(-c2cccc(N(c3cc(-n4c5ccccc5c5ccccc54)cc4ccccc34)c3cccc4c3sc3ccccc34)c2)cc1]

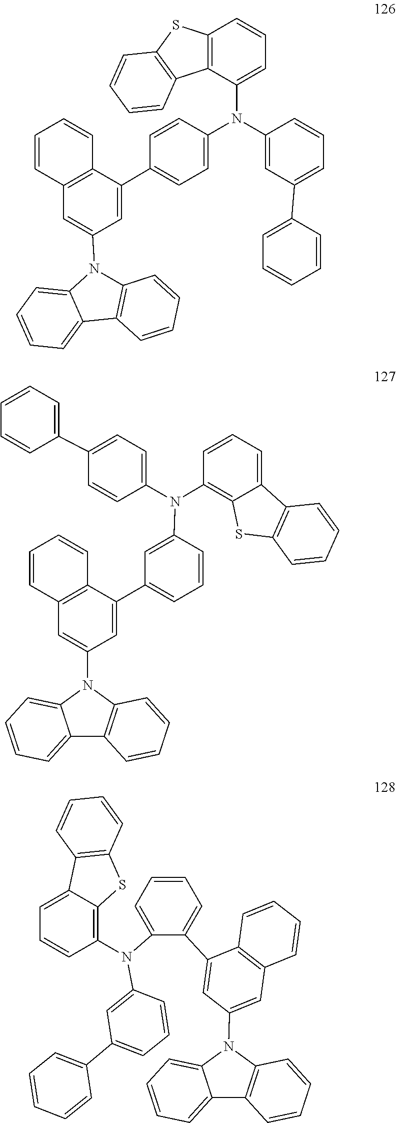

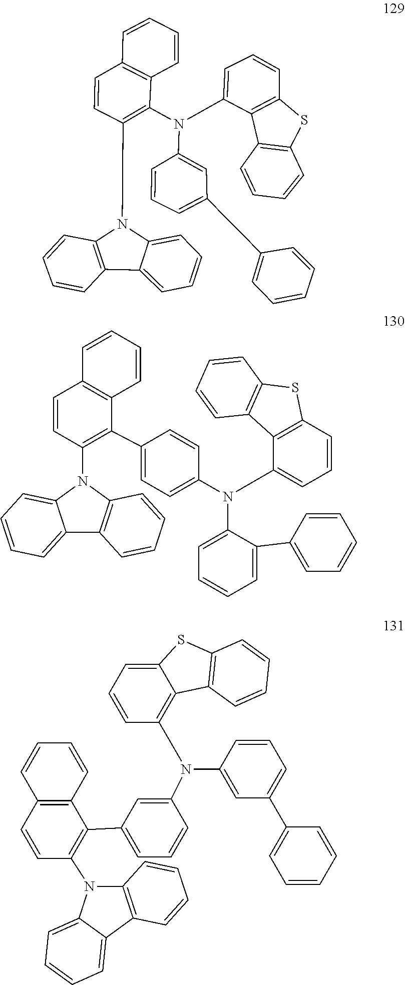

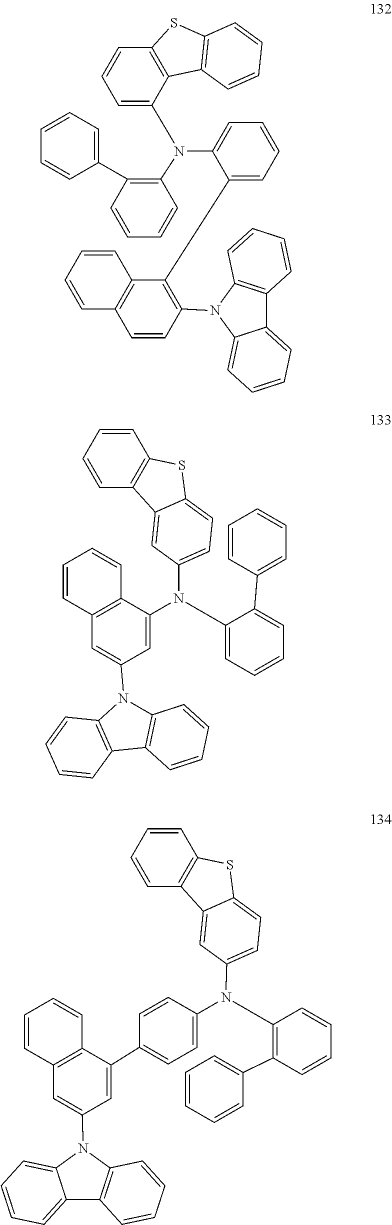 [SMILES: c1ccc(-c2ccccc2N(c2ccccc2-c2c(-n3c4ccccc4c4ccccc43)ccc3ccccc23)c2cccc3sc4ccccc4c23)cc1 ; c1ccc(-c2ccccc2N(c2ccc3sc4ccccc4c3c2)c2cc(-n3c4ccccc4c4ccccc43)cc3ccccc23)cc1 ; c1ccc(-c2ccccc2N(c2ccc(-c3cc(-n4c5ccccc5c5ccccc54)cc4ccccc34)cc2)c2ccc3sc4ccccc4c3c2)cc1]

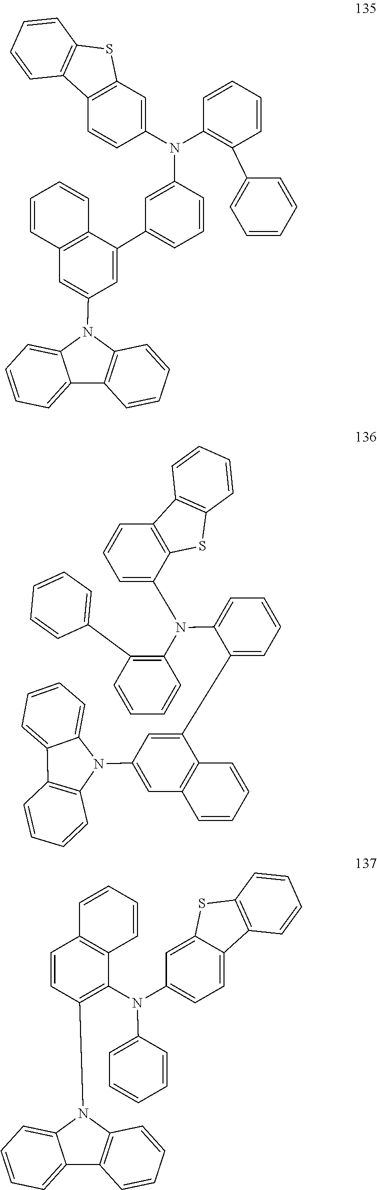 [SMILES: c1ccc(-c2ccccc2N(c2cccc(-c3cc(-n4c5ccccc5c5ccccc54)cc4ccccc34)c2)c2ccc3c(c2)sc2ccccc23)cc1 ; c1ccc(-c2ccccc2N(c2ccccc2-c2cc(-n3c4ccccc4c4ccccc43)cc3ccccc23)c2cccc3c2sc2ccccc23)cc1 ; c1ccc(N(c2ccc3c(c2)sc2ccccc23)c2c(-n3c4ccccc4c4ccccc43)ccc3ccccc23)cc1]

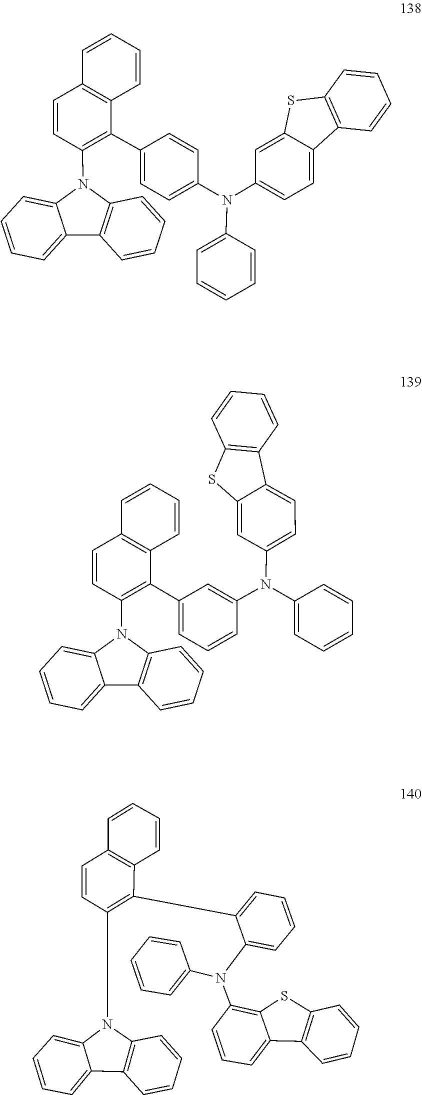 [SMILES: c1ccc(N(c2ccc(-c3c(-n4c5ccccc5c5ccccc54)ccc4ccccc34)cc2)c2ccc3c(c2)sc2ccccc23)cc1 ; c1ccc(N(c2cccc(-c3c(-n4c5ccccc5c5ccccc54)ccc4ccccc34)c2)c2ccc3c(c2)sc2ccccc23)cc1 ; c1ccc(N(c2ccccc2-c2c(-n3c4ccccc4c4ccccc43)ccc3ccccc23)c2cccc3c2sc2ccccc23)cc1]

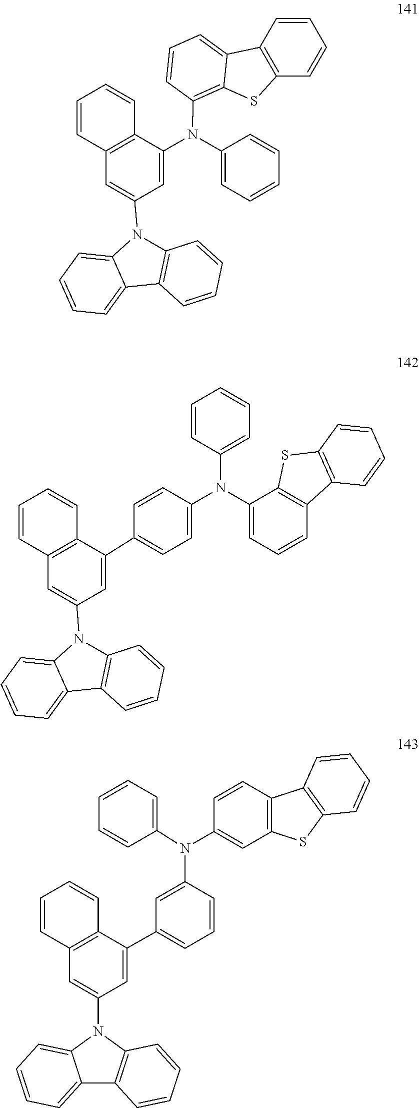 [SMILES: c1ccc(N(c2cc(-n3c4ccccc4c4ccccc43)cc3ccccc23)c2cccc3c2sc2ccccc23)cc1 ; c1ccc(N(c2ccc(-c3cc(-n4c5ccccc5c5ccccc54)cc4ccccc34)cc2)c2cccc3c2sc2ccccc23)cc1 ; c1ccc(N(c2cccc(-c3cc(-n4c5ccccc5c5ccccc54)cc4ccccc34)c2)c2ccc3c(c2)sc2ccccc23)cc1]

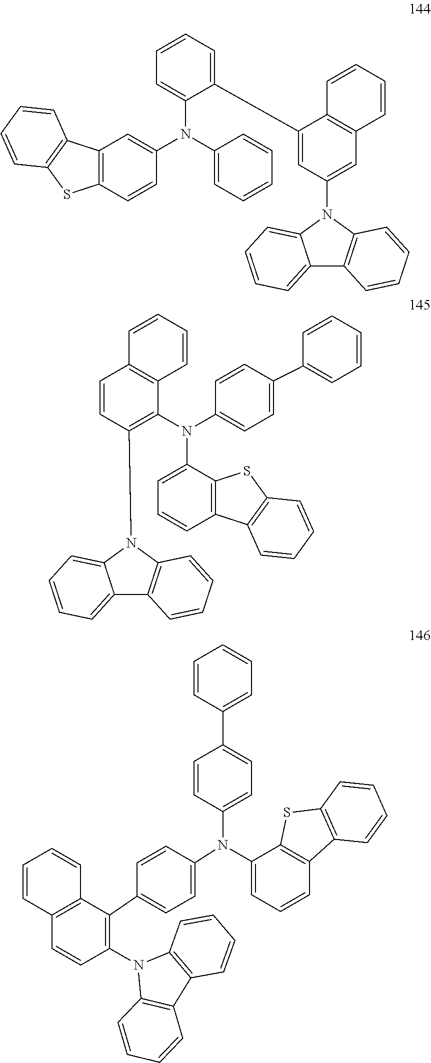

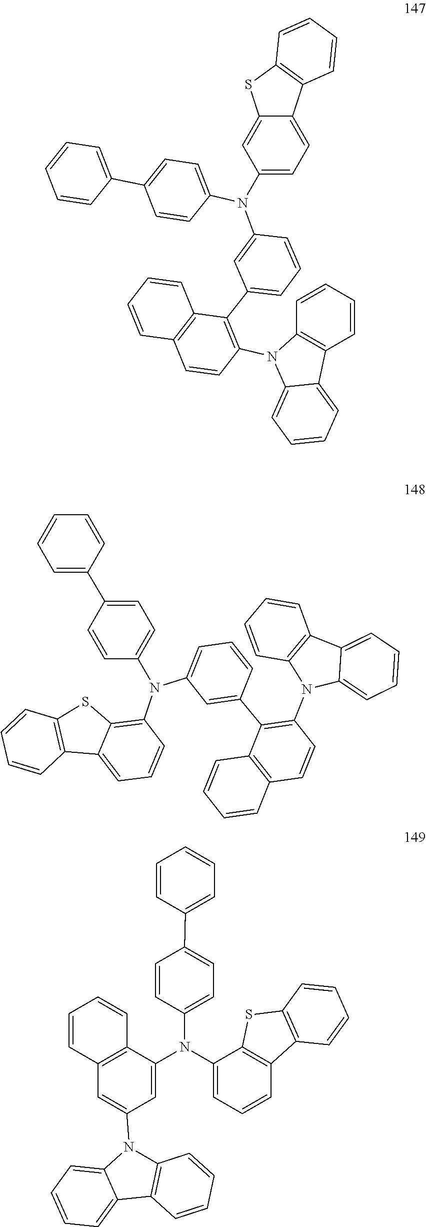 [SMILES: c1ccc(-c2ccc(N(c3cccc(-c4c(-n5c6ccccc6c6ccccc65)ccc5ccccc45)c3)c3ccc4c(c3)sc3ccccc34)cc2)cc1 ; c1ccc(-c2ccc(N(c3cccc(-c4c(-n5c6ccccc6c6ccccc65)ccc5ccccc45)c3)c3cccc4c3sc3ccccc34)cc2)cc1 ; c1ccc(-c2ccc(N(c3cc(-n4c5ccccc5c5ccccc54)cc4ccccc34)c3cccc4c3sc3ccccc34)cc2)cc1]

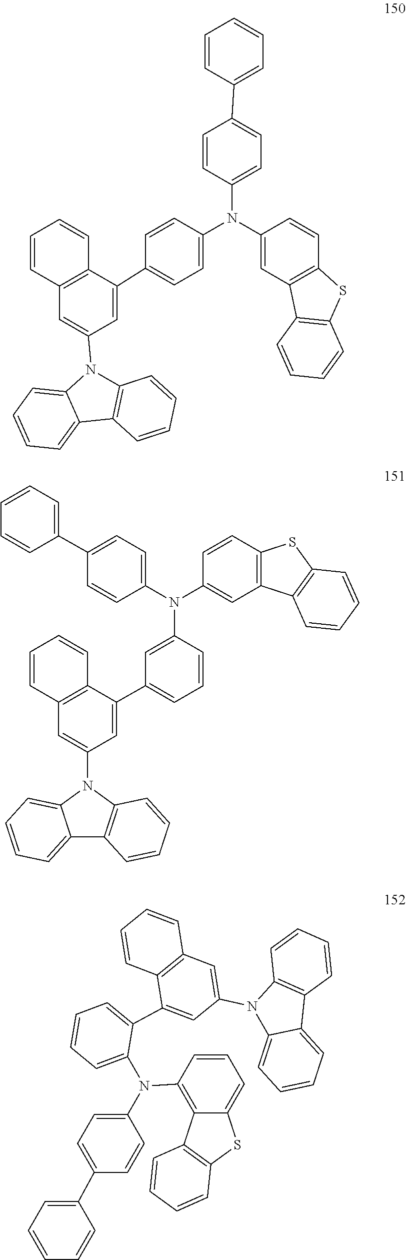

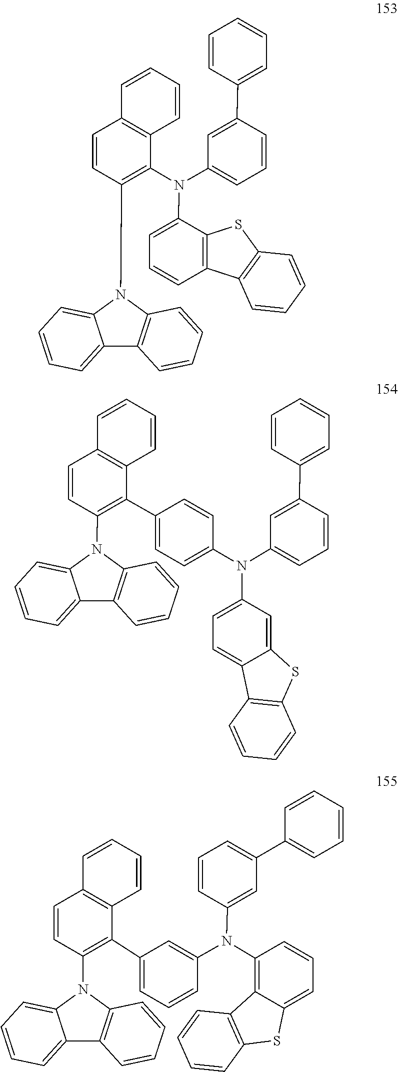

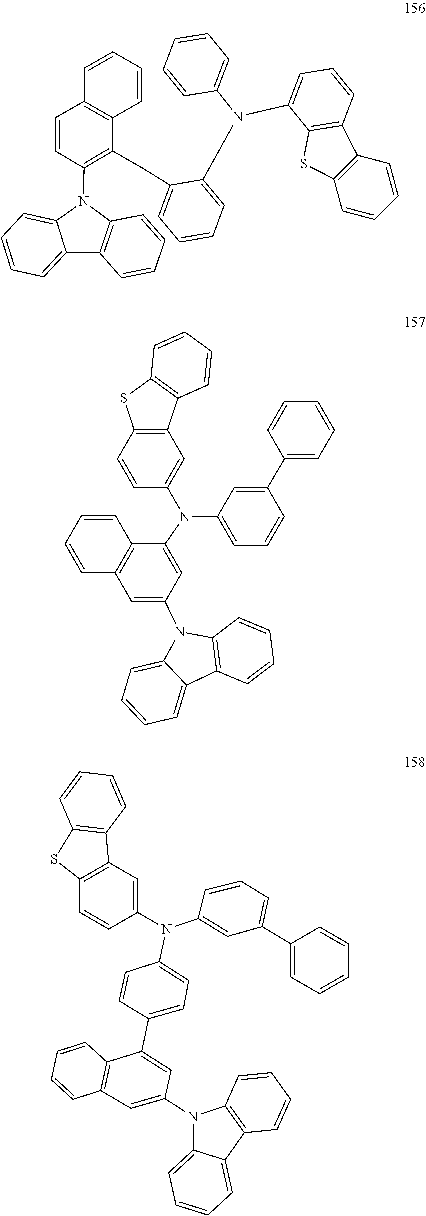 [SMILES: c1ccc(N(c2ccccc2Cc2c(-n3c4ccccc4c4ccccc43)ccc3ccccc23)c2cccc3c2sc2ccccc23)cc1 ; c1ccc(-c2cccc(N(c3ccc4sc5ccccc5c4c3)c3cc(-n4c5ccccc5c5ccccc54)cc4ccccc34)c2)cc1 ; c1ccc(-c2cccc(N(c3ccc(-c4cc(-n5c6ccccc6c6ccccc65)cc5ccccc45)cc3)c3ccc4sc5ccccc5c4c3)c2)cc1]

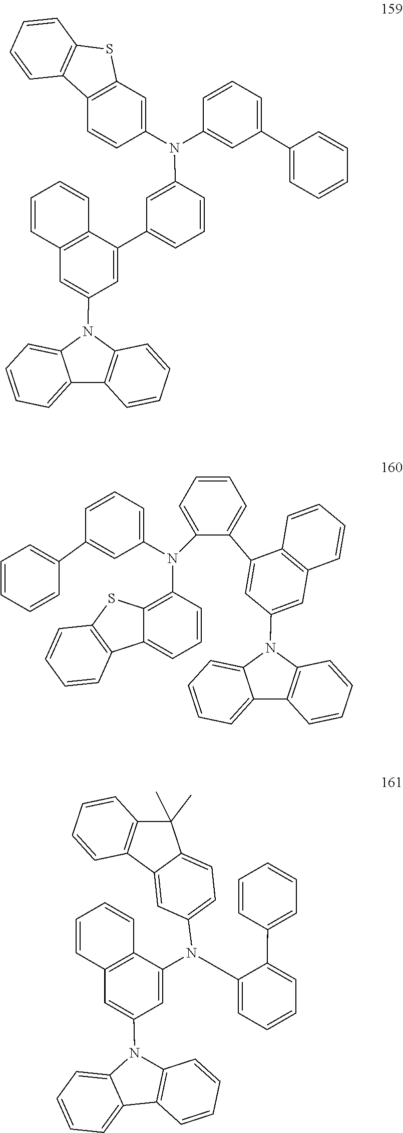 [SMILES: c1ccc(-c2cccc(N(c3cccc(-c4cc(-n5c6ccccc6c6ccccc65)cc5ccccc45)c3)c3ccc4c(c3)sc3ccccc34)c2)cc1 ; c1ccc(-c2cccc(N(c3ccccc3-c3cc(-n4c5ccccc5c5ccccc54)cc4ccccc34)c3cccc4c3sc3ccccc34)c2)cc1 ; CC1(C)c2ccccc2-c2cc(N(c3ccccc3-c3ccccc3)c3cc(-n4c5ccccc5c5ccccc54)cc4ccccc34)ccc21]

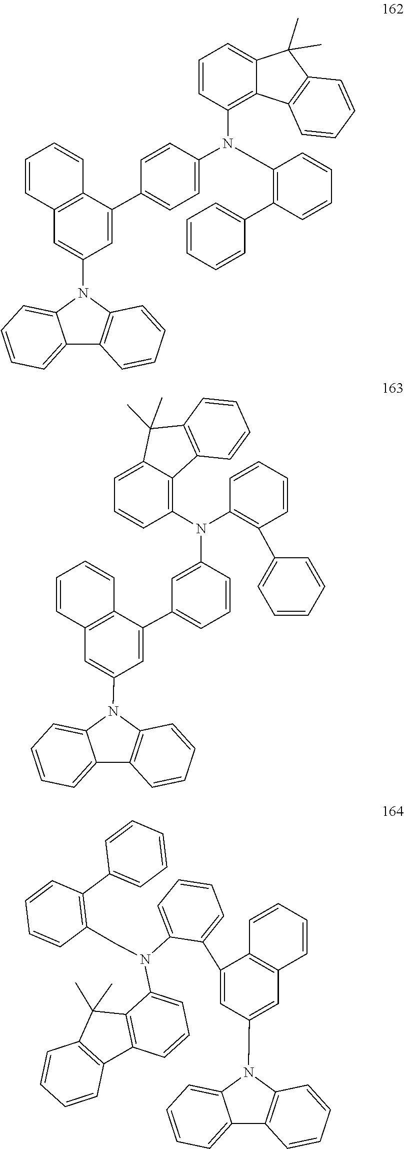

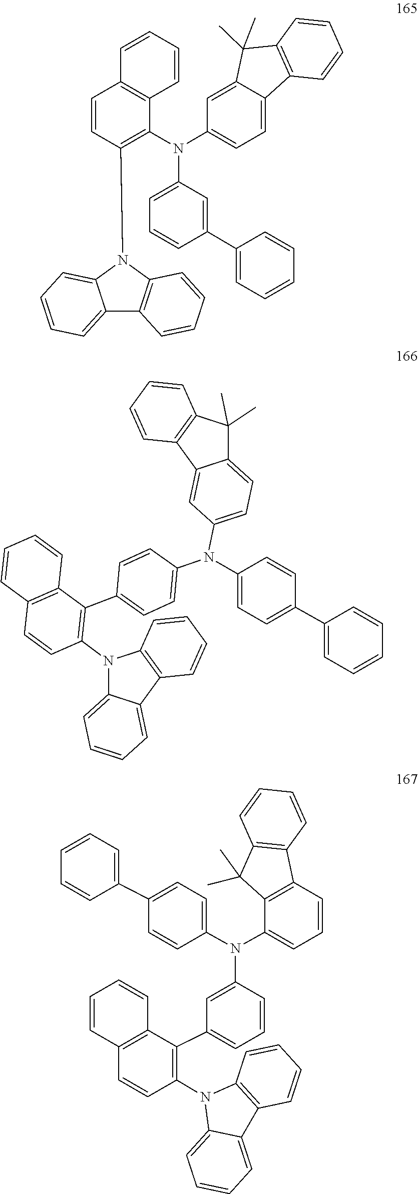 [SMILES: CC1(C)c2ccccc2-c2ccc(N(c3cccc(-c4ccccc4)c3)c3c(-n4c5ccccc5c5ccccc54)ccc4ccccc34)cc21 ; CC1(C)c2ccccc2-c2cc(N(c3ccc(-c4ccccc4)cc3)c3ccc(-c4c(-n5c6ccccc6c6ccccc65)ccc5ccccc45)cc3)ccc21 ; CC1(C)c2ccccc2-c2cccc(N(c3ccc(-c4ccccc4)cc3)c3cccc(-c4c(-n5c6ccccc6c6ccccc65)ccc5ccccc45)c3)c21]

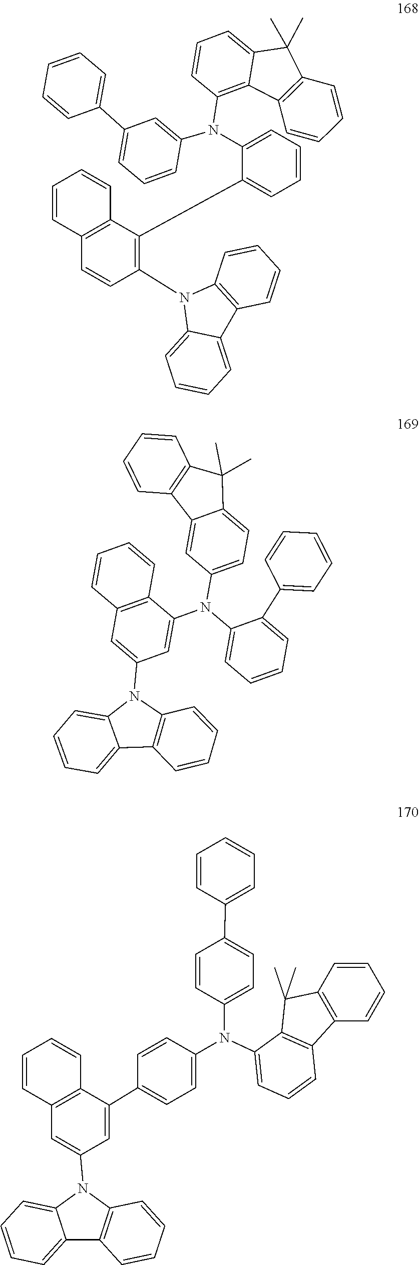 [SMILES: CC1(C)c2ccccc2-c2c(N(c3cccc(-c4ccccc4)c3)c3ccccc3-c3c(-n4c5ccccc5c5ccccc54)ccc4ccccc34)cccc21 ; CC1(C)c2ccccc2-c2cc(N(c3ccccc3-c3ccccc3)c3cc(-n4c5ccccc5c5ccccc54)cc4ccccc34)ccc21 ; CC1(C)c2ccccc2-c2cccc(N(c3ccc(-c4ccccc4)cc3)c3ccc(-c4cc(-n5c6ccccc6c6ccccc65)cc5ccccc45)cc3)c21]

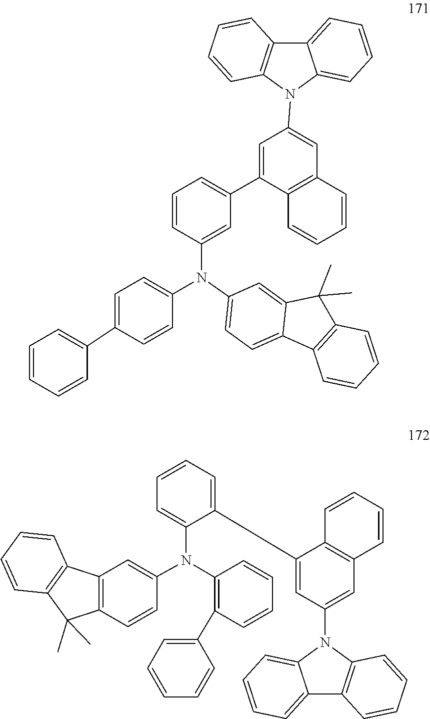 [SMILES: CC1(C)c2ccccc2-c2ccc(N(c3ccc(-c4ccccc4)cc3)c3cccc(-c4cc(-n5c6ccccc6c6ccccc65)cc5ccccc45)c3)cc21 ; CC1(C)c2ccccc2-c2cc(N(c3ccccc3-c3ccccc3)c3ccccc3-c3cc(-n4c5ccccc5c5ccccc54)cc4ccccc34)ccc21]

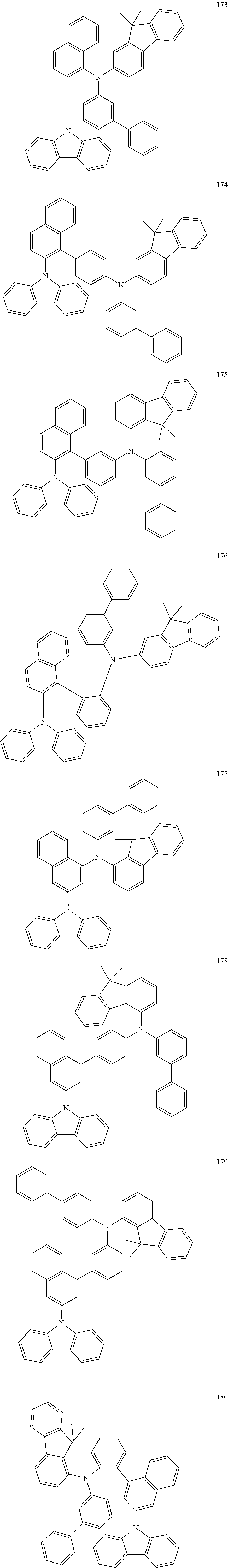

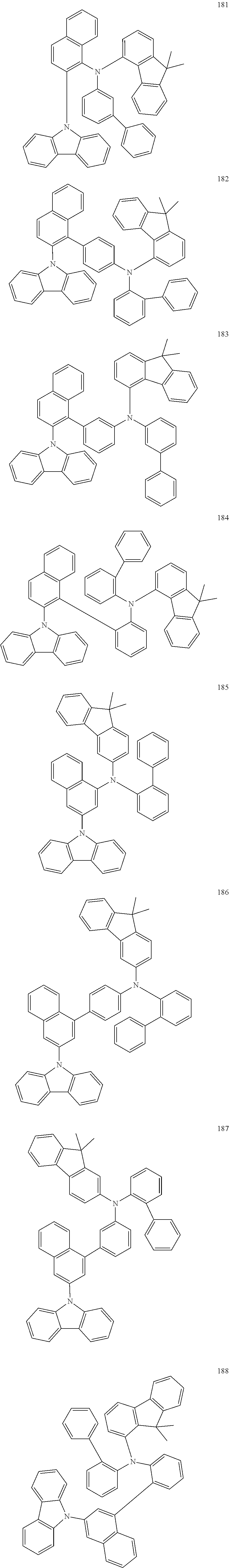

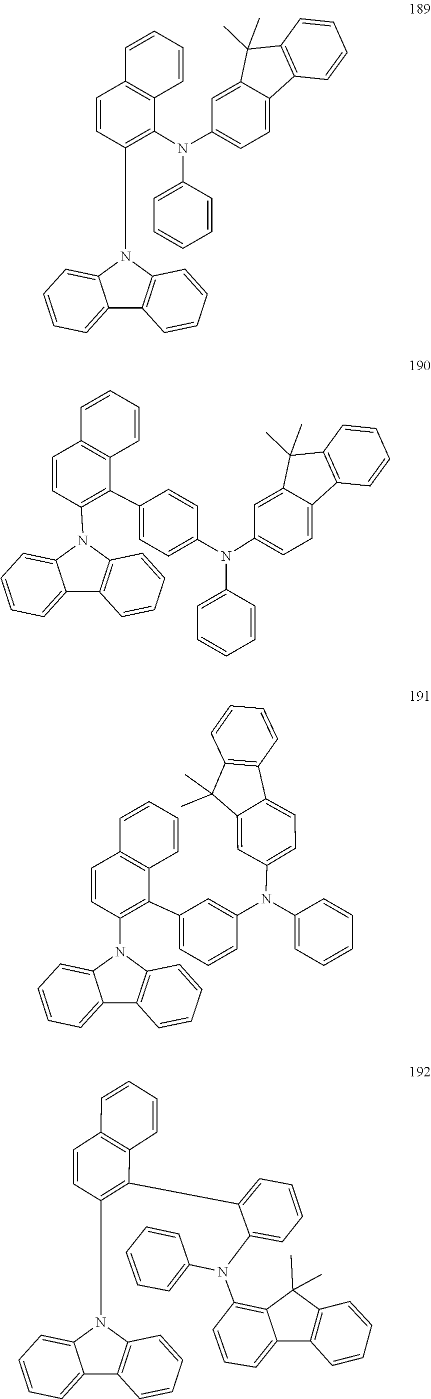 [SMILES: CC1(C)c2ccccc2-c2ccc(N(c3ccccc3)c3c(-n4c5ccccc5c5ccccc54)ccc4ccccc34)cc21 ; CC1(C)c2ccccc2-c2ccc(N(c3ccccc3)c3ccc(-c4c(-n5c6ccccc6c6ccccc65)ccc5ccccc45)cc3)cc21 ; CC1(C)c2ccccc2-c2ccc(N(c3ccccc3)c3cccc(-c4c(-n5c6ccccc6c6ccccc65)ccc5ccccc45)c3)cc21 ; CC1(C)c2ccccc2-c2cccc(N(c3ccccc3)c3ccccc3-c3c(-n4c5ccccc5c5ccccc54)ccc4ccccc34)c21]

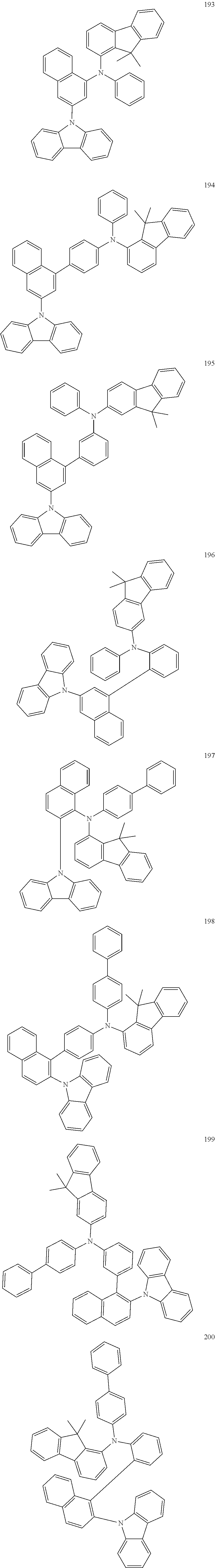

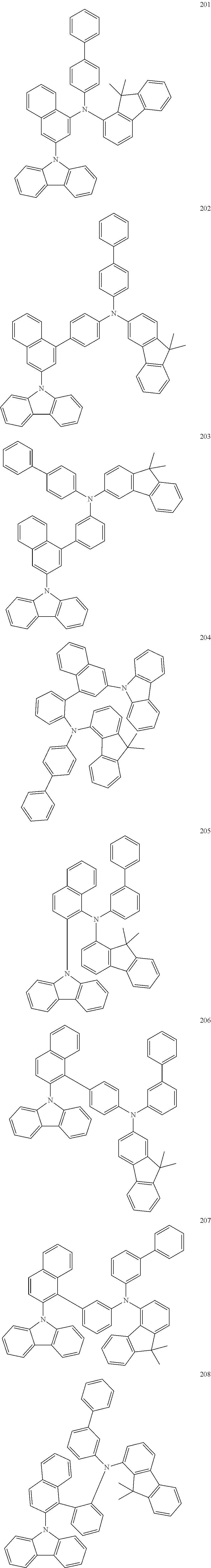

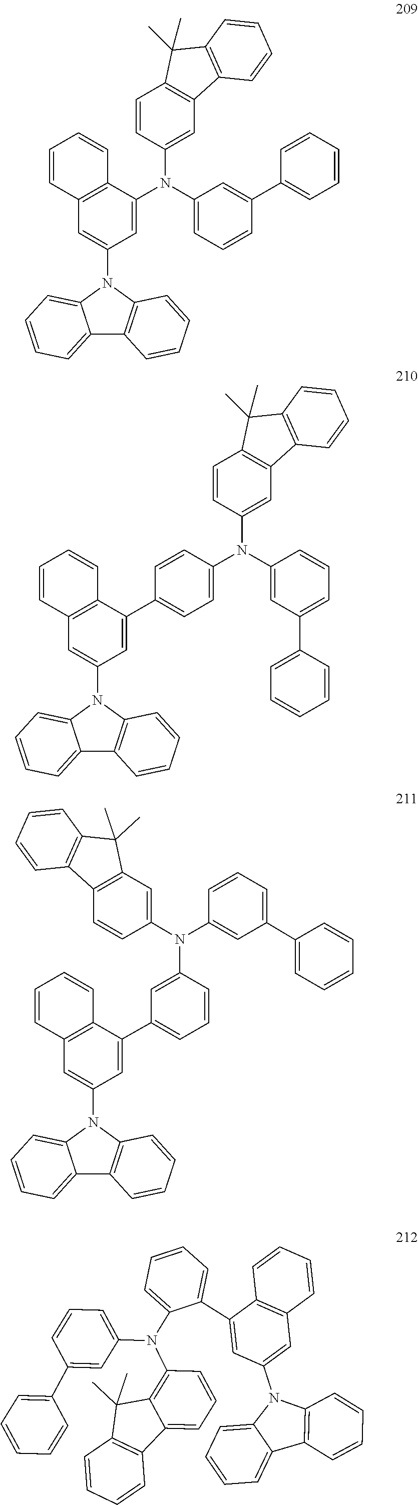

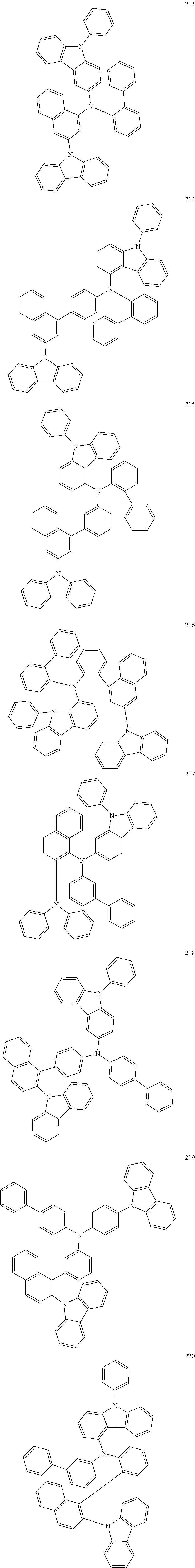

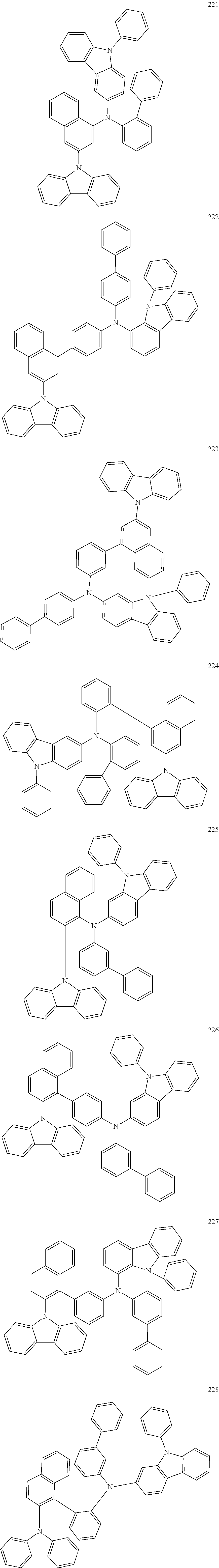

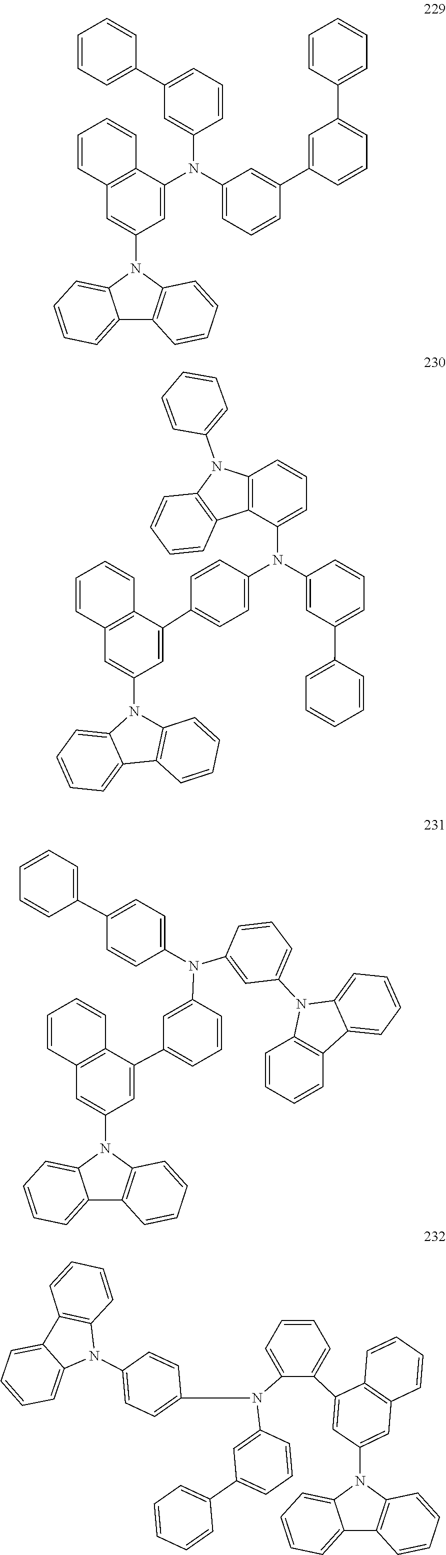

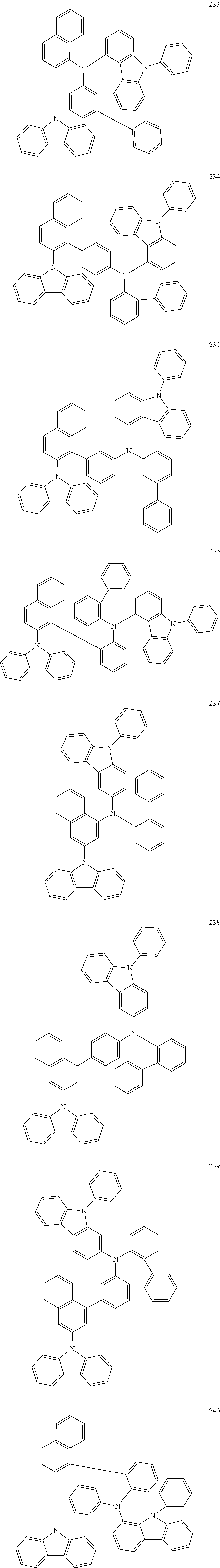

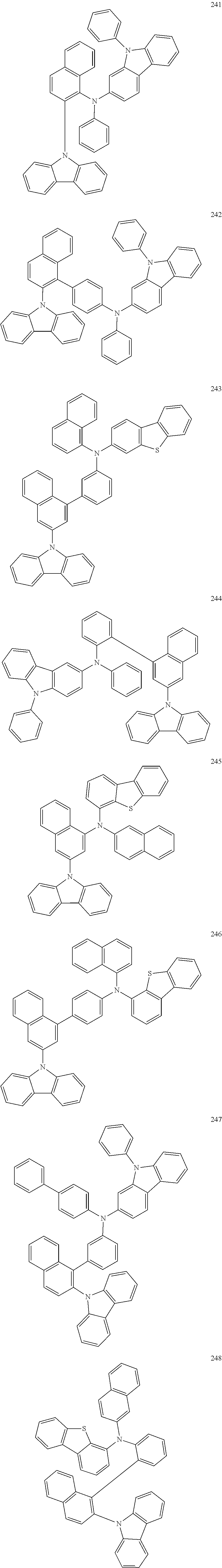

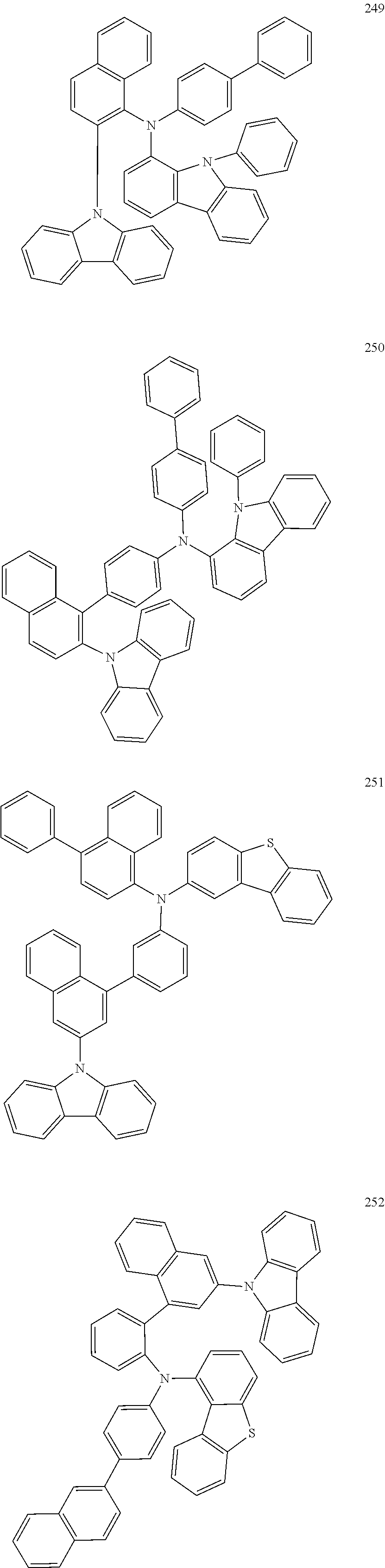 [SMILES: c1ccc(-c2ccc(N(c3c(-n4c5ccccc5c5ccccc54)ccc4ccccc34)c3cccc4c5ccccc5n(-c5ccccc5)c34)cc2)cc1 ; c1ccc(-c2ccc(N(c3ccc(-c4c(-n5c6ccccc6c6ccccc65)ccc5ccccc45)cc3)c3cccc4c5ccccc5n(-c5ccccc5)c34)cc2)cc1 ; c1ccc(-c2ccc(N(c3cccc(-c4cc(-n5c6ccccc6c6ccccc65)cc5ccccc45)c3)c3ccc4sc5ccccc5c4c3)c3ccccc23)cc1 ; c1ccc(N(c2ccc(-c3ccc4ccccc4c3)cc2)c2cccc3sc4ccccc4c23)c(-c2cc(-n3c4ccccc4c4ccccc43)cc3ccccc23)c1]

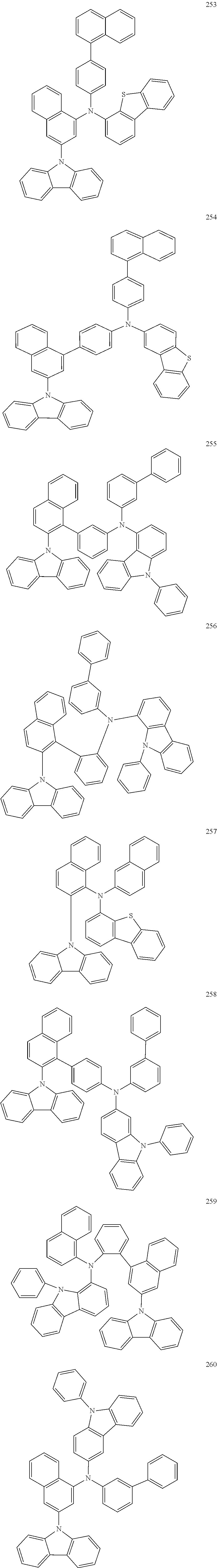

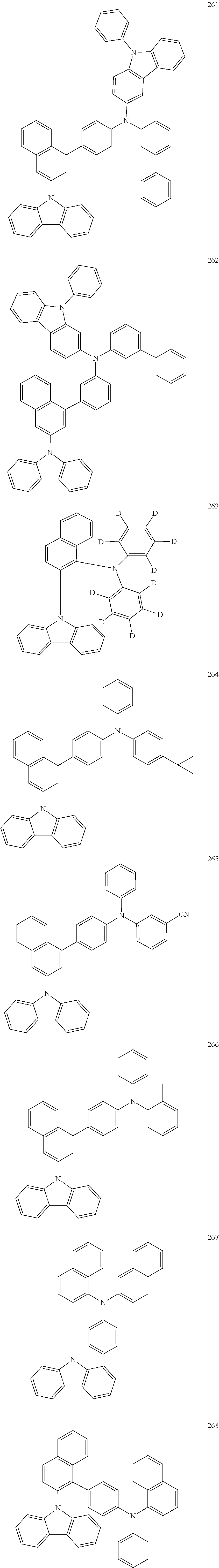

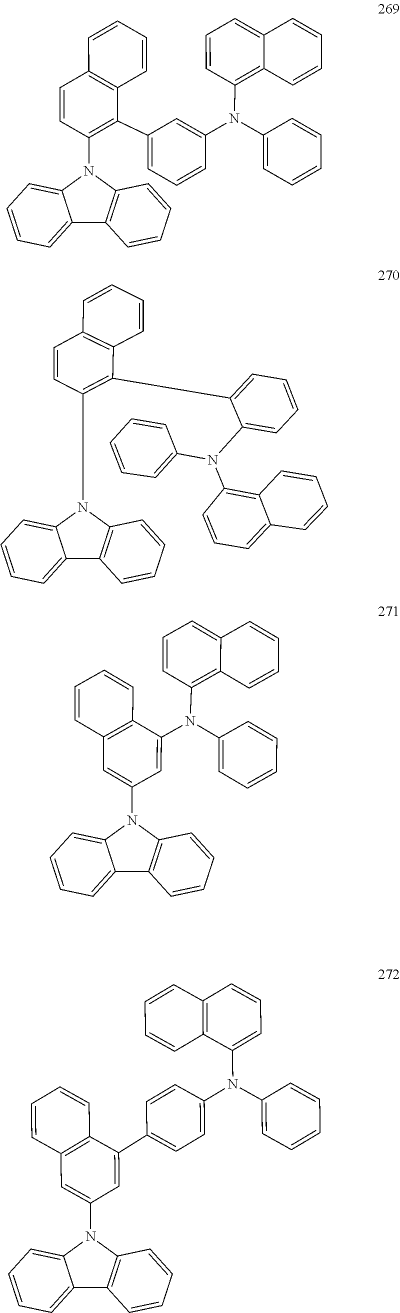

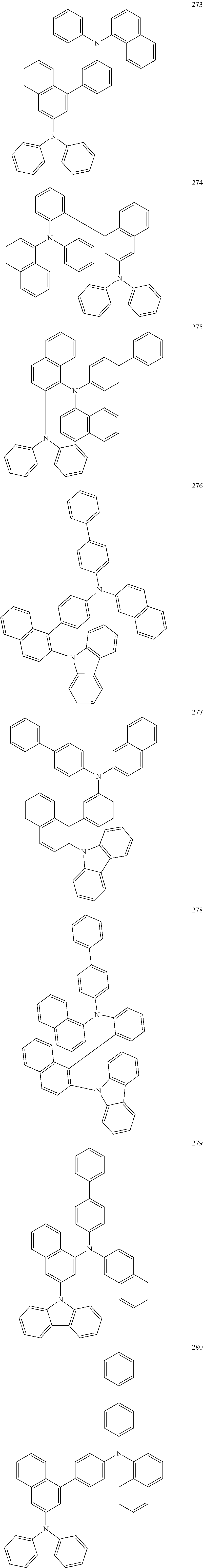

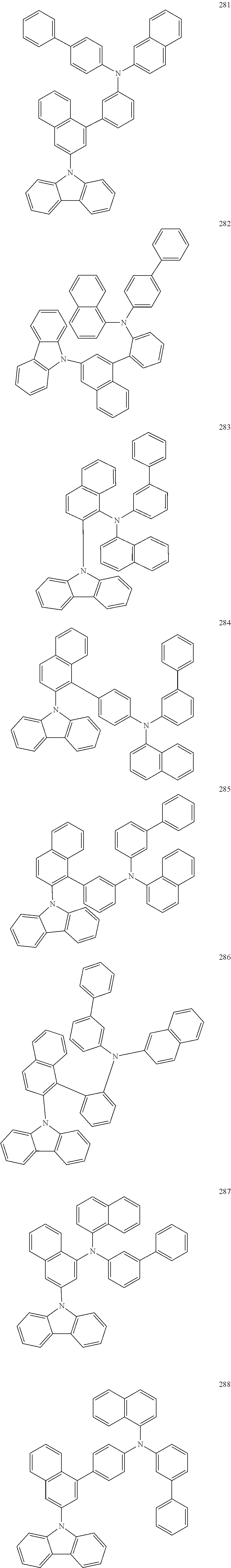

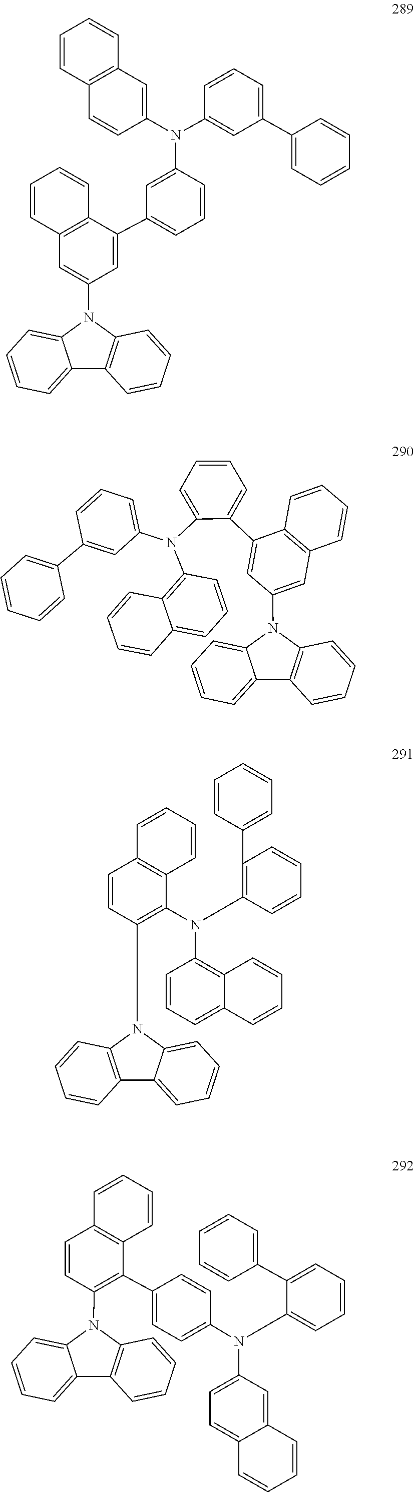 [SMILES: c1ccc(-c2cccc(N(c3cccc(-c4cc(-n5c6ccccc6c6ccccc65)cc5ccccc45)c3)c3ccc4ccccc4c3)c2)cc1 ; c1ccc(-c2cccc(N(c3ccccc3-c3cc(-n4c5ccccc5c5ccccc54)cc4ccccc34)c3cccc4ccccc34)c2)cc1 ; c1ccc(-c2ccccc2N(c2cccc3ccccc23)c2c(-n3c4ccccc4c4ccccc43)ccc3ccccc23)cc1 ; c1ccc(-c2ccccc2N(c2ccc(-c3c(-n4c5ccccc5c5ccccc54)ccc4ccccc34)cc2)c2ccc3ccccc3c2)cc1]

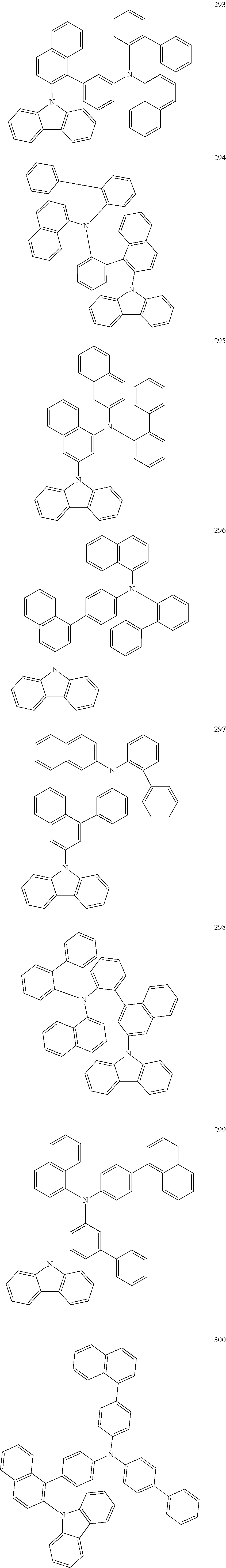

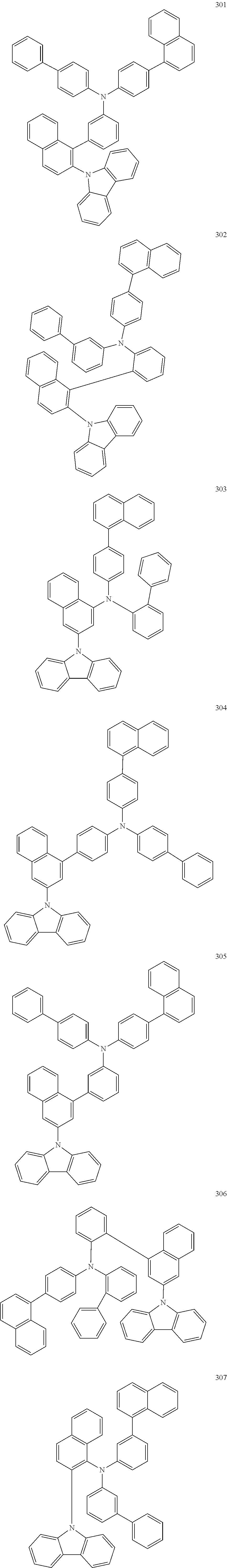

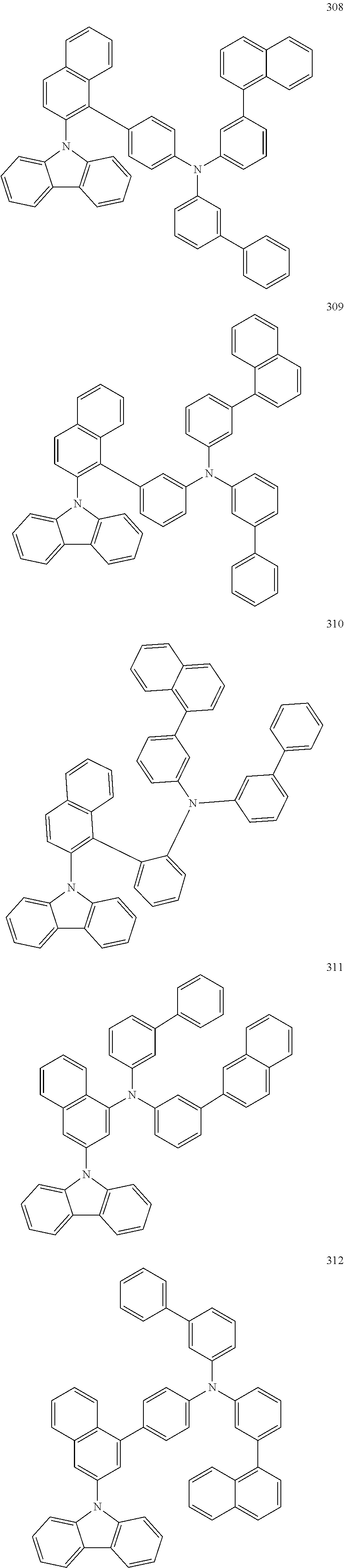

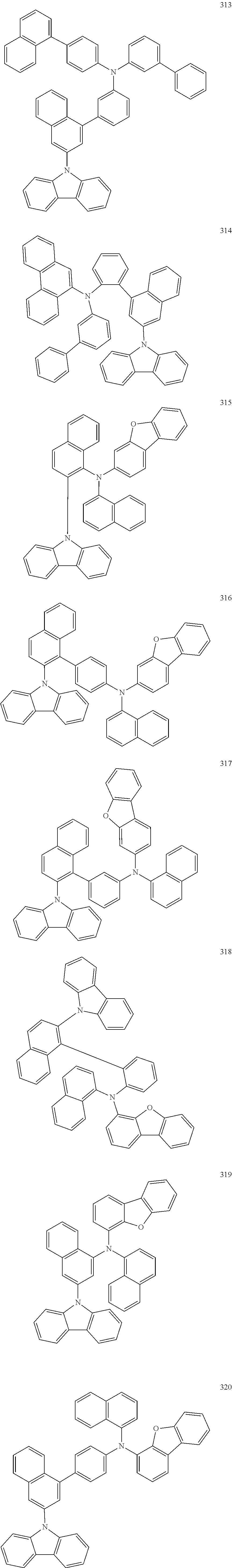

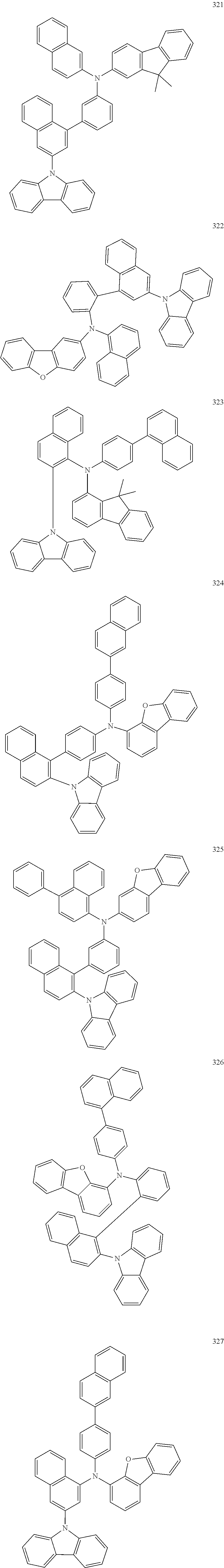

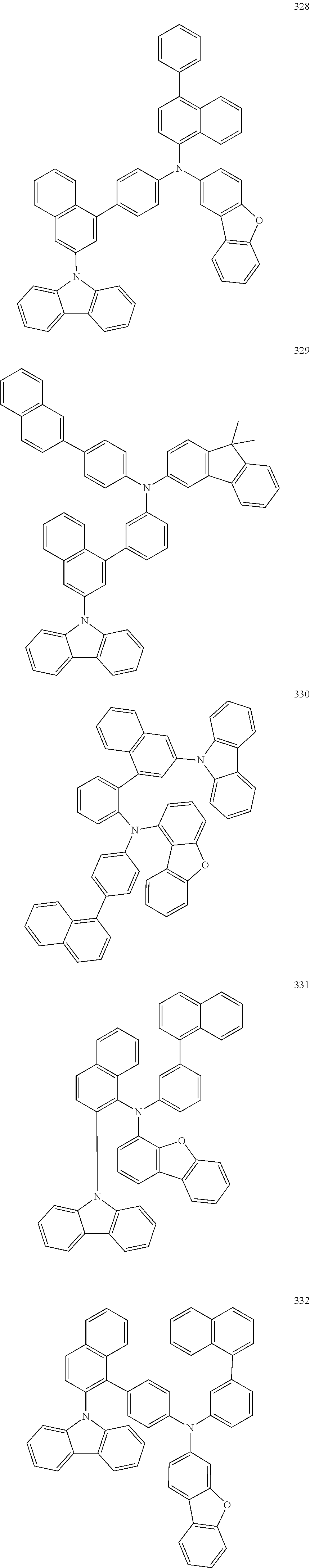

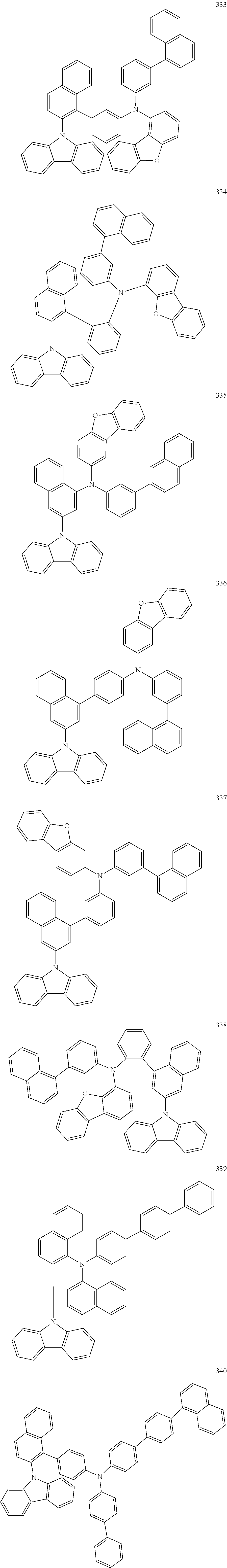

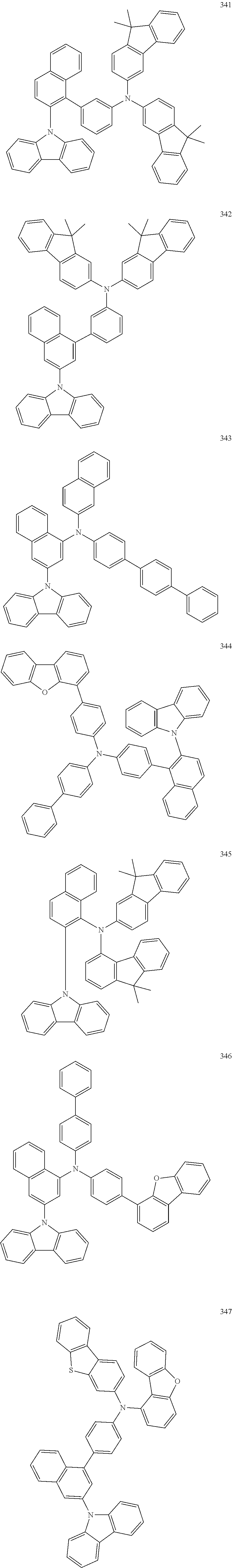

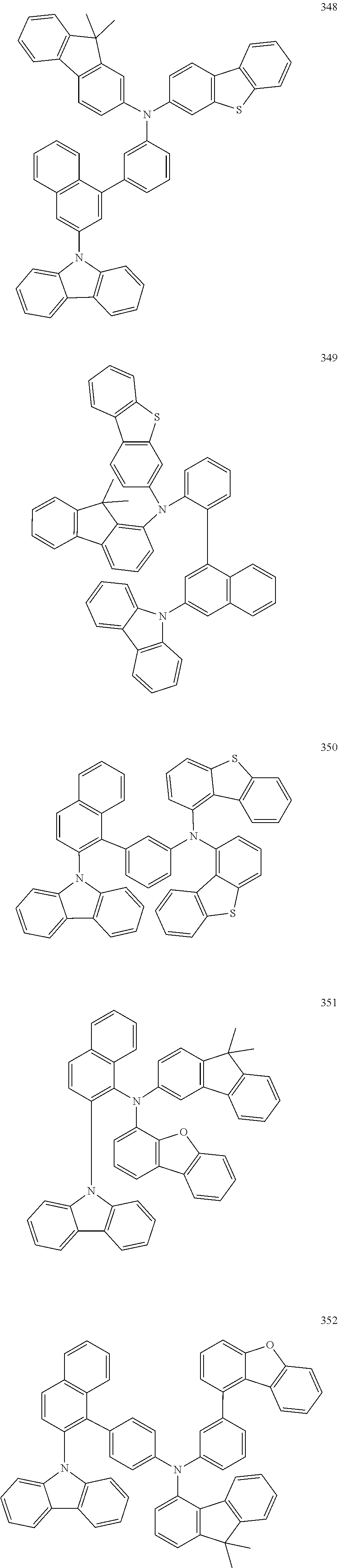 [SMILES: CC1(C)c2ccccc2-c2ccc(N(c3cccc(-c4cc(-n5c6ccccc6c6ccccc65)cc5ccccc45)c3)c3ccc4c(c3)sc3ccccc34)cc21 ; CC1(C)c2ccccc2-c2cccc(N(c3ccc4c(c3)sc3ccccc34)c3ccccc3-c3cc(-n4c5ccccc5c5ccccc54)cc4ccccc34)c21 ; c1cc(-c2c(-n3c4ccccc4c4ccccc43)ccc3ccccc23)cc(N(c2cccc3sc4ccccc4c23)c2cccc3sc4ccccc4c23)c1 ; CC1(C)c2ccccc2-c2cc(N(c3c(-n4c5ccccc5c5ccccc54)ccc4ccccc34)c3cccc4c3oc3ccccc34)ccc21 ; CC1(C)c2ccccc2-c2c(N(c3ccc(-c4c(-n5c6ccccc6c6ccccc65)ccc5ccccc45)cc3)c3cccc(-c4cccc5oc6ccccc6c45)c3)cccc21]

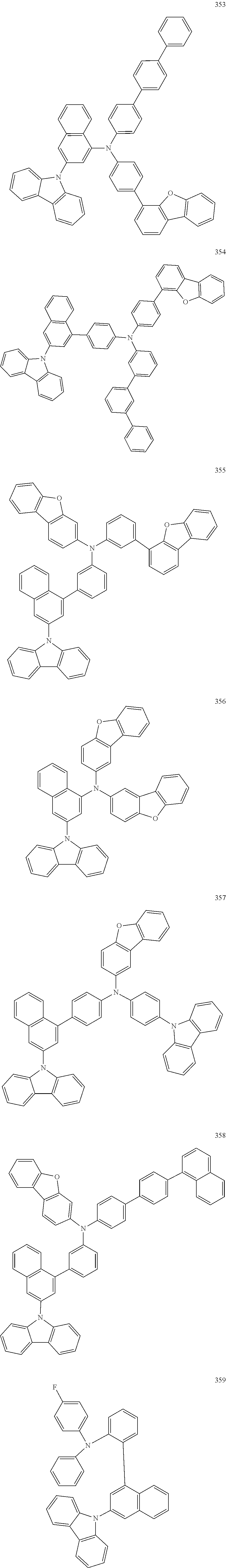

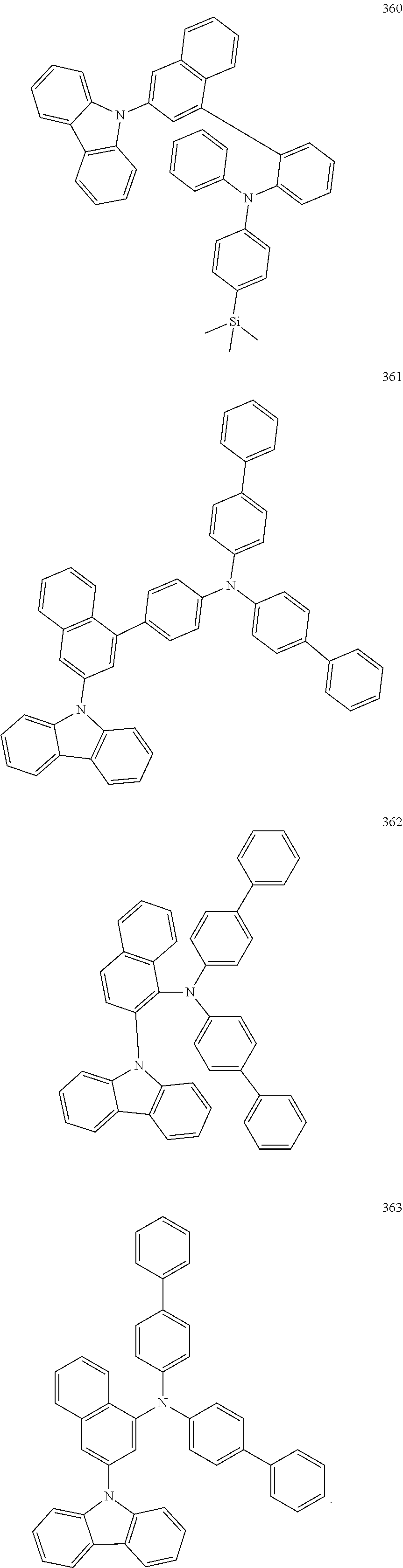 [SMILES: C[Si](C)(C)c1ccc(N(c2ccccc2)c2ccccc2-c2cc(-n3c4ccccc4c4ccccc43)cc3ccccc23)cc1 ; c1ccc(-c2ccc(N(c3ccc(-c4ccccc4)cc3)c3ccc(-c4cc(-n5c6ccccc6c6ccccc65)cc5ccccc45)cc3)cc2)cc1 ; c1ccc(-c2ccc(N(c3ccc(-c4ccccc4)cc3)c3c(-n4c5ccccc5c5ccccc54)ccc4ccccc34)cc2)cc1 ; c1ccc(-c2ccc(N(c3ccc(-c4ccccc4)cc3)c3cc(-n4c5ccccc5c5ccccc54)cc4ccccc34)cc2)cc1]

The present disclosure also provides an electronic component, comprising an anode and a cathode which is arranged oppositely to the anode, and a functional layer disposed between the anode and the cathode; and the functional layer comprises the nitrogen-containing compound of the present disclosure.

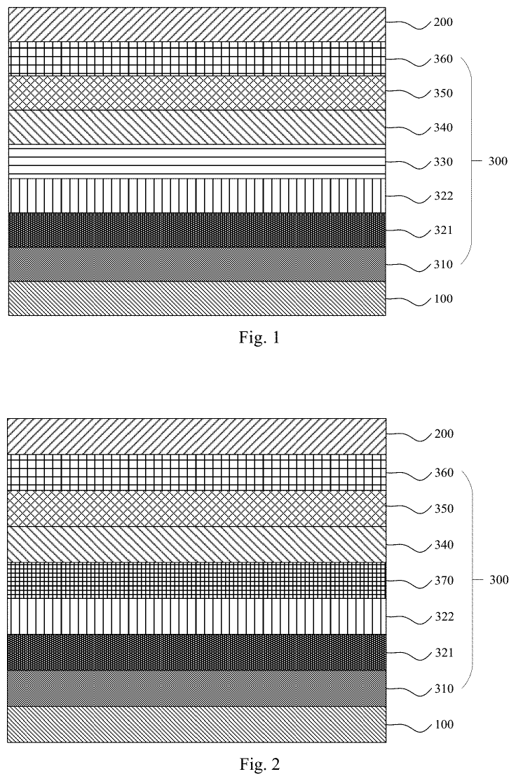

According to one embodiment, the electronic component is an organic electroluminescent device. As shown in FIG. 1, the organic electroluminescent device comprises an anode 100 and a cathode 200 which is arranged oppositely to the anode, and a functional layer 300 arranged between the anode 100 and the cathode 200; and the functional layer 300 comprises the nitrogen-containing compound provided by the present disclosure.

Optionally, the functional layer 300 comprises an electron blocking layer 322 which comprising the nitrogen-containing compound provided by the present disclosure. The electron blocking layer 322 may be composed of the nitrogen-containing compound provided by the present disclosure, or may be composed of the nitrogen-containing compound provided by the present disclosure together with other materials.

Optionally, the functional layer 300 comprises a hole transport layer 321 and/or a hole injection layer 310, and the hole transport layer 321 and/or the hole injection layer 310 may comprise the nitrogen-containing compound provided by the present disclosure to improve the transport ability of holes in the electronic component.

In one specific embodiment of the present disclosure, the organic electroluminescent device may comprise an anode 100, a hole transport layer 321, an electron blocking layer 322, an organic light-emitting layer 330 as an energy conversion layer, an electron transport layer 350, and a cathode 200 which are stacked in sequence. The nitrogen-containing compound provided by the present disclosure can be applied to the electron blocking layer 322 of the organic electroluminescent device, which can effectively improve the luminous efficiency and lifetime of the organic electroluminescent device and reduce the driving voltage of the organic electroluminescent device.

Optionally, the anode 100 comprises an anode material. Preferably, it is a material with a large work function that facilitates hole injection into the functional layer. Specific examples of the anode material include: metals such as nickel, platinum, vanadium, chromium, copper, zinc, and gold, or their alloys; metal oxides such as zinc oxide, indium oxide, indium tin oxide (ITO) and indium zinc oxide (IZO); combined metals and oxides, such as ZnO:Al or SnO₂:Sb; or a conductive polymer such as poly(3-methylthiophene), poly[3,4-(ethylene-1,2-dioxy)thiophene] (PEDT), polypyrrole, and polyaniline, but are not limited to this. A transparent electrode containing indium tin oxide (ITO) as the anode is preferably comprised.

Optionally, the hole transport layer 321 can comprise one or more hole transport materials, and the hole transport materials can be selected from a carbazole polymer, carbazole connected triarylamine compounds or other types of compounds, which are not specially limited in the present disclosure. For example, the hole transport layer 321 is composed of a compound NPB.

Optionally, the organic light-emitting layer 330 may be composed of a single light-emitting material or can comprise a host material and a guest material. Alternatively, the organic light-emitting layer 330 is composed of the host material and the guest material, and holes injected into the organic light-emitting layer 330 and electrons injected into the organic light-emitting layer 330 can be combined in the organic light-emitting layer 330 to form excitons, the excitons transfer energy to the host material, and the host material transfers energy to the guest material, so that the guest material can emit light.

The host material of the organic light-emitting layer 330 may be a metal chelate compound, a bis-styryl derivative, an aromatic amine derivative, a dibenzofuran derivative, or other types of materials, which is not specially limited in the present disclosure. For example, the host material of the organic light-emitting layer 330 may be BH-01.

The guest material of the organic light-emitting layer 330 may be a compound having a condensed aryl ring or its derivative, a compound having a heteroaryl ring or its derivative, an aromatic amine derivative, or other materials, which is not specially limited in the present disclosure. For example, the guest material of the organic light-emitting layer 330 may be BD-01.

The electron transport layer 350 may be of a single-layer structure or a multi-layer structure, which may comprise one or more electron transport materials, and the electron transport materials can be selected from a benzimidazole derivative, an oxadiazole derivative, a quinoxaline derivative, or other electron transport materials, which are not specially limited in the present disclosure. For example, the electron transport layer 350 may be composed of ET-06 and LiQ.

Optionally, the cathode 200 comprises a cathode material, which is a material having a small work function that facilitates electron injection into the functional layer.

Specific examples of the cathode material comprise metals such as magnesium, calcium, sodium, potassium, titanium, indium, yttrium, lithium, gadolinium, aluminum, silver, tin, and lead, or their alloys; or multilayer materials such as LiF/Al, Liq/Al, LiO₂/Al, LiF/Ca, LiF/Al, and BaF₂/Ca, but are not limited to this. A metal electrode containing silver and magnesium as the cathode is preferably comprised.

Optionally, as shown in FIG. 1, a hole injection layer 310 may also be arranged between the anode 100 and the hole transport layer 321 to enhance the ability to inject holes into the hole transport layer 321. The hole injection layer 310 can be made of a benzidine derivative, a starburst arylamine compound, a phthalocyanine derivative or other materials, which is not specially limited in the present disclosure. For example, the hole injection layer 310 may be composed of F4-TCNQ.

Optionally, as shown in FIG. 1, an electron injection layer 360 may also be arranged between the cathode 200 and the electron transport layer 350 to enhance the ability to inject electrons into the electron transport layer 350. The electron injection layer 360 may comprise an inorganic material such as an alkali metal sulfide and an alkali metal halide, or may comprise a complex of an alkali metal and an organic substance. For example, the electron injection layer 360 may be composed of Yb.

Optionally, a hole blocking layer 340 may also be arranged between the organic light-emitting layer 330 and the electron transport layer 350.

Optionally, the organic electroluminescent device is a blue light device.

According to another embodiment, the electronic component is a photoelectric conversion device, as shown in FIG. 2, the photoelectric conversion device may comprise an anode 100 and a cathode 200 which are disposed oppositely, and a functional layer 300 disposed between the anode 100 and the cathode 200; and the functional layer 300 comprises the nitrogen-containing compound provided in the present disclosure.

Optionally, the functional layer 300 comprises an electron blocking layer 322 comprising the nitrogen-containing compound provided by the present disclosure. The electron blocking layer 322 may be composed of the nitrogen-containing compound provided by the present disclosure, or may be composed of the nitrogen-containing compound provided by the present disclosure together with other materials.

Optionally, as shown in FIG. 2, the photoelectric conversion device may comprise an anode 100, a hole transport layer 321, an electron blocking layer 322, a photoelectric conversion layer 370 as an energy conversion layer, an electron transport layer 350, and a cathode 200 which are stacked in sequence. The nitrogen-containing compound provided in the present disclosure can be applied to the electron blocking layer 322 of the photoelectric conversion device, which can effectively improve the luminous efficiency and service life of the photoelectric conversion device and increase the open circuit voltage of the photoelectric conversion device.

Optionally, a hole injection layer 310 may also be arranged between the anode 100 and the hole transport layer 321.

Optionally, an electron injection layer 360 may also be arranged between the cathode 200 and the electron transport layer 350.

Optionally, a hole blocking layer 340 may also be arranged between the photoelectric conversion layer 370 and the electron transport layer 350.

Optionally, the photoelectric conversion device may be a solar cell, and in particular may be an organic thin film solar cell. According to one specific embodiment, as shown in FIG. 2, the solar cell comprises an anode 100, a hole transport layer 321, an electron blocking layer 322, a photoelectric conversion layer 370, an electron transport layer 350, and a cathode 200 which are stacked in sequence, and the electron blocking layer 322 comprises the nitrogen-containing compound of the present disclosure.

The present disclosure also provides an electronic device, comprising the electronic component according to the second aspect of the present disclosure.

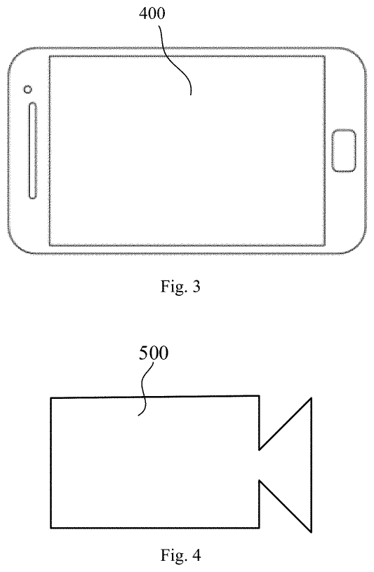

According to one embodiment, as shown in FIG. 3, the electronic device is a first electronic device 400 comprising the organic electroluminescent device described above. The first electronic device 400 may be a display device, a lighting device, an optical communication device, or other types of electronic devices, and may comprise, for example, but is not limited to, a computer screen, a mobile phone screen, a television, electronic paper, an emergency lighting lamp, an optical module, and the like.

According to another embodiment, as shown in FIG. 4, the electronic device is a second electronic device 500 comprising the photoelectric conversion device described above. The second electronic device 500 may be a solar power plant, a light detector, a fingerprint recognition device, an optical module, a CCD camera, or other types of electronic devices.

The nitrogen-containing compound of the present disclosure and its application are described below with reference to synthesis examples and examples. Unless otherwise indicated, the raw materials and materials used may be obtained commercially or obtained by methods well known in the art.

### Synthetic Example: Synthesis of Compounds

Preparation Example 1: Synthesis of Compound 2

### (1) Synthesis of Intermediate E-1

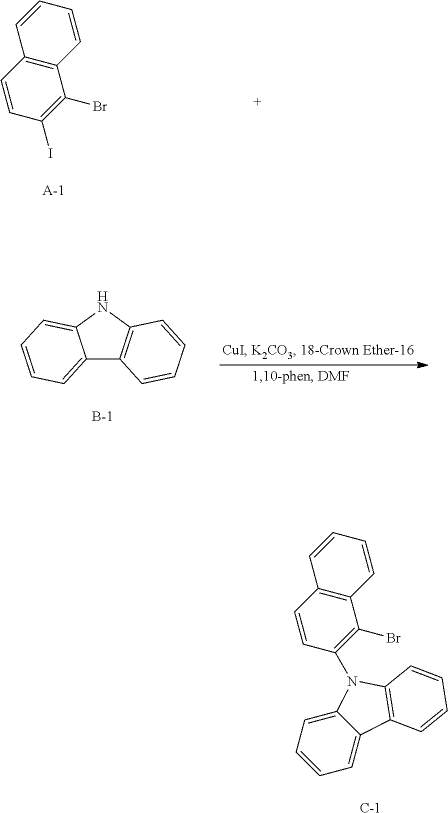

A-1 (10 g, 30.0 mmol), B-1 (5.02 g, 30.0 mmol), cuprous iodide (1.14 g, 6.0 mmol), potassium carbonate (9.13 g, 66.1 mmol), 18-crown-6 (2.16 g, 12.0 mmol), 1,10-phenanthroline (0.8 g, 3.0 mmol), and N,N-dimethylformamide (100 mL) were added into a reaction flask, and heated to 150° C. under a nitrogen atmosphere, a reaction was carried out for 12 h, after the reaction was completed, the resulting reaction solution was cooled to room temperature, then the obtained reaction solution was extracted with dichloromethane and water. The organic layer was dried over anhydrous magnesium sulfate, and filtered, the resulting filtrate was allowed to pass through a short silica gel column, a solvent was removed under reduced pressure, and a crude product was purified by recrystallization using a dichloromethane/n-heptane (a volume ratio of 1:3) system to give an intermediate C-1 (7.27 g, yield: 65%).

An intermediate C-X in Table 1 was synthesized with reference to the synthesis method of the intermediate C-1, except that A-X was used instead of A-1, where X may be 2, and the prepared intermediate C-X is as shown in Table 1.

TABLE 1

| A-X | Intermediate C-X | Yield (%) |
|---|---|---|
| 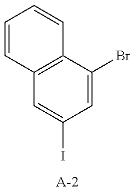 | 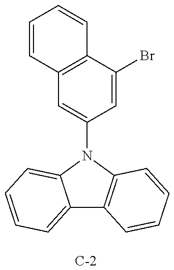 | 66 |

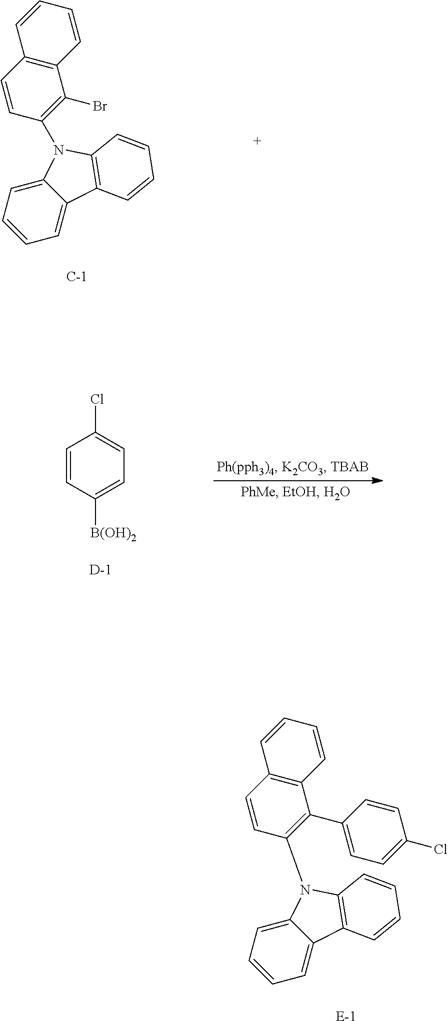

The intermediate C-1 (10 g, 26.9 mmol), D-1 (5.04 g, 32.2 mmol), tetrakis(triphenylphosphine)palladium (1.55 g, 1.34 mmol), potassium carbonate (5.57 g, 40.3 mmol), tetrabutylammonium bromide (0.37 g, 1.34 mmol), toluene (80 mL), ethanol (40 mL) and deionized water (20 mL) were added to a round bottom flask, heated to 80° C. under nitrogen protection, and stirred for 12 h; the obtained reaction solution was cooled to room temperature, and extracted with toluene (100 mL). The organic phases were combined, dried over anhydrous magnesium sulfate, and filtered. The solvent was removed under reduced pressure. The crude product obtained was purified by silica gel column chromatography using n-heptane as mobile phase, followed by recrystallization using a dichloromethane/ethyl acetate system (a volume ratio of 1:5) to obtain an intermediate E-1 (8.68 g, yield: 80%).

### (2) Synthesis of Compound 2

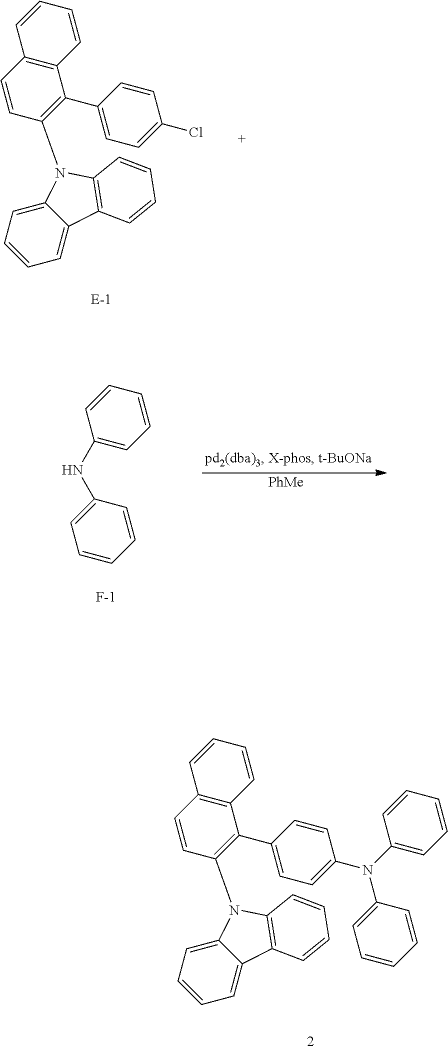

The intermediate E-1 (10 g, 24.8 mmol), F-1 (4.19 g, 24.8 mmol), tris(dibenzylideneacetone)dipalladium (0.23 g, 0.49 mmol), 2-dicyclohexylphosphino-2,4,6,-triisopropylbiphenyl (0.24 g, 0.5 mmol), sodium tert-butoxide (3.57 g, 37.1 mmol) and a toluene solvent (100 mL) were added into a reaction flask, heated to 110° C. under nitrogen protection, and stirred under heating and refluxing for 3 h. After the resulting reaction solution was cooled to room temperature, the reaction solution was extracted with dichloromethane and water, the organic layer was dried over anhydrous magnesium sulfate, and filtered, the obtained filtrate was allowed to pass through a short silica gel column, a solvent was removed under reduced pressure, and a crude product was purified by recrystallization using a dichloromethane/n-heptane system (a volume ratio of 1:3) to give a compound 2 (9.57 g, yield: 72%).

Intermediates E-X in Table 2 were synthesized with reference to the synthesis method of the intermediate E-1, except that D-X in Table 2 was used instead of D-1, and C-X in Table 2 was used instead of C-1, and each compound CAX and D-X were combined to prepare the only corresponding intermediate E-X, and the prepared intermediates E-X are as shown in Table 2.

TABLE 2

| C-X | D-X | Intermediate E-X | Yield (%) |
|---|---|---|---|
| 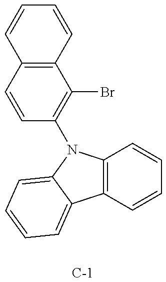 | 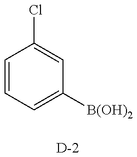 | 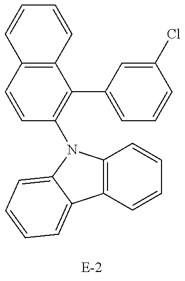 [SMILES: Clc1cccc(-c2c(-n3c4ccccc4c4ccccc43)ccc3ccccc23)c1] | 79 |
| | 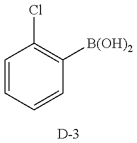 | 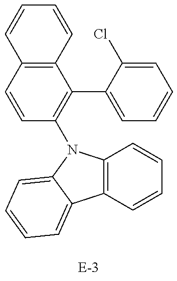 [SMILES: Clc1ccccc1-c1c(-n2c3ccccc3c3ccccc32)ccc2ccccc12] | 78 |
| 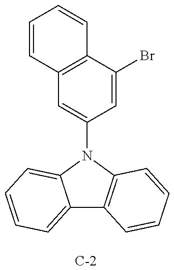 | 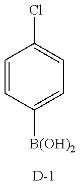 | 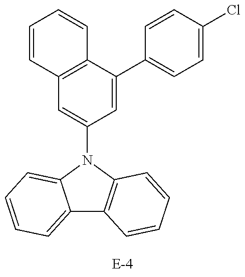 [SMILES: Clc1ccc(-c2cc(-n3c4ccccc4c4ccccc43)cc3ccccc23)cc1] | 76 |
| | 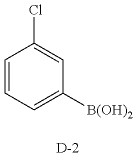 | 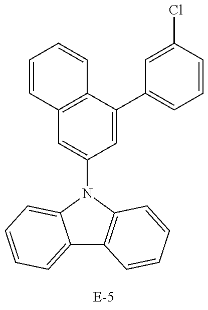 [SMILES: Clc1cccc(-c2cc(-n3c4ccccc4c4ccccc43)cc3ccccc23)c1] | 78 |
| | 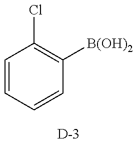 | 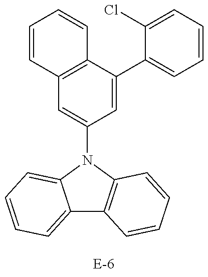 [SMILES: Clc1ccccc1-c1cc(-n2c3ccccc3c3ccccc32)cc2ccccc12] | 76 |

Compounds Y in Table 3 were synthesized with reference to the synthesis method of the compound 2, except that the intermediate CAX or the intermediate E-X in Table 3 was used instead of the intermediate E-1, and F-X in Table 3 was used instead of F-1, and each intermediate CAX or E-X and F-X were combined to prepare the only corresponding compound Y, and the prepared compounds Y are as shown in Table 3.

TABLE 3

| Intermediate | | | |
|---|---|---|---|
| E(C)-X | F-X | Compound Y | Yield (%) |
| 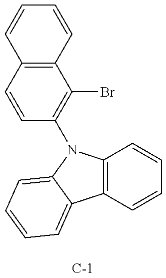 | 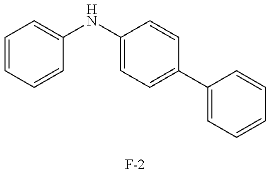 | 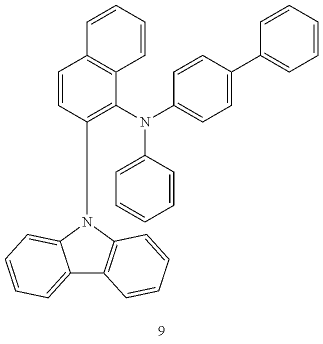 [SMILES: c1ccc(-c2ccc(N(c3ccccc3)c3c(-n4c5ccccc5c5ccccc54)ccc4ccccc34)cc2)cc1] | 70 |
| 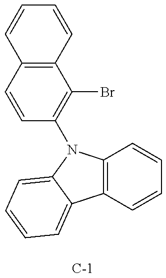 | 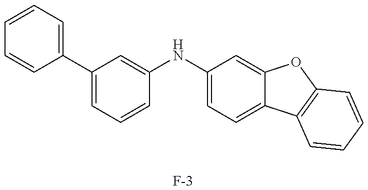 | 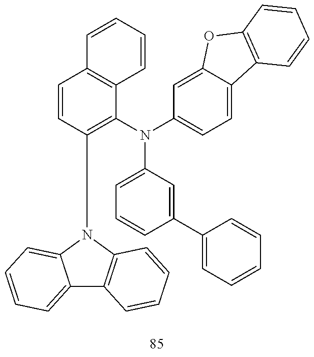 [SMILES: c1ccc(-c2cccc(N(c3ccc4c(c3)oc3ccccc34)c3c(-n4c5ccccc5c5ccccc54)ccc4ccccc34)c2)cc1] | 71 |
| 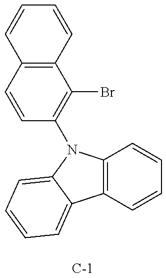 | 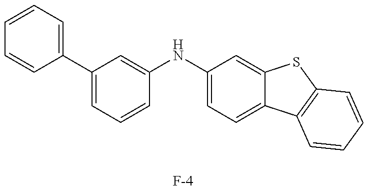 | 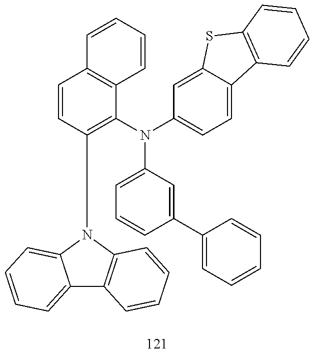 [SMILES: c1ccc(-c2cccc(N(c3ccc4c(c3)sc3ccccc34)c3c(-n4c5ccccc5c5ccccc54)ccc4ccccc34)c2)cc1] | 72 |
| 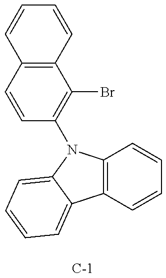 | 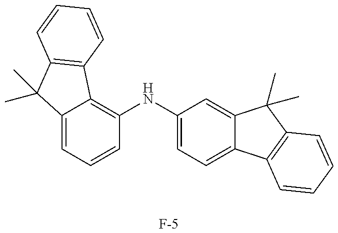 | 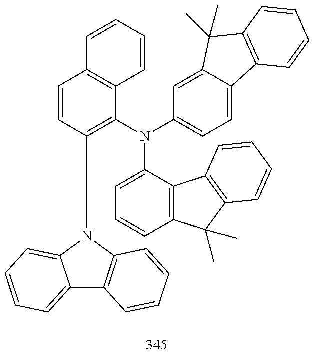 [SMILES: CC1(C)c2ccccc2-c2ccc(N(c3cccc4c3-c3ccccc3C4(C)C)c3c(-n4c5ccccc5c5ccccc54)ccc4ccccc34)cc21] | 74 |
| 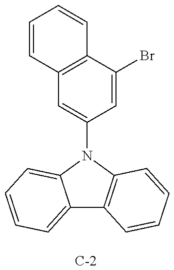 | 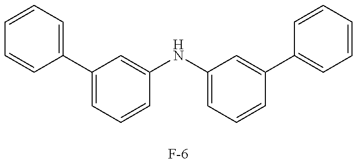 | 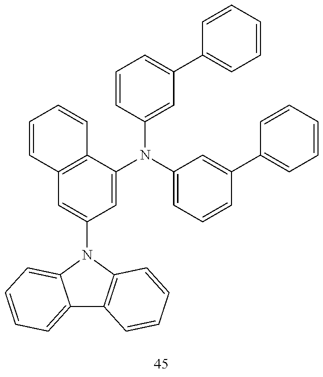 [SMILES: c1ccc(-c2cccc(N(c3cccc(-c4ccccc4)c3)c3cc(-n4c5ccccc5c5ccccc54)cc4ccccc34)c2)cc1] | 73 |
| 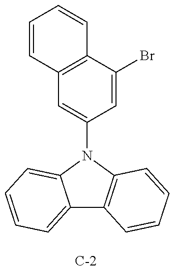 | 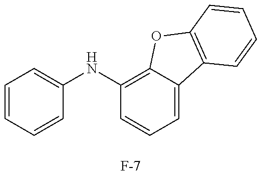 | 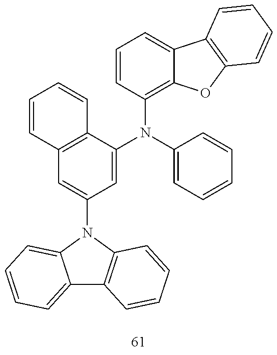 [SMILES: c1ccc(N(c2cc(-n3c4ccccc4c4ccccc43)cc3ccccc23)c2cccc3c2oc2ccccc23)cc1] | 72 |
| 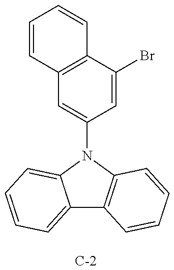 | 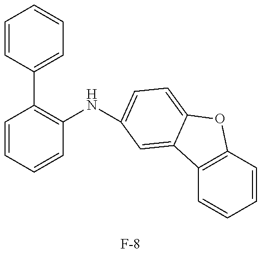 | 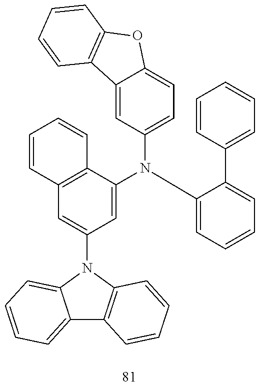 [SMILES: c1ccc(-c2ccccc2N(c2ccc3oc4ccccc4c3c2)c2cc(-n3c4ccccc4c4ccccc43)cc3ccccc23)cc1] | 73 |
| 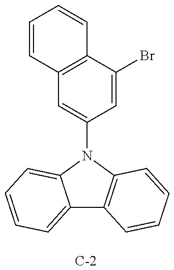 | 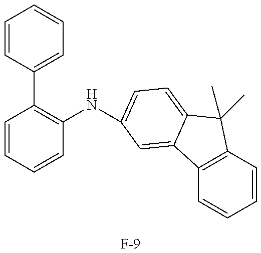 | 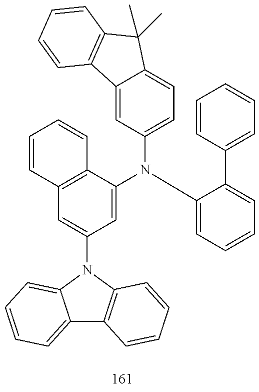 [SMILES: CC1(C)c2ccccc2-c2cc(N(c3ccccc3-c3ccccc3)c3cc(-n4c5ccccc5c5ccccc54)cc4ccccc34)ccc21] | 71 |
| 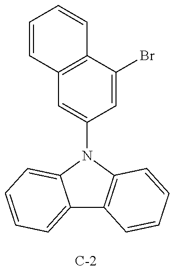 | 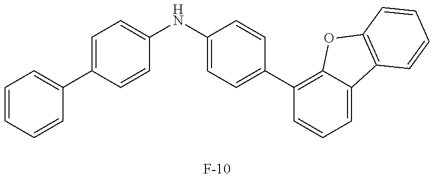 | 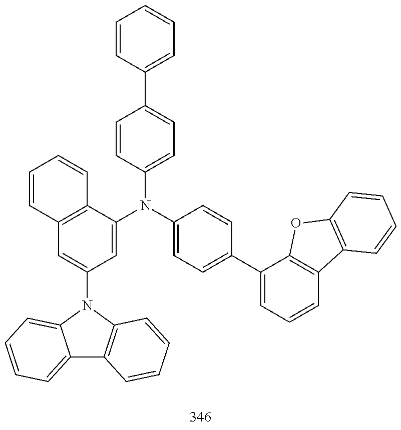 [SMILES: c1ccc(-c2ccc(N(c3ccc(-c4cccc5c4oc4ccccc45)cc3)c3cc(-n4c5ccccc5c5ccccc54)cc4ccccc34)cc2)cc1] | 72 |
| 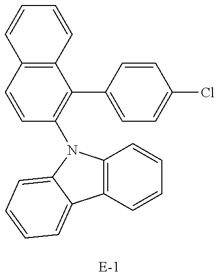 | 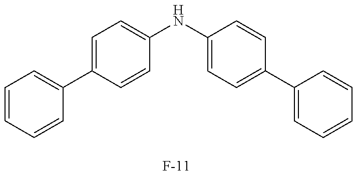 | 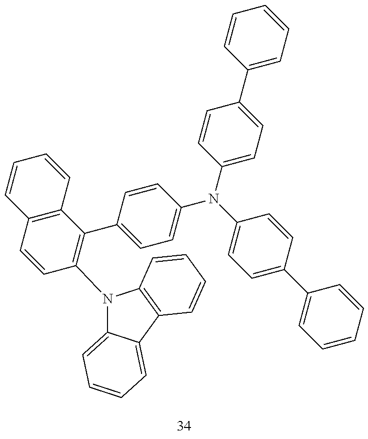 [SMILES: c1ccc(-c2ccc(N(c3ccc(-c4ccccc4)cc3)c3ccc(-c4c(-n5c6ccccc6c6ccccc65)ccc5ccccc45)cc3)cc2)cc1] | 73 |
| 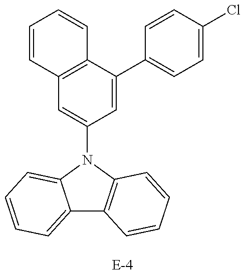 | 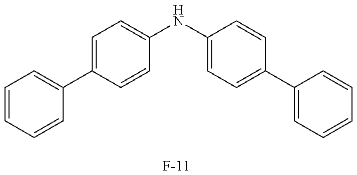 | 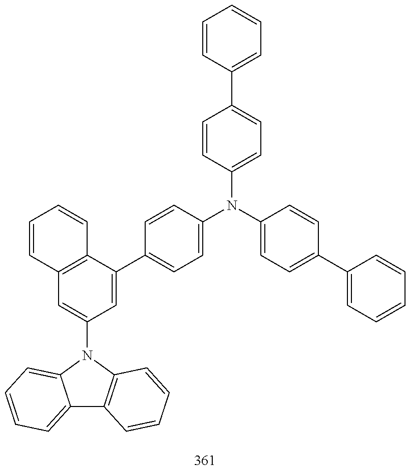 [SMILES: c1ccc(-c2ccc(N(c3ccc(-c4ccccc4)cc3)c3ccc(-c4cc(-n5c6ccccc6c6ccccc65)cc5ccccc45)cc3)cc2)cc1] | 74 |
| 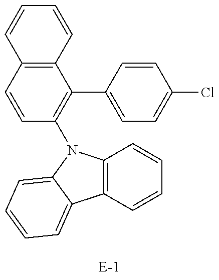 | 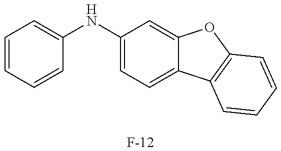 | 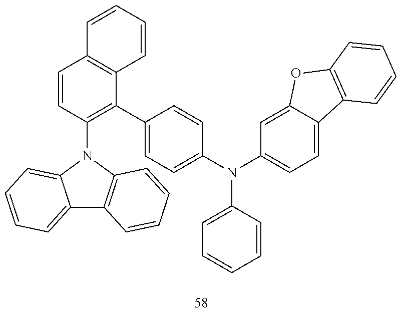 [SMILES: c1ccc(N(c2ccc(-c3c(-n4c5ccccc5c5ccccc54)ccc4ccccc34)cc2)c2ccc3c(c2)oc2ccccc23)cc1] | 72 |
| 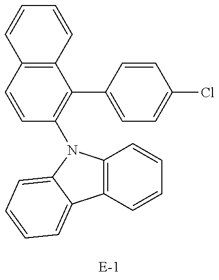 | 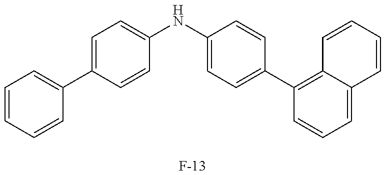 | 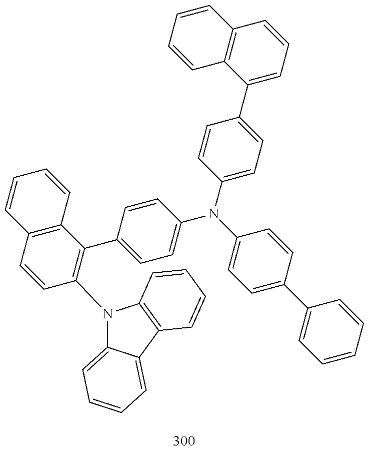 [SMILES: c1ccc(-c2ccc(N(c3ccc(-c4cccc5ccccc45)cc3)c3ccc(-c4c(-n5c6ccccc6c6ccccc65)ccc5ccccc45)cc3)cc2)cc1] | 71 |
| 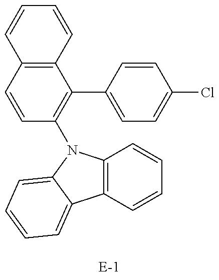 | 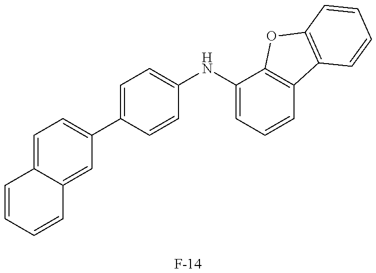 | 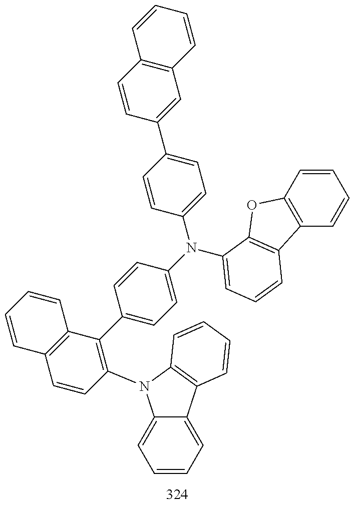 [SMILES: c1ccc2cc(-c3ccc(N(c4ccc(-c5c(-n6c7ccccc7c7ccccc76)ccc6ccccc56)cc4)c4cccc5c4oc4ccccc45)cc3)ccc2c1] | 73 |
| 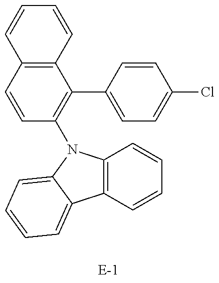 | 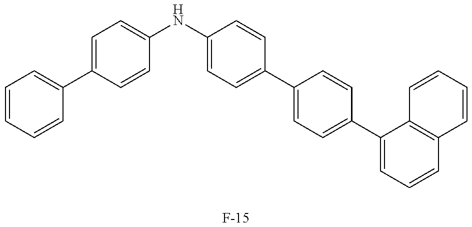 | 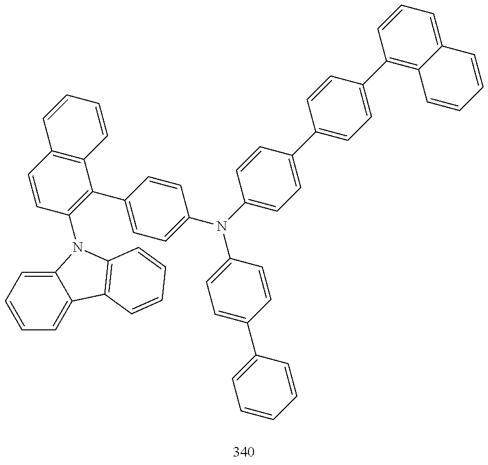 [SMILES: c1ccc(-c2ccc(N(c3ccc(-c4ccc(-c5cccc6ccccc56)cc4)cc3)c3ccc(-c4c(-n5c6ccccc6c6ccccc65)ccc5ccccc45)cc3)cc2)cc1] | 74 |
| 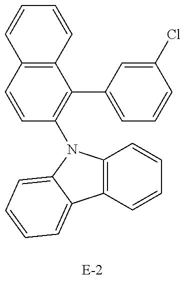 | 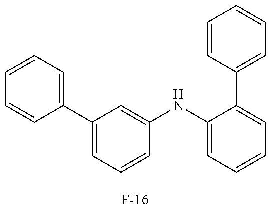 | 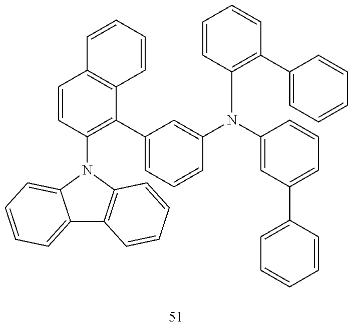 [SMILES: c1ccc(-c2cccc(N(c3cccc(-c4c(-n5c6ccccc6c6ccccc65)ccc5ccccc45)c3)c3ccccc3-c3ccccc3)c2)cc1] | 73 |
| 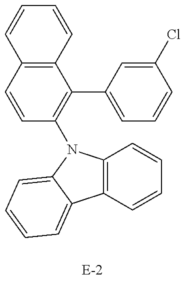 | 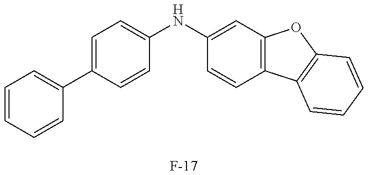 | 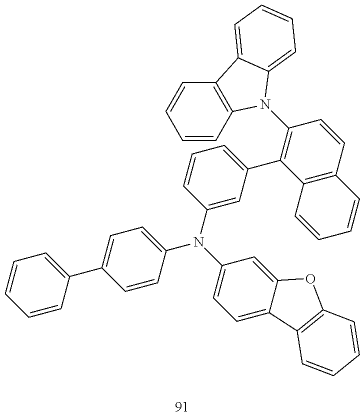 [SMILES: c1ccc(-c2ccc(N(c3cccc(-c4c(-n5c6ccccc6c6ccccc65)ccc5ccccc45)c3)c3ccc4c(c3)oc3ccccc34)cc2)cc1] | 74 |
| 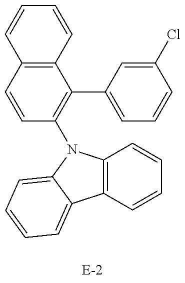 | 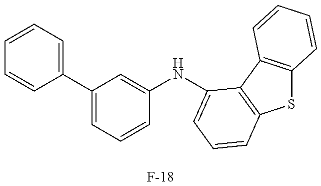 | 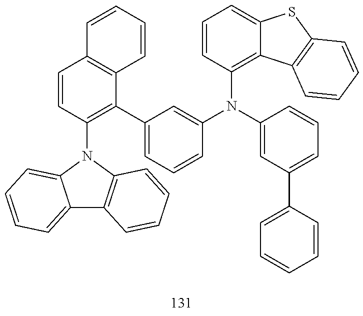 [SMILES: c1ccc(-c2cccc(N(c3cccc(-c4c(-n5c6ccccc6c6ccccc65)ccc5ccccc45)c3)c3cccc4sc5ccccc5c34)c2)cc1] | 73 |
| 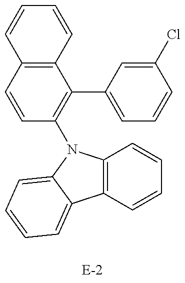 | 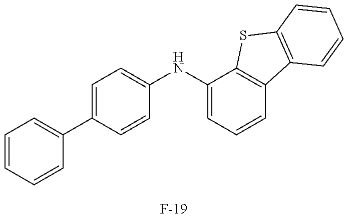 | 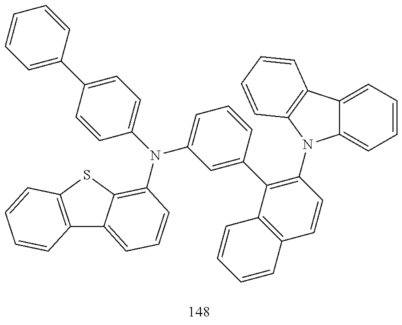 [SMILES: c1ccc(-c2ccc(N(c3cccc(-c4c(-n5c6ccccc6c6ccccc65)ccc5ccccc45)c3)c3cccc4c3sc3ccccc34)cc2)cc1] | 72 |
| 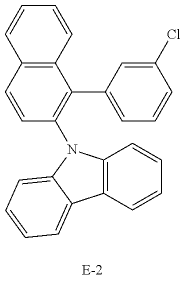 | 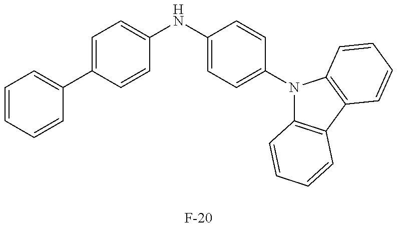 | 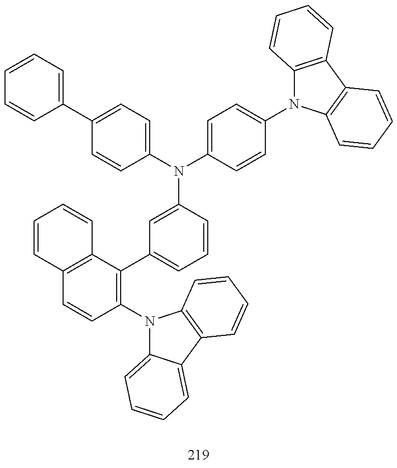 [SMILES: c1ccc(-c2ccc(N(c3ccc(-n4c5ccccc5c5ccccc54)cc3)c3cccc(-c4c(-n5c6ccccc6c6ccccc65)ccc5ccccc45)c3)cc2)cc1] | 71 |
| 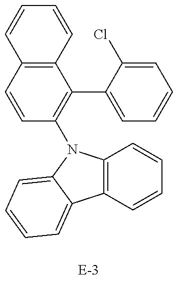 | 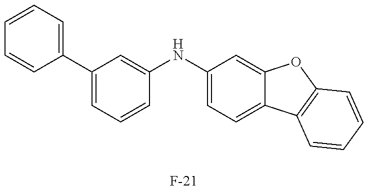 | 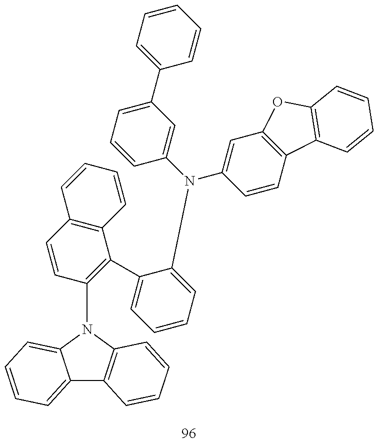 [SMILES: c1ccc(-c2cccc(N(c3ccc4c(c3)oc3ccccc34)c3ccccc3-c3c(-n4c5ccccc5c5ccccc54)ccc4ccccc34)c2)cc1] | 70 |
| 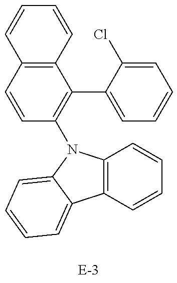 | 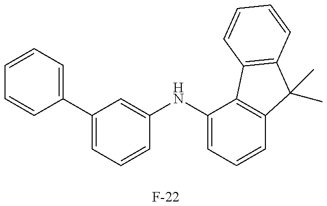 | 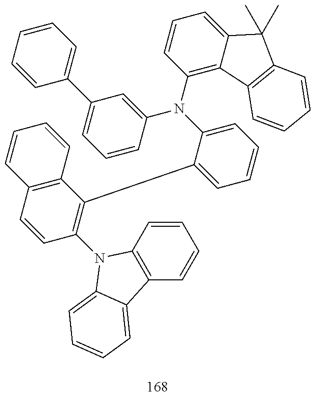 [SMILES: CC1(C)c2ccccc2-c2c(N(c3cccc(-c4ccccc4)c3)c3ccccc3-c3c(-n4c5ccccc5c5ccccc54)ccc4ccccc34)cccc21] | 72 |
| 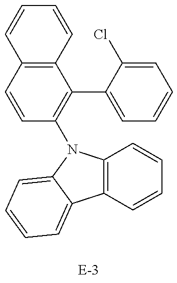 | 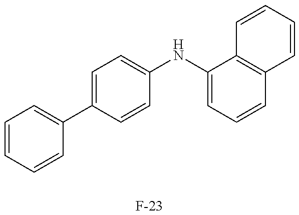 | 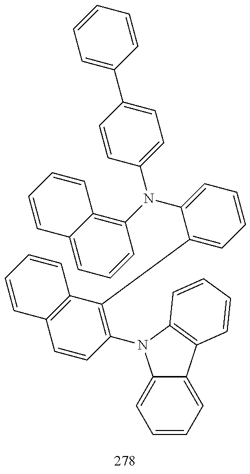 [SMILES: c1ccc(-c2ccc(N(c3ccccc3-c3c(-n4c5ccccc5c5ccccc54)ccc4ccccc34)c3cccc4ccccc34)cc2)cc1] | 73 |
| 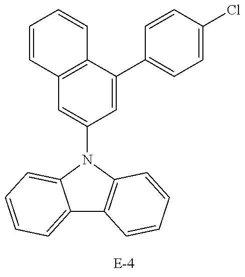 | 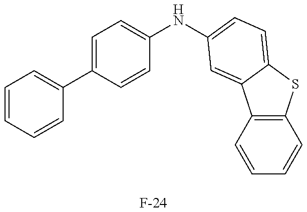 | 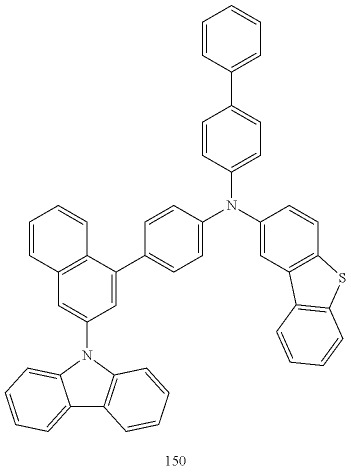 [SMILES: c1ccc(-c2ccc(N(c3ccc(-c4cc(-n5c6ccccc6c6ccccc65)cc5ccccc45)cc3)c3ccc4sc5ccccc5c4c3)cc2)cc1] | 72 |
| 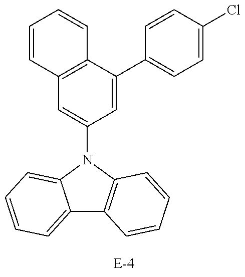 | 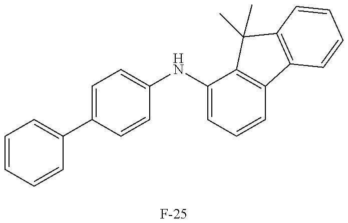 | 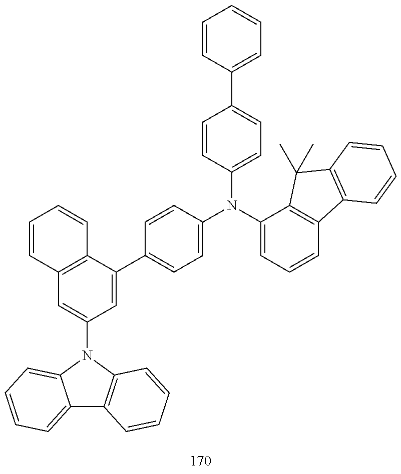 [SMILES: CC1(C)c2ccccc2-c2cccc(N(c3ccc(-c4ccccc4)cc3)c3ccc(-c4cc(-n5c6ccccc6c6ccccc65)cc5ccccc45)cc3)c21] | 73 |
| 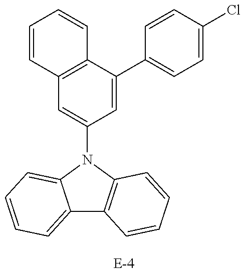 | 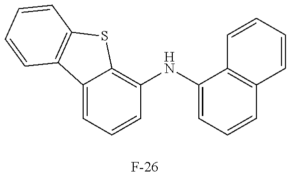 | 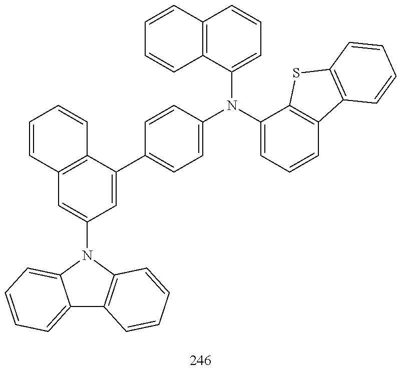 [SMILES: c1ccc2c(-c3ccc(N(c4cccc5ccccc45)c4cccc5c4sc4ccccc45)cc3)cc(-n3c4ccccc4c4ccccc43)cc2c1] | 72 |
| 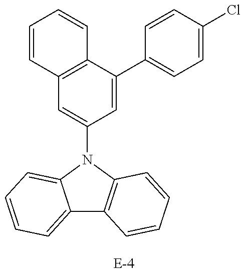 | 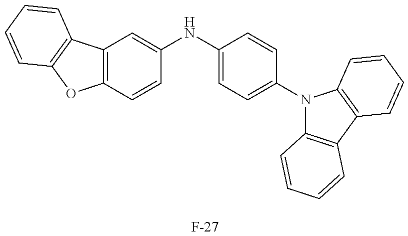 | 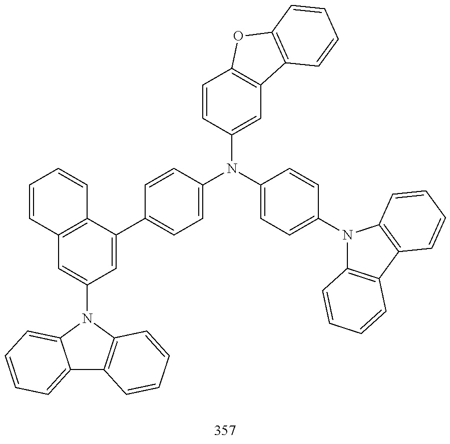 [SMILES: c1ccc2c(-c3ccc(N(c4ccc(-n5c6ccccc6c6ccccc65)cc4)c4ccc5oc6ccccc6c5c4)cc3)cc(-n3c4ccccc4c4ccccc43)cc2c1] | 73 |
| 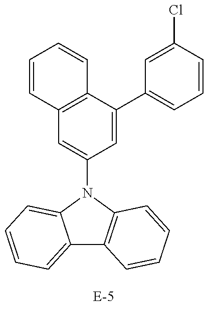 | 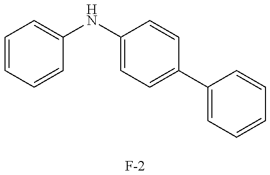 | 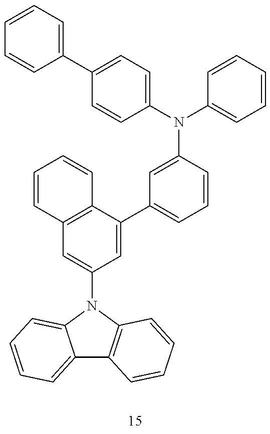 [SMILES: c1ccc(-c2ccc(N(c3ccccc3)c3cccc(-c4cc(-n5c6ccccc6c6ccccc65)cc5ccccc45)c3)cc2)cc1] | 72 |
| 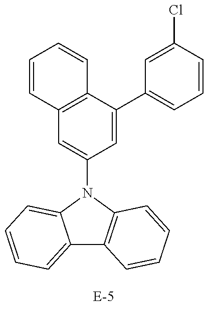 | 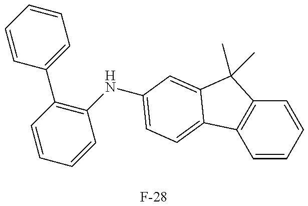 | 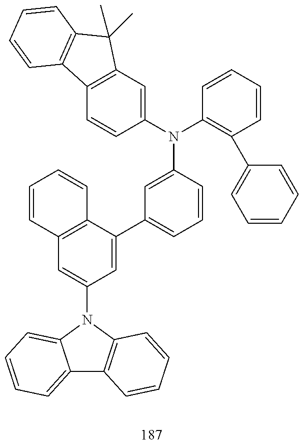 [SMILES: CC1(C)c2ccccc2-c2ccc(N(c3cccc(-c4cc(-n5c6ccccc6c6ccccc65)cc5ccccc45)c3)c3ccccc3-c3ccccc3)cc21] | 71 |
| 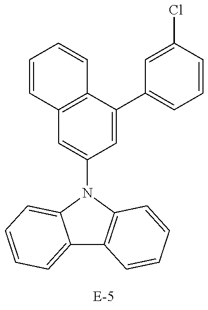 | 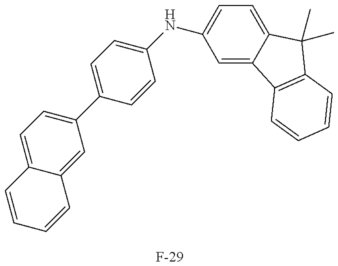 | 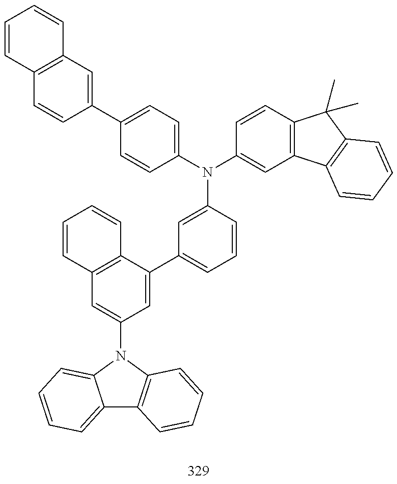 [SMILES: CC1(C)c2ccccc2-c2cc(N(c3ccc(-c4ccc5ccccc5c4)cc3)c3cccc(-c4cc(-n5c6ccccc6c6ccccc65)cc5ccccc45)c3)ccc21] | 73 |
| 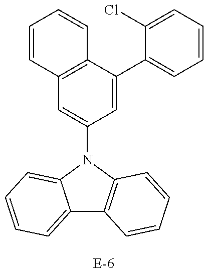 | 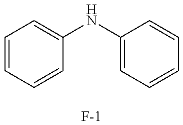 | 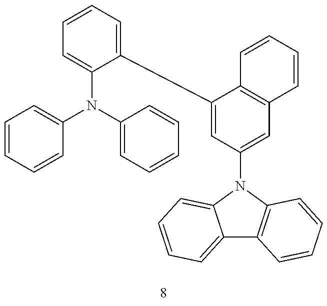 [SMILES: c1ccc(N(c2ccccc2)c2ccccc2-c2cc(-n3c4ccccc4c4ccccc43)cc3ccccc23)cc1] | 71 |
| 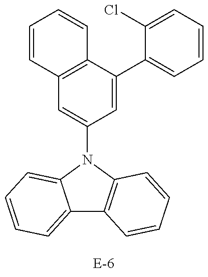 | 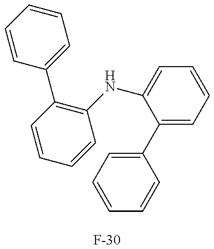 | 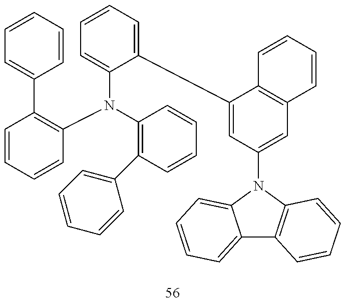 [SMILES: c1ccc(-c2ccccc2N(c2ccccc2-c2ccccc2)c2ccccc2-c2cc(-n3c4ccccc4c4ccccc43)cc3ccccc23)cc1] | 72 |
| 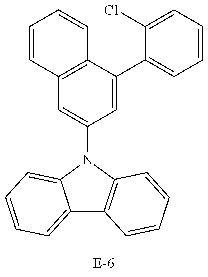 / | 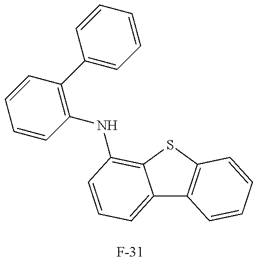 | 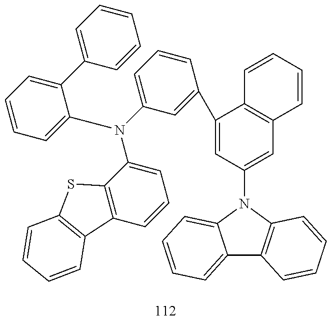 [SMILES: c1ccc(-c2ccccc2N(c2cccc(-c3cc(-n4c5ccccc5c5ccccc54)cc4ccccc34)c2)c2cccc3c2sc2ccccc23)cc1] | 73 |
| 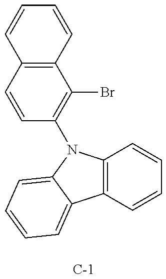 | 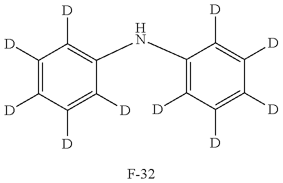 | 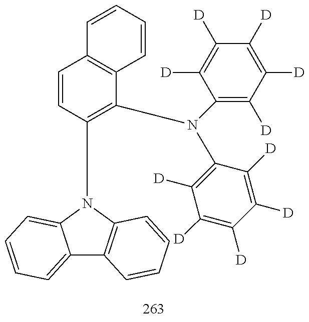 [SMILES: [2H]c1c([2H])c([2H])c(N(c2c([2H])c([2H])c([2H])c([2H])c2[2H])c2c(-n3c4ccccc4c4ccccc43)ccc3ccccc23)c([2H])c1[2H]] | 72 |
| 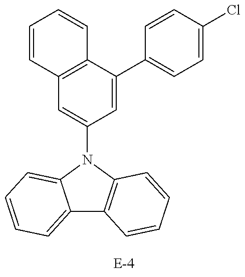 | 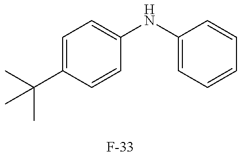 | 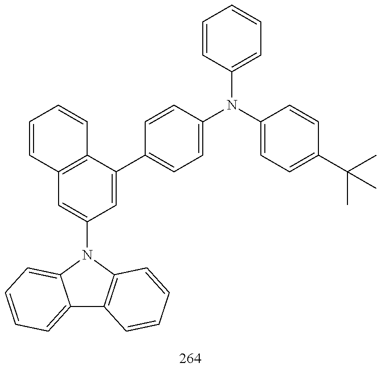 [SMILES: CC(C)(C)c1ccc(N(c2ccccc2)c2ccc(-c3cc(-n4c5ccccc5c5ccccc54)cc4ccccc34)cc2)cc1] | 73 |
| 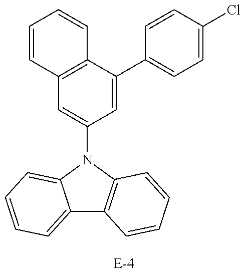 | 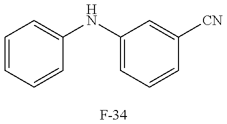 | 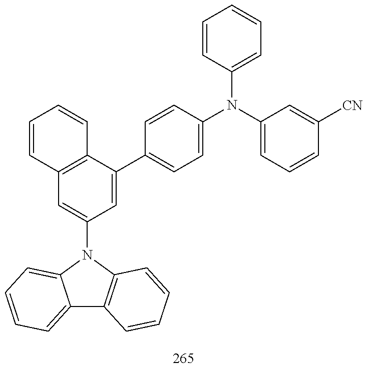 [SMILES: N#Cc1cccc(N(c2ccccc2)c2ccc(-c3cc(-n4c5ccccc5c5ccccc54)cc4ccccc34)cc2)c1] | 71 |
| 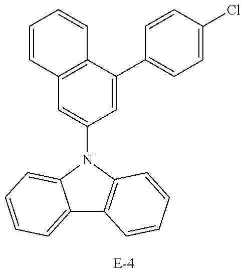 | 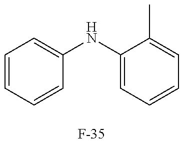 | 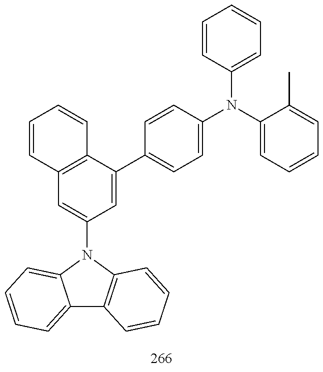 [SMILES: Cc1ccccc1N(c1ccccc1)c1ccc(-c2cc(-n3c4ccccc4c4ccccc43)cc3ccccc23)cc1] | 72 |
| 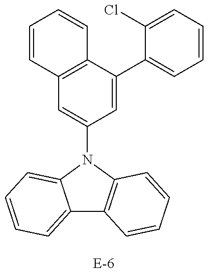 | 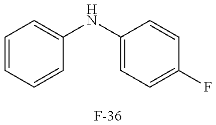 | 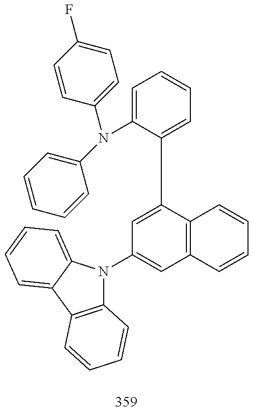 [SMILES: Fc1ccc(N(c2ccccc2)c2ccccc2-c2cc(-n3c4ccccc4c4ccccc43)cc3ccccc23)cc1] | 71 |
| 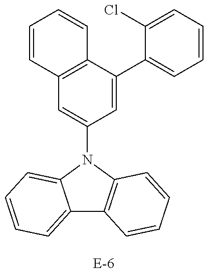 | 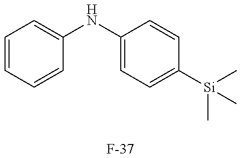 | 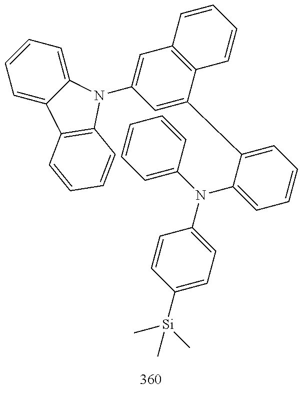 [SMILES: C[Si](C)(C)c1ccc(N(c2ccccc2)c2ccccc2-c2cc(-n3c4ccccc4c4ccccc43)cc3ccccc23)cc1] | 73 |

Mass spectrum data of some compounds are shown in Table 4 below:

TABLE 4

| | Mass spectrum | | Mass spectrum |
|---|---|---|---|
| Compound | [M + H]⁺ | Compound | [M+H]+ |
| 2 | 537.2 | 9 | 537.2 |
| 85 | 627.2 | 121 | 643.2 |
| 345 | 693.3 | 45 | 613.3 |
| 61 | 551.2 | 81 | 627.2 |
| 161 | 653.3 | 346 | 703.3 |
| 34 | 689.3 | 58 | 627.3 |
| 300 | 739.3 | 324 | 753.3 |
| 340 | 815.3 | 51 | 689.3 |
| 91 | 703.3 | 131 | 719.2 |
| 148 | 719.2 | 219 | 778.3 |
| 96 | 703.3 | 168 | 729.3 |
| 278 | 663.3 | 150 | 719.3 |
| 170 | 729.3 | 246 | 693.3 |
| 357 | 792.3 | 15 | 613.3 |
| 187 | 729.3 | 329 | 779.3 |
| 8 | 537.2 | 56 | 689.3 |
| 112 | 719.3 | 263 | 471.3 |
| 264 | 593.3 | 265 | 562.3 |
| 266 | 551.3 | 359 | 555.3 |
| 360 | 609.3 | 361 | 689.3 |

NMR data of some compoundsare shown in Table 5 below:

TABLE 5

| Compound | NMR data |
|---|---|
| 2 | ¹HNMR (400 MHz, CD₂Cl₂): 8.75-8.63 (m, 4H), |
| | 8.32 (d, 1H), 8.22-8.11 (m, 5H), 7.87-7.76 (m, 8H), |
| | 7.56-7.36 (m, 6H), 6.96 (d, 4H). |

Manufacture and Evaluation of Organic Electroluminescent Device

### Example 1

Blue Organic Electroluminescent Device

An anode was prepared by the following process: an ITO substrate (manufactured by Corning) having a thickness of 1500 Å was cut into a dimension of 40 mm×40 mm×0.7 mm to be prepared into an experimental substrate with a cathode pattern, an anode pattern and an insulating layer pattern by adopting a photoetching process, and surface treatment was performed by utilizing ultraviolet ozone and O₂:N₂ plasma so as to increase the work function of the anode (the experimental substrate) and remove scum.

F4-TCNQ was vacuum-evaporated on the experimental substrate (the anode) to form a hole injection layer (HIL) with a thickness of 100 Å, and NPB was evaporated on the hole injection layer to form a hole transport layer with a thickness of 1200 Å.

A compound 2 was vacuum-evaporated on the hole transport layer to form an electron blocking layer with a thickness of 100 Å.

BH-01 and BD-01 were co-evaporated on the electron blocking layer in a ratio of 98%:2% to form a blue organic light-emitting layer (EML) with a thickness of 220 Å.

ET-06 and LiQ were evaporated at a film thickness ratio of 1:1 to form an electron transport layer (ETL) with a thickness of 300 Å, Yb was evaporated on the electron transport layer to form an electron injection layer (EIL) with a thickness of 15 Å, and then magnesium (Mg) and silver (Ag) were vacuum-evaporated on the electron injection layer at a film thickness ratio of 1:9 to form a cathode with a thickness of 120 Å.

In addition, CP-5 was evaporated on the cathode with a thickness of 650 Å to form an organic capping layer (CPL), thus completing the manufacturing of the organic electroluminescent device.

### Examples 2 to 40

An organic electroluminescent device was manufactured by the same method as that in Example 1 except that compounds shown in Table 6 below were used instead of the compound 2 when the electron blocking layer was formed.

### Comparative Example 1

An organic electroluminescent device was manufactured by the same method as that in Example 1 except that the compound 2 was replaced by a compound A when the electron blocking layer was formed.

### Comparative Example 2

An organic electroluminescent device was manufactured by the same method as that in Example 1 except that the compound 2 was replaced by a compound B when the electron blocking layer was formed.

### Comparative Example 3

An organic electroluminescent device was manufactured by the same method as that in Example 1 except that the compound 2 was replaced by a compound C when the electron blocking layer was formed.

### Comparative Example 4

An organic electroluminescent device was manufactured by the same method as that in Example 1 except that the compound 2 was replaced by a compound D when the electron blocking layer was formed.

The structures of materials used in the above examples and comparative examples are shown below:

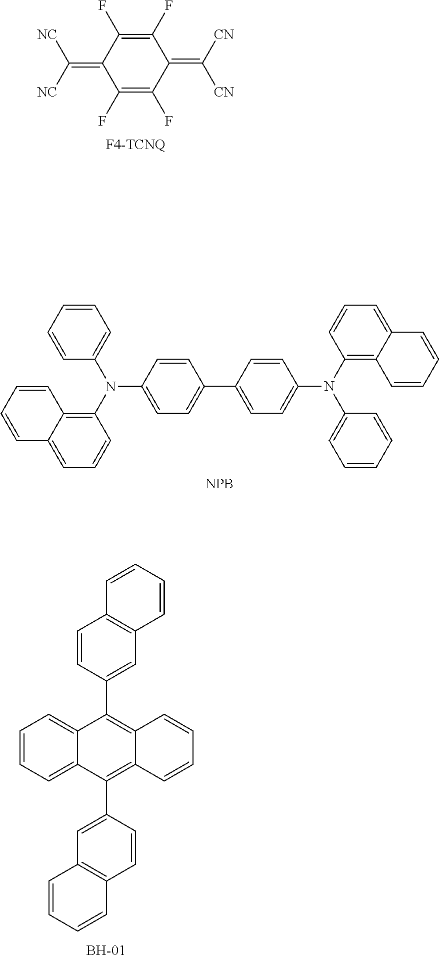

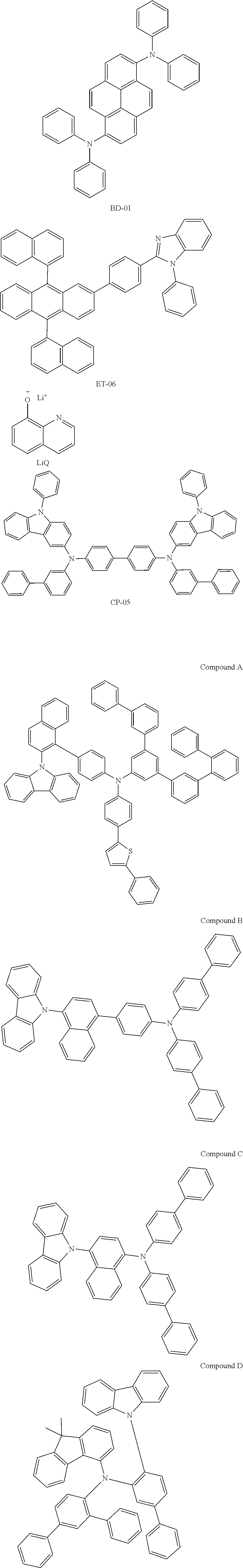

The performance of the blue organic electroluminescent devices manufactured in Examples 1 to 40 and Comparative Examples 1 to 4 was tested, and the performance of the devices was analyzed specifically under the condition of 20 mA/cm², and the test results are shown in Table 6.

TABLE 6

| | Electron | | | | | | | T95 |
|---|---|---|---|---|---|---|---|---|
| | blocking | Volt | | | | | EQE | (hrs)@20 |
| Example | layer | (V) | Cd/A | lm/W | CIE-x | CIE-Y | % | mA/cm² |
| Example 1 | Compound 2 | 3.87 | 6.98 | 5.67 | 0.14 | 0.05 | 14.36 | 242 |
| Example 2 | Compound 9 | 3.83 | 5.98 | 4.63 | 0.14 | 0.05 | 12.30 | 214 |
| Example 3 | Compound 85 | 3.90 | 5.98 | 4.55 | 0.14 | 0.05 | 12.30 | 225 |
| Example 4 | Compound 121 | 3.83 | 6.03 | 4.68 | 0.14 | 0.05 | 12.40 | 219 |
| Example 5 | Compound 345 | 3.88 | 5.99 | 4.65 | 0.14 | 0.05 | 12.32 | 246 |
| Example 6 | Compound 45 | 3.92 | 6.03 | 4.65 | 0.14 | 0.05 | 12.40 | 239 |
| Example 7 | Compound 61 | 3.92 | 6.00 | 4.63 | 0.14 | 0.05 | 12.34 | 240 |
| Example 8 | Compound 81 | 3.84 | 5.99 | 4.60 | 0.14 | 0.05 | 12.32 | 225 |
| Example 9 | Compound 161 | 3.92 | 5.99 | 4.63 | 0.14 | 0.05 | 12.32 | 225 |
| Example 10 | Compound 346 | 3.84 | 5.98 | 4.53 | 0.14 | 0.05 | 12.30 | 222 |
| Example 11 | Compound 34 | 3.83 | 6.67 | 5.17 | 0.14 | 0.05 | 13.72 | 229 |
| Example 12 | Compound 58 | 3.91 | 6.79 | 5.27 | 0.14 | 0.05 | 13.97 | 225 |
| Example 13 | Compound 300 | 3.85 | 6.95 | 5.36 | 0.14 | 0.05 | 14.30 | 222 |
| Example 14 | Compound 324 | 3.92 | 6.79 | 5.25 | 0.14 | 0.05 | 13.97 | 221 |
| Example 15 | Compound 51 | 3.89 | 6.96 | 5.32 | 0.14 | 0.05 | 14.32 | 230 |
| Example 16 | Compound 91 | 3.84 | 7.05 | 5.42 | 0.14 | 0.05 | 14.50 | 245 |
| Example 17 | Compound 131 | 3.88 | 7.00 | 5.42 | 0.14 | 0.05 | 14.40 | 216 |
| Example 18 | Compound 148 | 3.90 | 6.93 | 5.32 | 0.14 | 0.05 | 14.26 | 221 |
| Example 19 | Compound 219 | 3.83 | 6.89 | 5.29 | 0.14 | 0.05 | 14.17 | 246 |
| Example 20 | Compound 96 | 3.92 | 6.87 | 5.32 | 0.14 | 0.05 | 14.13 | 220 |
| Example 21 | Compound 168 | 3.87 | 6.92 | 5.37 | 0.14 | 0.05 | 14.23 | 230 |
| Example 22 | Compound 278 | 3.85 | 7.07 | 5.36 | 0.14 | 0.05 | 14.54 | 214 |
| Example 23 | Compound 150 | 3.84 | 6.69 | 5.18 | 0.14 | 0.05 | 13.76 | 243 |
| Example 24 | Compound 170 | 3.90 | 6.73 | 5.21 | 0.14 | 0.05 | 13.84 | 232 |
| Example 25 | Compound 246 | 3.90 | 6.85 | 5.24 | 0.14 | 0.05 | 14.09 | 229 |
| Example 26 | Compound 357 | 3.89 | 6.82 | 5.25 | 0.14 | 0.05 | 14.03 | 235 |
| Example 27 | Compound 15 | 3.88 | 6.65 | 5.05 | 0.14 | 0.05 | 13.68 | 241 |
| Example 28 | Compound 187 | 3.92 | 6.71 | 5.18 | 0.14 | 0.05 | 13.80 | 219 |
| Example 29 | Compound 329 | 3.86 | 6.67 | 5.06 | 0.14 | 0.05 | 13.72 | 226 |
| Example 30 | Compound 8 | 3.84 | 7.08 | 5.45 | 0.14 | 0.05 | 14.56 | 232 |
| Example 31 | Compound 56 | 3.89 | 6.75 | 5.21 | 0.14 | 0.05 | 13.88 | 231 |
| Example 32 | Compound 112 | 3.84 | 6.64 | 5.10 | 0.14 | 0.05 | 13.66 | 246 |
| Example 33 | Compound 340 | 3.91 | 6.88 | 5.26 | 0.14 | 0.05 | 14.15 | 225 |
| Example 34 | Compound 263 | 3.86 | 5.99 | 4.57 | 0.14 | 0.05 | 12.32 | 238 |
| Example 35 | Compound 264 | 3.91 | 6.66 | 5.14 | 0.14 | 0.05 | 13.70 | 229 |
| Example 36 | Compound 265 | 3.88 | 6.69 | 5.06 | 0.14 | 0.05 | 13.76 | 231 |
| Example 37 | Compound 266 | 3.85 | 6.79 | 5.19 | 0.14 | 0.05 | 13.97 | 227 |
| Example 38 | Compound 359 | 3.87 | 6.69 | 5.16 | 0.14 | 0.05 | 13.76 | 235 |
| Example 39 | Compound 360 | 3.86 | 7.01 | 5.41 | 0.14 | 0.05 | 14.42 | 229 |
| Example 40 | Compound 361 | 3.88 | 6.67 | 5.44 | 0.14 | 0.05 | 13.53 | 233 |
| Comparative | Compound A | 4.20 | 5.04 | 3.82 | 0.14 | 0.05 | 10.37 | 159 |
| Example 1 | | | | | | | | |
| Comparative | Compound B | 4.17 | 5.22 | 3.98 | 0.14 | 0.05 | 10.74 | 175 |
| Example 2 | | | | | | | | |
| Comparative | Compound C | 4.13 | 5.08 | 3.91 | 0.14 | 0.05 | 10.45 | 166 |
| Example 3 | | | | | | | | |
| Comparative | Compound D | 4.10 | 5.07 | 3.85 | 0.14 | 0.05 | 10.43 | 168 |
| Example 4 | | | | | | | | |

It can be seen from the results of Table 6 that according to Examples 1 to 40 of the nitrogen-containing compound as the electron blocking layer and device Comparative Examples 1 to 4 corresponding to known compounds, for the organic electroluminescent device described above manufactured with the nitrogen-containing compound as the electron blocking layer in the present disclosure, the driving voltage was reduced by at least 0.18 V, the luminous efficiency (Cd/A) was improved by at least 14.56%, the external quantum efficiency (EQE %) was improved by at least 14.53%, and the service life was improved by at least 22.29%.

It can be seen from the results of Table 6 that the nitrogen-containing compound of the present disclosure has lower voltage, better efficiency and service life compared with the compound A. The reason is that the nitrogen-containing compound of the present disclosure has an appropriate molecular weight, which makes the nitrogen-containing compound of the present disclosure have better thermal stability.

Table 7 below shows T1 energy levels calculation for some of the nitrogen-containing compounds of the present disclosure and the compounds in Comparative Examples, calculation software and version: Spartan 16, and calculation method: DFT/B3LYP/6-31G.

TABLE 7

| Compound | T1 value |
|---|---|
| Compound 34 | 2.69 (eV) |
| Compound 361 | 2.53 (eV) |
| Compound B | 2.30 (eV) |

It can be seen rom Tae that the nitrogen-containing compound of the present disclosure greatly enhances the steric hindrance due to the particular groups and the particular linking means, thus effectively increasing the T1 energy levels of the nitrogen-containing compound molecule compared with the compounds in Comparative Examples. Thus, when the nitrogen-containing compound is used as an electron blocking layer material, the performance of reducing the voltage, and improving the efficiency and service life is achieved.

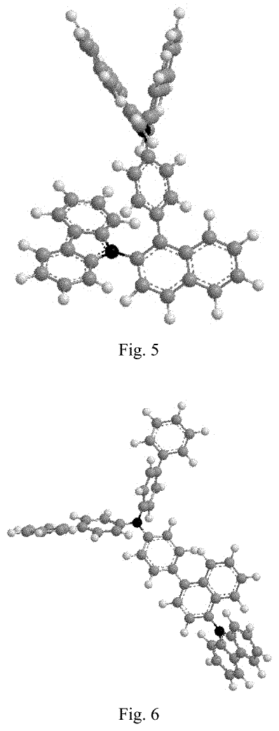

According to the comparison of the molecular structure model diagram of a compound 34 of the present disclosure (FIG. 5) and the molecular structure model diagram of the compound B in Comparative Example (FIG. 6), it can be seen that the particular linking means of the nitrogen-containing compound of the present disclosure greatly alters the spatial structure of the molecule, so that the nitrogen-containing compound has a higher steric hindrance, thus effectively improving the T1 energy levels of the compound molecule.

It should be understood that the present disclosure does not limit its application to the detailed structure and arrangement of the components proposed in the description. The present disclosure can have other examples, and can be implemented and executed in a variety of ways. The above deformation forms and modification forms fall within the scope of the present disclosure. It should be understood that the present disclosure, which is disclosed and defined in the description, extends to all alternative combinations of two or more individual features mentioned or apparent herein and/or in the accompanying drawings. All these different combinations constitute a plurality of alternative aspects of the present disclosure. The examples described in the description describe the best way known to implement the present disclosure and will enable those skilled in the art to utilize the present disclosure.

1. A nitrogen-containing compound, having a structure represented by a Formula 1: 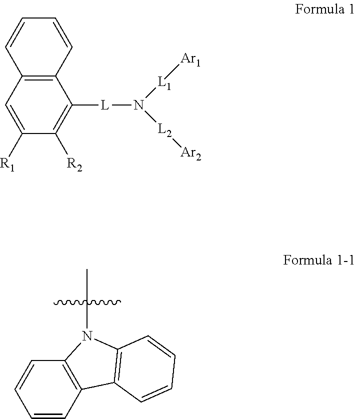
wherein R₁ and R₂ are respectively and independently selected from hydrogen or a group represented by a Formula 1-1, and only one of R₁ and R₂ is the group represented by a Formula 1-1;
L is selected from a single bond, and substituted or unsubstituted phenylene;
substituent in the L is selected from deuterium, a halogen group, and alkyl with 1 to 5 carbon atoms;
L₁ and L₂ are respectively and independently selected from a single bond, substituted or unsubstituted arylene with 6 to 30 carbon atoms, and substituted or unsubstituted heteroarylene with 3 to 30 carbon atoms;
Ar₁ and Ar₂ are selected from substituted or unsubstituted aryl with 6 to 30 carbon atoms, substituted or unsubstituted heteroaryl with 3 to 30 carbon atoms, substituted or unsubstituted alkyl with 1 to 20 carbon atoms, and substituted or unsubstituted cycloalkyl with 3 to 20 carbon atoms;
substituents in L₁, L₂, Ar₁ and Ar₂ are the same as or different from each other, and are each independently selected from deuterium, a halogen group, cyano, heteroaryl with 3 to 20 carbon atoms, aryl with 6 to 20 carbon atoms, trialkylsilyl with 3 to 12 carbon atoms, triarylsilyl with 18 to 24 carbon atoms, alkyl with 1 to 10 carbon atoms, haloalkyl with 1 to 10 carbon atoms, cycloalkyl with 3 to 10 carbon atoms, heterocycloalkyl with 2 to 10 carbon atoms, alkoxy with 1 to 10 carbon atoms, alkylthio with 1 to 10 carbon atoms, aryloxy with 6 to 18 carbon atoms, arylthio with 6 to 18 carbon atoms, and phosphinyloxy with 6 to 18 carbon atoms.

2. The nitrogen-containing compound of claim 1, wherein L is selected from a single bond or phenylene.

3. The nitrogen-containing compound of claim 1, wherein L₁ and L₂ are respectively and independently selected from a single bond, substituted or unsubstituted arylene with 6 to 20 carbon atoms, and substituted or unsubstituted heteroarylene with 5 to 20 carbon atoms.

4. The nitrogen-containing compound of claim 1, wherein L₁ and L₂ are respectively and independently selected from a single bond, substituted or unsubstituted phenylene, substituted or unsubstituted biphenylene, substituted or unsubstituted naphthylene, substituted or unsubstituted fluorenylene, substituted or unsubstituted carbazolylene, substituted or unsubstituted dibenzofurylene, and substituted or unsubstituted dibenzothienylene.

5. The nitrogen-containing compound of claim 1, wherein L₁ and L₂ are respectively and independently selected from a single bond, and a substituted or unsubstituted group V; the unsubstituted group V is selected from a group consisting of the following groups: 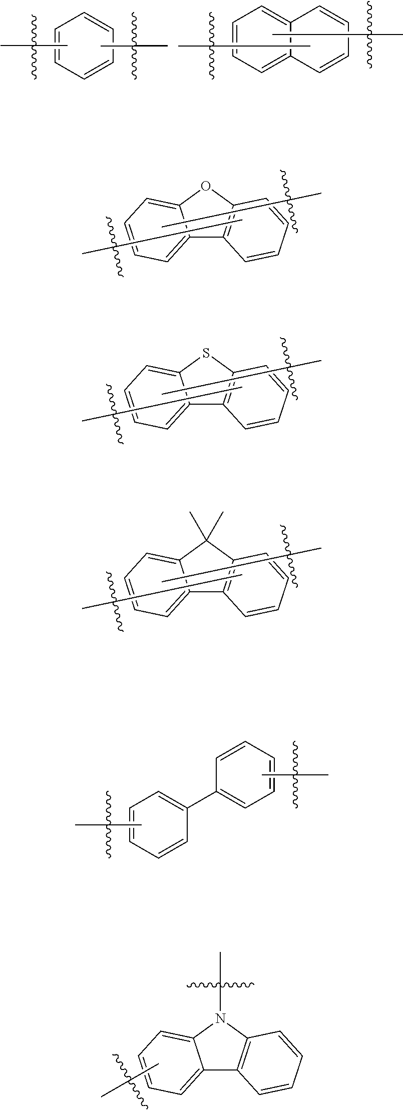
wherein, 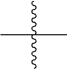
represents a chemical bond; substituted group V has one or more substituents, and the substituents are each independently selected from: deuterium, fluorine, cyano, methyl, ethyl, n-propyl, isopropyl, tert-butyl, phenyl, naphthyl, and biphenyl; when the number of the substituents in group V is greater than 1, the substituents are the same or different.

6. The nitrogen-containing compound of claim 1, wherein Ar₁ and Ar₂ are respectively and independently selected from substituted or unsubstituted aryl with 6 to 20 carbon atoms, and substituted or unsubstituted heteroaryl with 12 to 20 carbon atoms.

7. The nitrogen-containing compound of claim 1, wherein Ar₁ and Ar₂ are selected from a substituted or unsubstituted group W, the unsubstituted group W is selected from a group consisting of the following groups: 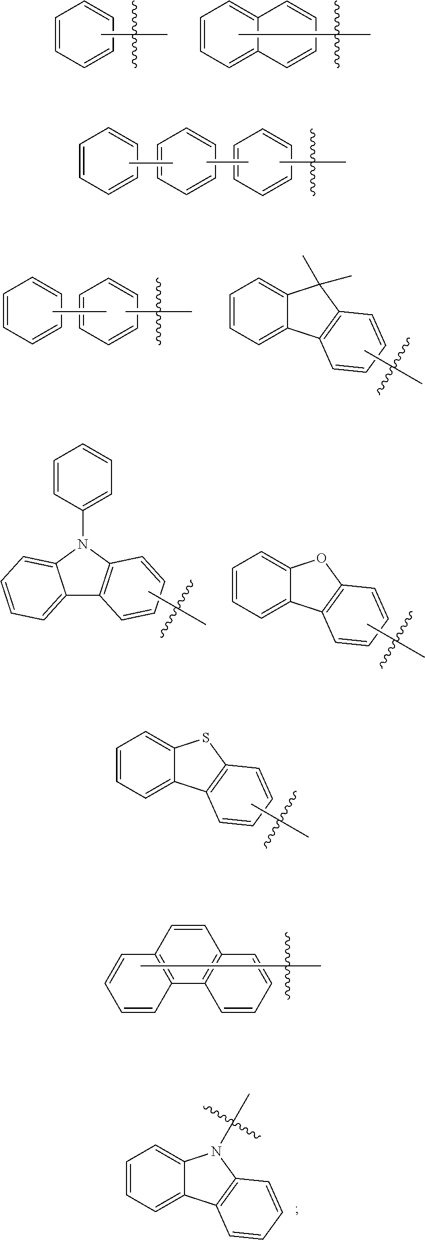
wherein, 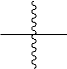
represents a chemical bond; substituted group W has one or more substituents, and the substituents are each independently selected from deuterium, fluorine, cyano, methyl, ethyl, n-propyl, isopropyl, tert-butyl, phenyl, naphthyl, biphenyl, and trimethylsilyl; when the number of the substituents in group W is greater than 1, the substituents are the same or different.

8. The nitrogen-containing compound of claim 1, wherein the nitrogen-containing compound is selected from a group consisting of the following compounds:

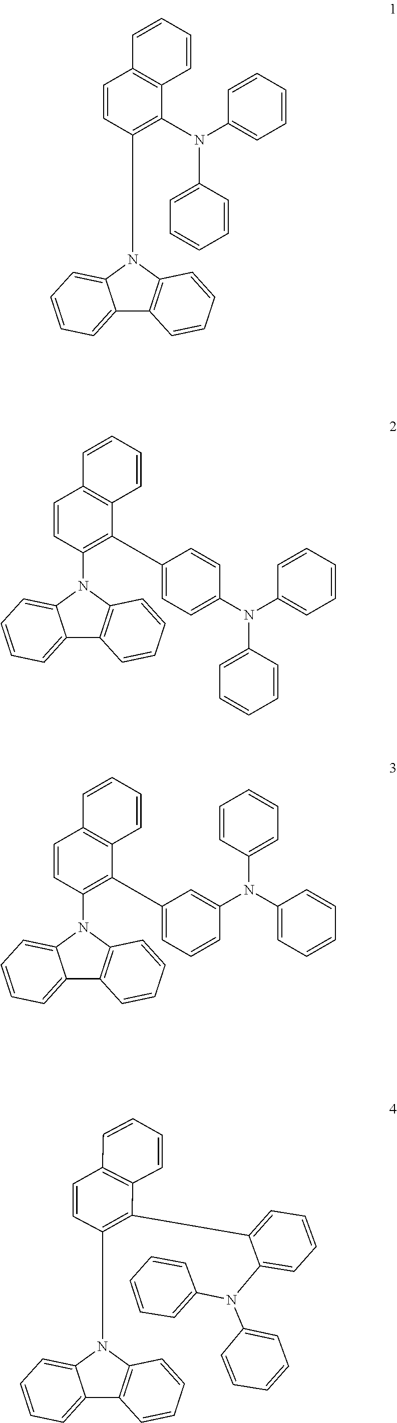 [SMILES: c1ccc(N(c2ccccc2)c2c(-n3c4ccccc4c4ccccc43)ccc3ccccc23)cc1 ; c1ccc(N(c2ccccc2)c2ccc(-c3c(-n4c5ccccc5c5ccccc54)ccc4ccccc34)cc2)cc1 ; c1ccc(N(c2ccccc2)c2cccc(-c3c(-n4c5ccccc5c5ccccc54)ccc4ccccc34)c2)cc1 ; c1ccc(N(c2ccccc2)c2ccccc2-c2c(-n3c4ccccc4c4ccccc43)ccc3ccccc23)cc1]

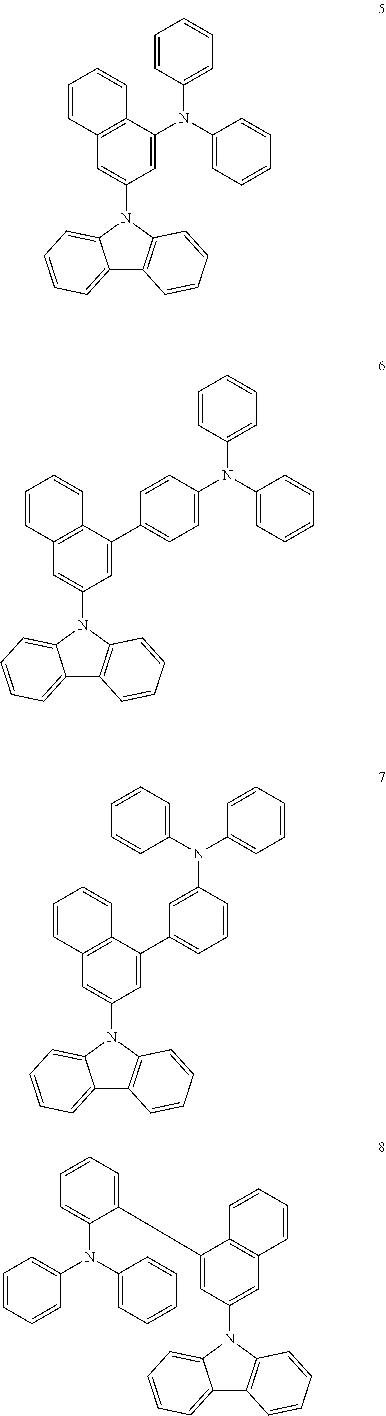 [SMILES: c1ccc(N(c2ccccc2)c2cc(-n3c4ccccc4c4ccccc43)cc3ccccc23)cc1 ; c1ccc(N(c2ccccc2)c2ccc(-c3cc(-n4c5ccccc5c5ccccc54)cc4ccccc34)cc2)cc1 ; c1ccc(N(c2ccccc2)c2cccc(-c3cc(-n4c5ccccc5c5ccccc54)cc4ccccc34)c2)cc1 ; c1ccc(N(c2ccccc2)c2ccccc2-c2cc(-n3c4ccccc4c4ccccc43)cc3ccccc23)cc1]

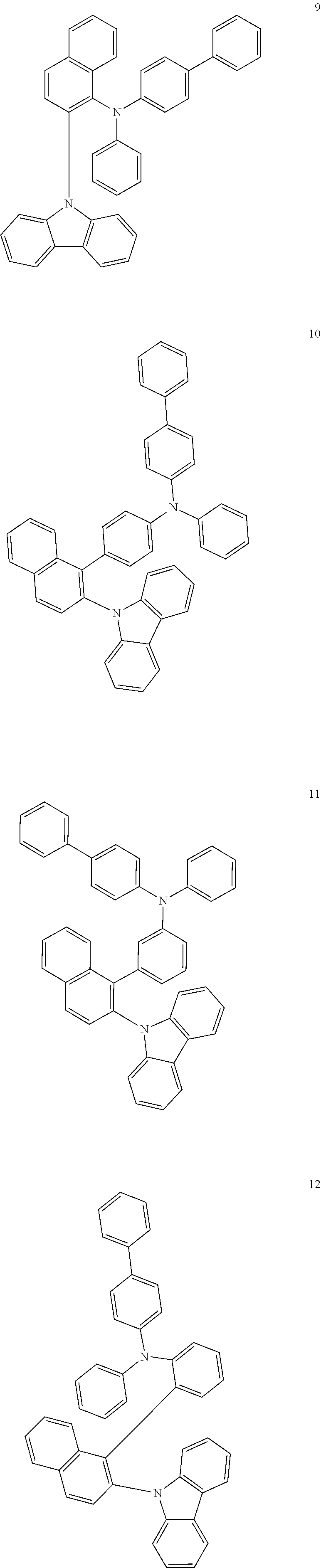 [SMILES: c1ccc(-c2ccc(N(c3ccccc3)c3c(-n4c5ccccc5c5ccccc54)ccc4ccccc34)cc2)cc1 ; c1ccc(-c2ccc(N(c3ccccc3)c3ccc(-c4c(-n5c6ccccc6c6ccccc65)ccc5ccccc45)cc3)cc2)cc1 ; c1ccc(-c2ccc(N(c3ccccc3)c3cccc(-c4c(-n5c6ccccc6c6ccccc65)ccc5ccccc45)c3)cc2)cc1 ; c1ccc(-c2ccc(N(c3ccccc3)c3ccccc3-c3c(-n4c5ccccc5c5ccccc54)ccc4ccccc34)cc2)cc1]

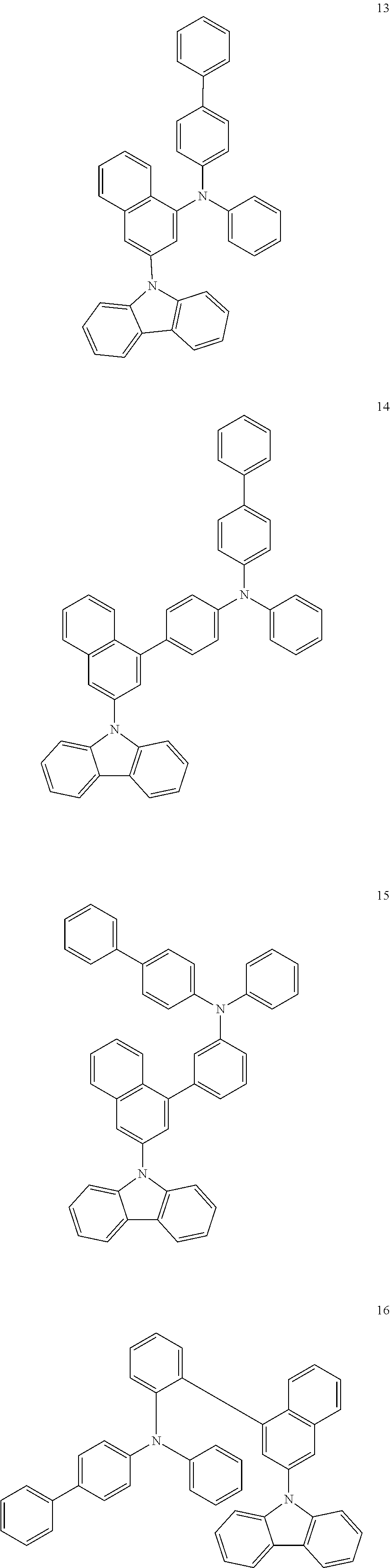 [SMILES: c1ccc(-c2ccc(N(c3ccccc3)c3cc(-n4c5ccccc5c5ccccc54)cc4ccccc34)cc2)cc1 ; c1ccc(-c2ccc(N(c3ccccc3)c3ccc(-c4cc(-n5c6ccccc6c6ccccc65)cc5ccccc45)cc3)cc2)cc1 ; c1ccc(-c2ccc(N(c3ccccc3)c3cccc(-c4cc(-n5c6ccccc6c6ccccc65)cc5ccccc45)c3)cc2)cc1 ; c1ccc(-c2ccc(N(c3ccccc3)c3ccccc3-c3cc(-n4c5ccccc5c5ccccc54)cc4ccccc34)cc2)cc1]

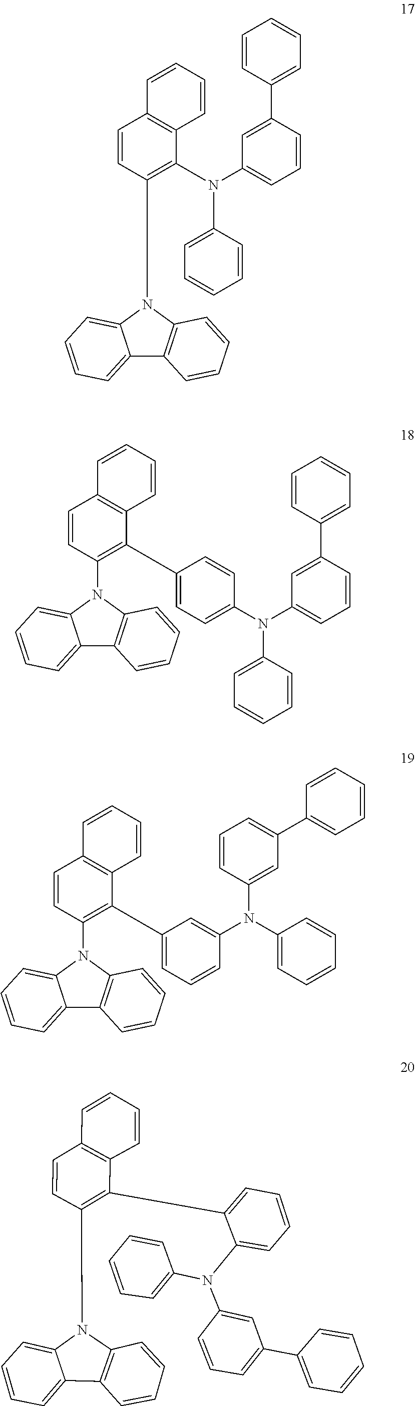 [SMILES: c1ccc(-c2cccc(N(c3ccccc3)c3c(-n4c5ccccc5c5ccccc54)ccc4ccccc34)c2)cc1 ; c1ccc(-c2cccc(N(c3ccccc3)c3ccc(-c4c(-n5c6ccccc6c6ccccc65)ccc5ccccc45)cc3)c2)cc1 ; c1ccc(-c2cccc(N(c3ccccc3)c3cccc(-c4c(-n5c6ccccc6c6ccccc65)ccc5ccccc45)c3)c2)cc1 ; c1ccc(-c2cccc(N(c3ccccc3)c3ccccc3-c3c(-n4c5ccccc5c5ccccc54)ccc4ccccc34)c2)cc1]

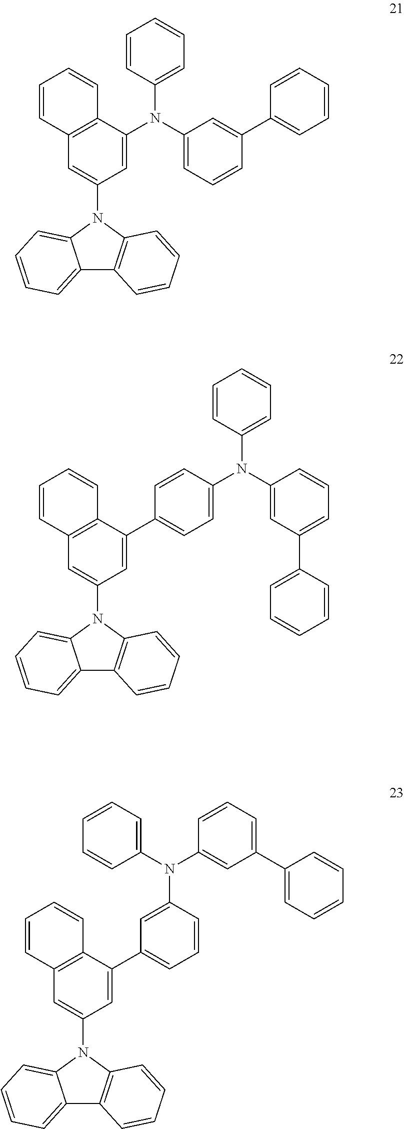 [SMILES: c1ccc(-c2cccc(N(c3ccccc3)c3cc(-n4c5ccccc5c5ccccc54)cc4ccccc34)c2)cc1 ; c1ccc(-c2cccc(N(c3ccccc3)c3ccc(-c4cc(-n5c6ccccc6c6ccccc65)cc5ccccc45)cc3)c2)cc1 ; c1ccc(-c2cccc(N(c3ccccc3)c3cccc(-c4cc(-n5c6ccccc6c6ccccc65)cc5ccccc45)c3)c2)cc1]

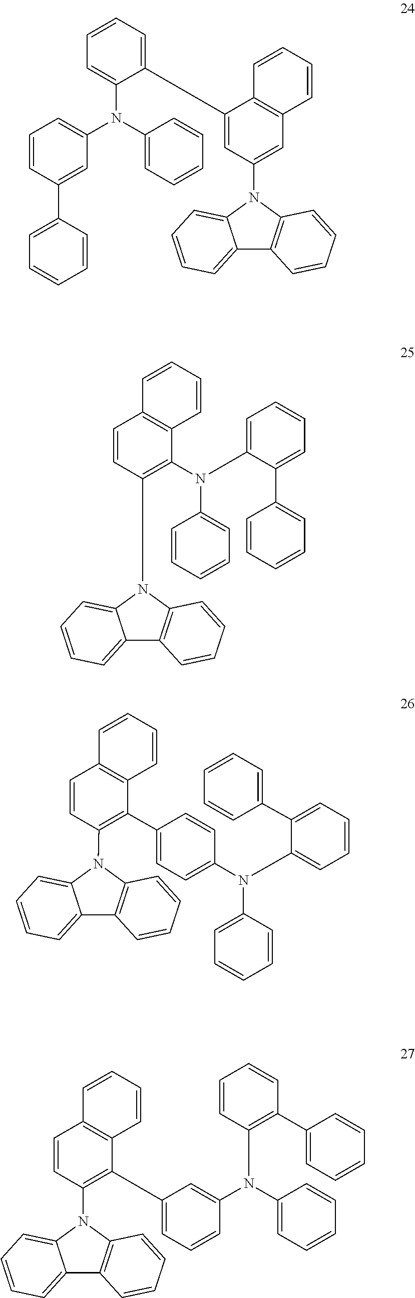 [SMILES: c1ccc(-c2cccc(N(c3ccccc3)c3ccccc3-c3cc(-n4c5ccccc5c5ccccc54)cc4ccccc34)c2)cc1 ; c1ccc(-c2ccccc2N(c2ccccc2)c2c(-n3c4ccccc4c4ccccc43)ccc3ccccc23)cc1 ; c1ccc(-c2ccccc2N(c2ccccc2)c2ccc(-c3c(-n4c5ccccc5c5ccccc54)ccc4ccccc34)cc2)cc1 ; c1ccc(-c2ccccc2N(c2ccccc2)c2cccc(-c3c(-n4c5ccccc5c5ccccc54)ccc4ccccc34)c2)cc1]

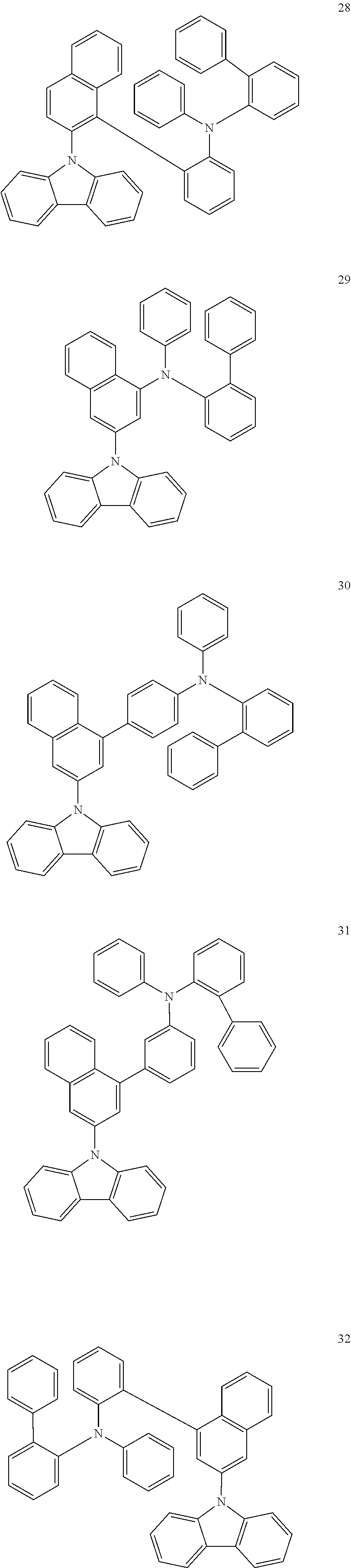 [SMILES: c1ccc(-c2ccccc2N(c2ccccc2)c2ccccc2-c2c(-n3c4ccccc4c4ccccc43)ccc3ccccc23)cc1 ; c1ccc(-c2ccccc2N(c2ccccc2)c2cc(-n3c4ccccc4c4ccccc43)cc3ccccc23)cc1 ; c1ccc(-c2ccccc2N(c2ccccc2)c2ccc(-c3cc(-n4c5ccccc5c5ccccc54)cc4ccccc34)cc2)cc1 ; c1ccc(-c2ccccc2N(c2ccccc2)c2cccc(-c3cc(-n4c5ccccc5c5ccccc54)cc4ccccc34)c2)cc1 ; c1ccc(-c2ccccc2N(c2ccccc2)c2ccccc2-c2cc(-n3c4ccccc4c4ccccc43)cc3ccccc23)cc1]

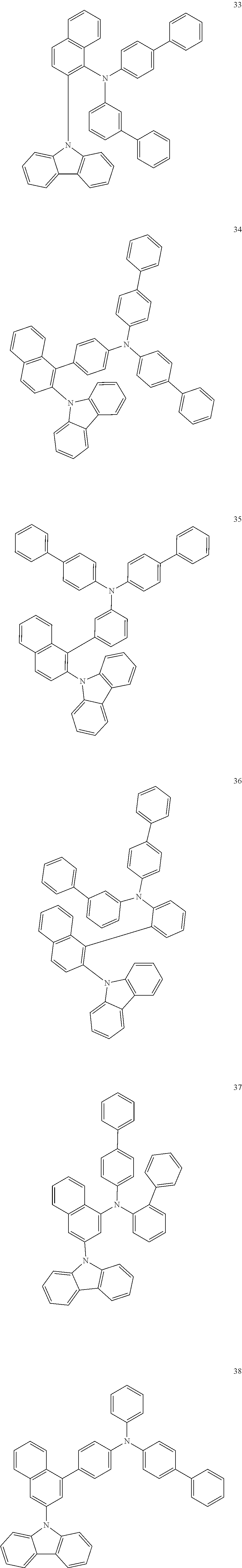

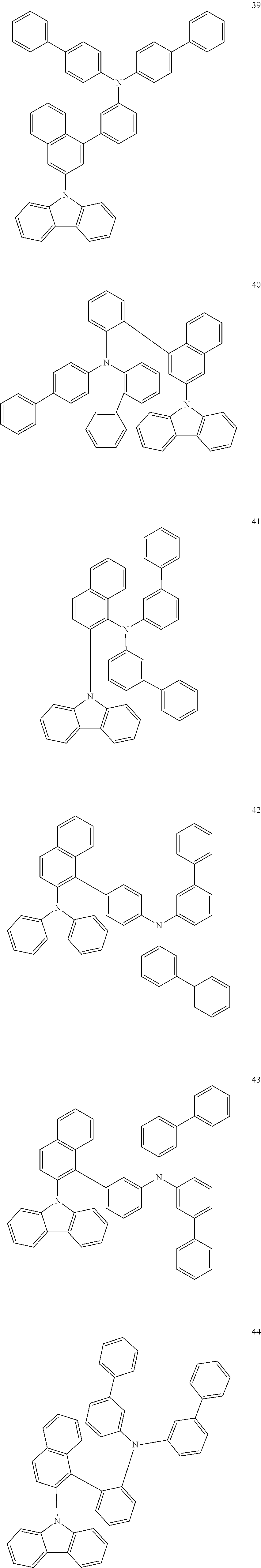

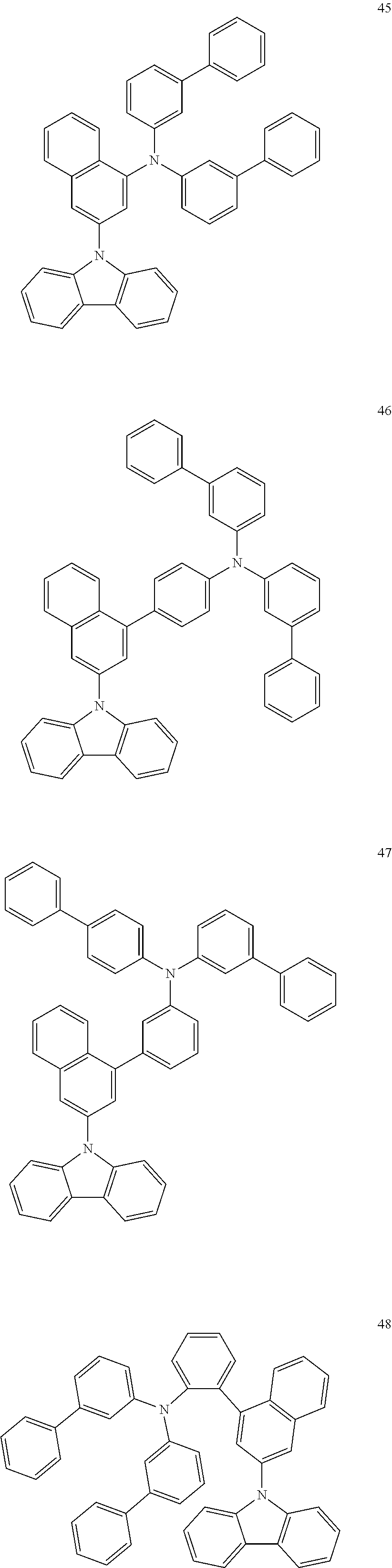 [SMILES: c1ccc(-c2cccc(N(c3cccc(-c4ccccc4)c3)c3cc(-n4c5ccccc5c5ccccc54)cc4ccccc34)c2)cc1 ; c1ccc(-c2cccc(N(c3ccc(-c4cc(-n5c6ccccc6c6ccccc65)cc5ccccc45)cc3)c3cccc(-c4ccccc4)c3)c2)cc1 ; c1ccc(-c2ccc(N(c3cccc(-c4ccccc4)c3)c3cccc(-c4cc(-n5c6ccccc6c6ccccc65)cc5ccccc45)c3)cc2)cc1 ; c1ccc(-c2cccc(N(c3cccc(-c4ccccc4)c3)c3ccccc3-c3cc(-n4c5ccccc5c5ccccc54)cc4ccccc34)c2)cc1]

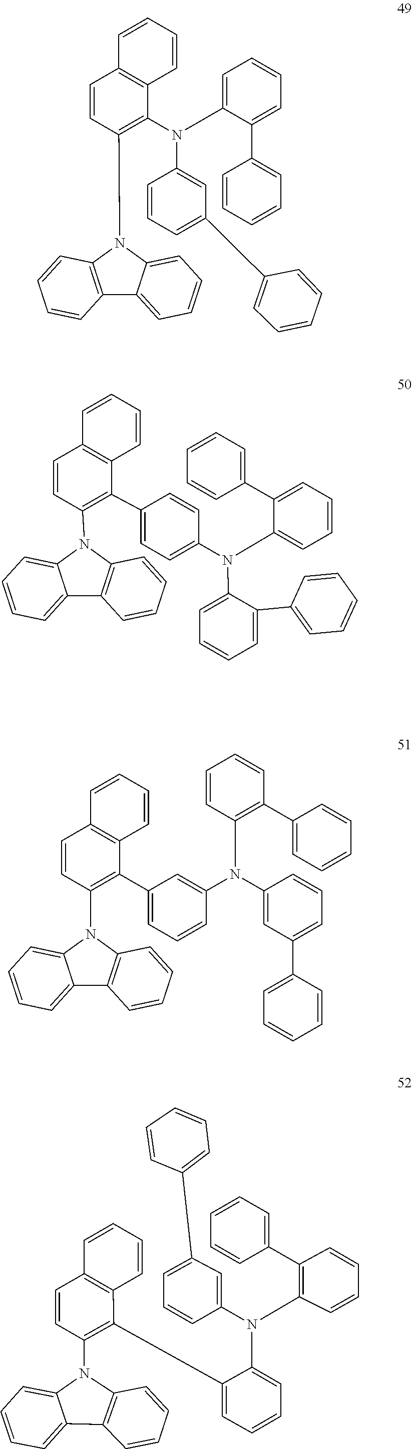 [SMILES: c1ccc(-c2cccc(N(c3ccccc3-c3ccccc3)c3c(-n4c5ccccc5c5ccccc54)ccc4ccccc34)c2)cc1 ; c1ccc(-c2ccccc2N(c2ccc(-c3c(-n4c5ccccc5c5ccccc54)ccc4ccccc34)cc2)c2ccccc2-c2ccccc2)cc1 ; c1ccc(-c2cccc(N(c3cccc(-c4c(-n5c6ccccc6c6ccccc65)ccc5ccccc45)c3)c3ccccc3-c3ccccc3)c2)cc1 ; c1ccc(-c2cccc(N(c3ccccc3-c3ccccc3)c3ccccc3-c3c(-n4c5ccccc5c5ccccc54)ccc4ccccc34)c2)cc1]

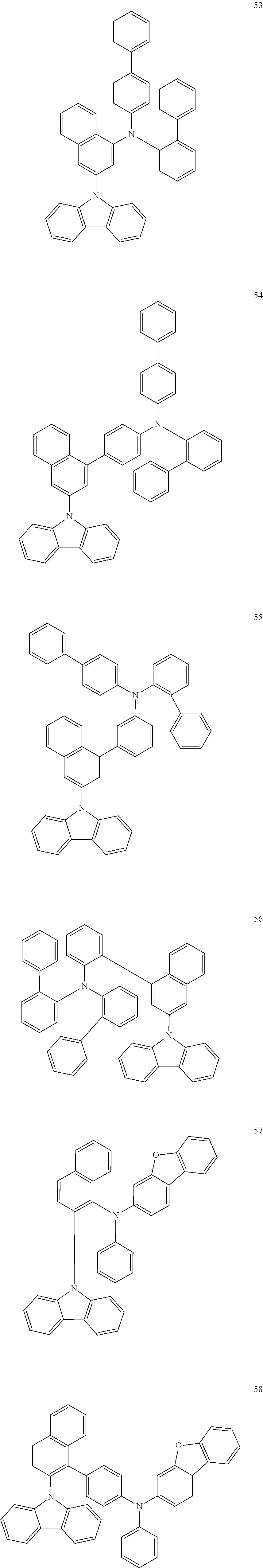

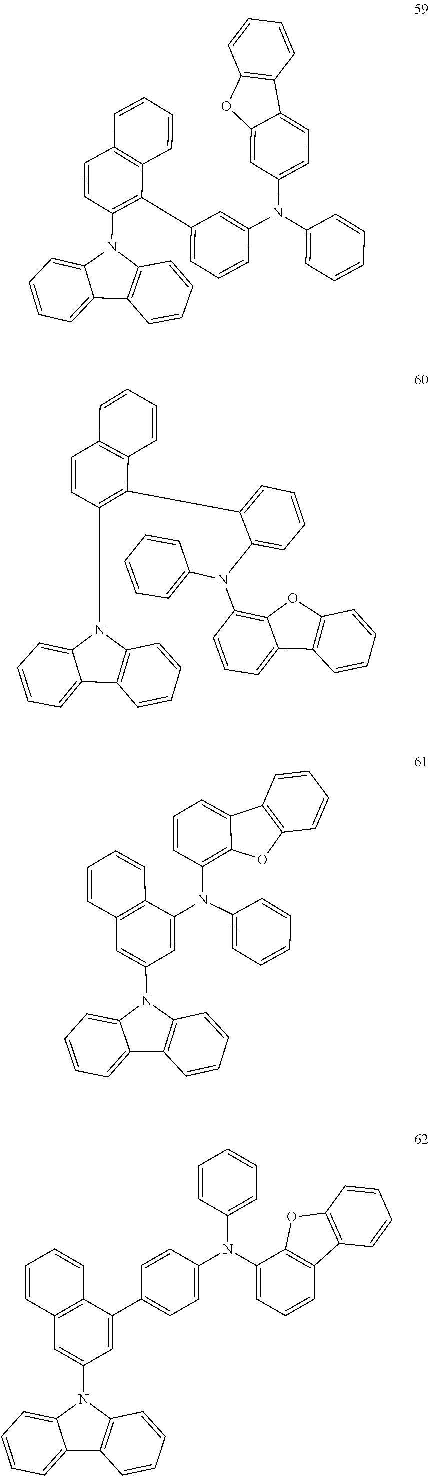 [SMILES: c1ccc(N(c2cccc(-c3c(-n4c5ccccc5c5ccccc54)ccc4ccccc34)c2)c2ccc3c(c2)oc2ccccc23)cc1 ; c1ccc(N(c2ccccc2-c2c(-n3c4ccccc4c4ccccc43)ccc3ccccc23)c2cccc3c2oc2ccccc23)cc1 ; c1ccc(N(c2cc(-n3c4ccccc4c4ccccc43)cc3ccccc23)c2cccc3c2oc2ccccc23)cc1 ; c1ccc(N(c2ccc(-c3cc(-n4c5ccccc5c5ccccc54)cc4ccccc34)cc2)c2cccc3c2oc2ccccc23)cc1]

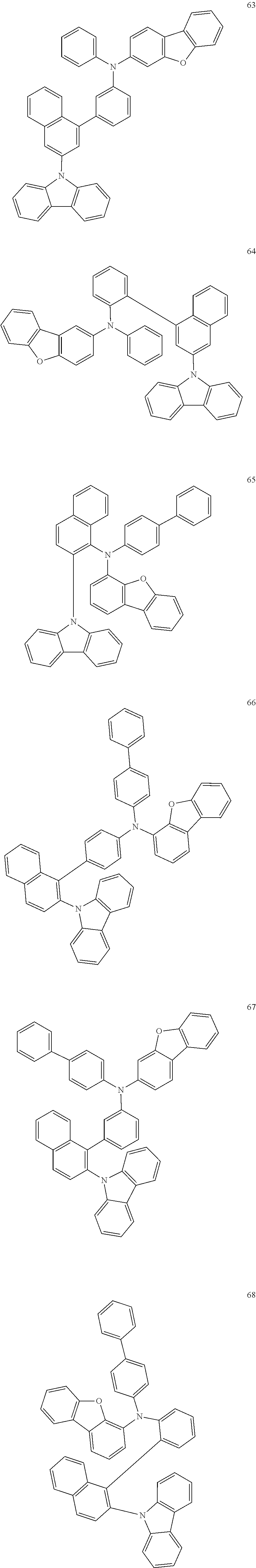

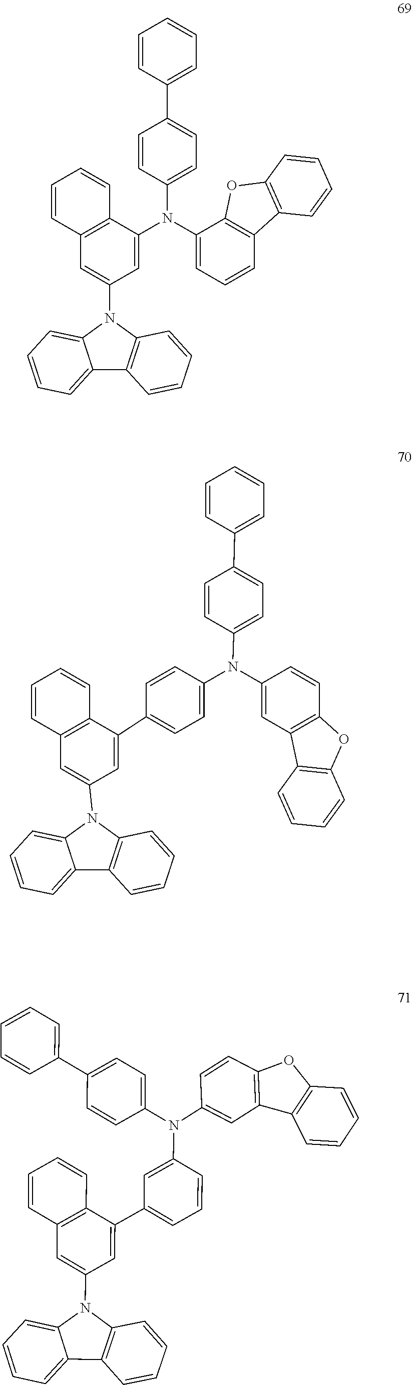 [SMILES: c1ccc(-c2ccc(N(c3cc(-n4c5ccccc5c5ccccc54)cc4ccccc34)c3cccc4c3oc3ccccc34)cc2)cc1 ; c1ccc(-c2ccc(N(c3ccc(-c4cc(-n5c6ccccc6c6ccccc65)cc5ccccc45)cc3)c3ccc4oc5ccccc5c4c3)cc2)cc1 ; c1ccc(-c2ccc(N(c3cccc(-c4cc(-n5c6ccccc6c6ccccc65)cc5ccccc45)c3)c3ccc4oc5ccccc5c4c3)cc2)cc1]

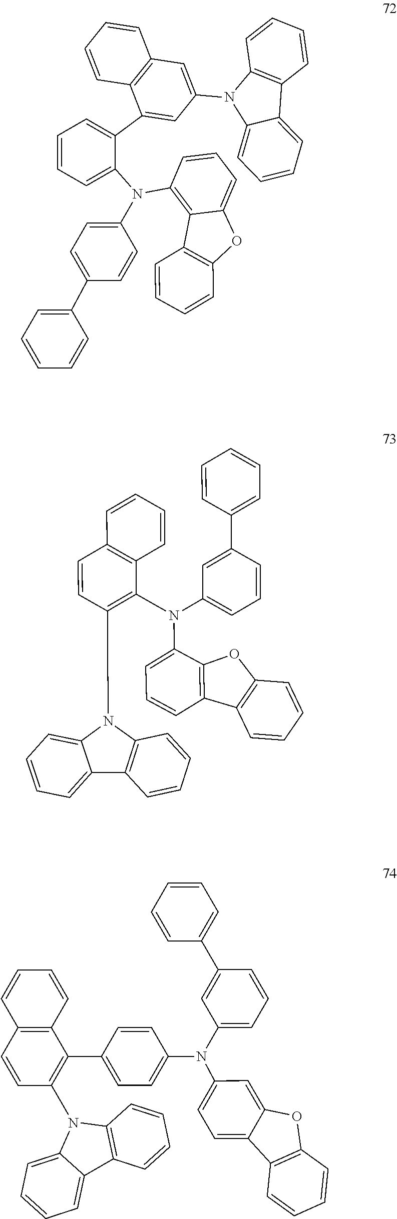 [SMILES: c1ccc(-c2ccc(N(c3ccccc3-c3cc(-n4c5ccccc5c5ccccc54)cc4ccccc34)c3cccc4oc5ccccc5c34)cc2)cc1 ; c1ccc(-c2cccc(N(c3c(-n4c5ccccc5c5ccccc54)ccc4ccccc34)c3cccc4c3oc3ccccc34)c2)cc1 ; c1ccc(-c2cccc(N(c3ccc(-c4c(-n5c6ccccc6c6ccccc65)ccc5ccccc45)cc3)c3ccc4c(c3)oc3ccccc34)c2)cc1]

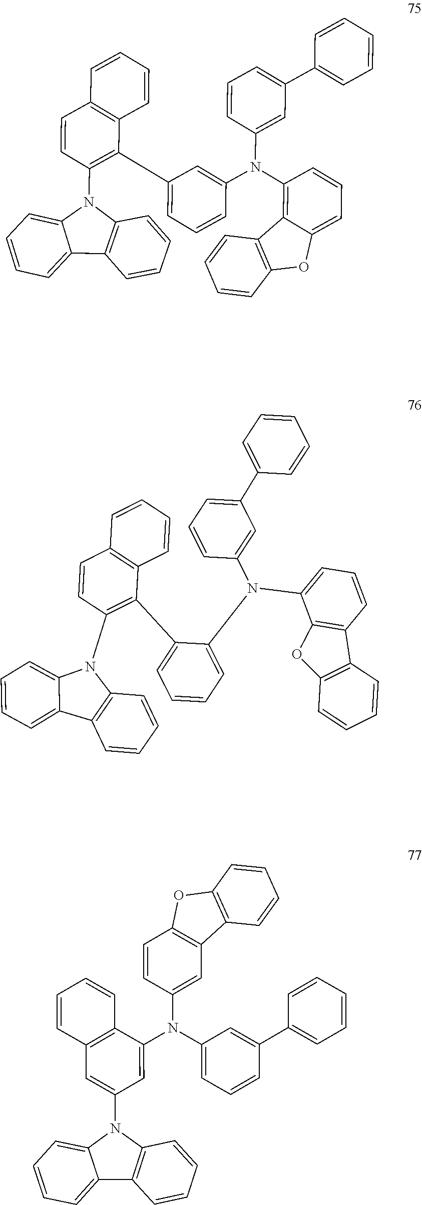 [SMILES: c1ccc(-c2cccc(N(c3cccc(-c4c(-n5c6ccccc6c6ccccc65)ccc5ccccc45)c3)c3cccc4oc5ccccc5c34)c2)cc1 ; c1ccc(-c2cccc(N(c3ccccc3-c3c(-n4c5ccccc5c5ccccc54)ccc4ccccc34)c3cccc4c3oc3ccccc34)c2)cc1 ; c1ccc(-c2cccc(N(c3ccc4oc5ccccc5c4c3)c3cc(-n4c5ccccc5c5ccccc54)cc4ccccc34)c2)cc1]

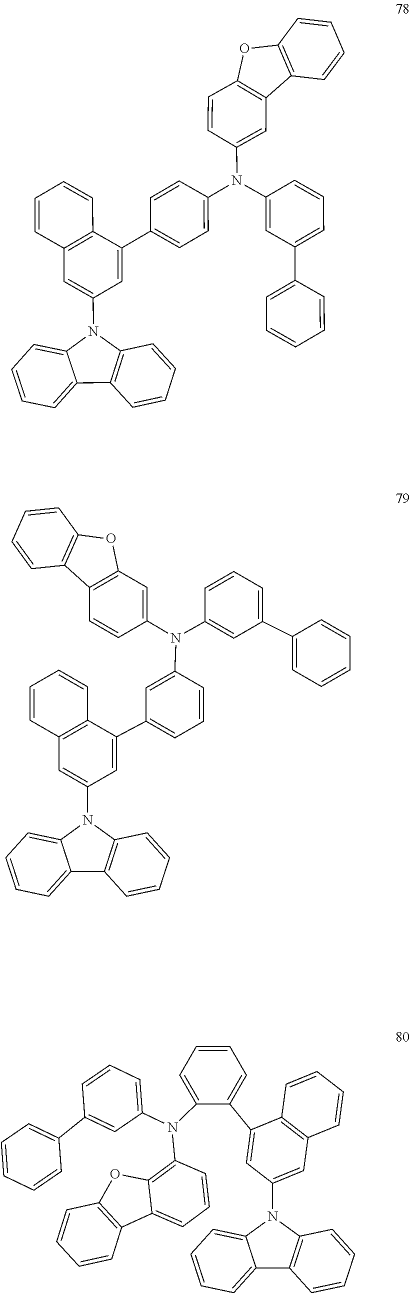 [SMILES: c1ccc(-c2cccc(N(c3ccc(-c4cc(-n5c6ccccc6c6ccccc65)cc5ccccc45)cc3)c3ccc4oc5ccccc5c4c3)c2)cc1 ; c1ccc(-c2cccc(N(c3cccc(-c4cc(-n5c6ccccc6c6ccccc65)cc5ccccc45)c3)c3ccc4c(c3)oc3ccccc34)c2)cc1 ; c1ccc(-c2cccc(N(c3ccccc3-c3cc(-n4c5ccccc5c5ccccc54)cc4ccccc34)c3cccc4c3oc3ccccc34)c2)cc1]

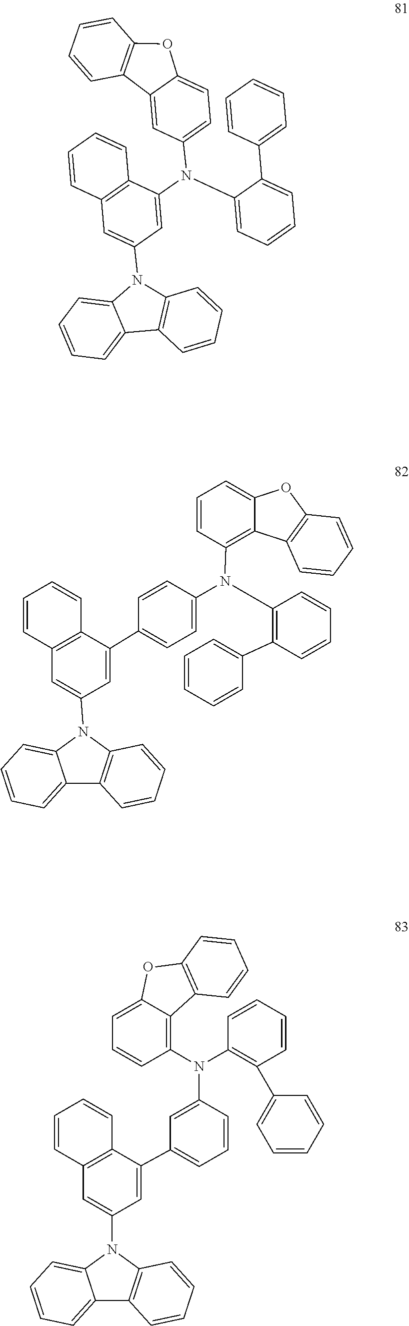 [SMILES: c1ccc(-c2ccccc2N(c2ccc3oc4ccccc4c3c2)c2cc(-n3c4ccccc4c4ccccc43)cc3ccccc23)cc1 ; c1ccc(-c2ccccc2N(c2ccc(-c3cc(-n4c5ccccc5c5ccccc54)cc4ccccc34)cc2)c2cccc3oc4ccccc4c23)cc1 ; c1ccc(-c2ccccc2N(c2cccc(-c3cc(-n4c5ccccc5c5ccccc54)cc4ccccc34)c2)c2cccc3oc4ccccc4c23)cc1]

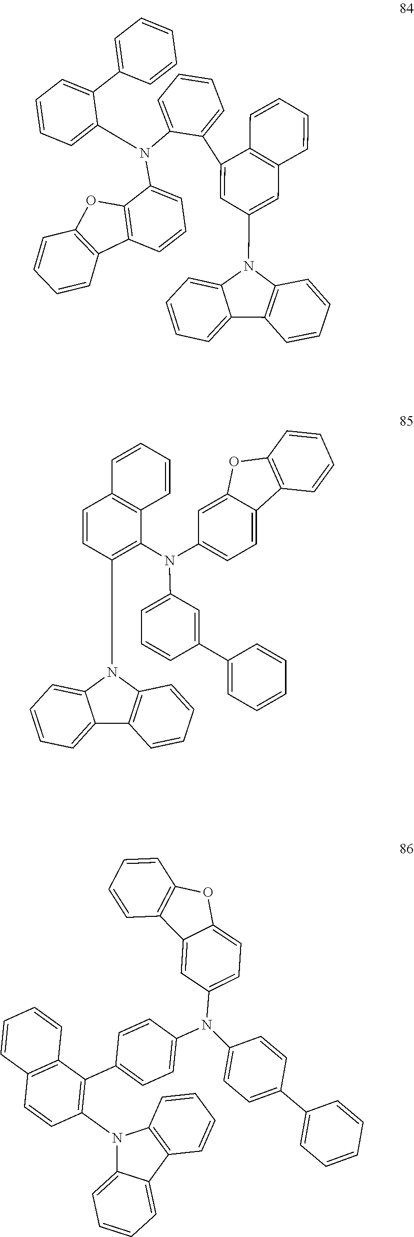 [SMILES: c1ccc(-c2ccccc2N(c2ccccc2-c2cc(-n3c4ccccc4c4ccccc43)cc3ccccc23)c2cccc3c2oc2ccccc23)cc1 ; c1ccc(-c2cccc(N(c3ccc4c(c3)oc3ccccc34)c3c(-n4c5ccccc5c5ccccc54)ccc4ccccc34)c2)cc1 ; c1ccc(-c2ccc(N(c3ccc(-c4c(-n5c6ccccc6c6ccccc65)ccc5ccccc45)cc3)c3ccc4oc5ccccc5c4c3)cc2)cc1]

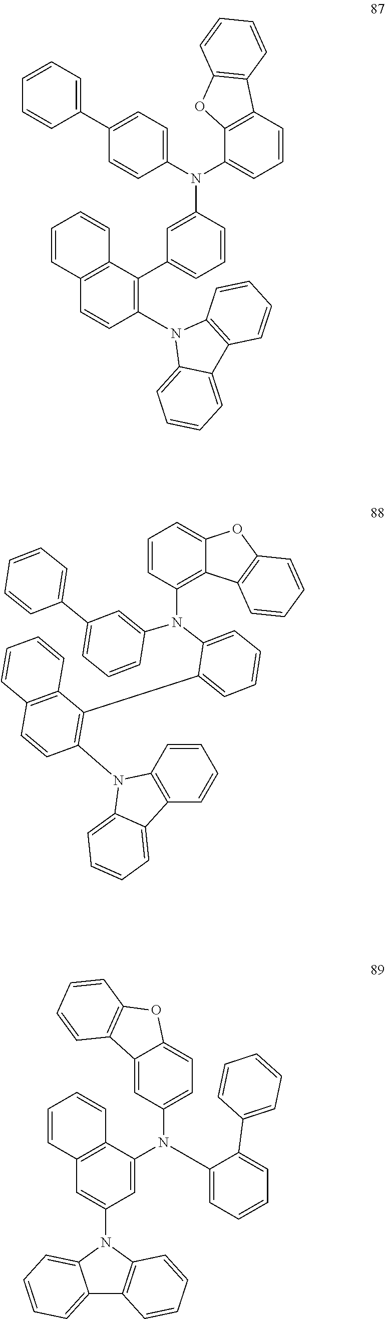 [SMILES: c1ccc(-c2ccc(N(c3cccc(-c4c(-n5c6ccccc6c6ccccc65)ccc5ccccc45)c3)c3cccc4c3oc3ccccc34)cc2)cc1 ; c1ccc(-c2cccc(N(c3ccccc3-c3c(-n4c5ccccc5c5ccccc54)ccc4ccccc34)c3cccc4oc5ccccc5c34)c2)cc1 ; c1ccc(-c2ccccc2N(c2ccc3oc4ccccc4c3c2)c2cc(-n3c4ccccc4c4ccccc43)cc3ccccc23)cc1]

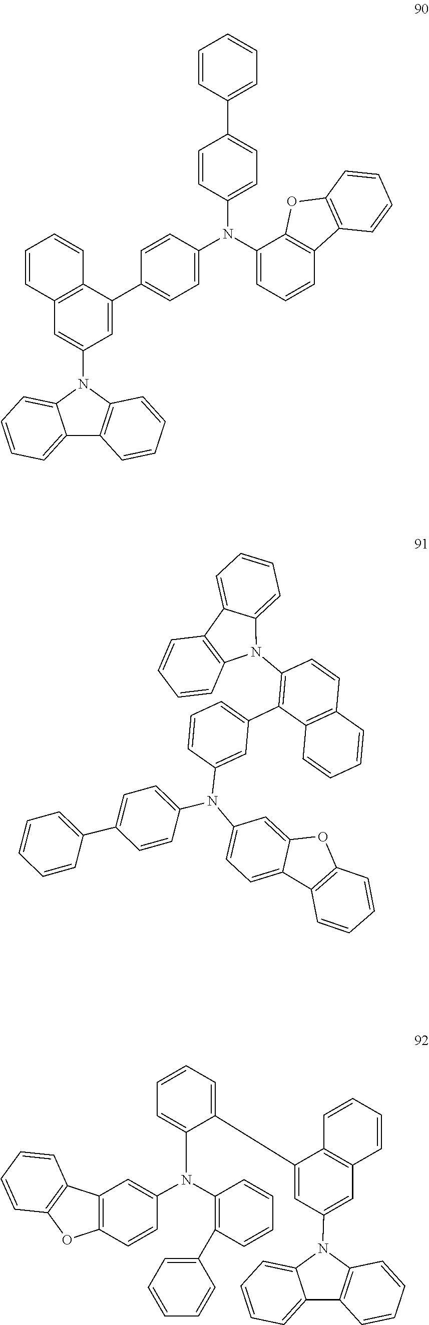 [SMILES: c1ccc(-c2ccc(N(c3ccc(-c4cc(-n5c6ccccc6c6ccccc65)cc5ccccc45)cc3)c3cccc4c3oc3ccccc34)cc2)cc1 ; c1ccc(-c2ccc(N(c3cccc(-c4c(-n5c6ccccc6c6ccccc65)ccc5ccccc45)c3)c3ccc4c(c3)oc3ccccc34)cc2)cc1 ; c1ccc(-c2ccccc2N(c2ccc3oc4ccccc4c3c2)c2ccccc2-c2cc(-n3c4ccccc4c4ccccc43)cc3ccccc23)cc1]

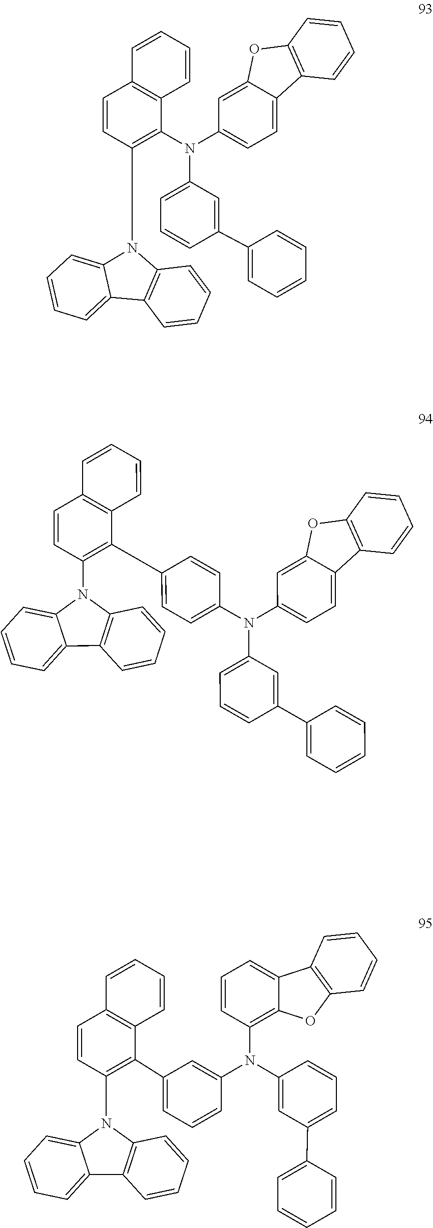 [SMILES: c1ccc(-c2cccc(N(c3ccc4c(c3)oc3ccccc34)c3c(-n4c5ccccc5c5ccccc54)ccc4ccccc34)c2)cc1 ; c1ccc(-c2cccc(N(c3ccc(-c4c(-n5c6ccccc6c6ccccc65)ccc5ccccc45)cc3)c3ccc4c(c3)oc3ccccc34)c2)cc1 ; c1ccc(-c2cccc(N(c3cccc(-c4c(-n5c6ccccc6c6ccccc65)ccc5ccccc45)c3)c3cccc4c3oc3ccccc34)c2)cc1]

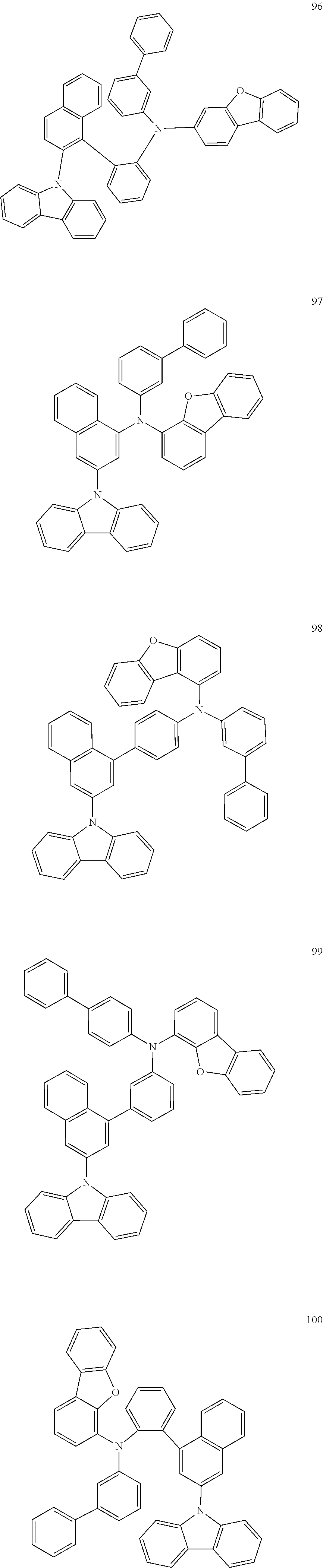 [SMILES: c1ccc(-c2cccc(N(c3ccc4c(c3)oc3ccccc34)c3ccccc3-c3c(-n4c5ccccc5c5ccccc54)ccc4ccccc34)c2)cc1 ; c1ccc(-c2cccc(N(c3cc(-n4c5ccccc5c5ccccc54)cc4ccccc34)c3cccc4c3oc3ccccc34)c2)cc1 ; c1ccc(-c2cccc(N(c3ccc(-c4cc(-n5c6ccccc6c6ccccc65)cc5ccccc45)cc3)c3cccc4oc5ccccc5c34)c2)cc1 ; c1ccc(-c2ccc(N(c3cccc(-c4cc(-n5c6ccccc6c6ccccc65)cc5ccccc45)c3)c3cccc4c3oc3ccccc34)cc2)cc1 ; c1ccc(-c2cccc(N(c3ccccc3-c3cc(-n4c5ccccc5c5ccccc54)cc4ccccc34)c3cccc4c3oc3ccccc34)c2)cc1]

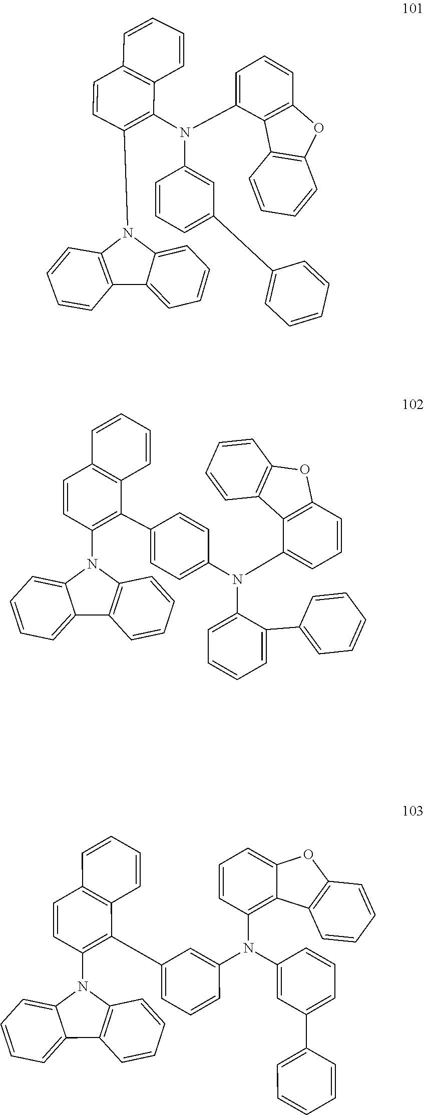 [SMILES: c1ccc(-c2cccc(N(c3c(-n4c5ccccc5c5ccccc54)ccc4ccccc34)c3cccc4oc5ccccc5c34)c2)cc1 ; c1ccc(-c2ccccc2N(c2ccc(-c3c(-n4c5ccccc5c5ccccc54)ccc4ccccc34)cc2)c2cccc3oc4ccccc4c23)cc1 ; c1ccc(-c2cccc(N(c3cccc(-c4c(-n5c6ccccc6c6ccccc65)ccc5ccccc45)c3)c3cccc4oc5ccccc5c34)c2)cc1]

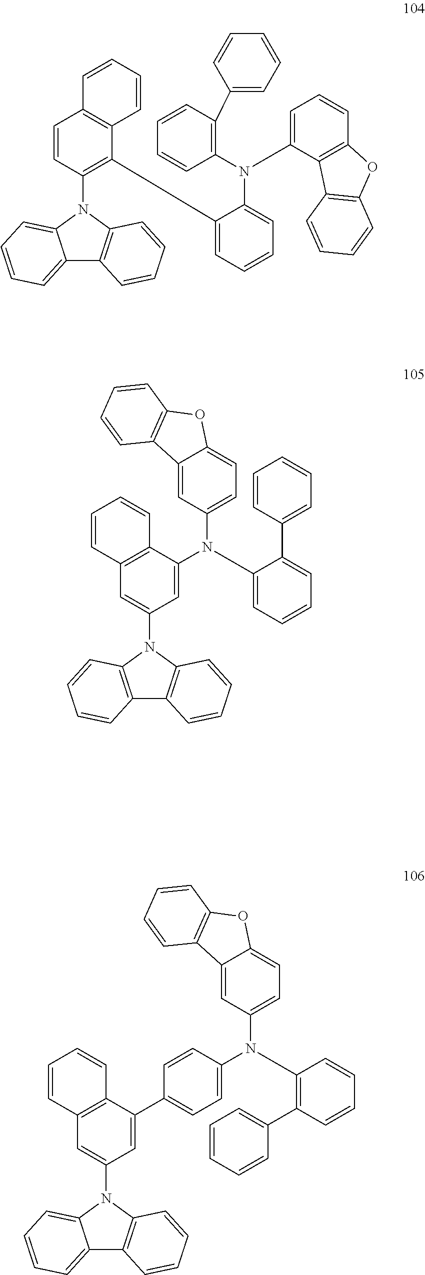 [SMILES: c1ccc(-c2ccccc2N(c2ccccc2-c2c(-n3c4ccccc4c4ccccc43)ccc3ccccc23)c2cccc3oc4ccccc4c23)cc1 ; c1ccc(-c2ccccc2N(c2ccc3oc4ccccc4c3c2)c2cc(-n3c4ccccc4c4ccccc43)cc3ccccc23)cc1 ; c1ccc(-c2ccccc2N(c2ccc(-c3cc(-n4c5ccccc5c5ccccc54)cc4ccccc34)cc2)c2ccc3oc4ccccc4c3c2)cc1]

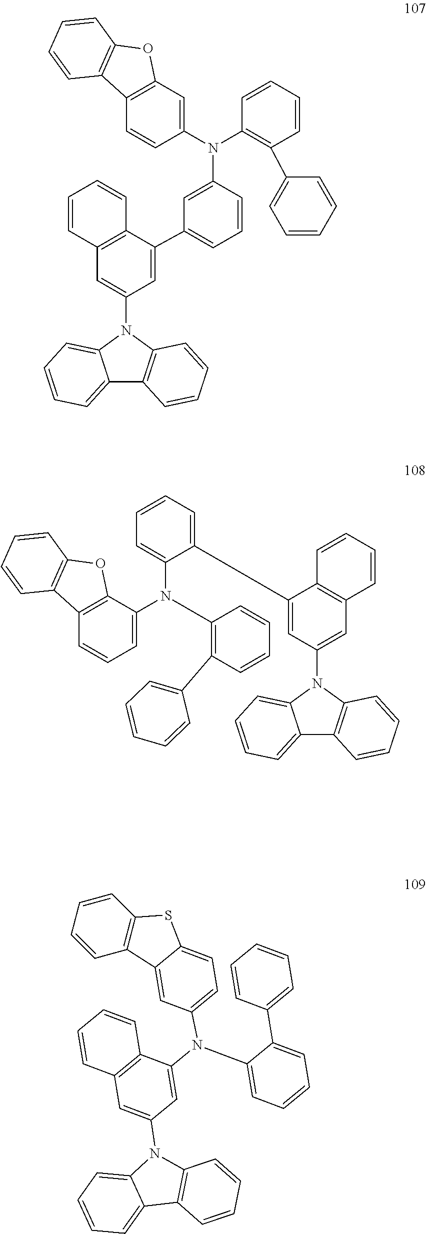 [SMILES: c1ccc(-c2ccccc2N(c2cccc(-c3cc(-n4c5ccccc5c5ccccc54)cc4ccccc34)c2)c2ccc3c(c2)oc2ccccc23)cc1 ; c1ccc(-c2ccccc2N(c2ccccc2-c2cc(-n3c4ccccc4c4ccccc43)cc3ccccc23)c2cccc3c2oc2ccccc23)cc1 ; c1ccc(-c2ccccc2N(c2ccc3sc4ccccc4c3c2)c2cc(-n3c4ccccc4c4ccccc43)cc3ccccc23)cc1]

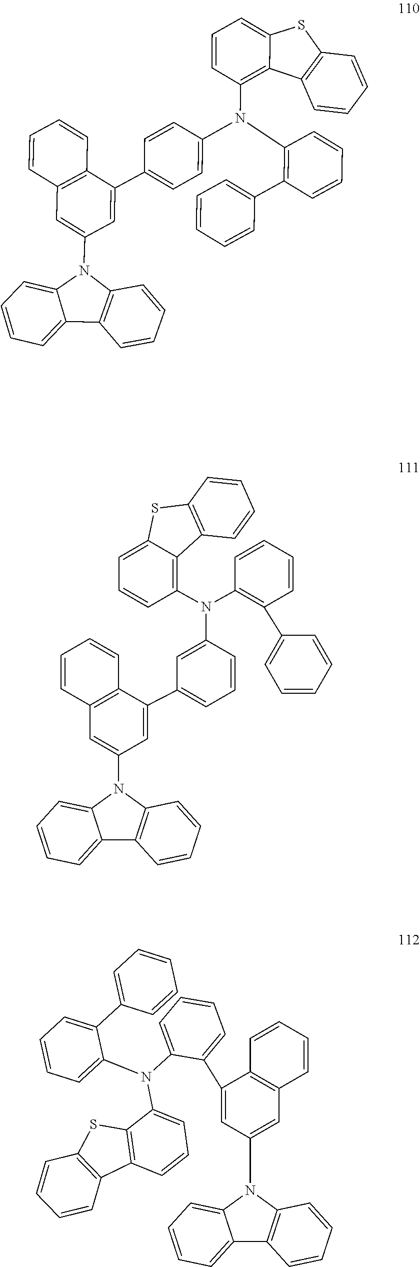 [SMILES: c1ccc(-c2ccccc2N(c2ccc(-c3cc(-n4c5ccccc5c5ccccc54)cc4ccccc34)cc2)c2cccc3sc4ccccc4c23)cc1 ; c1ccc(-c2ccccc2N(c2cccc(-c3cc(-n4c5ccccc5c5ccccc54)cc4ccccc34)c2)c2cccc3sc4ccccc4c23)cc1 ; c1ccc(-c2ccccc2N(c2ccccc2-c2cc(-n3c4ccccc4c4ccccc43)cc3ccccc23)c2cccc3c2sc2ccccc23)cc1]

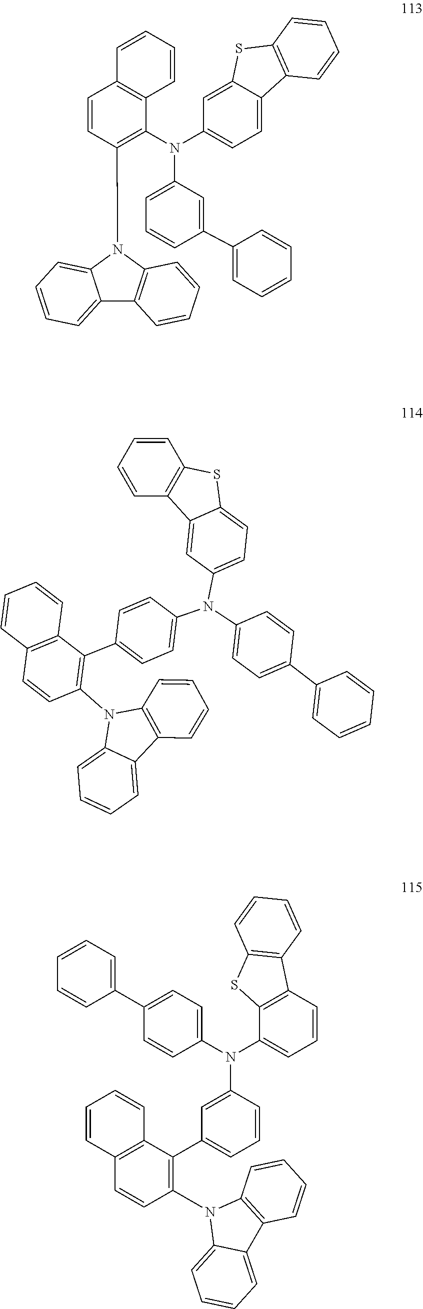 [SMILES: c1ccc(-c2cccc(N(c3ccc4c(c3)sc3ccccc34)c3c(-n4c5ccccc5c5ccccc54)ccc4ccccc34)c2)cc1 ; c1ccc(-c2ccc(N(c3ccc(-c4c(-n5c6ccccc6c6ccccc65)ccc5ccccc45)cc3)c3ccc4sc5ccccc5c4c3)cc2)cc1 ; c1ccc(-c2ccc(N(c3cccc(-c4c(-n5c6ccccc6c6ccccc65)ccc5ccccc45)c3)c3cccc4c3sc3ccccc34)cc2)cc1]

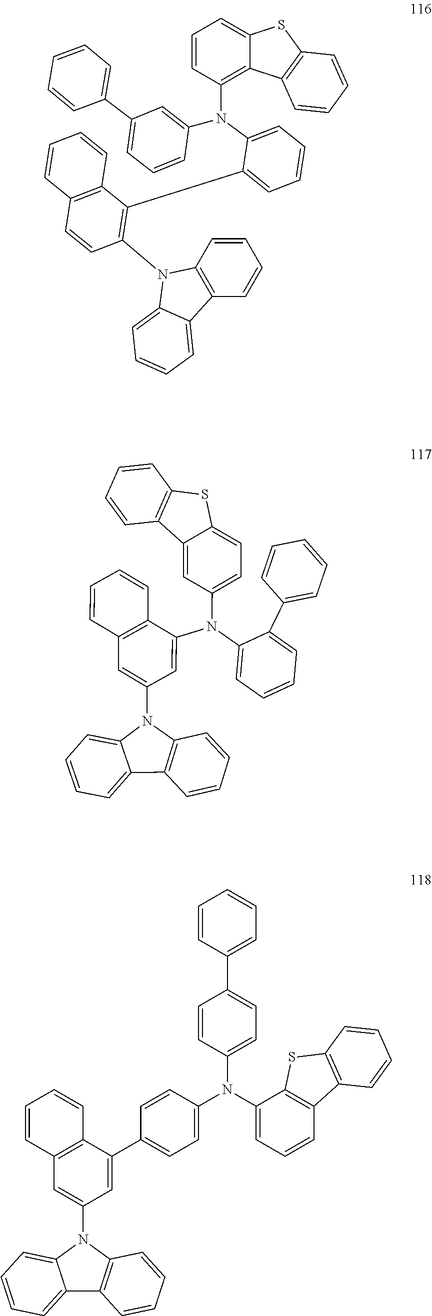 [SMILES: c1ccc(-c2cccc(N(c3ccccc3-c3c(-n4c5ccccc5c5ccccc54)ccc4ccccc34)c3cccc4sc5ccccc5c34)c2)cc1 ; c1ccc(-c2ccccc2N(c2ccc3sc4ccccc4c3c2)c2cc(-n3c4ccccc4c4ccccc43)cc3ccccc23)cc1 ; c1ccc(-c2ccc(N(c3ccc(-c4cc(-n5c6ccccc6c6ccccc65)cc5ccccc45)cc3)c3cccc4c3sc3ccccc34)cc2)cc1]

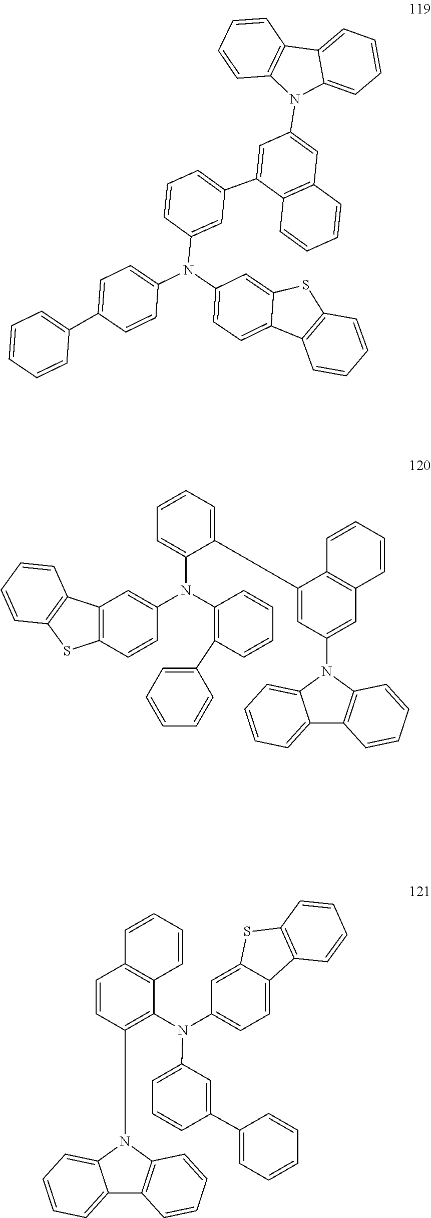 [SMILES: c1ccc(-c2ccc(N(c3cccc(-c4cc(-n5c6ccccc6c6ccccc65)cc5ccccc45)c3)c3ccc4c(c3)sc3ccccc34)cc2)cc1 ; c1ccc(-c2ccccc2N(c2ccc3sc4ccccc4c3c2)c2ccccc2-c2cc(-n3c4ccccc4c4ccccc43)cc3ccccc23)cc1 ; c1ccc(-c2cccc(N(c3ccc4c(c3)sc3ccccc34)c3c(-n4c5ccccc5c5ccccc54)ccc4ccccc34)c2)cc1]

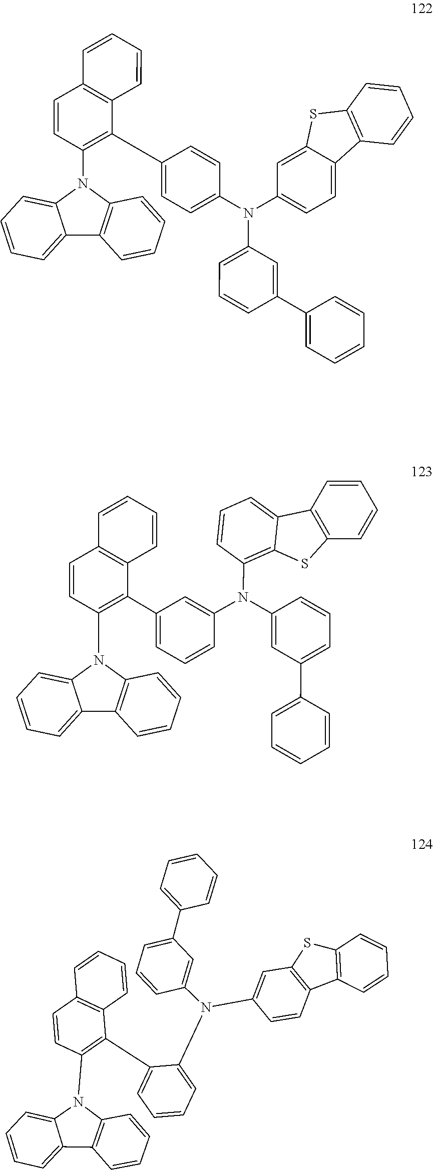 [SMILES: c1ccc(-c2cccc(N(c3ccc(-c4c(-n5c6ccccc6c6ccccc65)ccc5ccccc45)cc3)c3ccc4c(c3)sc3ccccc34)c2)cc1 ; c1ccc(-c2cccc(N(c3cccc(-c4c(-n5c6ccccc6c6ccccc65)ccc5ccccc45)c3)c3cccc4c3sc3ccccc34)c2)cc1 ; c1ccc(-c2cccc(N(c3ccc4c(c3)sc3ccccc34)c3ccccc3-c3c(-n4c5ccccc5c5ccccc54)ccc4ccccc34)c2)cc1]

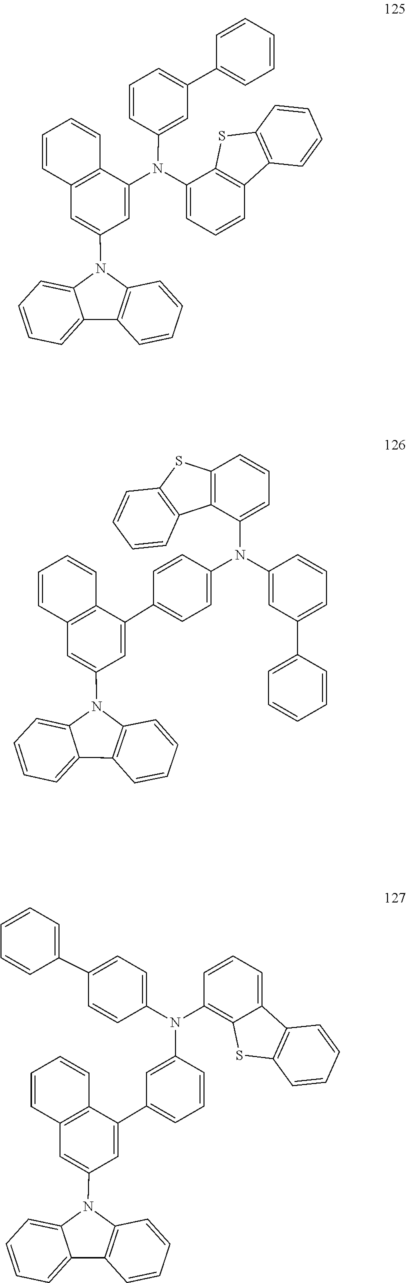 [SMILES: c1ccc(-c2cccc(N(c3cc(-n4c5ccccc5c5ccccc54)cc4ccccc34)c3cccc4c3sc3ccccc34)c2)cc1 ; c1ccc(-c2cccc(N(c3ccc(-c4cc(-n5c6ccccc6c6ccccc65)cc5ccccc45)cc3)c3cccc4sc5ccccc5c34)c2)cc1 ; c1ccc(-c2ccc(N(c3cccc(-c4cc(-n5c6ccccc6c6ccccc65)cc5ccccc45)c3)c3cccc4c3sc3ccccc34)cc2)cc1]

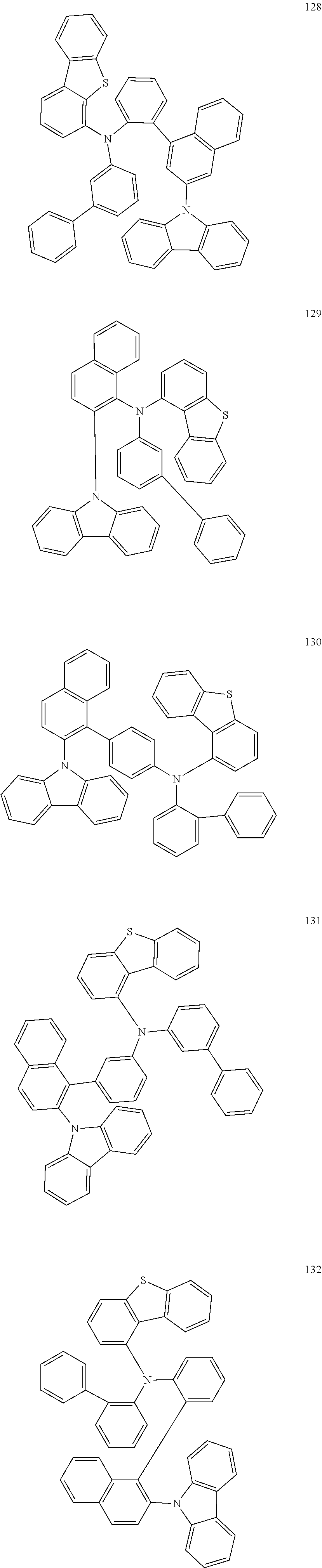 [SMILES: c1ccc(-c2cccc(N(c3ccccc3-c3cc(-n4c5ccccc5c5ccccc54)cc4ccccc34)c3cccc4c3sc3ccccc34)c2)cc1 ; c1ccc(-c2cccc(N(c3c(-n4c5ccccc5c5ccccc54)ccc4ccccc34)c3cccc4sc5ccccc5c34)c2)cc1 ; c1ccc(-c2ccccc2N(c2ccc(-c3c(-n4c5ccccc5c5ccccc54)ccc4ccccc34)cc2)c2cccc3sc4ccccc4c23)cc1 ; c1ccc(-c2cccc(N(c3cccc(-c4c(-n5c6ccccc6c6ccccc65)ccc5ccccc45)c3)c3cccc4sc5ccccc5c34)c2)cc1 ; c1ccc(-c2ccccc2N(c2ccccc2-c2c(-n3c4ccccc4c4ccccc43)ccc3ccccc23)c2cccc3sc4ccccc4c23)cc1]

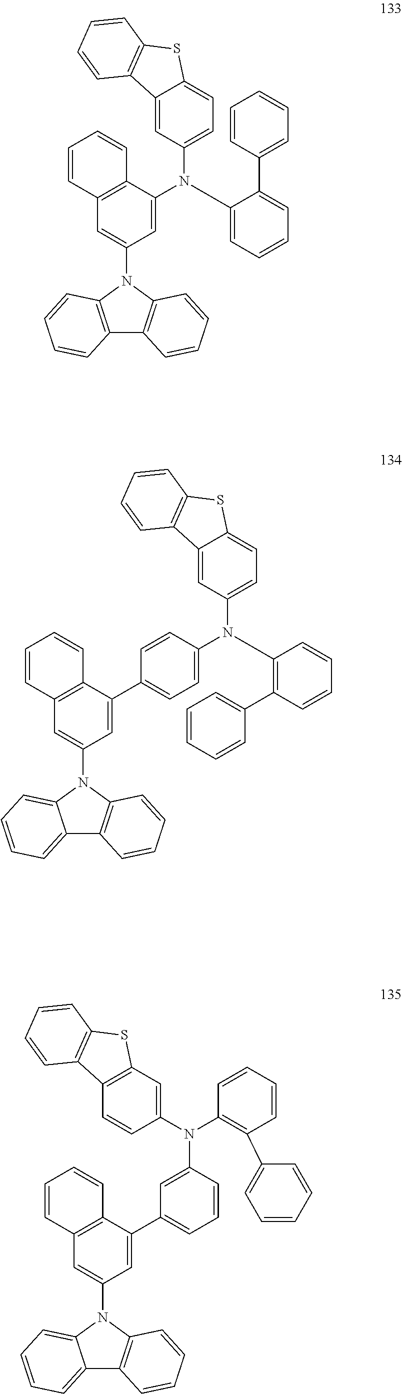 [SMILES: c1ccc(-c2ccccc2N(c2ccc3sc4ccccc4c3c2)c2cc(-n3c4ccccc4c4ccccc43)cc3ccccc23)cc1 ; c1ccc(-c2ccccc2N(c2ccc(-c3cc(-n4c5ccccc5c5ccccc54)cc4ccccc34)cc2)c2ccc3sc4ccccc4c3c2)cc1 ; c1ccc(-c2ccccc2N(c2cccc(-c3cc(-n4c5ccccc5c5ccccc54)cc4ccccc34)c2)c2ccc3c(c2)sc2ccccc23)cc1]

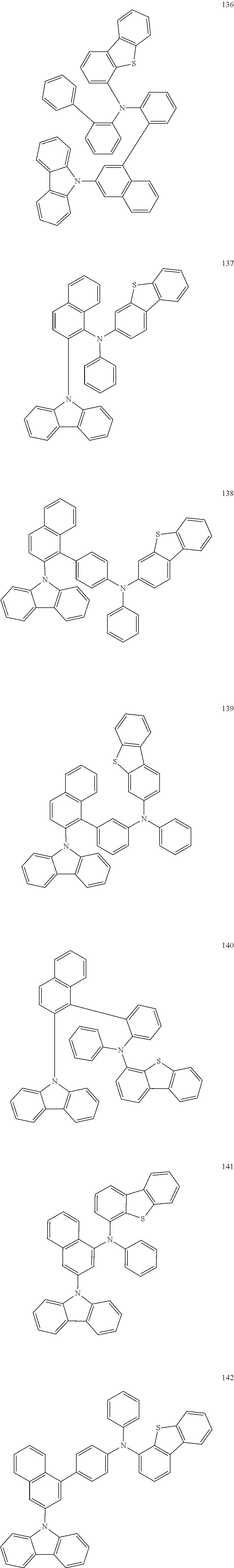

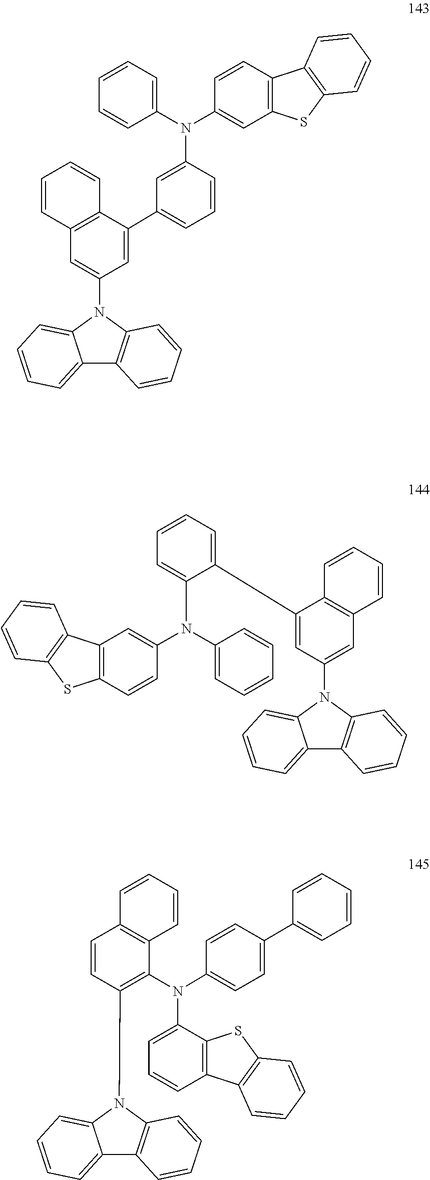 [SMILES: c1ccc(N(c2cccc(-c3cc(-n4c5ccccc5c5ccccc54)cc4ccccc34)c2)c2ccc3c(c2)sc2ccccc23)cc1 ; c1ccc(N(c2ccc3sc4ccccc4c3c2)c2ccccc2-c2cc(-n3c4ccccc4c4ccccc43)cc3ccccc23)cc1 ; c1ccc(-c2ccc(N(c3c(-n4c5ccccc5c5ccccc54)ccc4ccccc34)c3cccc4c3sc3ccccc34)cc2)cc1]

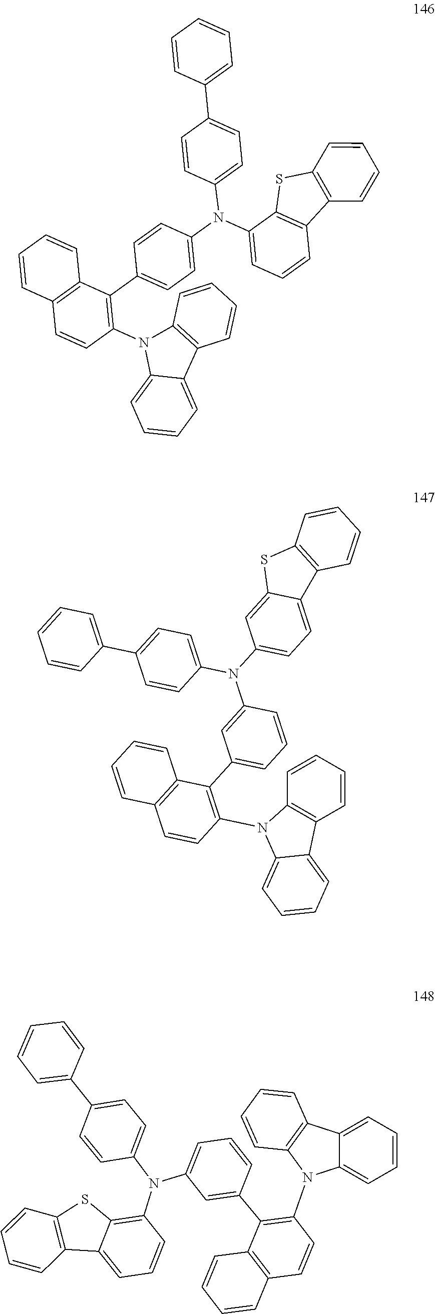 [SMILES: c1ccc(-c2ccc(N(c3ccc(-c4c(-n5c6ccccc6c6ccccc65)ccc5ccccc45)cc3)c3cccc4c3sc3ccccc34)cc2)cc1 ; c1ccc(-c2ccc(N(c3cccc(-c4c(-n5c6ccccc6c6ccccc65)ccc5ccccc45)c3)c3ccc4c(c3)sc3ccccc34)cc2)cc1 ; c1ccc(-c2ccc(N(c3cccc(-c4c(-n5c6ccccc6c6ccccc65)ccc5ccccc45)c3)c3cccc4c3sc3ccccc34)cc2)cc1]

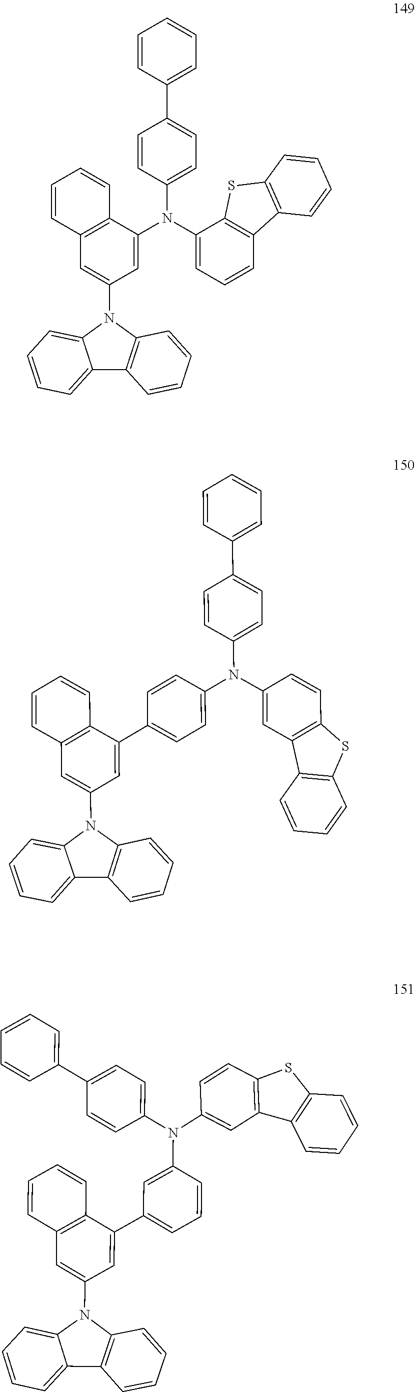 [SMILES: c1ccc(-c2ccc(N(c3cc(-n4c5ccccc5c5ccccc54)cc4ccccc34)c3cccc4c3sc3ccccc34)cc2)cc1 ; c1ccc(-c2ccc(N(c3ccc(-c4cc(-n5c6ccccc6c6ccccc65)cc5ccccc45)cc3)c3ccc4sc5ccccc5c4c3)cc2)cc1 ; c1ccc(-c2ccc(N(c3cccc(-c4cc(-n5c6ccccc6c6ccccc65)cc5ccccc45)c3)c3ccc4sc5ccccc5c4c3)cc2)cc1]

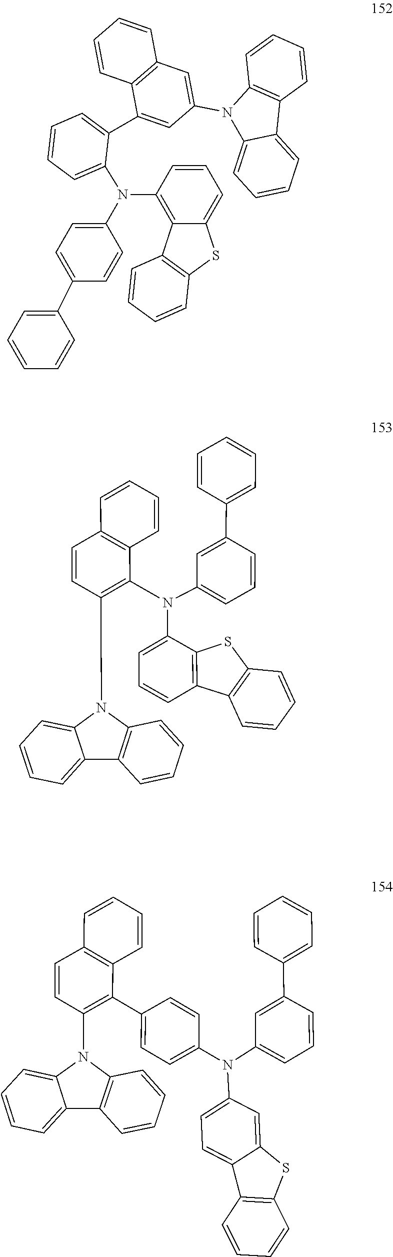 [SMILES: c1ccc(-c2ccc(N(c3ccccc3-c3cc(-n4c5ccccc5c5ccccc54)cc4ccccc34)c3cccc4sc5ccccc5c34)cc2)cc1 ; c1ccc(-c2cccc(N(c3c(-n4c5ccccc5c5ccccc54)ccc4ccccc34)c3cccc4c3sc3ccccc34)c2)cc1 ; c1ccc(-c2cccc(N(c3ccc(-c4c(-n5c6ccccc6c6ccccc65)ccc5ccccc45)cc3)c3ccc4c(c3)sc3ccccc34)c2)cc1]

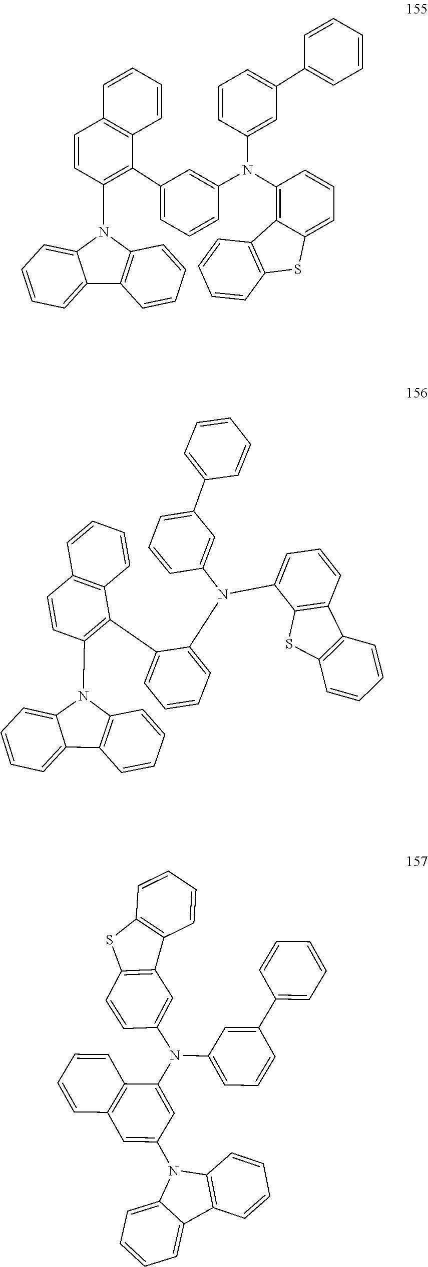 [SMILES: c1ccc(-c2cccc(N(c3cccc(-c4c(-n5c6ccccc6c6ccccc65)ccc5ccccc45)c3)c3cccc4sc5ccccc5c34)c2)cc1 ; c1ccc(-c2cccc(N(c3ccccc3-c3c(-n4c5ccccc5c5ccccc54)ccc4ccccc34)c3cccc4c3sc3ccccc34)c2)cc1 ; c1ccc(-c2cccc(N(c3ccc4sc5ccccc5c4c3)c3cc(-n4c5ccccc5c5ccccc54)cc4ccccc34)c2)cc1]

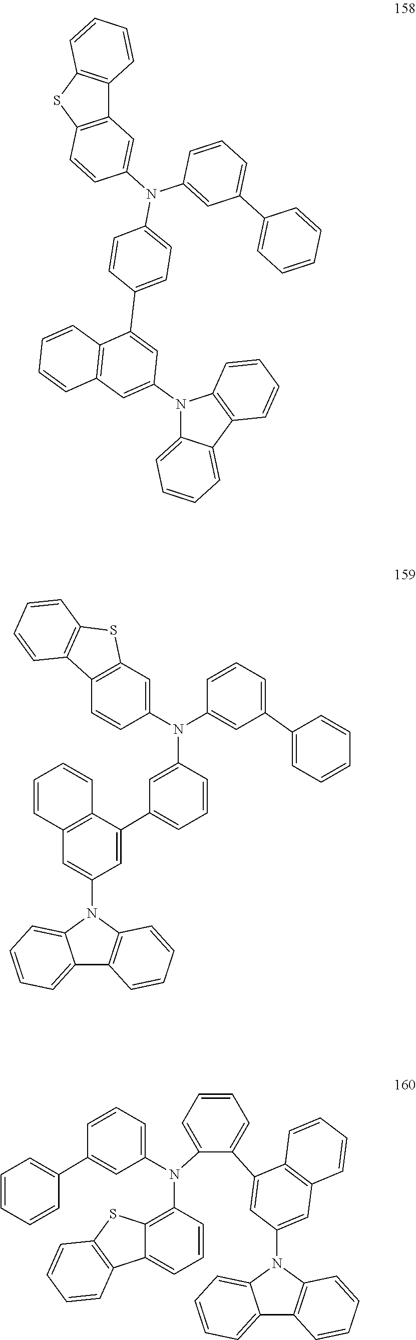 [SMILES: c1ccc(-c2cccc(N(c3ccc(-c4cc(-n5c6ccccc6c6ccccc65)cc5ccccc45)cc3)c3ccc4sc5ccccc5c4c3)c2)cc1 ; c1ccc(-c2cccc(N(c3cccc(-c4cc(-n5c6ccccc6c6ccccc65)cc5ccccc45)c3)c3ccc4c(c3)sc3ccccc34)c2)cc1 ; c1ccc(-c2cccc(N(c3ccccc3-c3cc(-n4c5ccccc5c5ccccc54)cc4ccccc34)c3cccc4c3sc3ccccc34)c2)cc1]

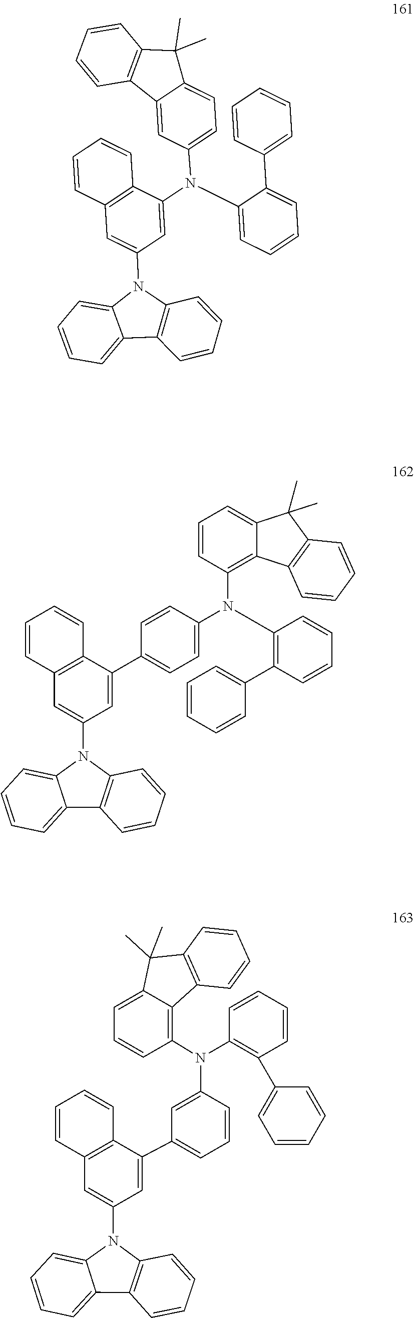 [SMILES: CC1(C)c2ccccc2-c2cc(N(c3ccccc3-c3ccccc3)c3cc(-n4c5ccccc5c5ccccc54)cc4ccccc34)ccc21 ; CC1(C)c2ccccc2-c2c(N(c3ccc(-c4cc(-n5c6ccccc6c6ccccc65)cc5ccccc45)cc3)c3ccccc3-c3ccccc3)cccc21 ; CC1(C)c2ccccc2-c2c(N(c3cccc(-c4cc(-n5c6ccccc6c6ccccc65)cc5ccccc45)c3)c3ccccc3-c3ccccc3)cccc21]

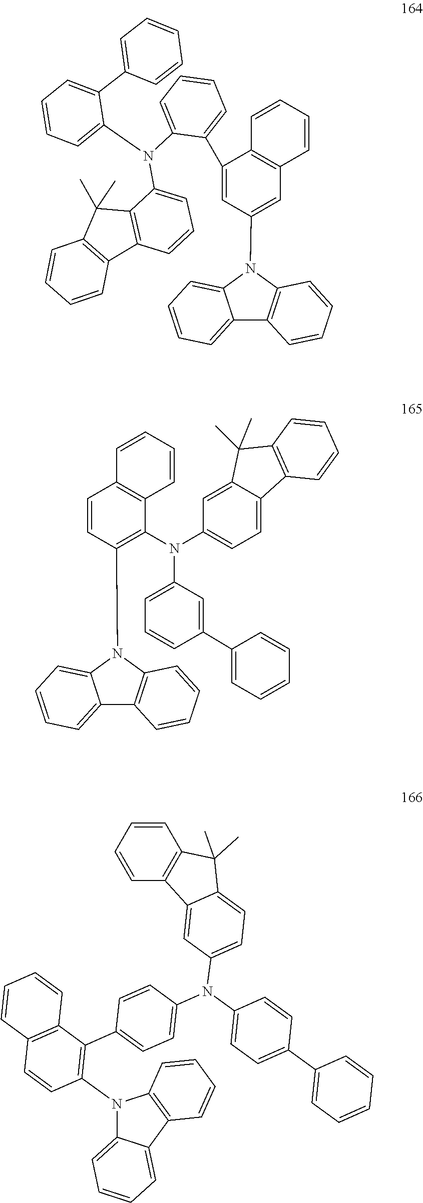 [SMILES: CC1(C)c2ccccc2-c2cccc(N(c3ccccc3-c3ccccc3)c3ccccc3-c3cc(-n4c5ccccc5c5ccccc54)cc4ccccc34)c21 ; CC1(C)c2ccccc2-c2ccc(N(c3cccc(-c4ccccc4)c3)c3c(-n4c5ccccc5c5ccccc54)ccc4ccccc34)cc21 ; CC1(C)c2ccccc2-c2cc(N(c3ccc(-c4ccccc4)cc3)c3ccc(-c4c(-n5c6ccccc6c6ccccc65)ccc5ccccc45)cc3)ccc21]

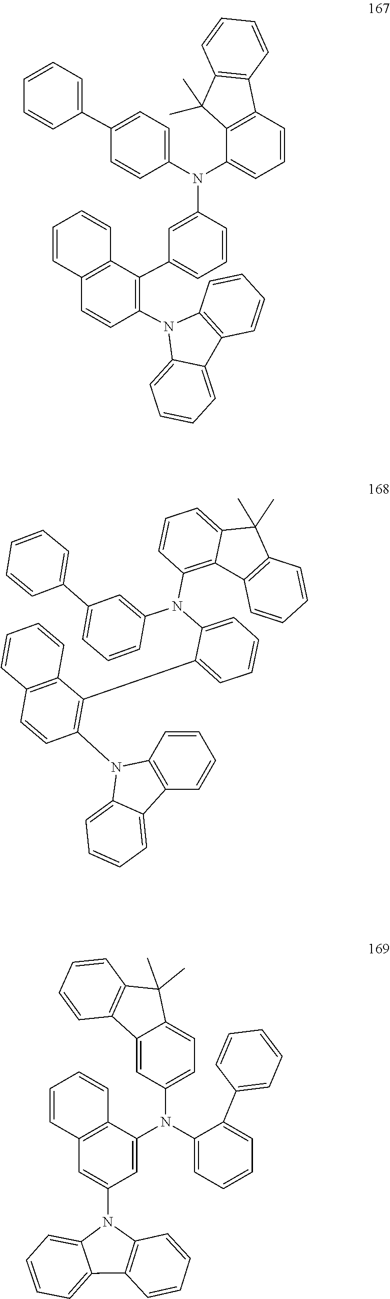 [SMILES: CC1(C)c2ccccc2-c2cccc(N(c3ccc(-c4ccccc4)cc3)c3cccc(-c4c(-n5c6ccccc6c6ccccc65)ccc5ccccc45)c3)c21 ; CC1(C)c2ccccc2-c2c(N(c3cccc(-c4ccccc4)c3)c3ccccc3-c3c(-n4c5ccccc5c5ccccc54)ccc4ccccc34)cccc21 ; CC1(C)c2ccccc2-c2cc(N(c3ccccc3-c3ccccc3)c3cc(-n4c5ccccc5c5ccccc54)cc4ccccc34)ccc21]

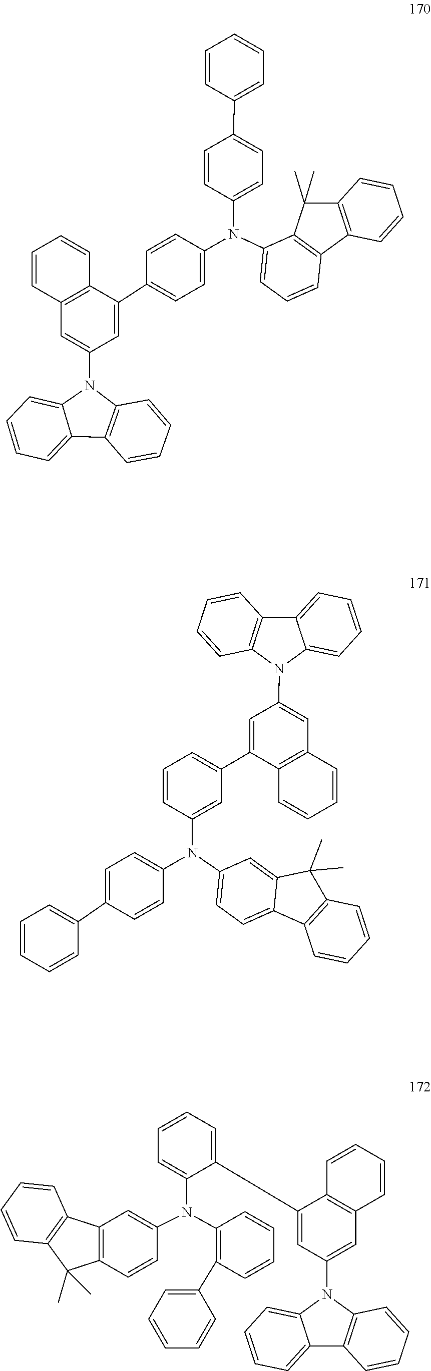 [SMILES: CC1(C)c2ccccc2-c2cccc(N(c3ccc(-c4ccccc4)cc3)c3ccc(-c4cc(-n5c6ccccc6c6ccccc65)cc5ccccc45)cc3)c21 ; CC1(C)c2ccccc2-c2ccc(N(c3ccc(-c4ccccc4)cc3)c3cccc(-c4cc(-n5c6ccccc6c6ccccc65)cc5ccccc45)c3)cc21 ; CC1(C)c2ccccc2-c2cc(N(c3ccccc3-c3ccccc3)c3ccccc3-c3cc(-n4c5ccccc5c5ccccc54)cc4ccccc34)ccc21]

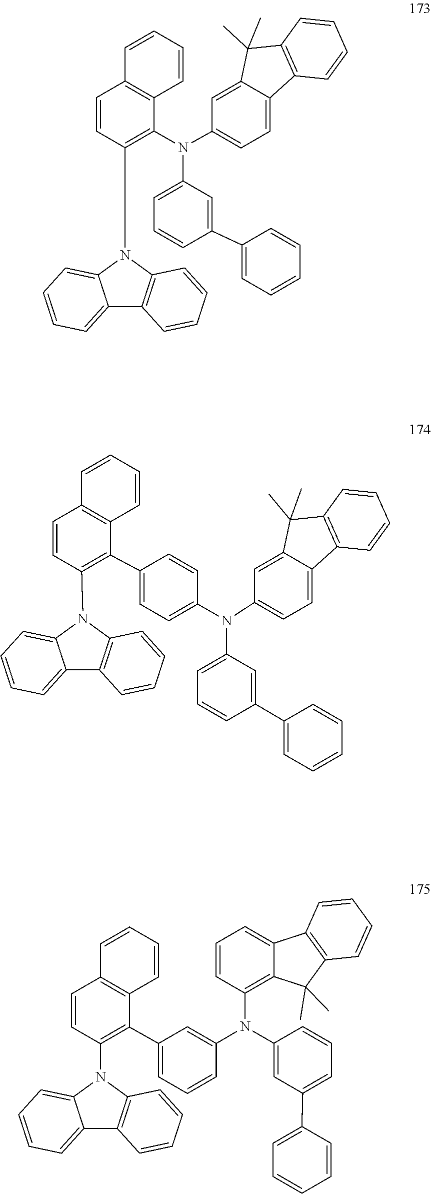 [SMILES: CC1(C)c2ccccc2-c2ccc(N(c3cccc(-c4ccccc4)c3)c3c(-n4c5ccccc5c5ccccc54)ccc4ccccc34)cc21 ; CC1(C)c2ccccc2-c2ccc(N(c3ccc(-c4c(-n5c6ccccc6c6ccccc65)ccc5ccccc45)cc3)c3cccc(-c4ccccc4)c3)cc21 ; CC1(C)c2ccccc2-c2cccc(N(c3cccc(-c4ccccc4)c3)c3cccc(-c4c(-n5c6ccccc6c6ccccc65)ccc5ccccc45)c3)c21]

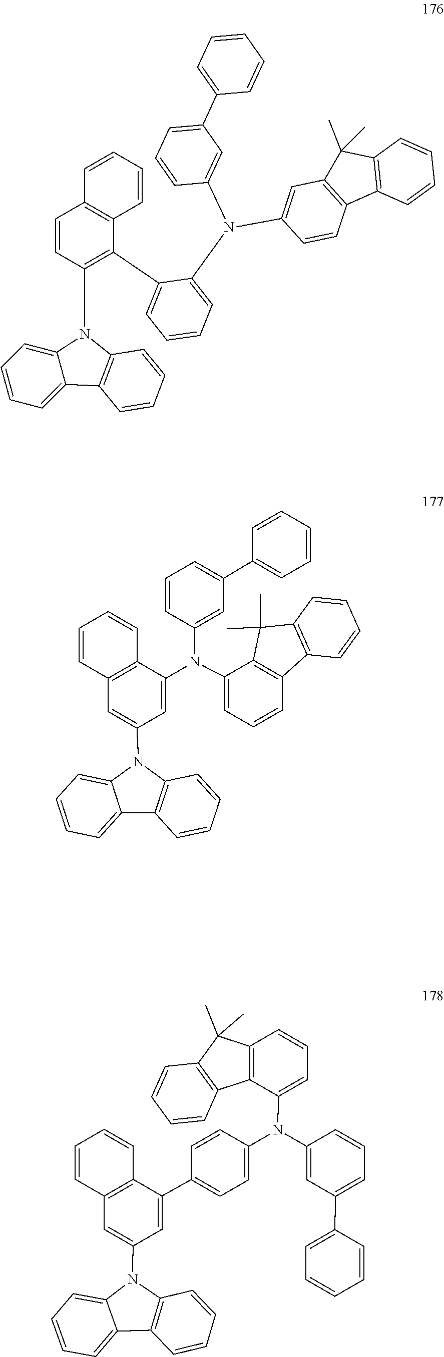 [SMILES: CC1(C)c2ccccc2-c2ccc(N(c3cccc(-c4ccccc4)c3)c3ccccc3-c3c(-n4c5ccccc5c5ccccc54)ccc4ccccc34)cc21 ; CC1(C)c2ccccc2-c2cccc(N(c3cccc(-c4ccccc4)c3)c3cc(-n4c5ccccc5c5ccccc54)cc4ccccc34)c21 ; CC1(C)c2ccccc2-c2c(N(c3ccc(-c4cc(-n5c6ccccc6c6ccccc65)cc5ccccc45)cc3)c3cccc(-c4ccccc4)c3)cccc21]

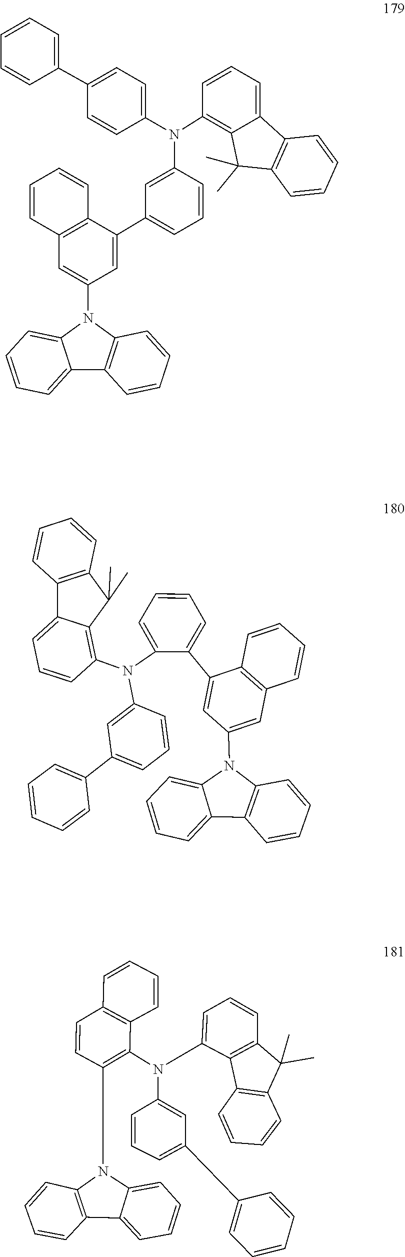 [SMILES: CC1(C)c2ccccc2-c2cccc(N(c3ccc(-c4ccccc4)cc3)c3cccc(-c4cc(-n5c6ccccc6c6ccccc65)cc5ccccc45)c3)c21 ; CC1(C)c2ccccc2-c2cccc(N(c3cccc(-c4ccccc4)c3)c3ccccc3-c3cc(-n4c5ccccc5c5ccccc54)cc4ccccc34)c21 ; CC1(C)c2ccccc2-c2c(N(c3cccc(-c4ccccc4)c3)c3c(-n4c5ccccc5c5ccccc54)ccc4ccccc34)cccc21]

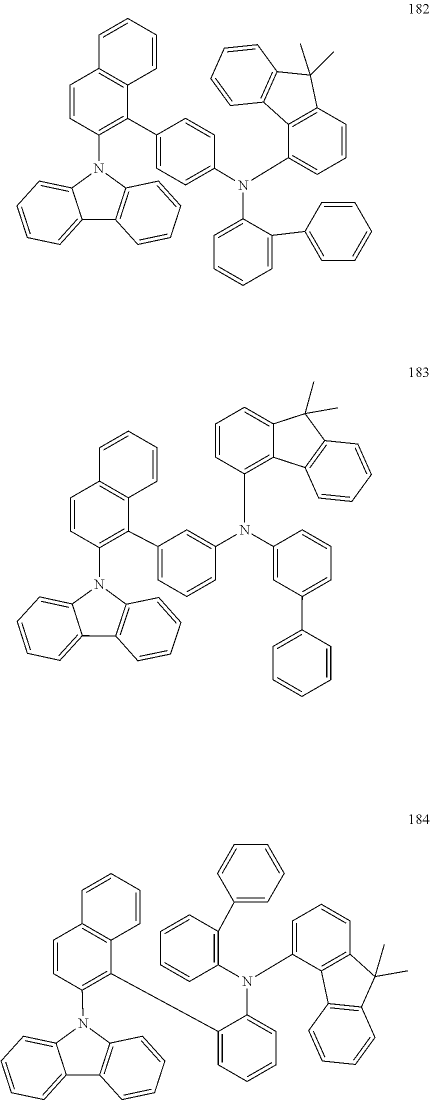 [SMILES: CC1(C)c2ccccc2-c2c(N(c3ccc(-c4c(-n5c6ccccc6c6ccccc65)ccc5ccccc45)cc3)c3ccccc3-c3ccccc3)cccc21 ; CC1(C)c2ccccc2-c2c(N(c3cccc(-c4ccccc4)c3)c3cccc(-c4c(-n5c6ccccc6c6ccccc65)ccc5ccccc45)c3)cccc21 ; CC1(C)c2ccccc2-c2c(N(c3ccccc3-c3ccccc3)c3ccccc3-c3c(-n4c5ccccc5c5ccccc54)ccc4ccccc34)cccc21]

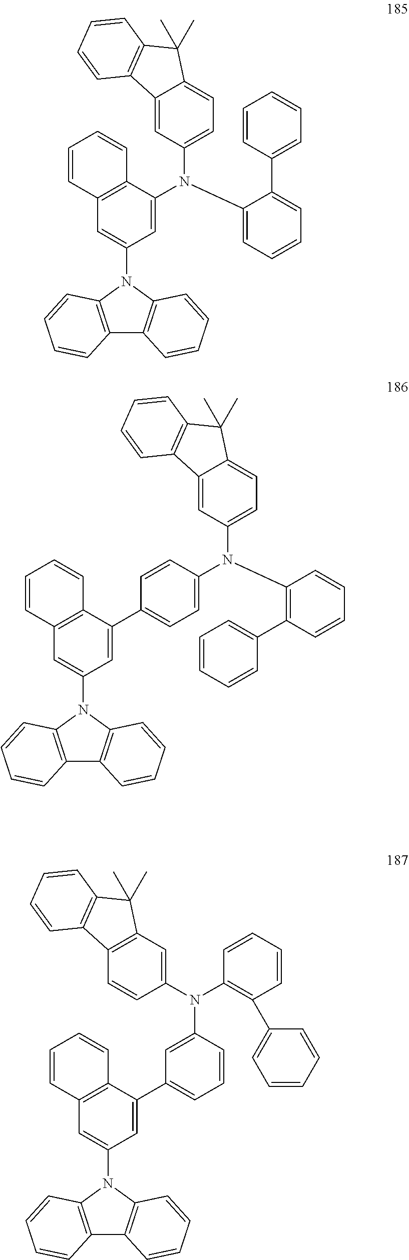

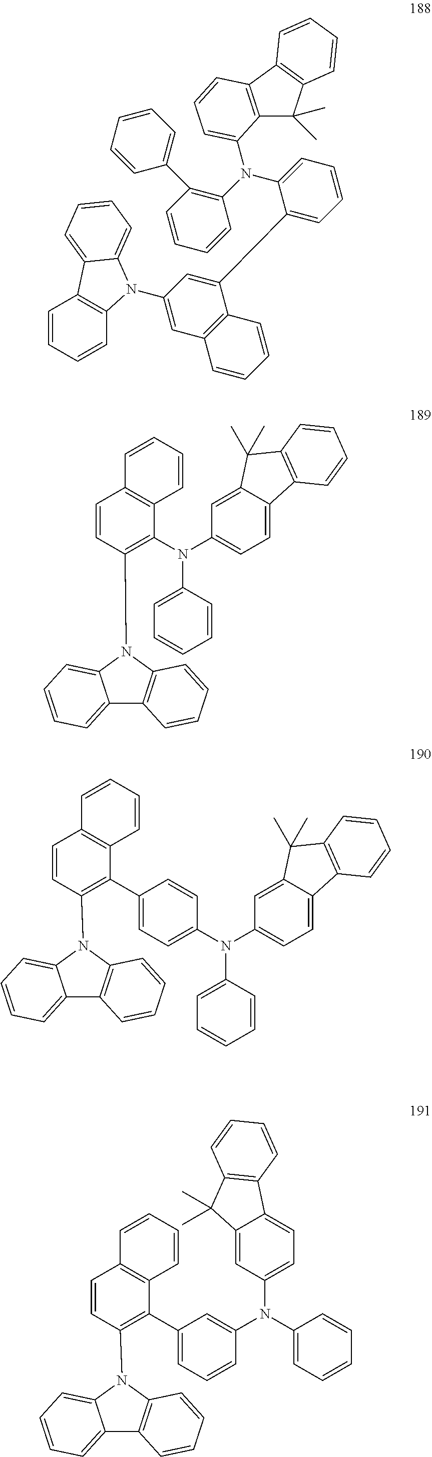 [SMILES: CC1(C)c2ccccc2-c2cccc(N(c3ccccc3-c3ccccc3)c3ccccc3-c3cc(-n4c5ccccc5c5ccccc54)cc4ccccc34)c21 ; CC1(C)c2ccccc2-c2ccc(N(c3ccccc3)c3c(-n4c5ccccc5c5ccccc54)ccc4ccccc34)cc21 ; CC1(C)c2ccccc2-c2ccc(N(c3ccccc3)c3ccc(-c4c(-n5c6ccccc6c6ccccc65)ccc5ccccc45)cc3)cc21 ; CC1(C)c2ccccc2-c2ccc(N(c3ccccc3)c3cccc(-c4c(-n5c6ccccc6c6ccccc65)ccc5ccccc45)c3)cc21]

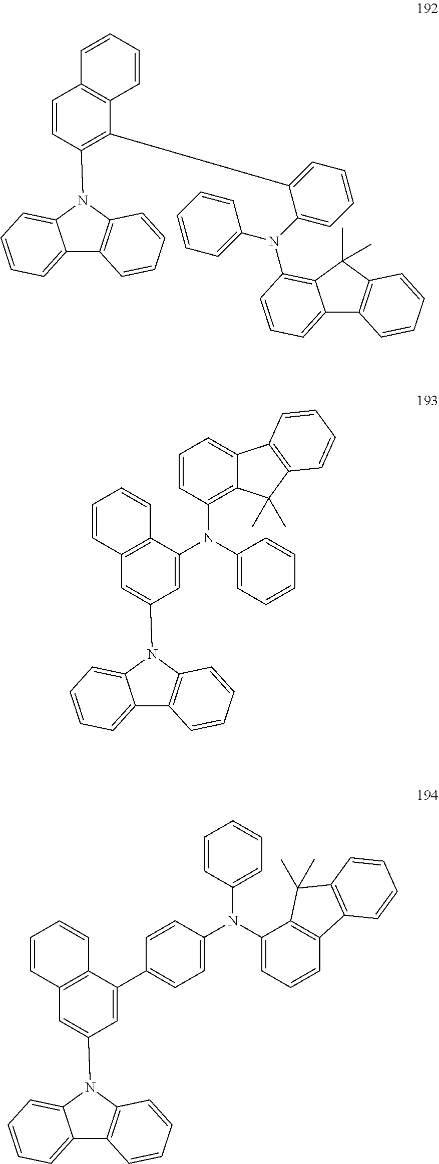 [SMILES: CC1(C)c2ccccc2-c2cccc(N(c3ccccc3)c3ccccc3-c3c(-n4c5ccccc5c5ccccc54)ccc4ccccc34)c21 ; CC1(C)c2ccccc2-c2cccc(N(c3ccccc3)c3cc(-n4c5ccccc5c5ccccc54)cc4ccccc34)c21 ; CC1(C)c2ccccc2-c2cccc(N(c3ccccc3)c3ccc(-c4cc(-n5c6ccccc6c6ccccc65)cc5ccccc45)cc3)c21]

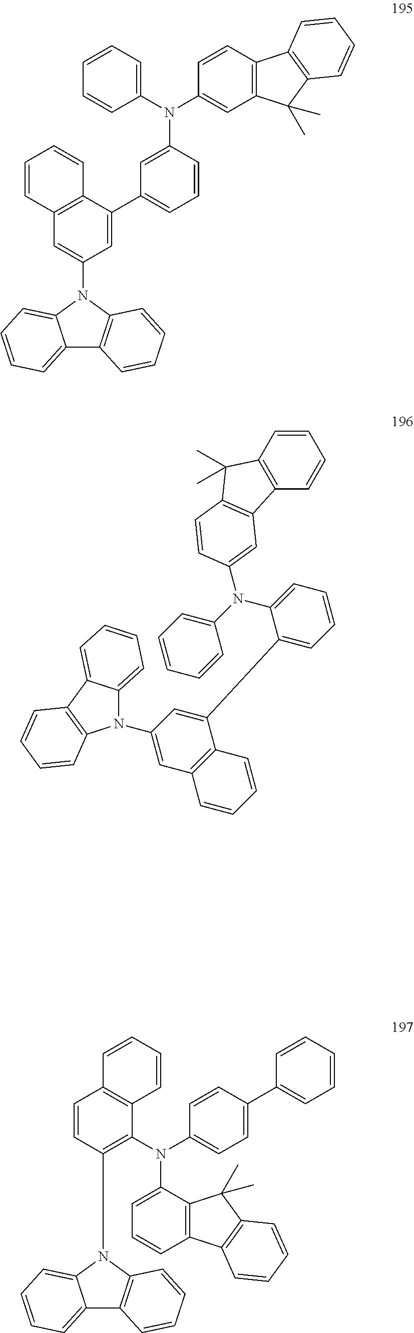 [SMILES: CC1(C)c2ccccc2-c2ccc(N(c3ccccc3)c3cccc(-c4cc(-n5c6ccccc6c6ccccc65)cc5ccccc45)c3)cc21 ; CC1(C)c2ccccc2-c2cc(N(c3ccccc3)c3ccccc3-c3cc(-n4c5ccccc5c5ccccc54)cc4ccccc34)ccc21 ; CC1(C)c2ccccc2-c2cccc(N(c3ccc(-c4ccccc4)cc3)c3c(-n4c5ccccc5c5ccccc54)ccc4ccccc34)c21]

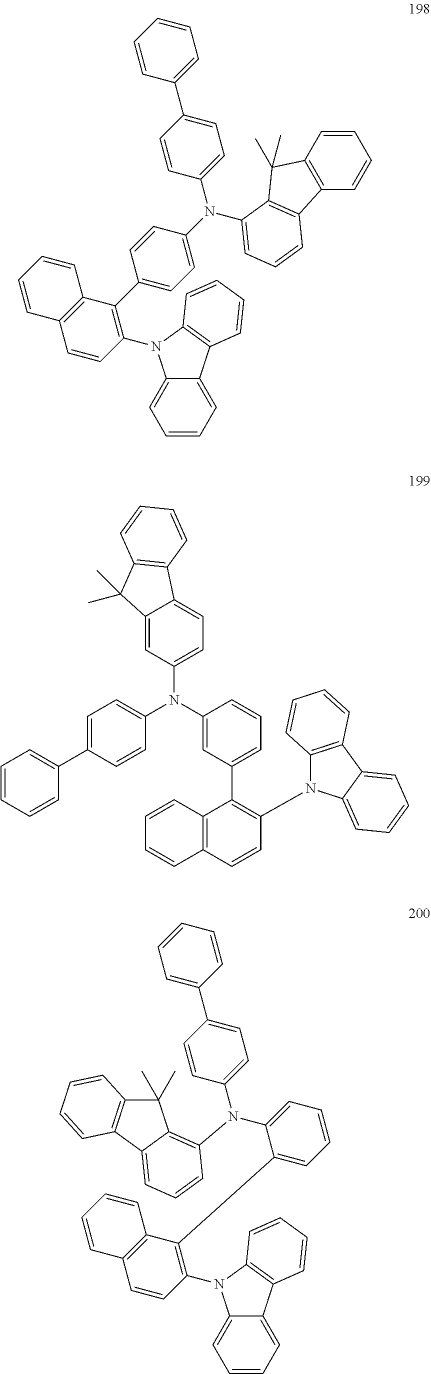 [SMILES: CC1(C)c2ccccc2-c2cccc(N(c3ccc(-c4ccccc4)cc3)c3ccc(-c4c(-n5c6ccccc6c6ccccc65)ccc5ccccc45)cc3)c21 ; CC1(C)c2ccccc2-c2ccc(N(c3ccc(-c4ccccc4)cc3)c3cccc(-c4c(-n5c6ccccc6c6ccccc65)ccc5ccccc45)c3)cc21 ; CC1(C)c2ccccc2-c2cccc(N(c3ccc(-c4ccccc4)cc3)c3ccccc3-c3c(-n4c5ccccc5c5ccccc54)ccc4ccccc34)c21]

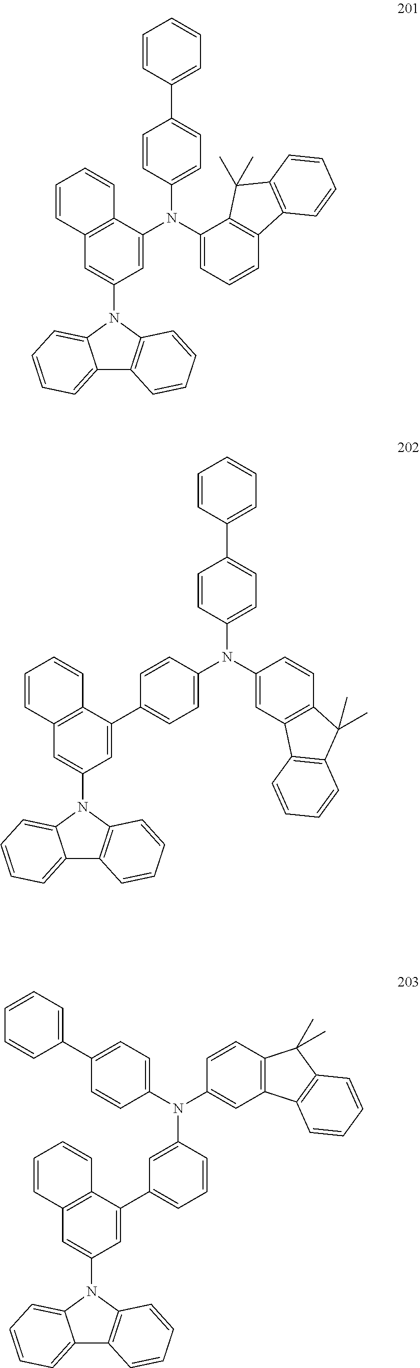 [SMILES: CC1(C)c2ccccc2-c2cccc(N(c3ccc(-c4ccccc4)cc3)c3cc(-n4c5ccccc5c5ccccc54)cc4ccccc34)c21 ; CC1(C)c2ccccc2-c2cc(N(c3ccc(-c4ccccc4)cc3)c3ccc(-c4cc(-n5c6ccccc6c6ccccc65)cc5ccccc45)cc3)ccc21 ; CC1(C)c2ccccc2-c2cc(N(c3ccc(-c4ccccc4)cc3)c3cccc(-c4cc(-n5c6ccccc6c6ccccc65)cc5ccccc45)c3)ccc21]

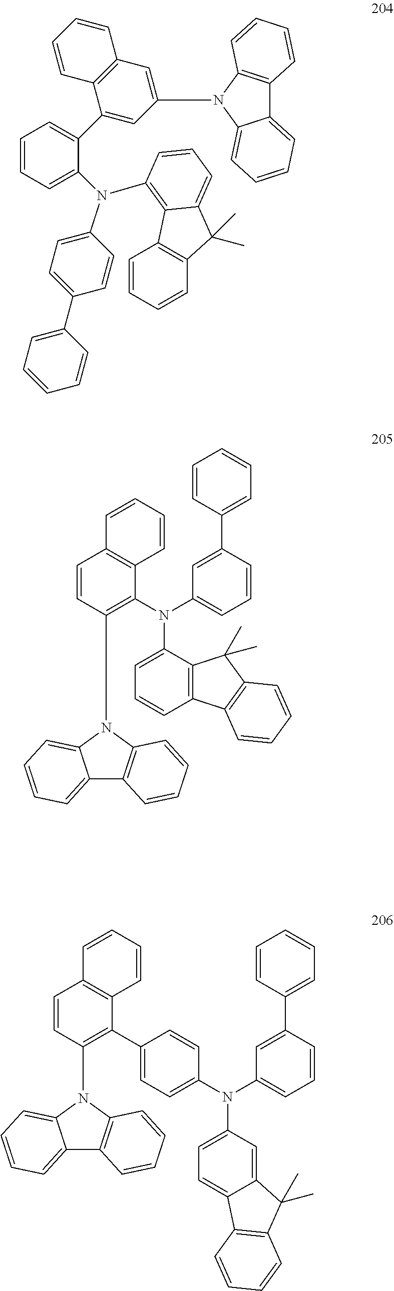 [SMILES: CC1(C)c2ccccc2-c2c(N(c3ccc(-c4ccccc4)cc3)c3ccccc3-c3cc(-n4c5ccccc5c5ccccc54)cc4ccccc34)cccc21 ; CC1(C)c2ccccc2-c2cccc(N(c3cccc(-c4ccccc4)c3)c3c(-n4c5ccccc5c5ccccc54)ccc4ccccc34)c21 ; CC1(C)c2ccccc2-c2ccc(N(c3ccc(-c4c(-n5c6ccccc6c6ccccc65)ccc5ccccc45)cc3)c3cccc(-c4ccccc4)c3)cc21]

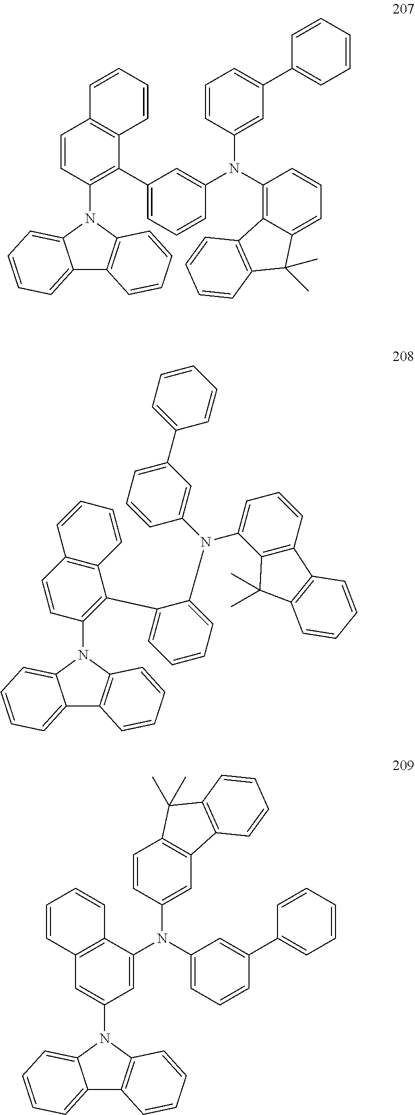 [SMILES: CC1(C)c2ccccc2-c2c(N(c3cccc(-c4ccccc4)c3)c3cccc(-c4c(-n5c6ccccc6c6ccccc65)ccc5ccccc45)c3)cccc21 ; CC1(C)c2ccccc2-c2cccc(N(c3cccc(-c4ccccc4)c3)c3ccccc3-c3c(-n4c5ccccc5c5ccccc54)ccc4ccccc34)c21 ; CC1(C)c2ccccc2-c2cc(N(c3cccc(-c4ccccc4)c3)c3cc(-n4c5ccccc5c5ccccc54)cc4ccccc34)ccc21]

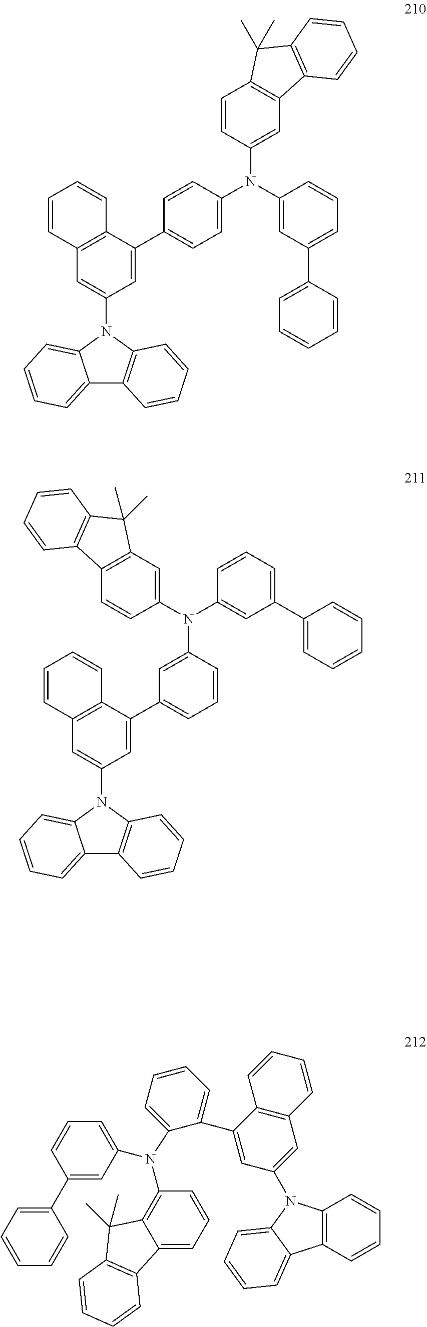 [SMILES: CC1(C)c2ccccc2-c2cc(N(c3ccc(-c4cc(-n5c6ccccc6c6ccccc65)cc5ccccc45)cc3)c3cccc(-c4ccccc4)c3)ccc21 ; CC1(C)c2ccccc2-c2ccc(N(c3cccc(-c4ccccc4)c3)c3cccc(-c4cc(-n5c6ccccc6c6ccccc65)cc5ccccc45)c3)cc21 ; CC1(C)c2ccccc2-c2cccc(N(c3cccc(-c4ccccc4)c3)c3ccccc3-c3cc(-n4c5ccccc5c5ccccc54)cc4ccccc34)c21]

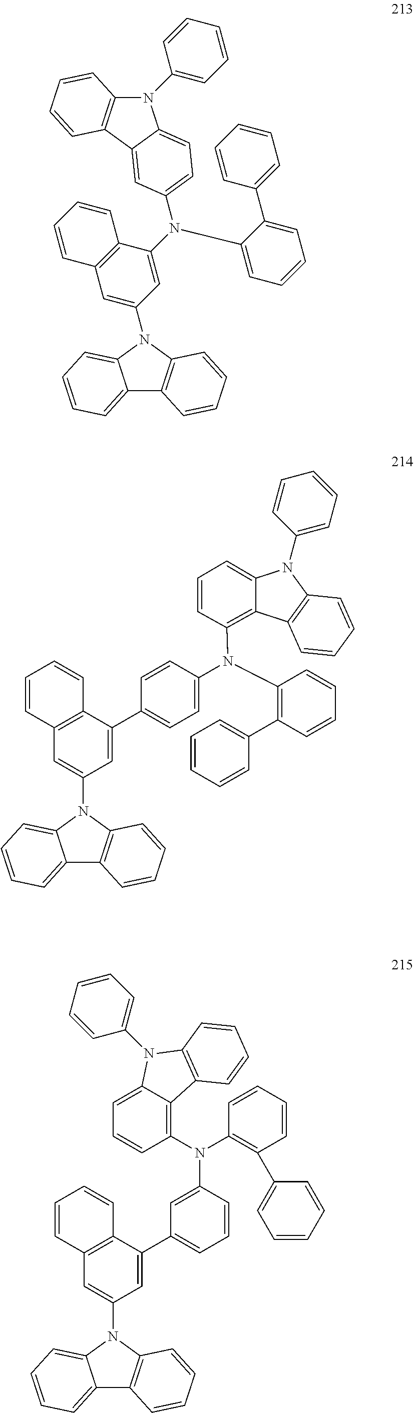 [SMILES: c1ccc(-c2ccccc2N(c2ccc3c(c2)c2ccccc2n3-c2ccccc2)c2cc(-n3c4ccccc4c4ccccc43)cc3ccccc23)cc1 ; c1ccc(-c2ccccc2N(c2ccc(-c3cc(-n4c5ccccc5c5ccccc54)cc4ccccc34)cc2)c2cccc3c2c2ccccc2n3-c2ccccc2)cc1 ; c1ccc(-c2ccccc2N(c2cccc(-c3cc(-n4c5ccccc5c5ccccc54)cc4ccccc34)c2)c2cccc3c2c2ccccc2n3-c2ccccc2)cc1]

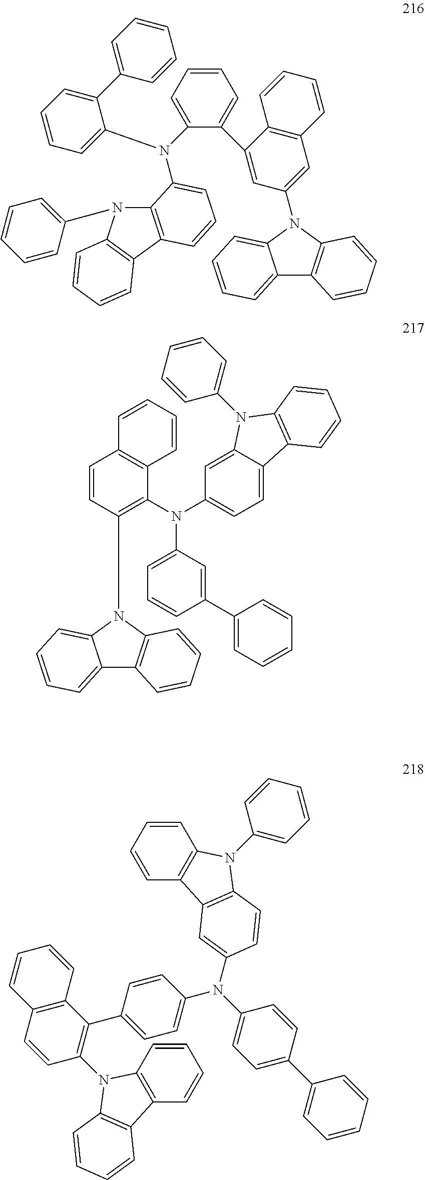 [SMILES: c1ccc(-c2ccccc2N(c2ccccc2-c2cc(-n3c4ccccc4c4ccccc43)cc3ccccc23)c2cccc3c4ccccc4n(-c4ccccc4)c23)cc1 ; c1ccc(-c2cccc(N(c3ccc4c5ccccc5n(-c5ccccc5)c4c3)c3c(-n4c5ccccc5c5ccccc54)ccc4ccccc34)c2)cc1 ; c1ccc(-c2ccc(N(c3ccc(-c4c(-n5c6ccccc6c6ccccc65)ccc5ccccc45)cc3)c3ccc4c(c3)c3ccccc3n4-c3ccccc3)cc2)cc1]

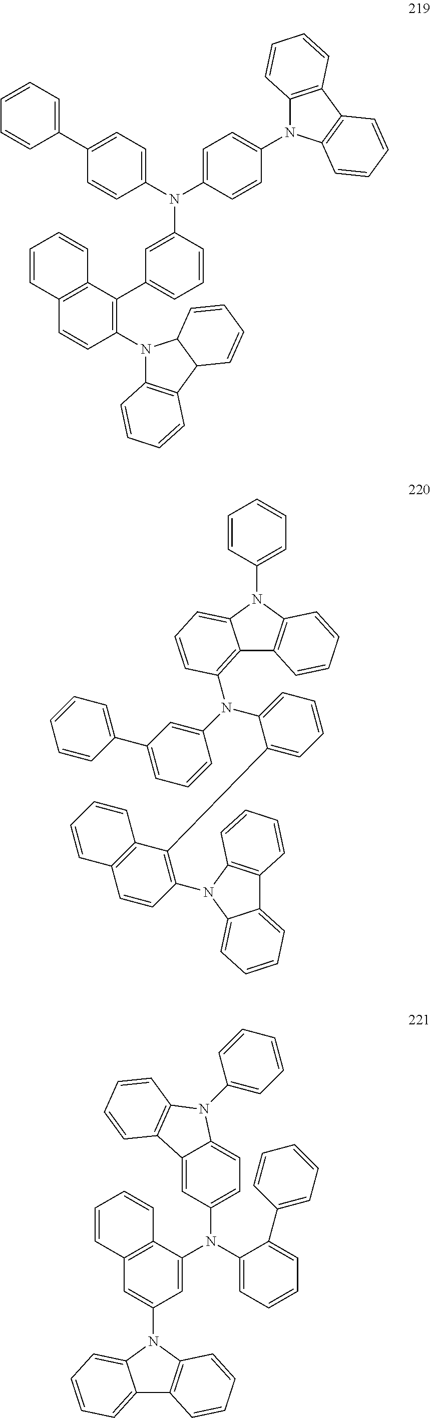 [SMILES: C1=CC2c3ccccc3N(c3ccc4ccccc4c3-c3cccc(N(c4ccc(-c5ccccc5)cc4)c4ccc(-n5c6ccccc6c6ccccc65)cc4)c3)C2C=C1 ; c1ccc(-c2cccc(N(c3ccccc3-c3c(-n4c5ccccc5c5ccccc54)ccc4ccccc34)c3cccc4c3c3ccccc3n4-c3ccccc3)c2)cc1 ; c1ccc(-c2ccccc2N(c2ccc3c(c2)c2ccccc2n3-c2ccccc2)c2cc(-n3c4ccccc4c4ccccc43)cc3ccccc23)cc1]

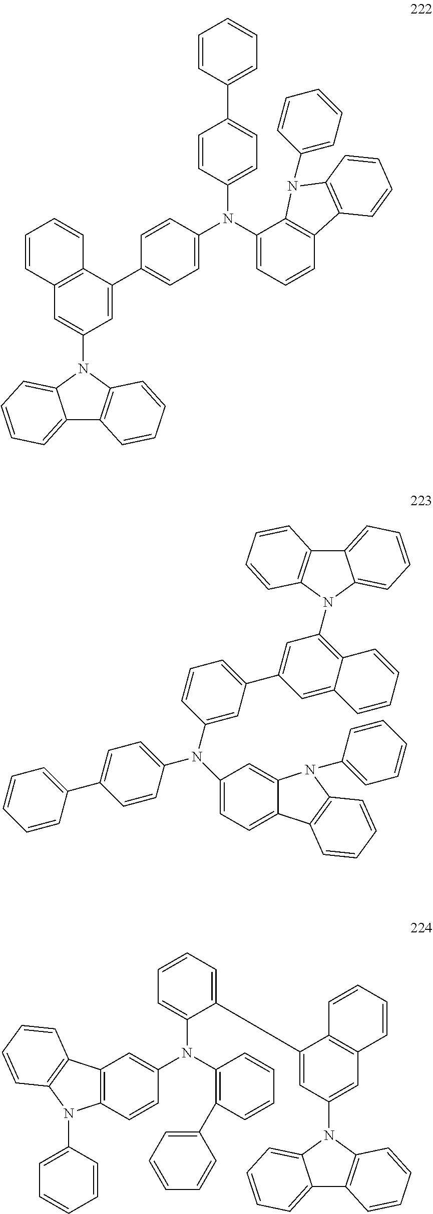 [SMILES: c1ccc(-c2ccc(N(c3ccc(-c4cc(-n5c6ccccc6c6ccccc65)cc5ccccc45)cc3)c3cccc4c5ccccc5n(-c5ccccc5)c34)cc2)cc1 ; c1ccc(-c2ccc(N(c3cccc(-c4cc(-n5c6ccccc6c6ccccc65)c5ccccc5c4)c3)c3ccc4c5ccccc5n(-c5ccccc5)c4c3)cc2)cc1 ; c1ccc(-c2ccccc2N(c2ccc3c(c2)c2ccccc2n3-c2ccccc2)c2ccccc2-c2cc(-n3c4ccccc4c4ccccc43)cc3ccccc23)cc1]

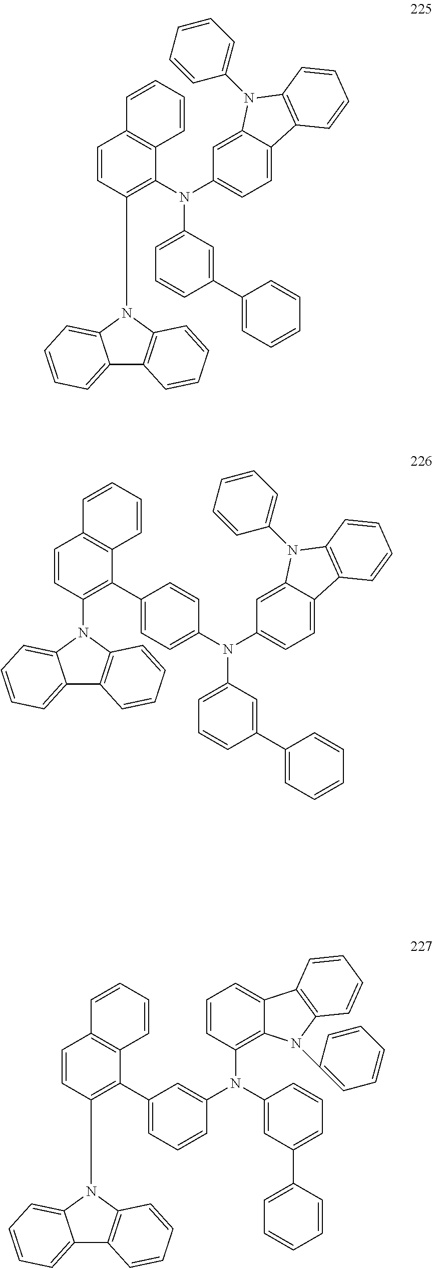 [SMILES: c1ccc(-c2cccc(N(c3ccc4c5ccccc5n(-c5ccccc5)c4c3)c3c(-n4c5ccccc5c5ccccc54)ccc4ccccc34)c2)cc1 ; c1ccc(-c2cccc(N(c3ccc(-c4c(-n5c6ccccc6c6ccccc65)ccc5ccccc45)cc3)c3ccc4c5ccccc5n(-c5ccccc5)c4c3)c2)cc1 ; c1ccc(-c2cccc(N(c3cccc(-c4c(-n5c6ccccc6c6ccccc65)ccc5ccccc45)c3)c3cccc4c5ccccc5n(-c5ccccc5)c34)c2)cc1]

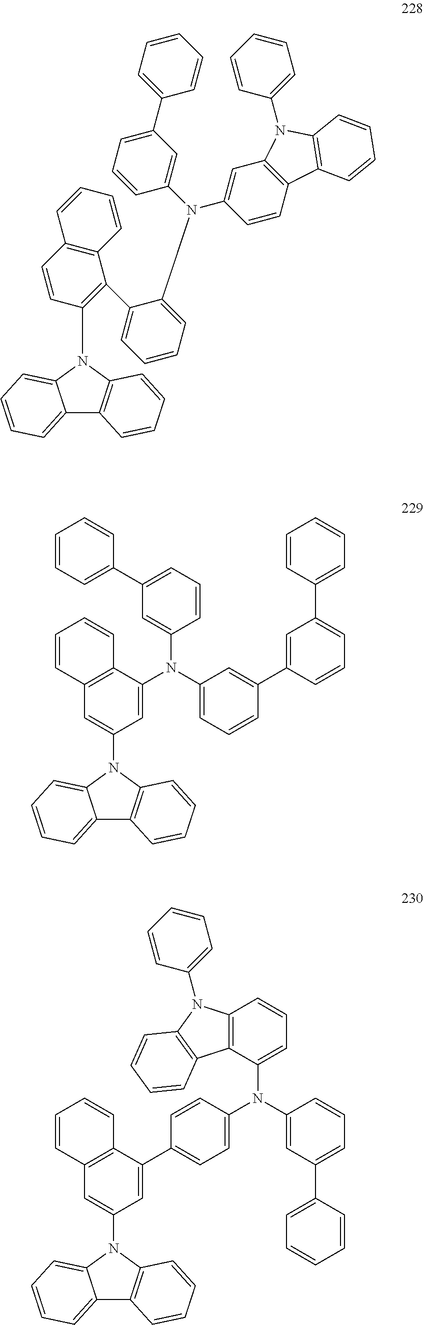 [SMILES: c1ccc(-c2cccc(N(c3ccc4c5ccccc5n(-c5ccccc5)c4c3)c3ccccc3-c3c(-n4c5ccccc5c5ccccc54)ccc4ccccc34)c2)cc1 ; c1ccc(-c2cccc(-c3cccc(N(c4cccc(-c5ccccc5)c4)c4cc(-n5c6ccccc6c6ccccc65)cc5ccccc45)c3)c2)cc1 ; c1ccc(-c2cccc(N(c3ccc(-c4cc(-n5c6ccccc6c6ccccc65)cc5ccccc45)cc3)c3cccc4c3c3ccccc3n4-c3ccccc3)c2)cc1]

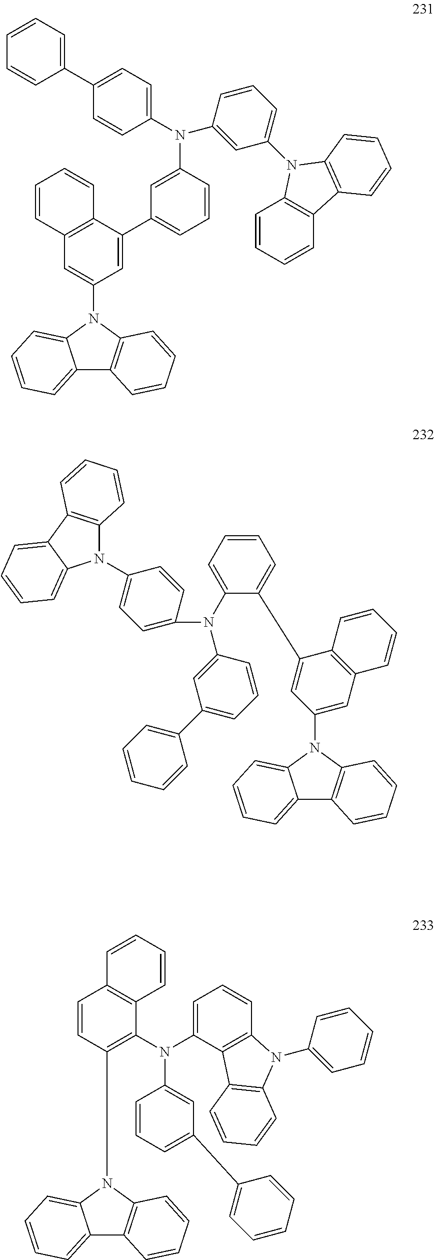 [SMILES: c1ccc(-c2ccc(N(c3cccc(-c4cc(-n5c6ccccc6c6ccccc65)cc5ccccc45)c3)c3cccc(-n4c5ccccc5c5ccccc54)c3)cc2)cc1 ; c1ccc(-c2cccc(N(c3ccc(-n4c5ccccc5c5ccccc54)cc3)c3ccccc3-c3cc(-n4c5ccccc5c5ccccc54)cc4ccccc34)c2)cc1 ; c1ccc(-c2cccc(N(c3c(-n4c5ccccc5c5ccccc54)ccc4ccccc34)c3cccc4c3c3ccccc3n4-c3ccccc3)c2)cc1]

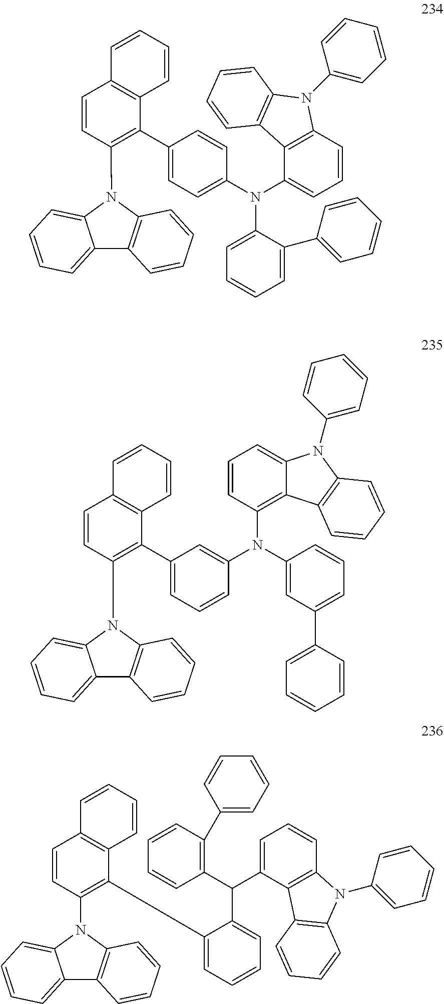 [SMILES: c1ccc(-c2ccccc2N(c2ccc(-c3c(-n4c5ccccc5c5ccccc54)ccc4ccccc34)cc2)c2cccc3c2c2ccccc2n3-c2ccccc2)cc1 ; c1ccc(-c2cccc(N(c3cccc(-c4c(-n5c6ccccc6c6ccccc65)ccc5ccccc45)c3)c3cccc4c3c3ccccc3n4-c3ccccc3)c2)cc1 ; c1ccc(-c2ccccc2C(c2ccccc2-c2c(-n3c4ccccc4c4ccccc43)ccc3ccccc23)c2cccc3c2c2ccccc2n3-c2ccccc2)cc1]

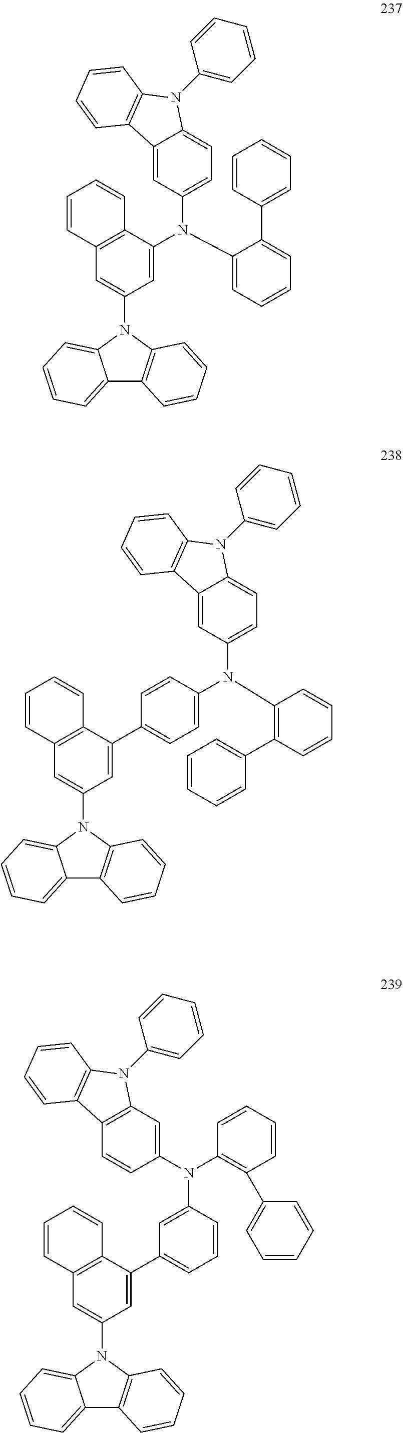 [SMILES: c1ccc(-c2ccccc2N(c2ccc3c(c2)c2ccccc2n3-c2ccccc2)c2cc(-n3c4ccccc4c4ccccc43)cc3ccccc23)cc1 ; c1ccc(-c2ccccc2N(c2ccc(-c3cc(-n4c5ccccc5c5ccccc54)cc4ccccc34)cc2)c2ccc3c(c2)c2ccccc2n3-c2ccccc2)cc1 ; c1ccc(-c2ccccc2N(c2cccc(-c3cc(-n4c5ccccc5c5ccccc54)cc4ccccc34)c2)c2ccc3c4ccccc4n(-c4ccccc4)c3c2)cc1]

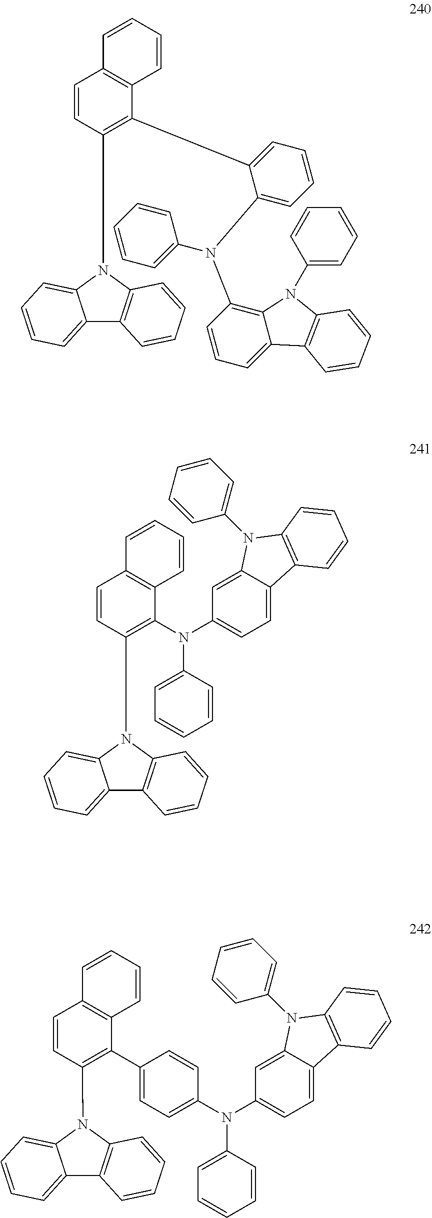 [SMILES: c1ccc(N(c2ccccc2-c2c(-n3c4ccccc4c4ccccc43)ccc3ccccc23)c2cccc3c4ccccc4n(-c4ccccc4)c23)cc1 ; c1ccc(N(c2ccc3c4ccccc4n(-c4ccccc4)c3c2)c2c(-n3c4ccccc4c4ccccc43)ccc3ccccc23)cc1 ; c1ccc(N(c2ccc(-c3c(-n4c5ccccc5c5ccccc54)ccc4ccccc34)cc2)c2ccc3c4ccccc4n(-c4ccccc4)c3c2)cc1]

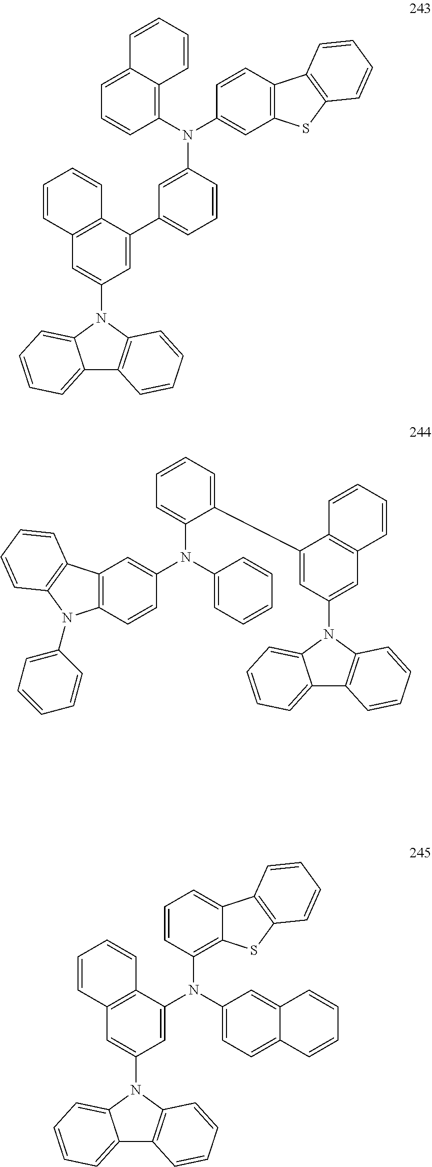 [SMILES: c1cc(-c2cc(-n3c4ccccc4c4ccccc43)cc3ccccc23)cc(N(c2ccc3c(c2)sc2ccccc23)c2cccc3ccccc23)c1 ; c1ccc(N(c2ccc3c(c2)c2ccccc2n3-c2ccccc2)c2ccccc2-c2cc(-n3c4ccccc4c4ccccc43)cc3ccccc23)cc1 ; c1ccc2cc(N(c3cc(-n4c5ccccc5c5ccccc54)cc4ccccc34)c3cccc4c3sc3ccccc34)ccc2c1]

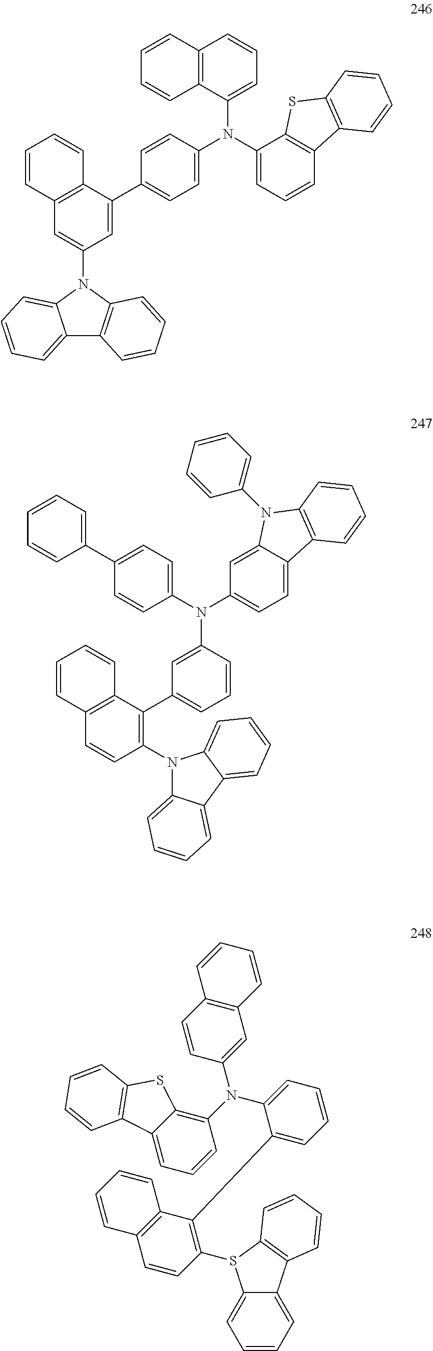

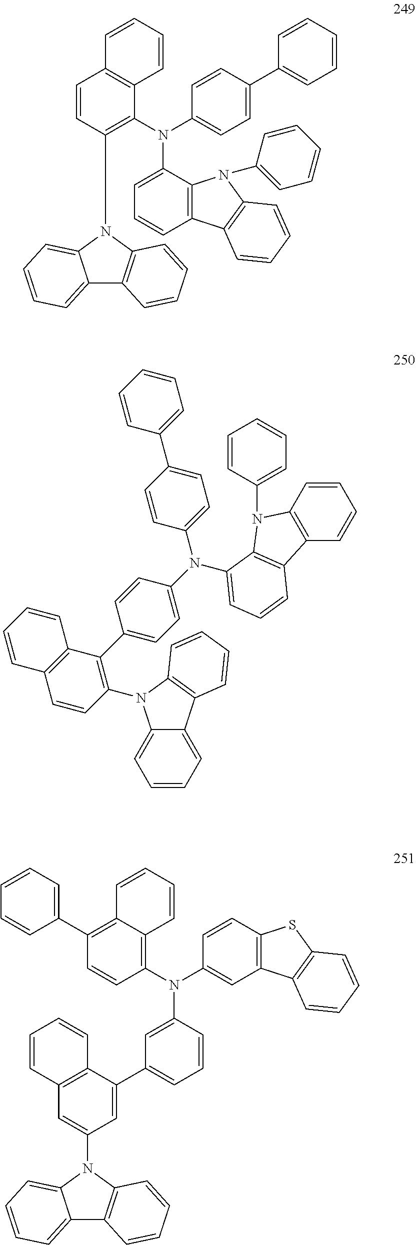 [SMILES: c1ccc(-c2ccc(N(c3c(-n4c5ccccc5c5ccccc54)ccc4ccccc34)c3cccc4c5ccccc5n(-c5ccccc5)c34)cc2)cc1 ; c1ccc(-c2ccc(N(c3ccc(-c4c(-n5c6ccccc6c6ccccc65)ccc5ccccc45)cc3)c3cccc4c5ccccc5n(-c5ccccc5)c34)cc2)cc1 ; c1ccc(-c2ccc(N(c3cccc(-c4cc(-n5c6ccccc6c6ccccc65)cc5ccccc45)c3)c3ccc4sc5ccccc5c4c3)c3ccccc23)cc1]

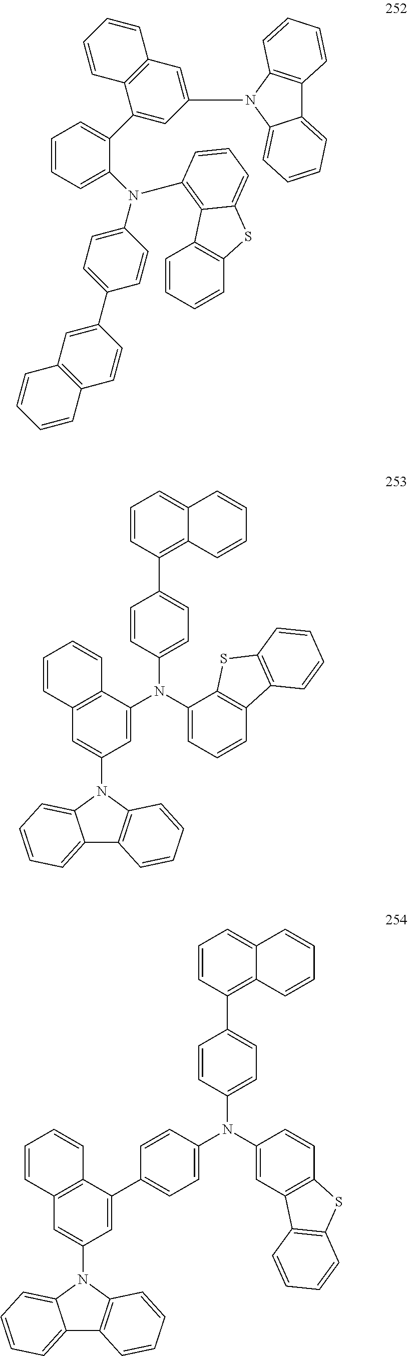 [SMILES: c1ccc(N(c2ccc(-c3ccc4ccccc4c3)cc2)c2cccc3sc4ccccc4c23)c(-c2cc(-n3c4ccccc4c4ccccc43)cc3ccccc23)c1 ; c1ccc2c(-c3ccc(N(c4cc(-n5c6ccccc6c6ccccc65)cc5ccccc45)c4cccc5c4sc4ccccc45)cc3)cccc2c1 ; c1ccc2c(-c3ccc(N(c4ccc(-c5cc(-n6c7ccccc7c7ccccc76)cc6ccccc56)cc4)c4ccc5sc6ccccc6c5c4)cc3)cccc2c1]

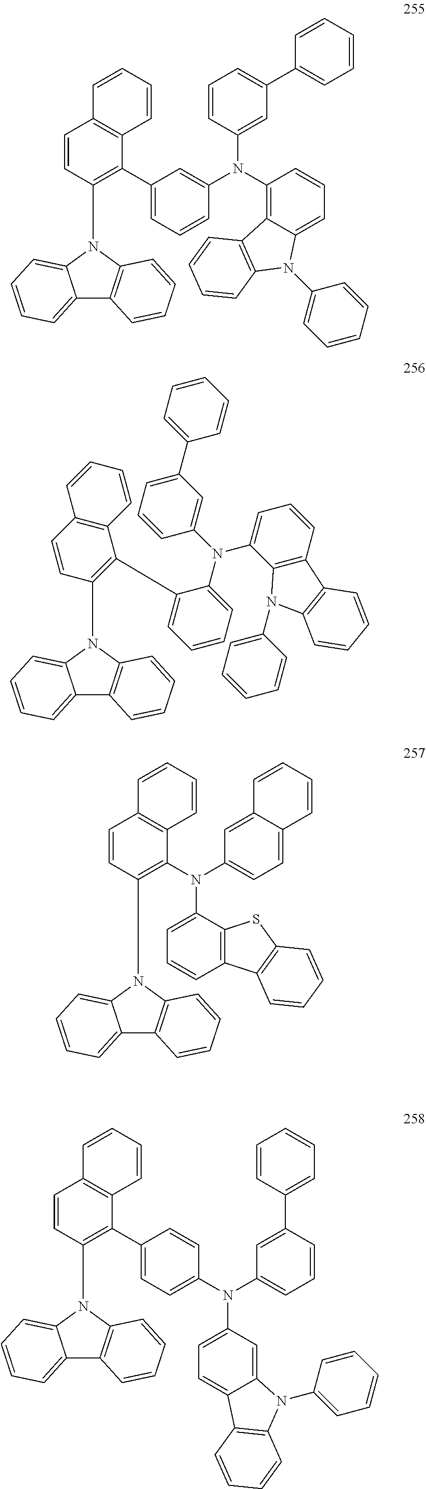 [SMILES: c1ccc(-c2cccc(N(c3cccc(-c4c(-n5c6ccccc6c6ccccc65)ccc5ccccc45)c3)c3cccc4c3c3ccccc3n4-c3ccccc3)c2)cc1 ; c1ccc(-c2cccc(N(c3ccccc3-c3c(-n4c5ccccc5c5ccccc54)ccc4ccccc34)c3cccc4c5ccccc5n(-c5ccccc5)c34)c2)cc1 ; c1ccc2cc(N(c3c(-n4c5ccccc5c5ccccc54)ccc4ccccc34)c3cccc4c3sc3ccccc34)ccc2c1 ; c1ccc(-c2cccc(N(c3ccc(-c4c(-n5c6ccccc6c6ccccc65)ccc5ccccc45)cc3)c3ccc4c5ccccc5n(-c5ccccc5)c4c3)c2)cc1]

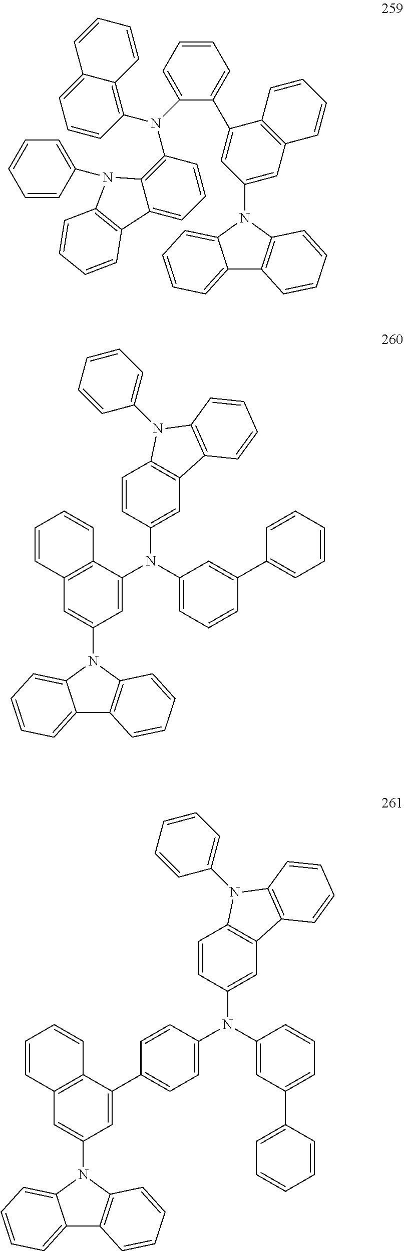 [SMILES: c1ccc(-n2c3ccccc3c3cccc(N(c4ccccc4-c4cc(-n5c6ccccc6c6ccccc65)cc5ccccc45)c4cccc5ccccc45)c32)cc1 ; c1ccc(-c2cccc(N(c3ccc4c(c3)c3ccccc3n4-c3ccccc3)c3cc(-n4c5ccccc5c5ccccc54)cc4ccccc34)c2)cc1 ; c1ccc(-c2cccc(N(c3ccc(-c4cc(-n5c6ccccc6c6ccccc65)cc5ccccc45)cc3)c3ccc4c(c3)c3ccccc3n4-c3ccccc3)c2)cc1]

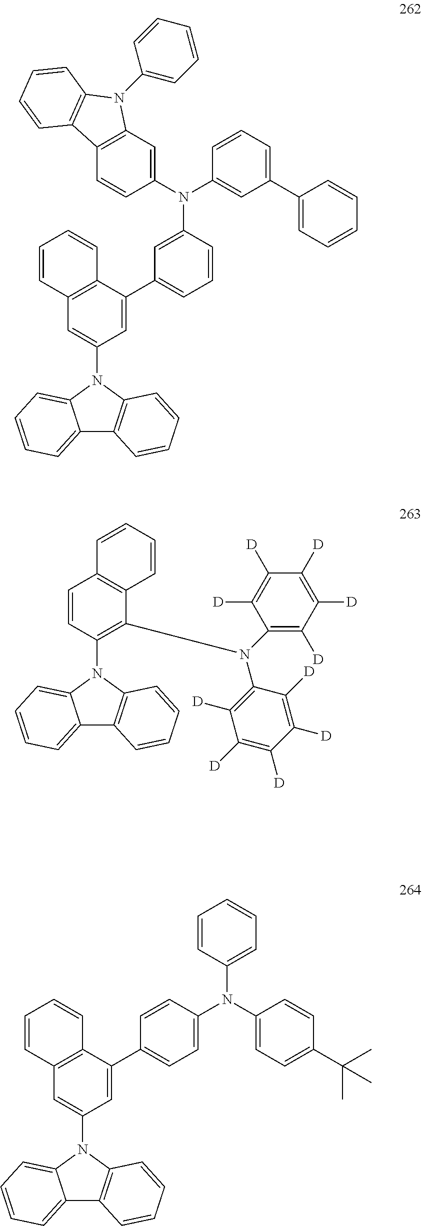 [SMILES: c1ccc(-c2cccc(N(c3cccc(-c4cc(-n5c6ccccc6c6ccccc65)cc5ccccc45)c3)c3ccc4c5ccccc5n(-c5ccccc5)c4c3)c2)cc1 ; [2H]c1c([2H])c([2H])c(N(c2c([2H])c([2H])c([2H])c([2H])c2[2H])c2c(-n3c4ccccc4c4ccccc43)ccc3ccccc23)c([2H])c1[2H] ; CC(C)(C)c1ccc(N(c2ccccc2)c2ccc(-c3cc(-n4c5ccccc5c5ccccc54)cc4ccccc34)cc2)cc1]

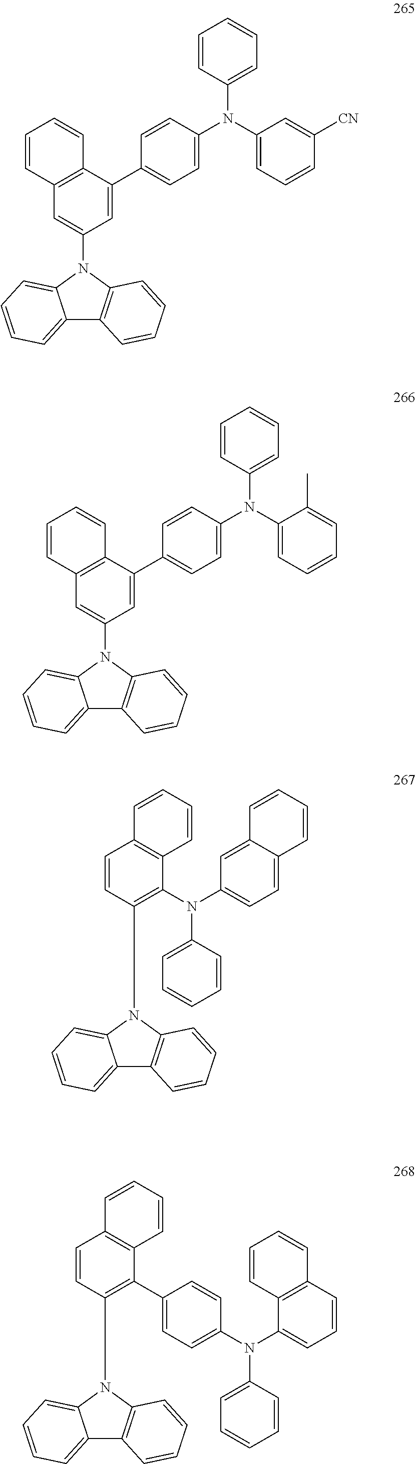 [SMILES: N#Cc1cccc(N(c2ccccc2)c2ccc(-c3cc(-n4c5ccccc5c5ccccc54)cc4ccccc34)cc2)c1 ; Cc1ccccc1N(c1ccccc1)c1ccc(-c2cc(-n3c4ccccc4c4ccccc43)cc3ccccc23)cc1 ; c1ccc(N(c2ccc3ccccc3c2)c2c(-n3c4ccccc4c4ccccc43)ccc3ccccc23)cc1 ; c1ccc(N(c2ccc(-c3c(-n4c5ccccc5c5ccccc54)ccc4ccccc34)cc2)c2cccc3ccccc23)cc1]

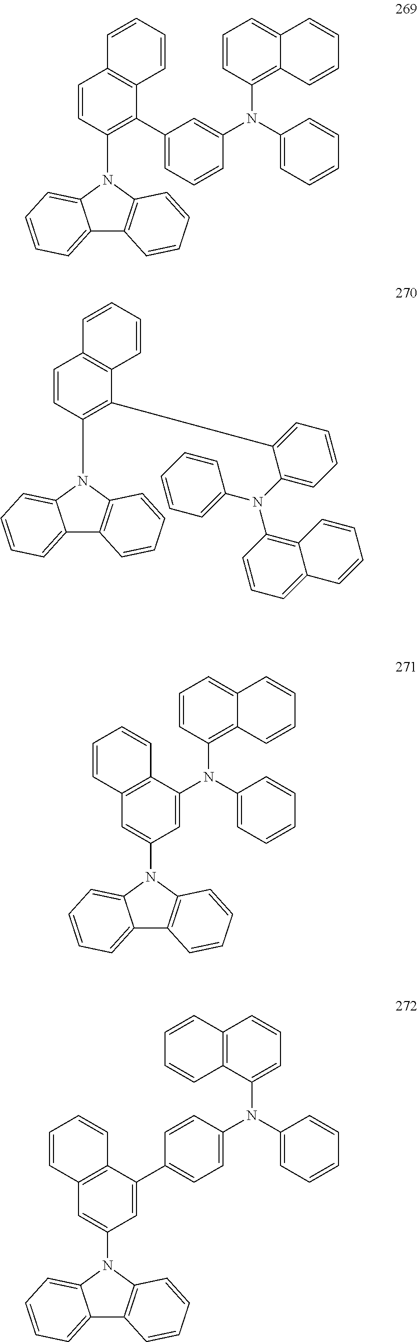 [SMILES: c1ccc(N(c2cccc(-c3c(-n4c5ccccc5c5ccccc54)ccc4ccccc34)c2)c2cccc3ccccc23)cc1 ; c1ccc(N(c2ccccc2-c2c(-n3c4ccccc4c4ccccc43)ccc3ccccc23)c2cccc3ccccc23)cc1 ; c1ccc(N(c2cccc3ccccc23)c2cc(-n3c4ccccc4c4ccccc43)cc3ccccc23)cc1 ; c1ccc(N(c2ccc(-c3cc(-n4c5ccccc5c5ccccc54)cc4ccccc34)cc2)c2cccc3ccccc23)cc1]

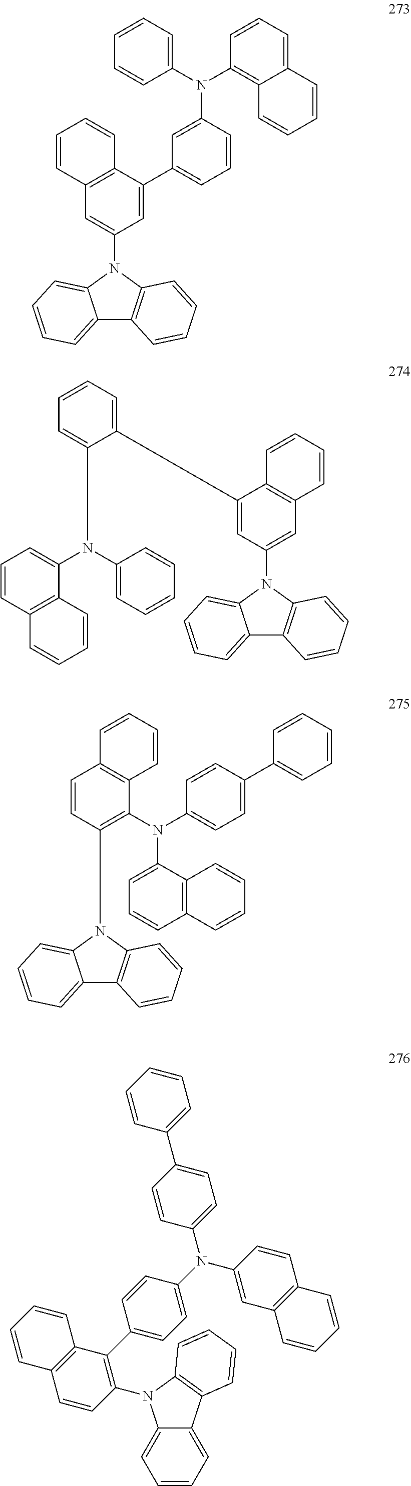

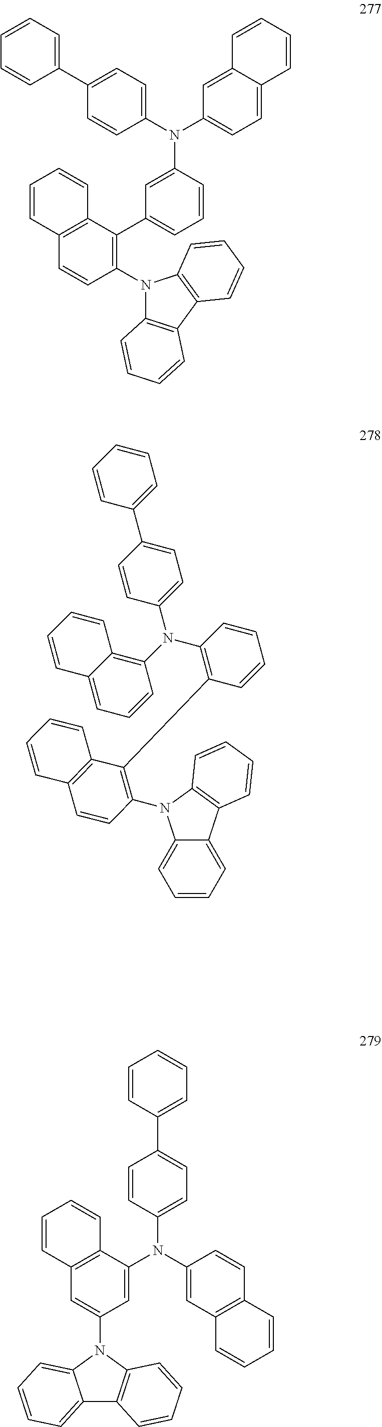 [SMILES: c1ccc(-c2ccc(N(c3cccc(-c4c(-n5c6ccccc6c6ccccc65)ccc5ccccc45)c3)c3ccc4ccccc4c3)cc2)cc1 ; c1ccc(-c2ccc(N(c3ccccc3-c3c(-n4c5ccccc5c5ccccc54)ccc4ccccc34)c3cccc4ccccc34)cc2)cc1 ; c1ccc(-c2ccc(N(c3ccc4ccccc4c3)c3cc(-n4c5ccccc5c5ccccc54)cc4ccccc34)cc2)cc1]

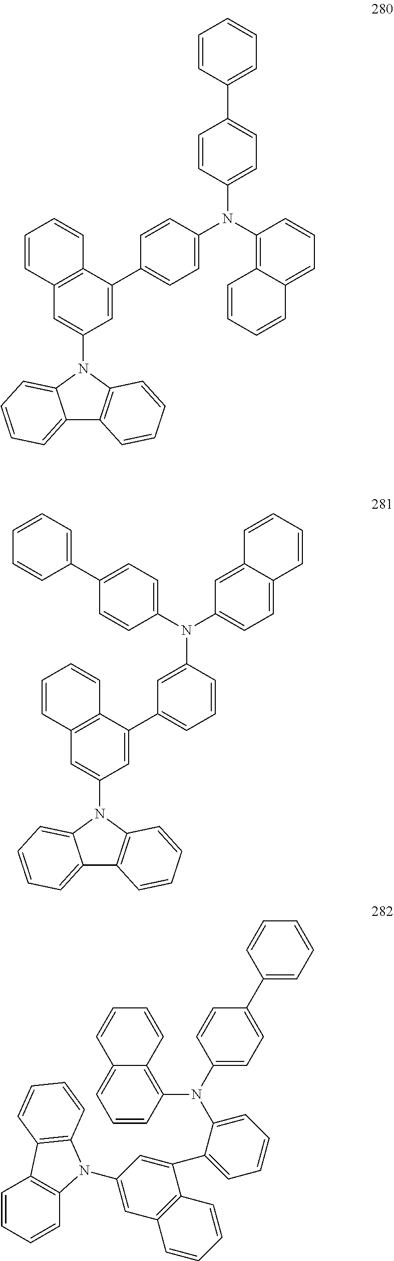 [SMILES: c1ccc(-c2ccc(N(c3ccc(-c4cc(-n5c6ccccc6c6ccccc65)cc5ccccc45)cc3)c3cccc4ccccc34)cc2)cc1 ; c1ccc(-c2ccc(N(c3cccc(-c4cc(-n5c6ccccc6c6ccccc65)cc5ccccc45)c3)c3ccc4ccccc4c3)cc2)cc1 ; c1ccc(-c2ccc(N(c3ccccc3-c3cc(-n4c5ccccc5c5ccccc54)cc4ccccc34)c3cccc4ccccc34)cc2)cc1]

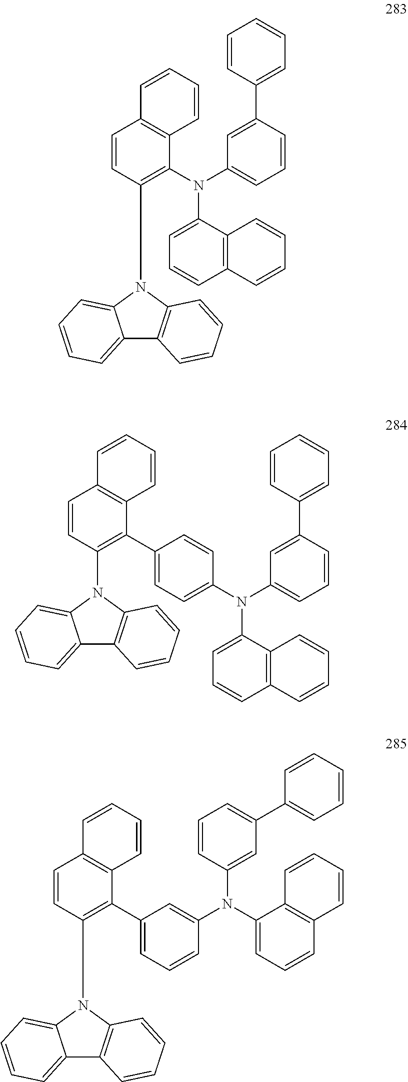 [SMILES: c1ccc(-c2cccc(N(c3cccc4ccccc34)c3c(-n4c5ccccc5c5ccccc54)ccc4ccccc34)c2)cc1 ; c1ccc(-c2cccc(N(c3ccc(-c4c(-n5c6ccccc6c6ccccc65)ccc5ccccc45)cc3)c3cccc4ccccc34)c2)cc1 ; c1ccc(-c2cccc(N(c3cccc(-c4c(-n5c6ccccc6c6ccccc65)ccc5ccccc45)c3)c3cccc4ccccc34)c2)cc1]

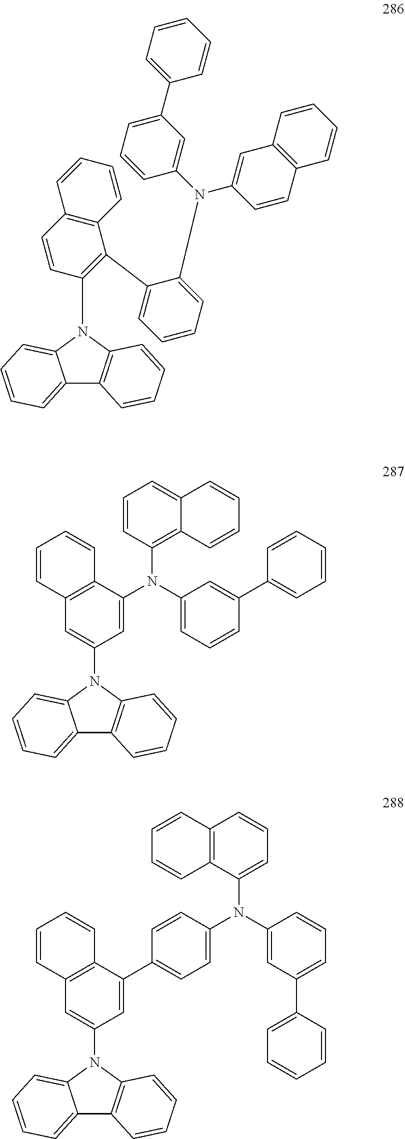 [SMILES: c1ccc(-c2cccc(N(c3ccc4ccccc4c3)c3ccccc3-c3c(-n4c5ccccc5c5ccccc54)ccc4ccccc34)c2)cc1 ; c1ccc(-c2cccc(N(c3cccc4ccccc34)c3cc(-n4c5ccccc5c5ccccc54)cc4ccccc34)c2)cc1 ; c1ccc(-c2cccc(N(c3ccc(-c4cc(-n5c6ccccc6c6ccccc65)cc5ccccc45)cc3)c3cccc4ccccc34)c2)cc1]

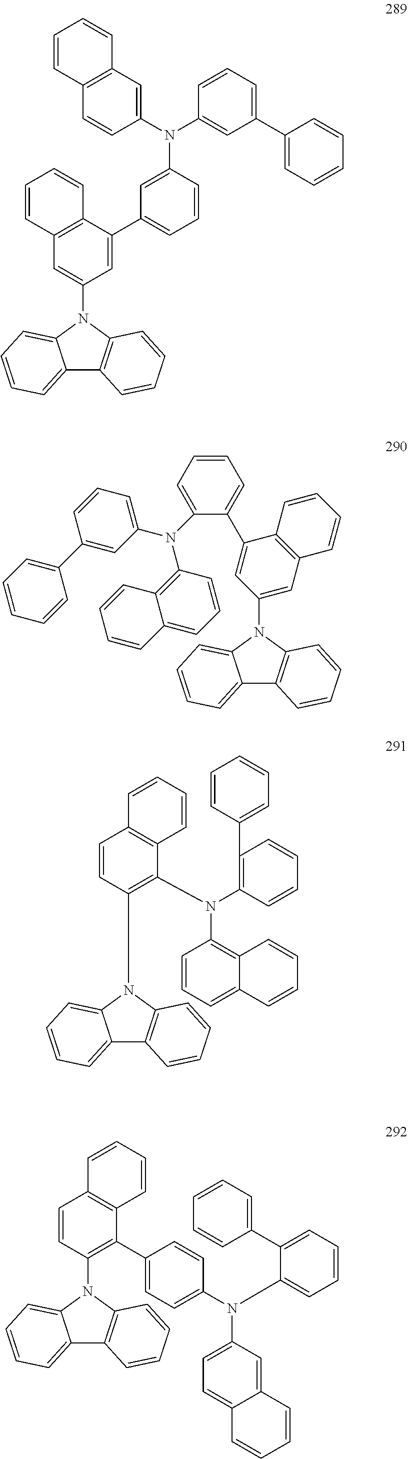 [SMILES: c1ccc(-c2cccc(N(c3cccc(-c4cc(-n5c6ccccc6c6ccccc65)cc5ccccc45)c3)c3ccc4ccccc4c3)c2)cc1 ; c1ccc(-c2cccc(N(c3ccccc3-c3cc(-n4c5ccccc5c5ccccc54)cc4ccccc34)c3cccc4ccccc34)c2)cc1 ; c1ccc(-c2ccccc2N(c2cccc3ccccc23)c2c(-n3c4ccccc4c4ccccc43)ccc3ccccc23)cc1 ; c1ccc(-c2ccccc2N(c2ccc(-c3c(-n4c5ccccc5c5ccccc54)ccc4ccccc34)cc2)c2ccc3ccccc3c2)cc1]

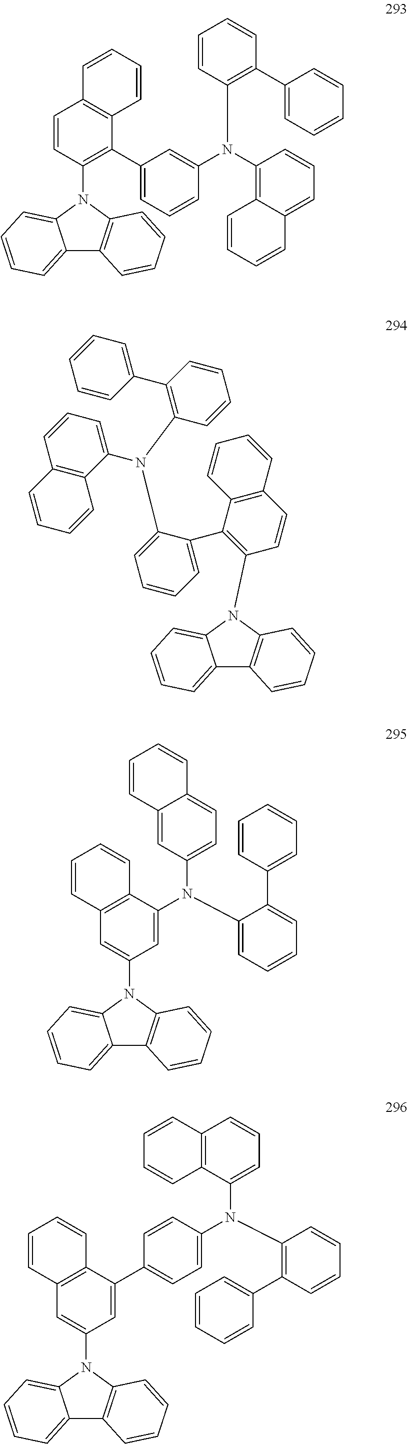 [SMILES: c1ccc(-c2ccccc2N(c2cccc(-c3c(-n4c5ccccc5c5ccccc54)ccc4ccccc34)c2)c2cccc3ccccc23)cc1 ; c1ccc(-c2ccccc2N(c2ccccc2-c2c(-n3c4ccccc4c4ccccc43)ccc3ccccc23)c2cccc3ccccc23)cc1 ; c1ccc(-c2ccccc2N(c2ccc3ccccc3c2)c2cc(-n3c4ccccc4c4ccccc43)cc3ccccc23)cc1 ; c1ccc(-c2ccccc2N(c2ccc(-c3cc(-n4c5ccccc5c5ccccc54)cc4ccccc34)cc2)c2cccc3ccccc23)cc1]

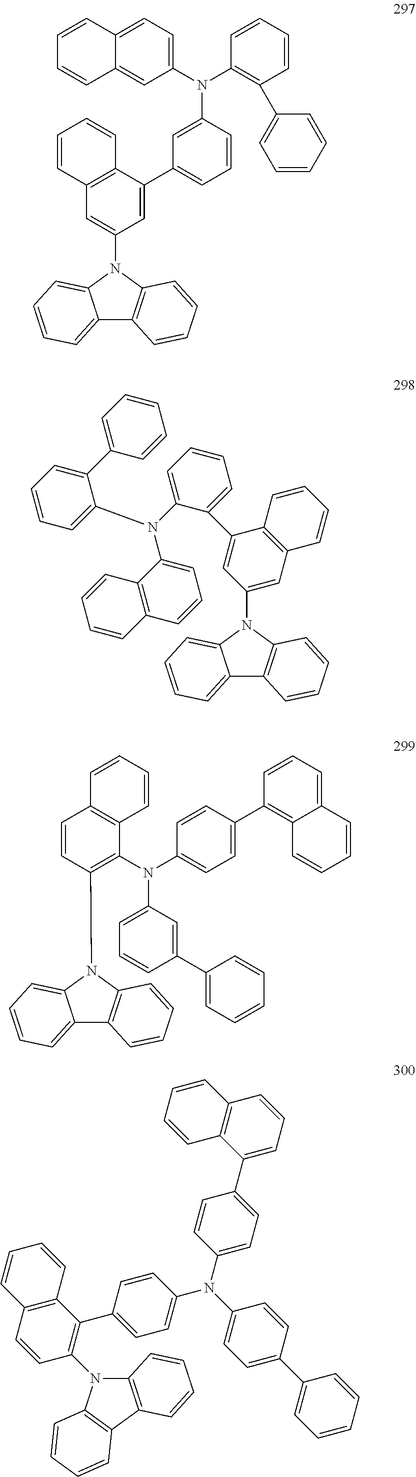 [SMILES: c1ccc(-c2ccccc2N(c2cccc(-c3cc(-n4c5ccccc5c5ccccc54)cc4ccccc34)c2)c2ccc3ccccc3c2)cc1 ; c1ccc(-c2ccccc2N(c2ccccc2-c2cc(-n3c4ccccc4c4ccccc43)cc3ccccc23)c2cccc3ccccc23)cc1 ; c1ccc(-c2cccc(N(c3ccc(-c4cccc5ccccc45)cc3)c3c(-n4c5ccccc5c5ccccc54)ccc4ccccc34)c2)cc1 ; c1ccc(-c2ccc(N(c3ccc(-c4cccc5ccccc45)cc3)c3ccc(-c4c(-n5c6ccccc6c6ccccc65)ccc5ccccc45)cc3)cc2)cc1]

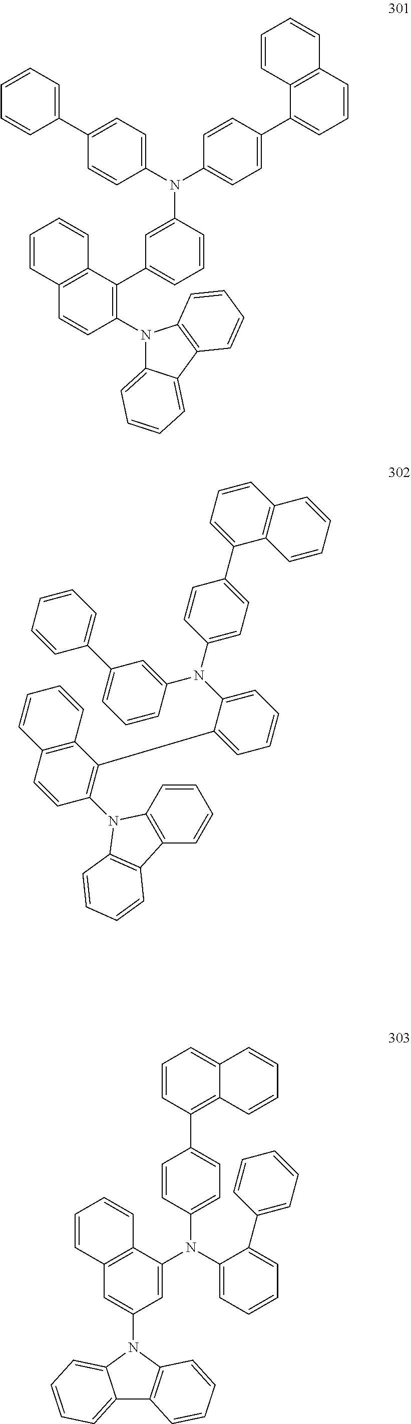 [SMILES: c1ccc(-c2ccc(N(c3ccc(-c4cccc5ccccc45)cc3)c3cccc(-c4c(-n5c6ccccc6c6ccccc65)ccc5ccccc45)c3)cc2)cc1 ; c1ccc(-c2cccc(N(c3ccc(-c4cccc5ccccc45)cc3)c3ccccc3-c3c(-n4c5ccccc5c5ccccc54)ccc4ccccc34)c2)cc1 ; c1ccc(-c2ccccc2N(c2ccc(-c3cccc4ccccc34)cc2)c2cc(-n3c4ccccc4c4ccccc43)cc3ccccc23)cc1]

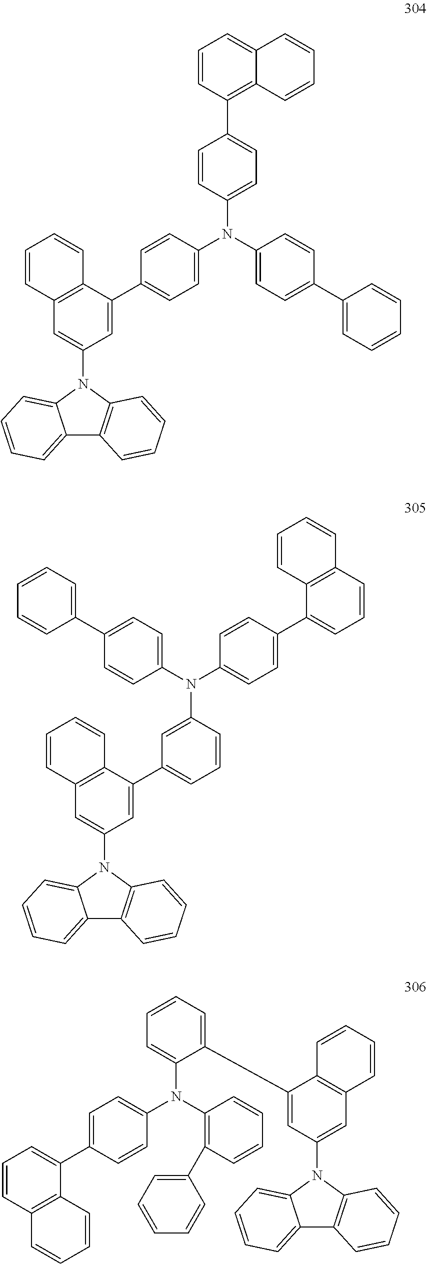 [SMILES: c1ccc(-c2ccc(N(c3ccc(-c4cccc5ccccc45)cc3)c3ccc(-c4cc(-n5c6ccccc6c6ccccc65)cc5ccccc45)cc3)cc2)cc1 ; c1ccc(-c2ccc(N(c3ccc(-c4cccc5ccccc45)cc3)c3cccc(-c4cc(-n5c6ccccc6c6ccccc65)cc5ccccc45)c3)cc2)cc1 ; c1ccc(-c2ccccc2N(c2ccc(-c3cccc4ccccc34)cc2)c2ccccc2-c2cc(-n3c4ccccc4c4ccccc43)cc3ccccc23)cc1]

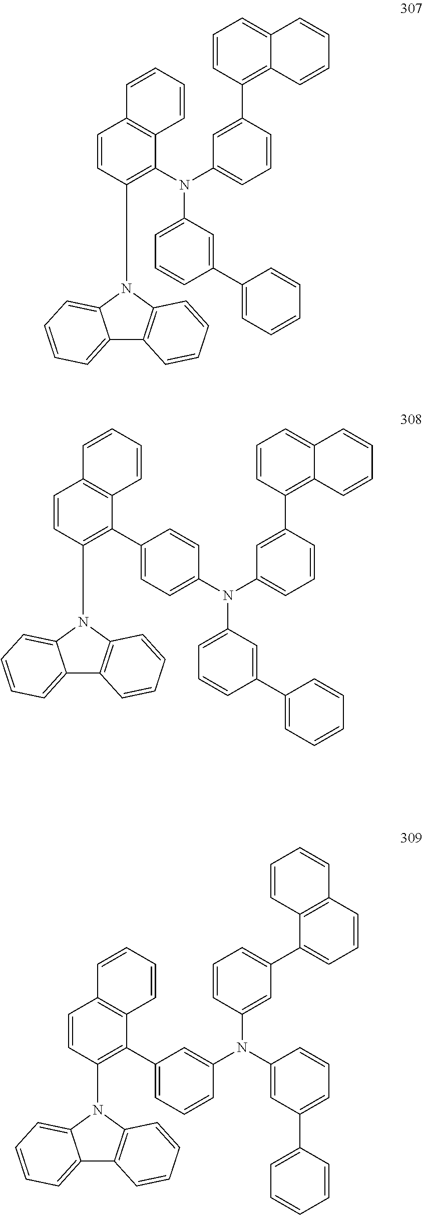 [SMILES: c1ccc(-c2cccc(N(c3cccc(-c4cccc5ccccc45)c3)c3c(-n4c5ccccc5c5ccccc54)ccc4ccccc34)c2)cc1 ; c1ccc(-c2cccc(N(c3ccc(-c4c(-n5c6ccccc6c6ccccc65)ccc5ccccc45)cc3)c3cccc(-c4cccc5ccccc45)c3)c2)cc1 ; c1ccc(-c2cccc(N(c3cccc(-c4cccc5ccccc45)c3)c3cccc(-c4c(-n5c6ccccc6c6ccccc65)ccc5ccccc45)c3)c2)cc1]

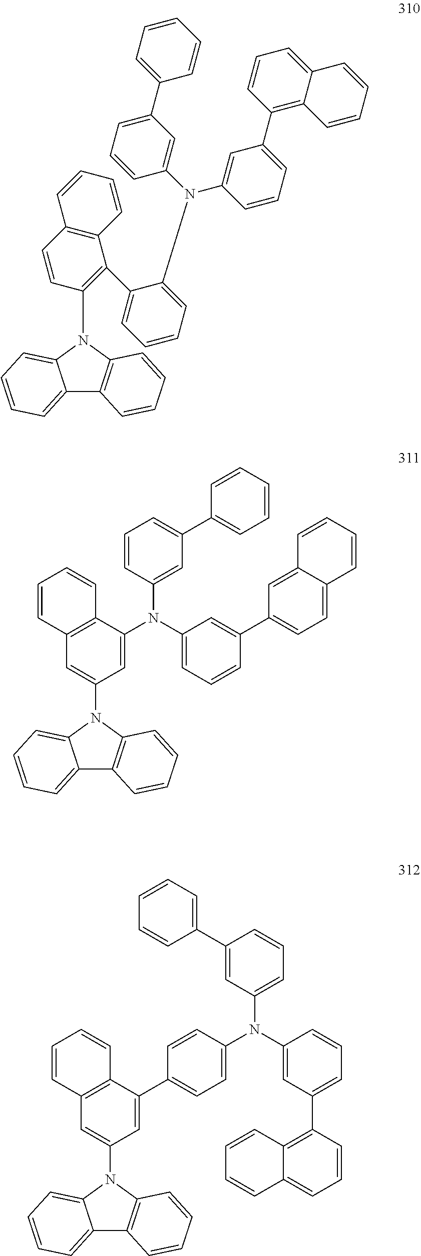 [SMILES: c1ccc(-c2cccc(N(c3cccc(-c4cccc5ccccc45)c3)c3ccccc3-c3c(-n4c5ccccc5c5ccccc54)ccc4ccccc34)c2)cc1 ; c1ccc(-c2cccc(N(c3cccc(-c4ccc5ccccc5c4)c3)c3cc(-n4c5ccccc5c5ccccc54)cc4ccccc34)c2)cc1 ; c1ccc(-c2cccc(N(c3ccc(-c4cc(-n5c6ccccc6c6ccccc65)cc5ccccc45)cc3)c3cccc(-c4cccc5ccccc45)c3)c2)cc1]

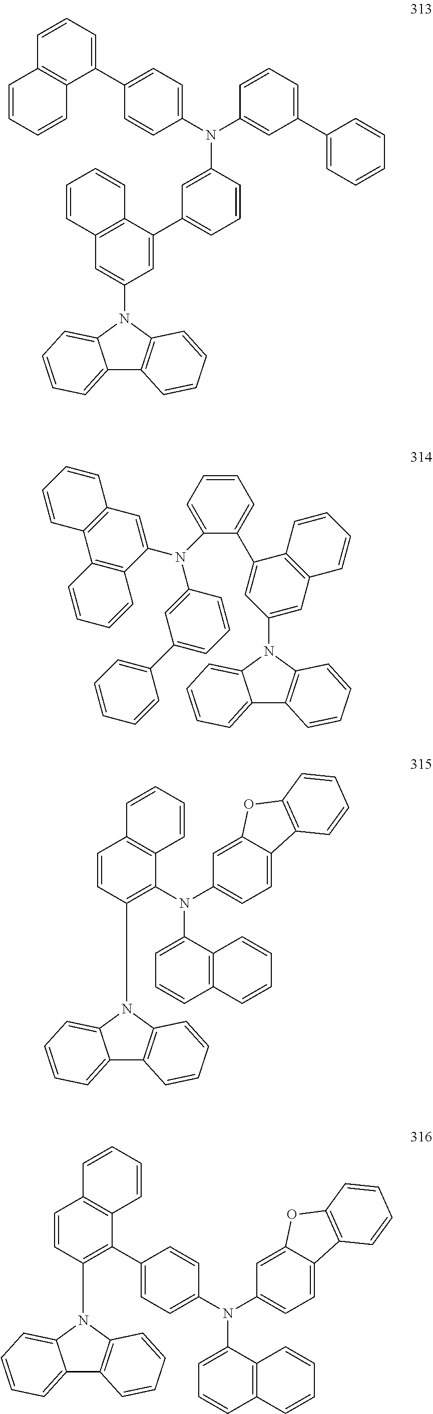

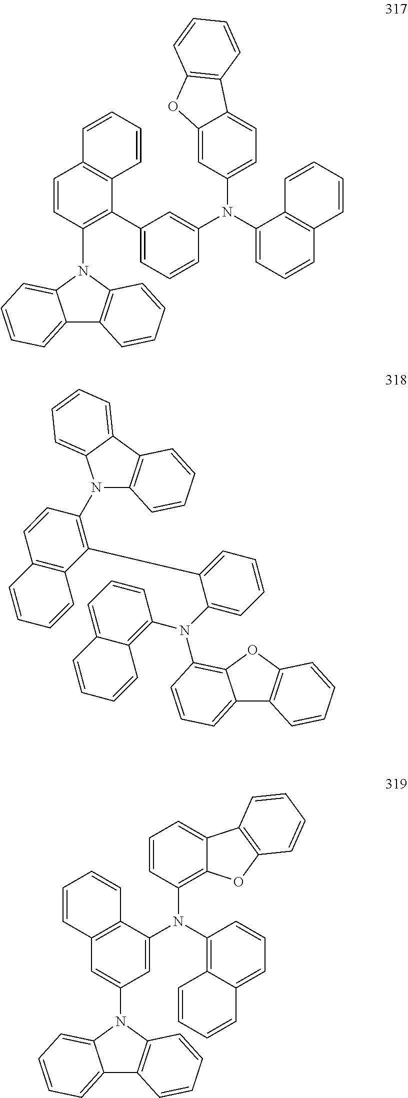 [SMILES: c1cc(-c2c(-n3c4ccccc4c4ccccc43)ccc3ccccc23)cc(N(c2ccc3c(c2)oc2ccccc23)c2cccc3ccccc23)c1 ; c1ccc(N(c2cccc3ccccc23)c2cccc3c2oc2ccccc23)c(-c2c(-n3c4ccccc4c4ccccc43)ccc3ccccc23)c1 ; c1ccc2c(N(c3cc(-n4c5ccccc5c5ccccc54)cc4ccccc34)c3cccc4c3oc3ccccc34)cccc2c1]

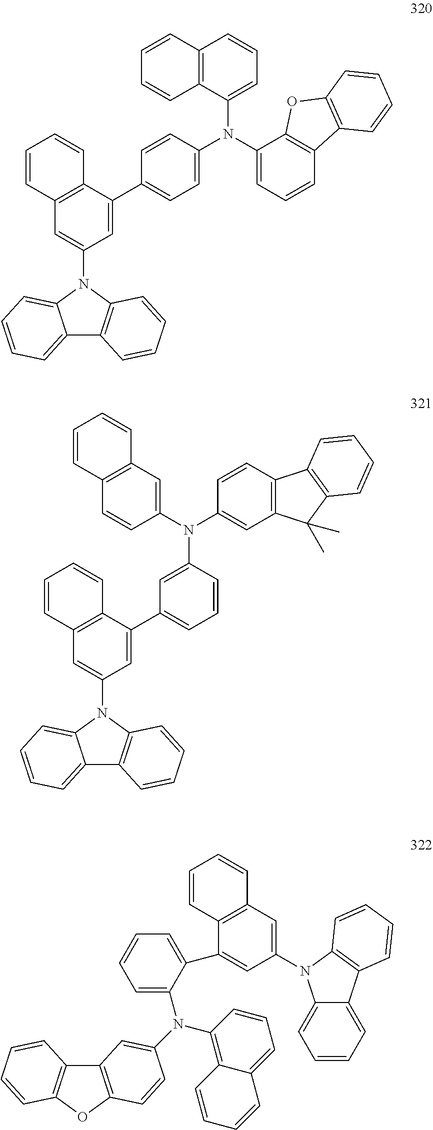 [SMILES: c1ccc2c(-c3ccc(N(c4cccc5ccccc45)c4cccc5c4oc4ccccc45)cc3)cc(-n3c4ccccc4c4ccccc43)cc2c1 ; CC1(C)c2ccccc2-c2ccc(N(c3cccc(-c4cc(-n5c6ccccc6c6ccccc65)cc5ccccc45)c3)c3ccc4ccccc4c3)cc21 ; c1ccc(N(c2ccc3oc4ccccc4c3c2)c2cccc3ccccc23)c(-c2cc(-n3c4ccccc4c4ccccc43)cc3ccccc23)c1]

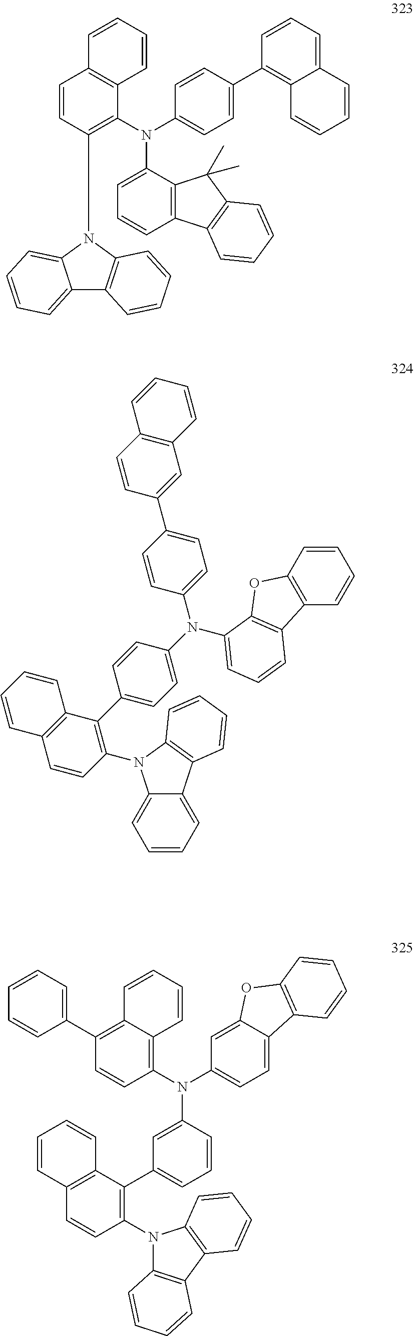 [SMILES: CC1(C)c2ccccc2-c2cccc(N(c3ccc(-c4cccc5ccccc45)cc3)c3c(-n4c5ccccc5c5ccccc54)ccc4ccccc34)c21 ; c1ccc2cc(-c3ccc(N(c4ccc(-c5c(-n6c7ccccc7c7ccccc76)ccc6ccccc56)cc4)c4cccc5c4oc4ccccc45)cc3)ccc2c1 ; c1ccc(-c2ccc(N(c3cccc(-c4c(-n5c6ccccc6c6ccccc65)ccc5ccccc45)c3)c3ccc4c(c3)oc3ccccc34)c3ccccc23)cc1]

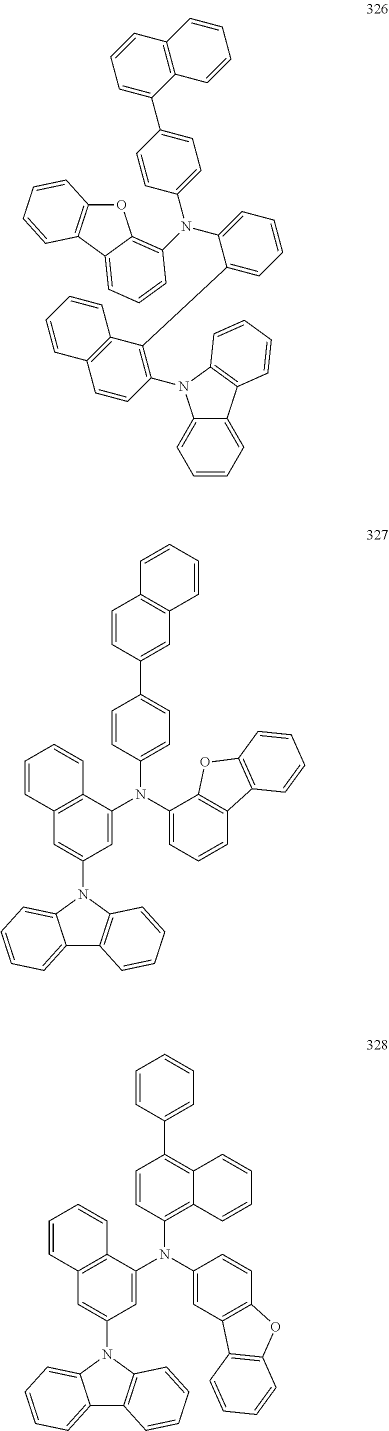 [SMILES: c1ccc(N(c2ccc(-c3cccc4ccccc34)cc2)c2cccc3c2oc2ccccc23)c(-c2c(-n3c4ccccc4c4ccccc43)ccc3ccccc23)c1 ; c1ccc2cc(-c3ccc(N(c4cc(-n5c6ccccc6c6ccccc65)cc5ccccc45)c4cccc5c4oc4ccccc45)cc3)ccc2c1 ; c1ccc(-c2ccc(N(c3ccc4oc5ccccc5c4c3)c3cc(-n4c5ccccc5c5ccccc54)cc4ccccc34)c3ccccc23)cc1]

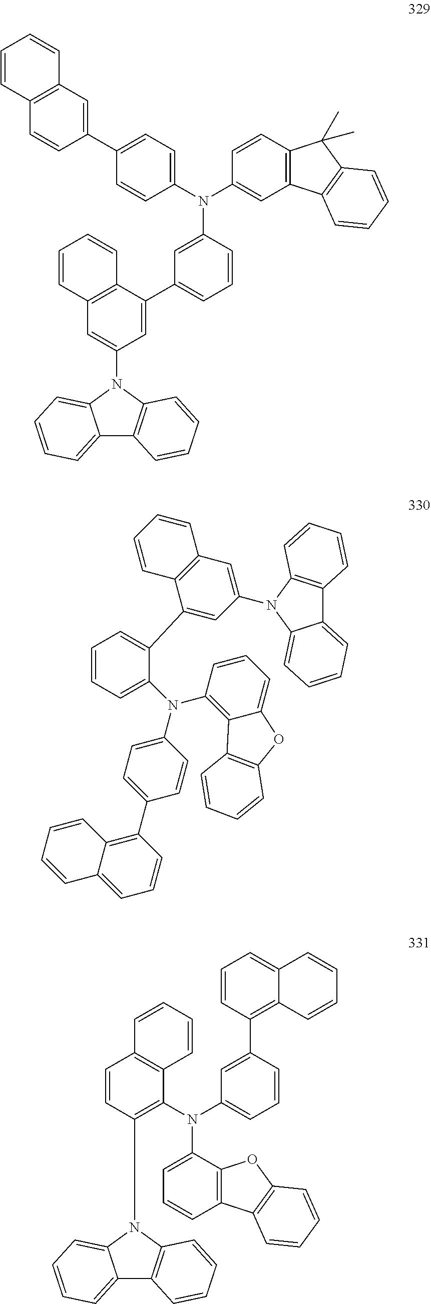 [SMILES: CC1(C)c2ccccc2-c2cc(N(c3ccc(-c4ccc5ccccc5c4)cc3)c3cccc(-c4cc(-n5c6ccccc6c6ccccc65)cc5ccccc45)c3)ccc21 ; c1ccc(N(c2ccc(-c3cccc4ccccc34)cc2)c2cccc3oc4ccccc4c23)c(-c2cc(-n3c4ccccc4c4ccccc43)cc3ccccc23)c1 ; c1cc(-c2cccc3ccccc23)cc(N(c2c(-n3c4ccccc4c4ccccc43)ccc3ccccc23)c2cccc3c2oc2ccccc23)c1]

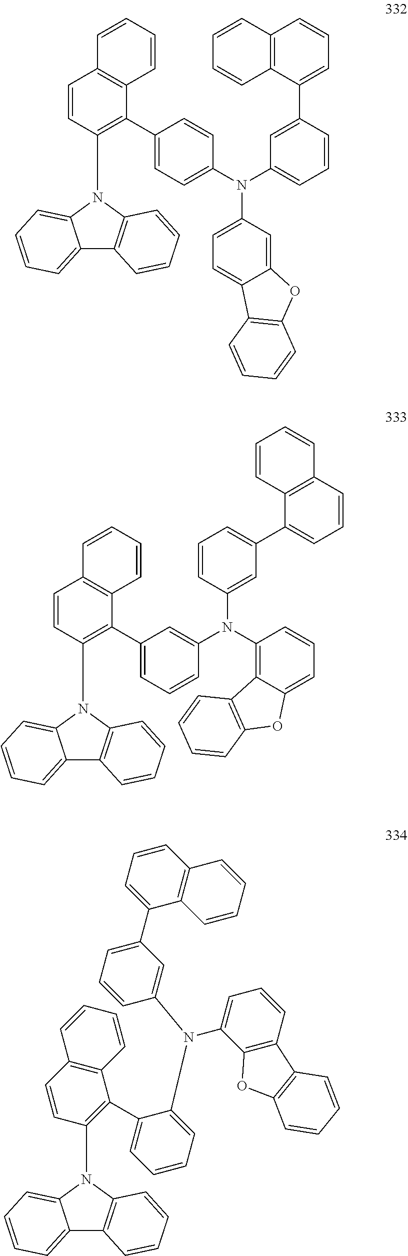 [SMILES: c1cc(-c2cccc3ccccc23)cc(N(c2ccc(-c3c(-n4c5ccccc5c5ccccc54)ccc4ccccc34)cc2)c2ccc3c(c2)oc2ccccc23)c1 ; c1cc(-c2cccc3ccccc23)cc(N(c2cccc(-c3c(-n4c5ccccc5c5ccccc54)ccc4ccccc34)c2)c2cccc3oc4ccccc4c23)c1 ; c1cc(-c2cccc3ccccc23)cc(N(c2ccccc2-c2c(-n3c4ccccc4c4ccccc43)ccc3ccccc23)c2cccc3c2oc2ccccc23)c1]

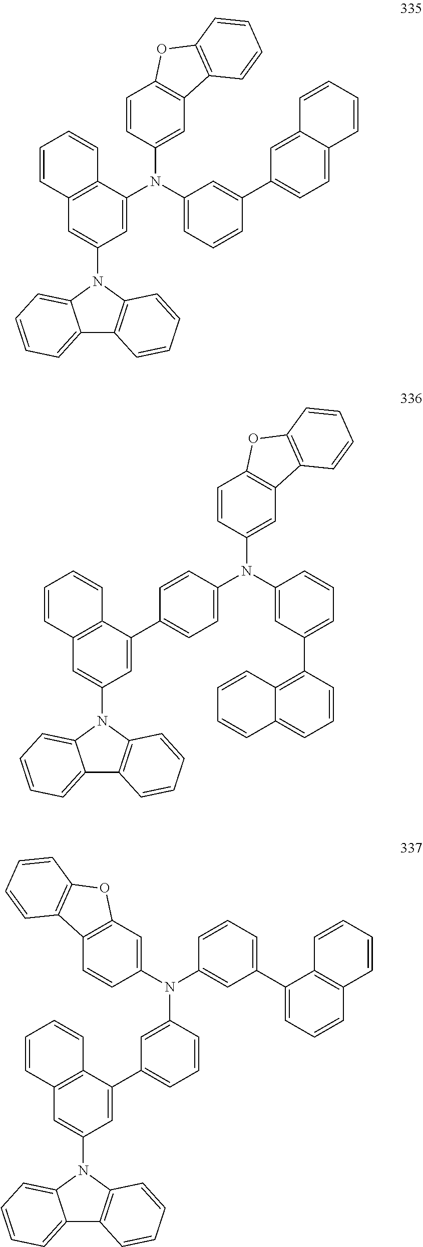 [SMILES: c1cc(-c2ccc3ccccc3c2)cc(N(c2ccc3oc4ccccc4c3c2)c2cc(-n3c4ccccc4c4ccccc43)cc3ccccc23)c1 ; c1cc(-c2cccc3ccccc23)cc(N(c2ccc(-c3cc(-n4c5ccccc5c5ccccc54)cc4ccccc34)cc2)c2ccc3oc4ccccc4c3c2)c1 ; c1cc(-c2cccc3ccccc23)cc(N(c2cccc(-c3cc(-n4c5ccccc5c5ccccc54)cc4ccccc34)c2)c2ccc3c(c2)oc2ccccc23)c1]

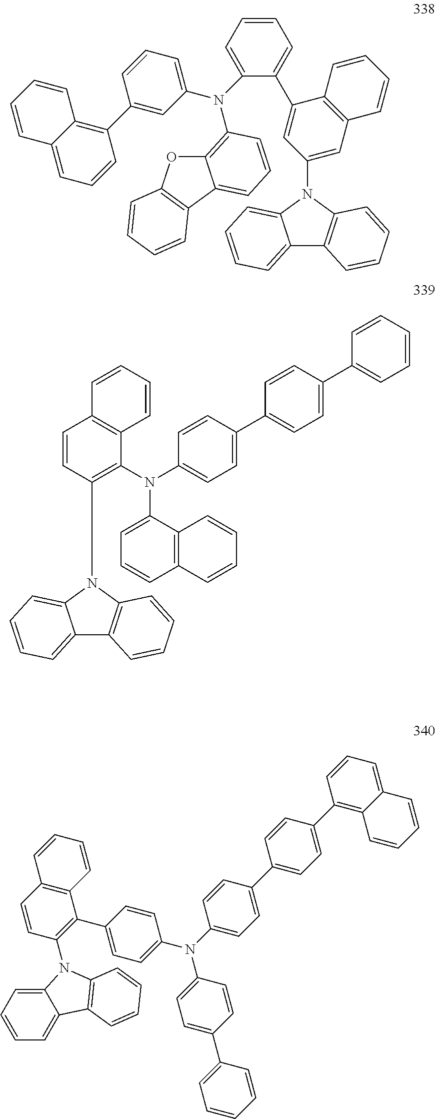 [SMILES: c1cc(-c2cccc3ccccc23)cc(N(c2ccccc2-c2cc(-n3c4ccccc4c4ccccc43)cc3ccccc23)c2cccc3c2oc2ccccc23)c1 ; c1ccc(-c2ccc(-c3ccc(N(c4cccc5ccccc45)c4c(-n5c6ccccc6c6ccccc65)ccc5ccccc45)cc3)cc2)cc1 ; c1ccc(-c2ccc(N(c3ccc(-c4ccc(-c5cccc6ccccc56)cc4)cc3)c3ccc(-c4c(-n5c6ccccc6c6ccccc65)ccc5ccccc45)cc3)cc2)cc1]

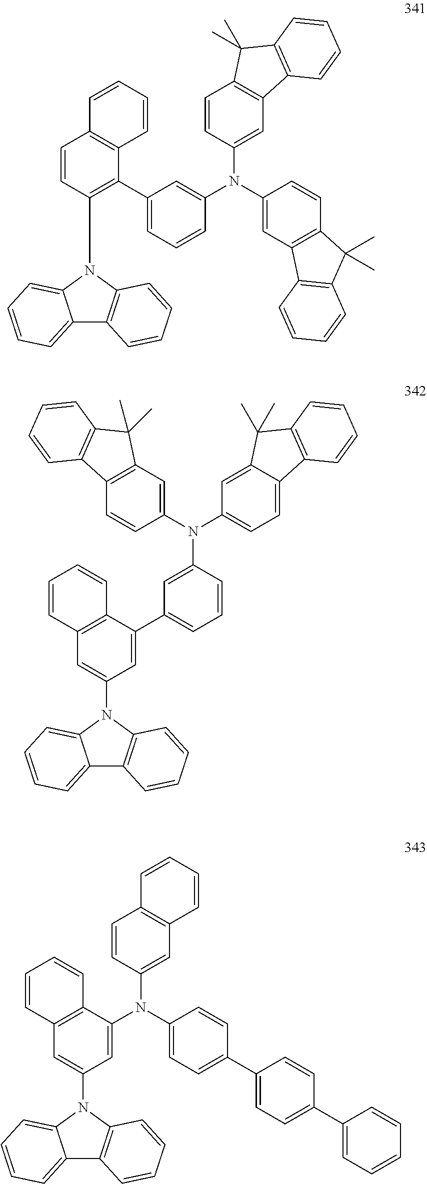 [SMILES: CC1(C)c2ccccc2-c2cc(N(c3cccc(-c4c(-n5c6ccccc6c6ccccc65)ccc5ccccc45)c3)c3ccc4c(c3)-c3ccccc3C4(C)C)ccc21 ; CC1(C)c2ccccc2-c2ccc(N(c3cccc(-c4cc(-n5c6ccccc6c6ccccc65)cc5ccccc45)c3)c3ccc4c(c3)C(C)(C)c3ccccc3-4)cc21 ; c1ccc(-c2ccc(-c3ccc(N(c4ccc5ccccc5c4)c4cc(-n5c6ccccc6c6ccccc65)cc5ccccc45)cc3)cc2)cc1]

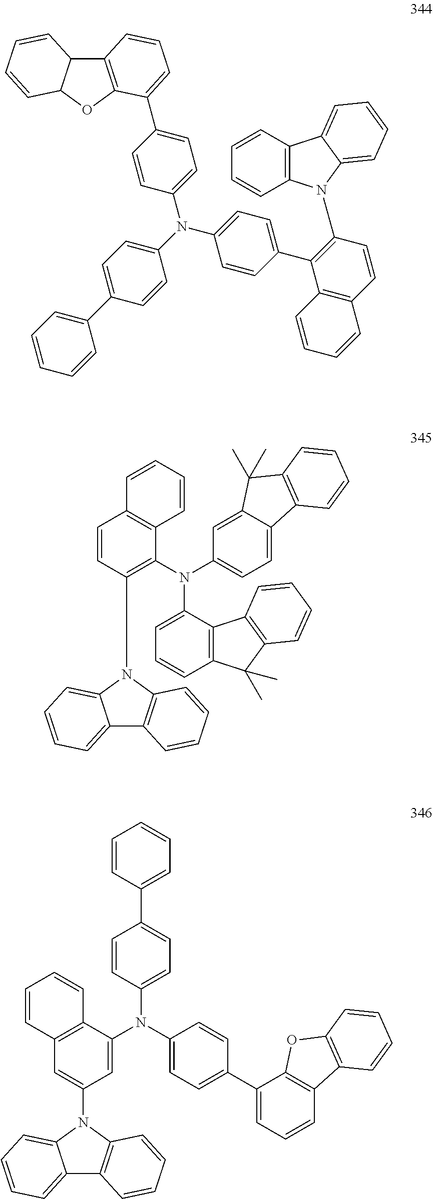 [SMILES: C1=CC2Oc3c(-c4ccc(N(c5ccc(-c6ccccc6)cc5)c5ccc(-c6c(-n7c8ccccc8c8ccccc87)ccc7ccccc67)cc5)cc4)cccc3C2C=C1 ; CC1(C)c2ccccc2-c2ccc(N(c3cccc4c3-c3ccccc3C4(C)C)c3c(-n4c5ccccc5c5ccccc54)ccc4ccccc34)cc21 ; c1ccc(-c2ccc(N(c3ccc(-c4cccc5c4oc4ccccc45)cc3)c3cc(-n4c5ccccc5c5ccccc54)cc4ccccc34)cc2)cc1]

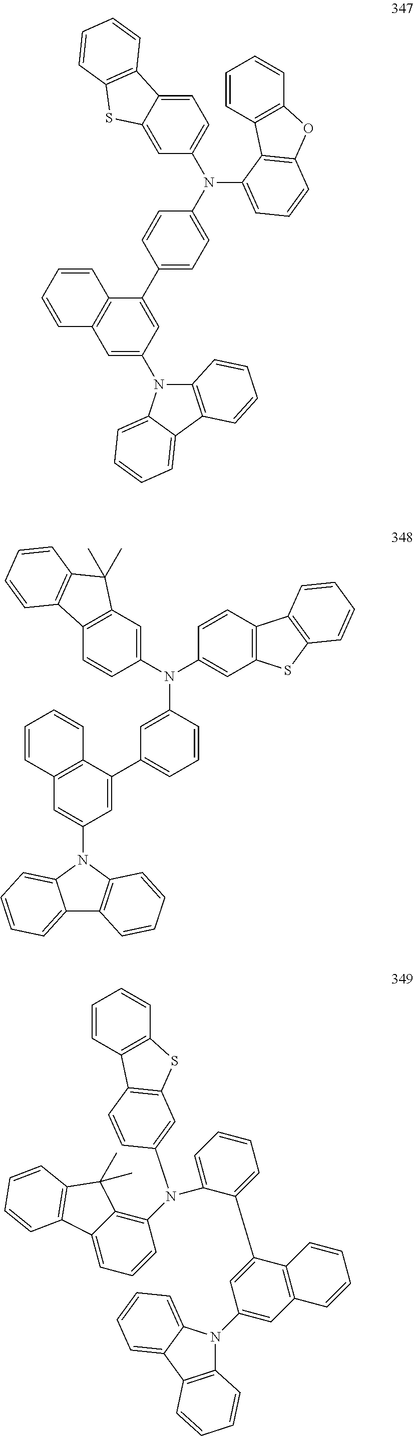 [SMILES: c1ccc2c(-c3ccc(N(c4ccc5c(c4)sc4ccccc45)c4cccc5oc6ccccc6c45)cc3)cc(-n3c4ccccc4c4ccccc43)cc2c1 ; CC1(C)c2ccccc2-c2ccc(N(c3cccc(-c4cc(-n5c6ccccc6c6ccccc65)cc5ccccc45)c3)c3ccc4c(c3)sc3ccccc34)cc21 ; CC1(C)c2ccccc2-c2cccc(N(c3ccc4c(c3)sc3ccccc34)c3ccccc3-c3cc(-n4c5ccccc5c5ccccc54)cc4ccccc34)c21]

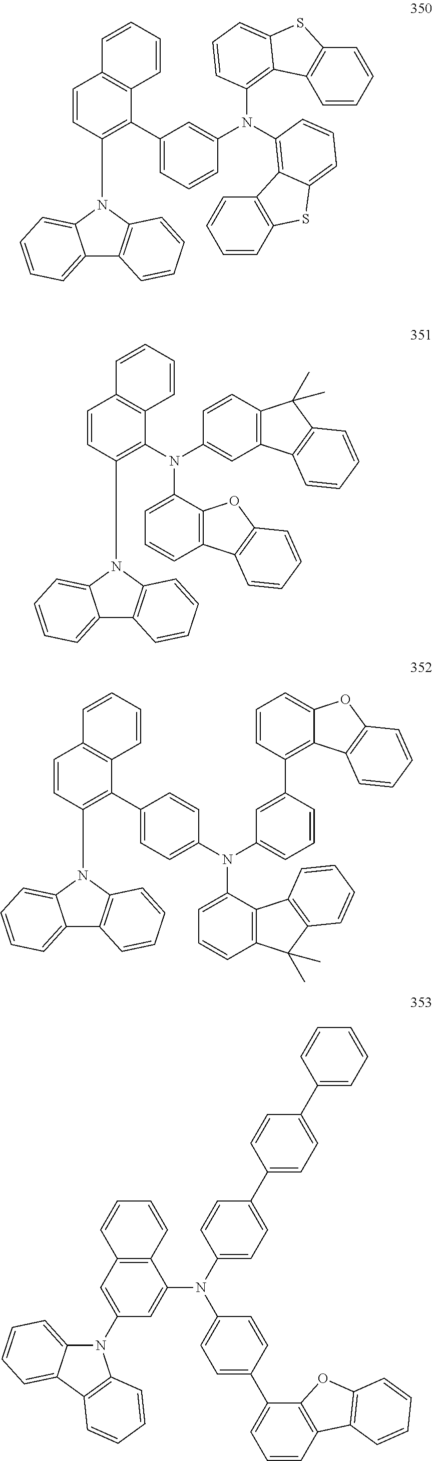 [SMILES: c1cc(-c2c(-n3c4ccccc4c4ccccc43)ccc3ccccc23)cc(N(c2cccc3sc4ccccc4c23)c2cccc3sc4ccccc4c23)c1 ; CC1(C)c2ccccc2-c2cc(N(c3c(-n4c5ccccc5c5ccccc54)ccc4ccccc34)c3cccc4c3oc3ccccc34)ccc21 ; CC1(C)c2ccccc2-c2c(N(c3ccc(-c4c(-n5c6ccccc6c6ccccc65)ccc5ccccc45)cc3)c3cccc(-c4cccc5oc6ccccc6c45)c3)cccc21 ; c1ccc(-c2ccc(-c3ccc(N(c4ccc(-c5cccc6c5oc5ccccc56)cc4)c4cc(-n5c6ccccc6c6ccccc65)cc5ccccc45)cc3)cc2)cc1]

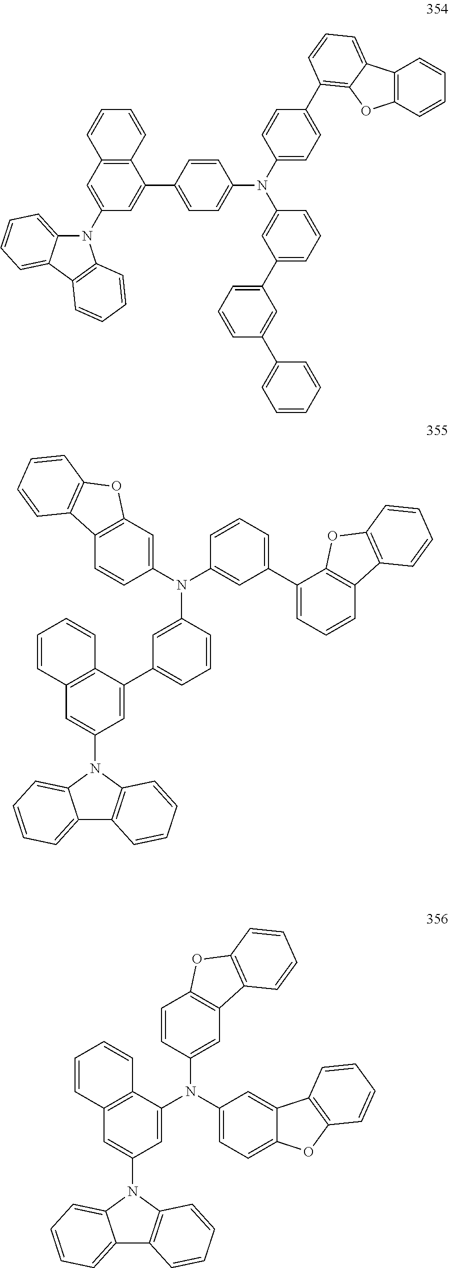 [SMILES: c1ccc(-c2cccc(-c3cccc(N(c4ccc(-c5cc(-n6c7ccccc7c7ccccc76)cc6ccccc56)cc4)c4ccc(-c5cccc6c5oc5ccccc56)cc4)c3)c2)cc1 ; c1cc(-c2cc(-n3c4ccccc4c4ccccc43)cc3ccccc23)cc(N(c2cccc(-c3cccc4c3oc3ccccc34)c2)c2ccc3c(c2)oc2ccccc23)c1 ; c1ccc2c(N(c3ccc4oc5ccccc5c4c3)c3ccc4oc5ccccc5c4c3)cc(-n3c4ccccc4c4ccccc43)cc2c1]

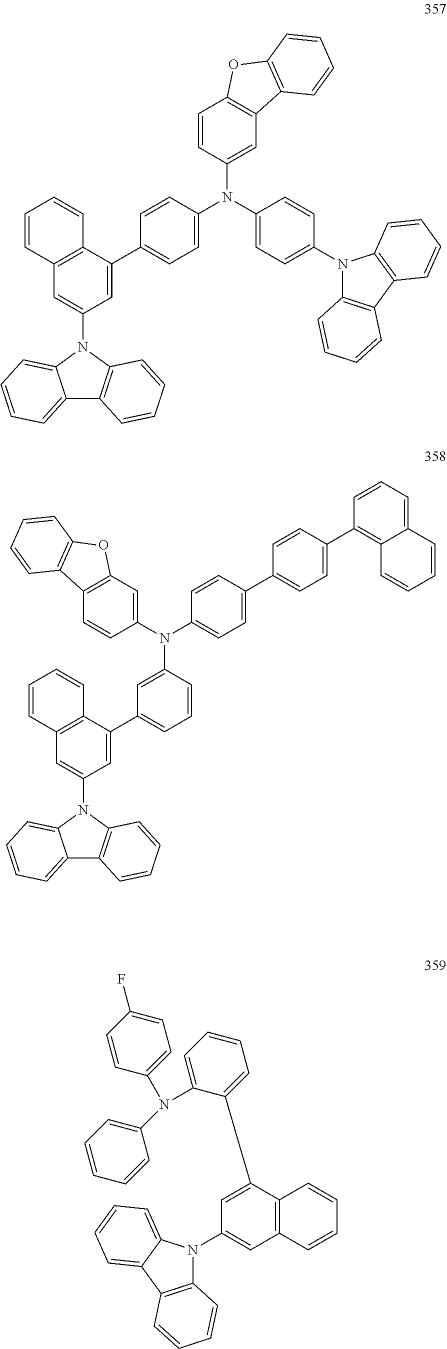 [SMILES: c1ccc2c(-c3ccc(N(c4ccc(-n5c6ccccc6c6ccccc65)cc4)c4ccc5oc6ccccc6c5c4)cc3)cc(-n3c4ccccc4c4ccccc43)cc2c1 ; c1cc(-c2cc(-n3c4ccccc4c4ccccc43)cc3ccccc23)cc(N(c2ccc(-c3ccc(-c4cccc5ccccc45)cc3)cc2)c2ccc3c(c2)oc2ccccc23)c1 ; Fc1ccc(N(c2ccccc2)c2ccccc2-c2cc(-n3c4ccccc4c4ccccc43)cc3ccccc23)cc1]

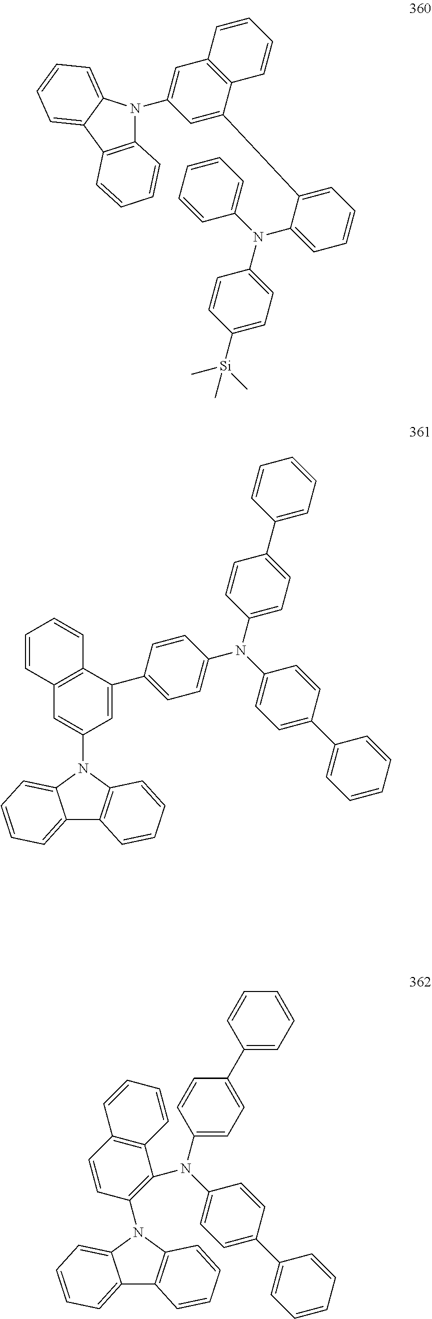 [SMILES: C[Si](C)(C)c1ccc(N(c2ccccc2)c2ccccc2-c2cc(-n3c4ccccc4c4ccccc43)cc3ccccc23)cc1 ; c1ccc(-c2ccc(N(c3ccc(-c4ccccc4)cc3)c3ccc(-c4cc(-n5c6ccccc6c6ccccc65)cc5ccccc45)cc3)cc2)cc1 ; c1ccc(-c2ccc(N(c3ccc(-c4ccccc4)cc3)c3c(-n4c5ccccc5c5ccccc54)ccc4ccccc34)cc2)cc1]

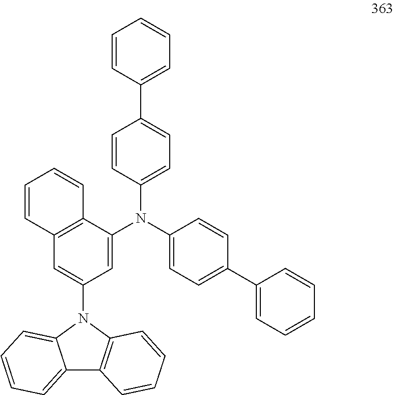 [SMILES: c1ccc(-c2ccc(N(c3ccc(-c4ccccc4)cc3)c3cc(-n4c5ccccc5c5ccccc54)cc4ccccc34)cc2)cc1]

9. An electronic component, comprising an anode and a cathode which is arranged oppositely to the anode, and a functional layer disposed between the anode and the cathode; wherein
the functional layer comprises the nitrogen-containing compound of claim 1.

10. The electronic component of claim 9, wherein the electronic component is an organic electroluminescent device or a photoelectric conversion device.

11. An electronic device, comprising the electronic component of claim 9.

12. The nitrogen-containing compound of claim 2, wherein L is selected from phenylene.

13. The nitrogen-containing compound of claim 3, wherein substituents in the L₁ and L₂ are respectively and independently selected from deuterium, a halogen group, cyano, alkyl with 1 to 5 carbon atoms, and aryl with 6 to 12 carbon atoms.

14. The nitrogen-containing compound of claim 4, wherein substituents in the L₁ and L₂ are respectively and independently selected from deuterium, fluorine, cyano, methyl, ethyl, n-propyl, isopropyl, tert-butyl, phenyl, naphthyl, and biphenyl.

15. The nitrogen-containing compound of claim 6, wherein substituents in the Ar₁ and Ar₂ are respectively and independently selected from deuterium, a halogen group, cyano, alkyl with 1 to 5 carbon atoms, aryl with 6 to 12 carbon atoms, and trialkylsilyl with 3 to 6 carbon atoms.

16. The electronic component of claim 9, wherein the functional layer comprises an electron blocking layer, the electron blocking layer comprises the nitrogen-containing compound.
